(19) United States
(12) Patent Application Publication
(10) Pub. No.: US 2019/0352691 A1
(43) Pub. Date: Nov. 21, 2019

(54) PATHOGEN DETECTION

(71) Applicant: UNIVERSITY OF NOTRE DAME DU LAC, South Bend, IN (US)

(72) Inventors: Marvin J. MILLER, South Bend, IN (US); Cheng JI, Buffalo Grove, IL (US); Paul BOHN, Grainger, IN (US); Sean BRANAGAN, Amsterdam, NY (US); Yang YANG, Devon, PA (US)

(73) Assignee: UNIVERSITY OF NOTRE DAME DU LAC, South Bend, IN (US)

(21) Appl. No.: 16/218,417

(22) Filed: Dec. 12, 2018

Related U.S. Application Data
(63) Continuation of application No. 15/032,276, filed on Apr. 26, 2016, now abandoned.
(60) Provisional application No. 61/899,154, filed on Nov. 1, 2013.

(51) Int. Cl. C12Q 1/04; C12Q 1/14; C12Q 1/06; G01N 33/543; G01N 33/553

(52) U.S. Cl. CPC C12Q 1/04; C12Q 1/14; G01N 33/553; G01N 33/54366; G01N 33/54386; C12Q 1/06

(57) ABSTRACT

A device for detecting bacteria in a sample, comprising:

a substrate having a surface; and

a plurality of Fe(III)-bound or Fe(III)-binding siderophores specific to the bacteria and covalently attached to the surface;

wherein the siderophores are selected from the group consisting of one or more natural siderophores, siderophores having one or more of the formulas described herein, or combination thereof. Methods of detection are also provided.

### CROSS-REFERENCE TO RELATED APPLICATIONS

This application claims priority to U.S. Provisional Application No. 61/899,154, filed November 1, 2013, the entire contents of which are hereby incorporated by reference.

### FIELD OF THE INVENTION

The present application relates to devices and methods for pathogen detection.

### BACKGROUND

Iron is essential for the growth of virtually all forms of life including Mycobacterium tuberculosis (Mtb), Acinetobacter baumannii, Pseudomonas aeruginosa and methicillin-resistant Staphylococcus aureus (MRSA). Since Fe(III) is very insoluble at physiological pH, microbes have evolved exquisitely specific processes for iron sequestration that often involve active transport through an otherwise impermeable outer membrane. Bacterial iron acquisition is essential for pathogenicity, and provides an attractive and heretofore little-used target for the development of microbe-selective biomarkers for selective detection. Acquisition of iron by bacteria at the infection site depends on the presence of soluble Fe(III) complexes generated from iron sources. These solubilized Fe(III)-complexes (generically called siderophores) must then be sequestered by the bacteria to initiate iron transport across the cell envelope. Siderophores have been used in a number of applications requiring bacterial recognition due to their inherent specificity.

Physicians are in need of an improved method for identifying pathogenic bacteria, especially those drug-resistant strains which currently cause the majority of deaths within health care facilities. Examples include methicillin-resistant Staphylococcus aureus (MRSA), multidrug resistant Myobacterium tuberculosis (Mtb), Pseudomonas aeruginosa, and multidrug-resistant Acinetohacter baumannii (MDRAB). Since any delay in treatment of an infection increases the likelihood of a fatality, physicians frequently begin treatment before the exact strain is identified. This leads to sub-optimal care, where for example a broad spectrum antibiotic is prescribed where a tailored drug is necessary, or an insufficient dose is prescribed, both of which contribute to further drug resistance by the pathogen.

Current diagnostic methods generally fall into one of four categories: (1) specific culturing of the organism followed by visual inspection for identifying phenotypic characteristics, (2) detection of pathogen-specific antibodies produced by the patient, (3) immunological-based detection of specific pathogen products, e.g., exotoxins, and (4) genetic sequencing. All of these methods require a lot of time, up to several days, in order to reach an accurate diagnosis. For example, the fastest rapid-screening technique for MRSA currently available, quick multiplex immunocapture-coupled PCR (qMRSA), produces a diagnosis using as few as 5 genome copies in approximately 22 hours, versus up to 4 days using conventional culture. Even this four-fold improvement is insufficient to allow point-of-care diagnostics, which is ideal for patient care. Similarly, two of the above methods, antibody detection and immunological-based detection of infection byproducts, require an immune response by the patient after infection; patients with compromised immune systems are among those most at risk of death from MRSA and tuberculosis. Although some rapid diagnostic tests have been developed in recent years, accurate clinical diagnosis (identification & characterization) still requires confirmation by another (slow) technique. Therefore, initial treatment of a bacterial infection is typically begun without confirmation of the specific infection type, since any delay in treatment could result in a fatal infection. Some initial work on siderophore-based detection has been done, but these techniques require microscopic imaging and/or significant post-processing in order to detect a bacterial strain. Therefore, a significant need exists for an improved detection technique, based on microbial affinity, which is fast, selective, and analytically efficient.

Correct initial treatment (which requires a fast and accurate initial diagnosis) has been found to significantly improve patient outcomes, especially among drug-resistant infections acquired in hospitals. In contrast, failure to quickly recognize and treat patients with MTB leads to increased mortality, nosocomial infections, and further resistance to antimicrobial drugs. Patients with traumatic injuries are especially prone to wound colonization and infection with strains of both Gram positive and Gram negative forms of bacteria. Proper treatment requires rapid and accurate diagnosis of the infectious organism, preferably in the field with minimal delay. The diagnostic method disclosed here, in its most portable practice, is intended to have an immediate and positive impact on survival of such patients. The need to reduce the evolutionary forces driving antibiotic resistance is another utility for fast and accurate bacterial diagnosis. Mistaken prescriptions of antibiotics to treat viral infections, for example, could be reduced by the availability of a cheap and user-friendly bacterial diagnostic test. Significant economic growth within point-of-care diagnostics has already been realized, and the market is projected to approximately double within the next decade.

### BRIEF DESCRIPTION OF THE FIGURES

FIG. 1 shows an example of a general schematic of the Pseudomonas “pull down,” Pseudomonas binding detection and signal amplification.

FIG. 2 shows an example of a tripodal catecholate siderophore coupled to an aminopenicillin, has outstanding antimicrobial activity against several P. aeruginosa strains even when the base penicillins were inactive.

FIG. 3 shows an example in which the surface bound siderophore is able to specifically immobilize the target Pseudomonas strain (PA01), while resisting both non-specific adsorption (PEG only) and capture of the non-target strain (PA06) on solid scaffolds

FIG. 4 shows an example of a surface chemistry/enhancement scheme along with images of the substrate before (3) and after (4) Au nanoparticle decoration, and after enhancement by Ag(I) reduction (5).

FIGS. 5A and 5B show an example of a variations of a sandwich technique and preliminary results.

FIGS. 6A, 6B and 6C show examples of variations of a sandwich technique, including FIG. 6A: AuNP-Ag(1) aggregation technique; FIG. 6B: Avidin-Biotin-Enzyme (Peroxidase) reporter; and FIG. 6C: Siderophore Sandwich with AuNP and Ag Crystal Reporter.

FIG. 7a shows a general schematic overview of a prototype polymethymethacrylate solid scaffold. Representative diagnostic siderophores will be immobilized on the surface of the PMMA in the outside lanes (below). FIG. 7b shows a depiction of a postive response.

FIG. 8 shows an example of synthesis of tri-catechol HD-01.

FIG. 9 shows an example of synthesis of tri-catechol HD-01 precursors.

FIG. 10a shows an example of a standard approach to construct the siderophore-biotin conjugagtes. FIG. 10b shows an example of a depiction of the biotin attached to the sandwiched siderophore.

FIG. 11 shows examples of synthetic siderophore-antibiotic conjugate (III), fimsbactin A(II), HD-02A and HD-02.

FIG. 12 shows an example of a synthesis of fimsbacins A and B.

FIG. 13 shows an example of a synthesis using L-serine as the starting material.

FIG. 14 shows one example of a detection platform and a schematic diagram of a simple four-lane surface plasmon reader construct with the Au NPs fabricated into the PMMA scaffold.

FIG. 15 shows examples of mycobactin T analogs for immobilization on PMMA/

FIG. 16 shows an example of a) chemically modify yersinabactin to bind to scaffold, b) bind functionalized yersinabactin to scaffold, c) apply bacterial sample to scaffold, d) sandwich trapped yersinabactin with Au nanoparticles coated with functionalized yersiniabactin, e) develop visual signal with Ag nanocrystals.

FIG. 17 shows an example of a synthetic sequence for the synthesis of yersiniabactin.

FIG. 18 shows an example in schematic form of a setup for SPR-PI quantification and transduction.

FIG. 19 shows an example of an assembly of the layers into a multilayer PMMA-polycarbonate multilayer microfluidic device.

FIG. 20 shows various examples of semi-synthetic or synthetic siderophores.

### DETAILED DESCRIPTION OF THE SEVERAL EMBODIMENTS

An improved method of detecting pathogenic bacteria based on microbial iron chelators is disclosed. The technology uses selective recognition of siderophores to identify and characterize different types of bacteria. Combining these tasks enables the development of a rapid diagnostic test for use in health care laboratories or at the point-of-care. The technology can be adapted for single strains of bacteria or multiple bacterial analyses from the same microfluid sample. In practice, the device is realized in one of two formats: (1) a microfluidic multichannel affinity chromatography and detection system based on covalent attachment of bacteria to siderophores and analogs to the surface of separate channels in the microfluidic device; and (2) affinity-based pulldown onto a solid substrate followed by complementary recognition by gold nanoparticles and subsequent amplification by Ag particle nucleation. In format (1) passage of sub-microliter volumes of sample through the device will allow exposure to the adsorbed siderophores that specifically recognize and tightly bind the respective bacteria. The bacteria thus pulled down will be detected using one of various sensing techniques. In a primary development of the invention, label-free surface-plasmon (SPR) detection with an external reader is used. In format (2) the primary recognition event, which results in a surface bound bacterium, is followed by a second affinity recognition event using Au nanoparticles tagged with the same siderophore. Subsequently, these nanoparticles are used as nucleation sites for the growth of high optical density Ag particles by reduction of solution-phase Ag(I) via electroless deposition. Format (1) is envisioned to target hospital or public health applications, whereas format (2) is aimed at resource-limited settings, such as found in the developing world. The optimal device will be low cost, easy to use and extraordinarily sensitive. The following describes a representative application focusing on rapid diagnosis of tuberculosis to demonstrate the potential of the plan and then illustrates planned applications to detect multidrug-resistant organisms (MDROs) and/or nosocomial pathogens, particularly Acinetobacter baumannii, Pseudomonas aeruginosa and methicillin-resistant Staphylococcus aureus (MRSA).

In one embodiment, a device is provided for detecting bacteria in a sample, comprising:

a substrate having a surface comprising an interdigitated Au electrode array; and

a plurality of Fe(III)-bound or Fe(III)-binding siderophores specific to the bacteria and covalently attached to the surface;

wherein the siderophores are selected from the group consisting of one or more natural siderophores, siderophores having one or more of the following formulas, or combination thereof:

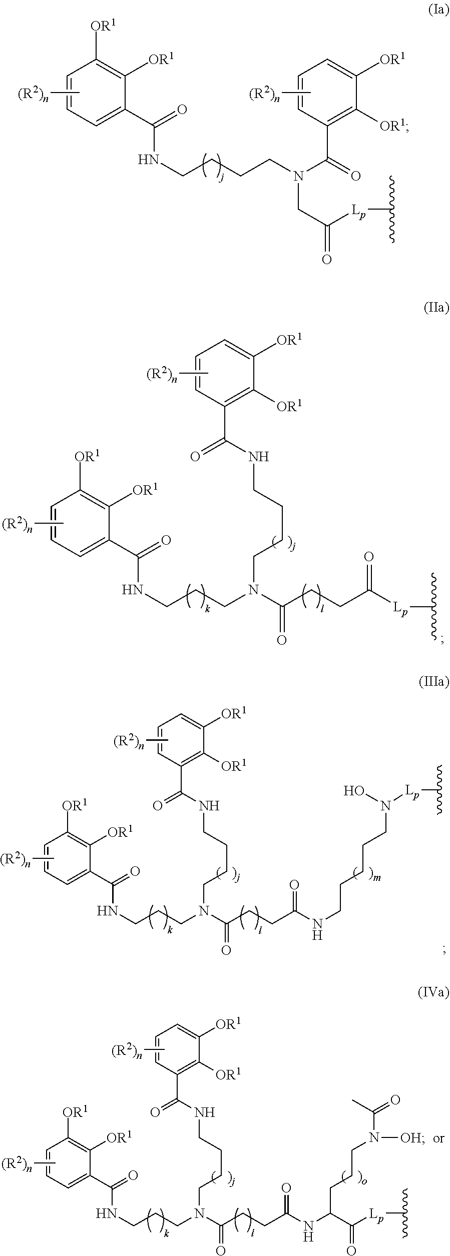

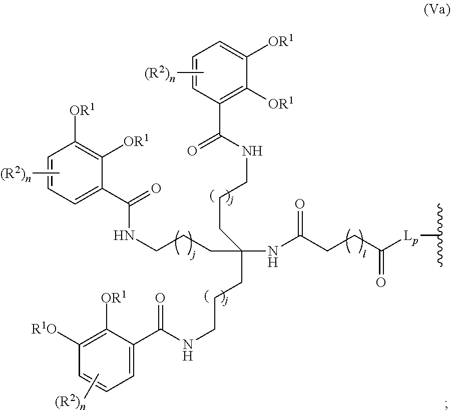

wherein

each L is independently a linker;

each R¹ is independently H, —C(═O)alkyl, —C(═O)aryl, or —C(═O)O-alkyl;

each R² is independently H, alkyl, alkoxy, hydroxy, carboxy, halo, nitro, amino, or cyano;

each n is independently 1, 2, or 3;

each p is independently 0-11;

each j is independently 0-11;

each k is independently 1-11;

each l is independently 1-11;

each o is independently 0-11; and

each m is independently 0-11;

pharmaceutically acceptable salt thereof, or combination thereof.

In one embodiment, the surface further comprises paper, polymer, silica, quartz, glass, or a combination thereof.

In one embodiment, the siderophores are attached directly or indirectly through a linking group.

In one embodiment, the siderophore is a naturally occurring or synthetic siderophore.

In one embodiment, a diagnostic test strip is provided for detecting bacteria in a sample, comprising:

a substrate having a surface other than gold or glass; and

a plurality of Fe(III)-bound or Fe(III)-binding siderophores specific to the bacteria and covalently attached to the surface;

wherein the siderophores are selected from the group consisting of one or more natural siderophores, siderophores having one or more of the following formulas, or combination thereof:

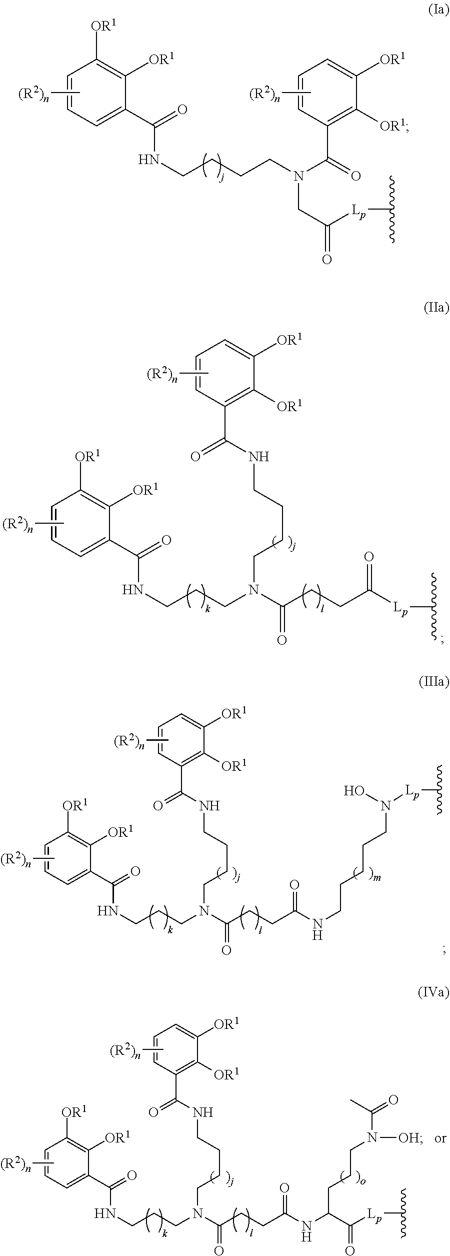

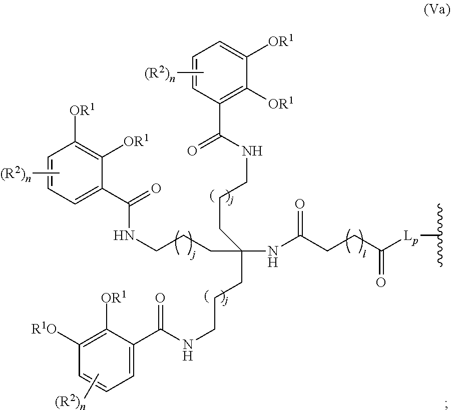

wherein

each L is independently a linker;

each R¹ is independently H, —C(═O)alkyl, —C(═O)aryl, or —C(═O)O-alkyl;

each R² is independently H, alkyl, alkoxy, hydroxy, carboxy, halo, nitro, amino, or cyano;

each n is independently 1, 2, or 3;

each p is independently 0-11;

each j is independently 0-11;

each k is independently 1-11;

each l is independently 1-11;

each o is independently 0-11; and

each m is independently 0-11;

pharmaceutically acceptable salt thereof, or combination thereof.

In one embodiment, the substrate surface is paper, polymer, silica, quartz, or combination thereof.

In one embodiment, a method is provided for detecting bacteria in a sample, comprising:

contacting the sample with a substrate having a surface comprising an interdigitated Au electrode array (IDE) and a plurality of Fe(III)-bound or Fe(III)-binding siderophores specific to the bacteria and covalently attached to the surface;

dielectrophoresing the sample over the IDE, to effect a binding of the bacteria, if present in the sample, to one or more of the siderophores;

detecting the presence or absence of the bacteria so bound using Surface Plasmon Resonance;

wherein the siderophores are selected from the group consisting of one or more natural siderophores, siderophores having one or more of the following formulas, or combination thereof:

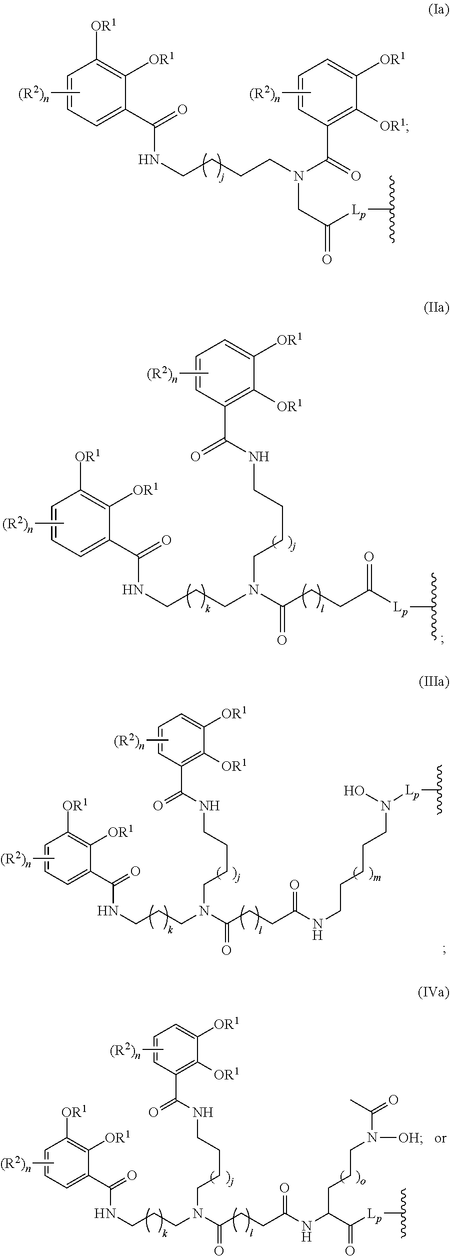

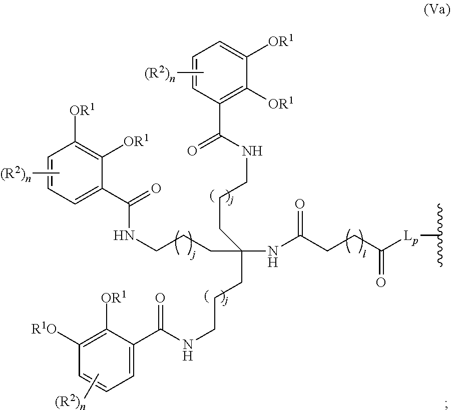

wherein

each L is independently a linker;

each R¹ is independently H, —C(═O)alkyl, —C(═O)aryl, or —C(═O)O-alkyl;

each R² is independently H, alkyl, alkoxy, hydroxy, carboxy, halo, nitro, amino, or cyano;

each n is independently 1, 2, or 3;

each p is independently 0-11;

each j is independently 0-11;

each k is independently 1-11;

each l is independently 1-11;

each o is independently 0-11; and

each m is independently 0-11;

pharmaceutically acceptable salt thereof, or combination thereof.

In one embodiment, the surface further comprises paper, polymer, silica, quartz, glass, or a combination thereof.

In one embodiment, the bacteria is present in the sample and is detected.

In one embodiment, the bacteria is not present in the sample and is not detected.

In one embodiment, the sample comprises a mixture of bacteria for which the siderophore is specific and bacteria for which the siderophore is not specific, and wherein the bacteria for which the siderophore is specific is detected and bacteria for which the siderophore is not specific is not detected.

In one embodiment, the detected bacteria is quantified.

In one embodiment, one or more washing steps are carried out between one or more of the contacting, dielectrophoresing, and detecting.

In one embodiment, a method is provided for detecting bacteria in a sample, comprising:

contacting the sample with a substrate having a surface comprising a plurality of covalently attached Fe(III)-bound or Fe(III)-binding siderophores specific to the bacteria, to effect a binding of one or more of the bacteria, if present in the sample, to one or more of the siderophores;

detecting the presence or absence of the bacteria so bound using Surface Plasmon Resonance;

wherein the siderophores are selected from the group consisting of one or more natural siderophores, siderophores having one or more of the following formulas, or combination thereof:

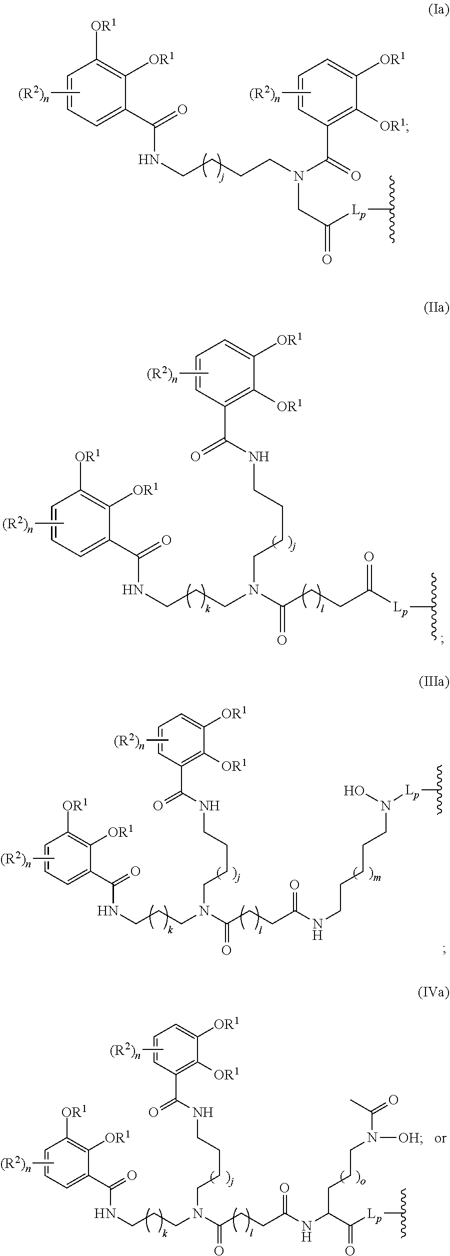

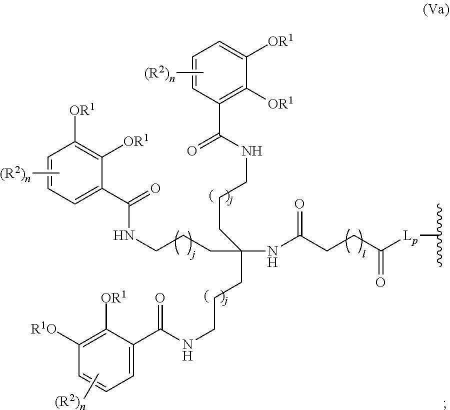

wherein

each L is independently a linker;

each R¹ is independently H, —C(═O)alkyl, —C(═O)aryl, or —C(═O)O-alkyl;

each R² is independently H, alkyl, alkoxy, hydroxy, carboxy, halo, nitro, amino, or cyano;

each n is independently 1, 2, or 3;

each p is independently 0-11;

each j is independently 0-11;

each k is independently 1-11;

each l is independently 1-11;

each o is independently 0-11; and

each m is independently 0-11;

pharmaceutically acceptable salt thereof, or combination thereof.

In one embodiment, a method is provided for detecting bacteria in a sample, comprising:

contacting the sample with a substrate surface comprising a plurality of covalently-attached first Fe(III)-bound or Fe(III)-binding siderophores specific to the bacteria, to effect a first binding of one or more of the bacteria, if present in the sample, to one or more of the first siderophores;

introducing a detection fluid comprising a plurality of gold nanoparticles, the nanoparticles comprising one or more covalently-attached second Fe(III)-bound or Fe(III)-binding siderophores specific to the bacteria, to effect a second binding of one or more of the bacteria, if bound to the first siderophores, to one or more of the second siderophores;

detecting the presence or absence of the nanoparticles so bound, to thereby detect the present or absence of the bacteria;

wherein the siderophores are selected from the group consisting of one or more natural siderophores, siderophores having one or more of the following formulas, or combination thereof:

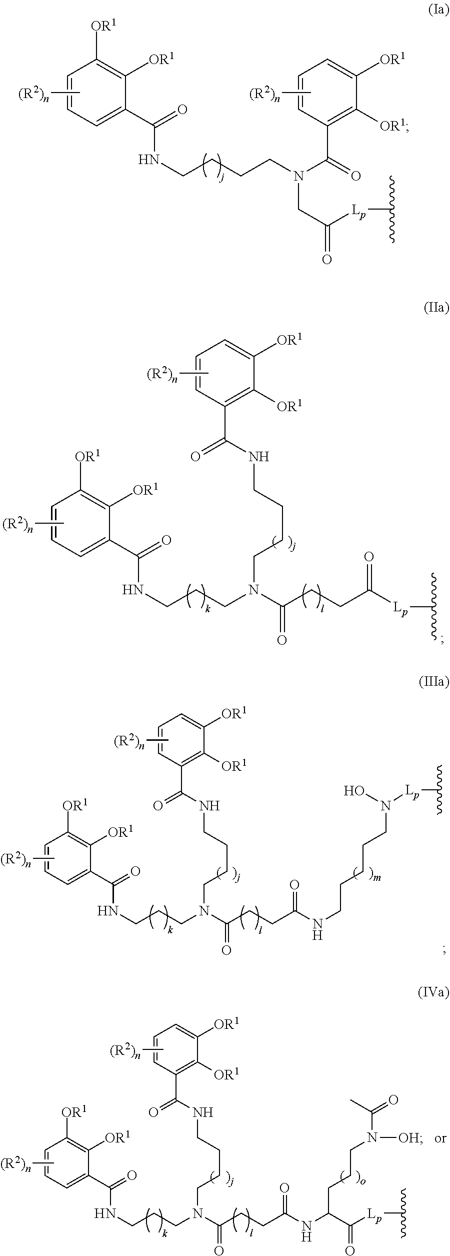

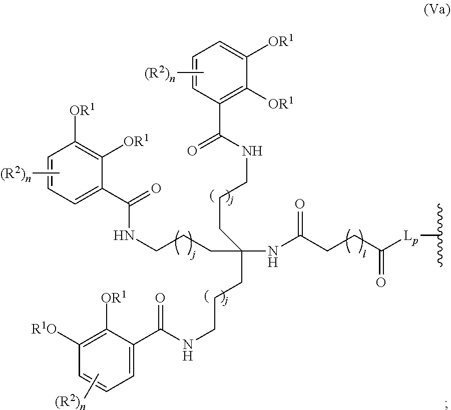

wherein

each L is independently a linker;

each R¹ is independently H, —C(═O)alkyl, —C(═O)aryl, or —C(═O)O-alkyl;

each R² is independently H, alkyl, alkoxy, hydroxy, carboxy, halo, nitro, amino, or cyano;

each n is independently 1, 2, or 3;

each p is independently 0-11;

each j is independently 0-11;

each k is independently 1-11;

each l is independently 1-11;

each o is independently 0-11; and

each m is independently 0-11;

pharmaceutically acceptable salt thereof, or combination thereof.

In one embodiment, the gold nanoparticles have a size ranging from 1 nm to 2 microns. This range includes all values and subranges therebetween, including 1, 2, 3, 4, 5, 6, 7, 8, 9, 10, 20, 30, 40, 50, 60, 100, 200, 300, 400, 500, 600, 700, 800, 900, 1000 nm, 1.2, 1.3, 1.4, 1.5, 1.6, 1.7, 1.8, 1.9, and 2 microns, or any combination thereof.

In one embodiment, the gold nanoparticles further comprise a label for detection, for example

a radiolabel, a fluorescent label, a colorimetric label, a UV-Vis label, or combination thereof.

In one embodiment, the detection comprises radiodetection, fluorescent detection, colorimetric analysis, UV-Vis analysis, or combination thereof.

In one embodiment, a method is provided for detecting bacteria in a sample, comprising:

contacting the sample with a substrate surface comprising a plurality of covalently-attached first Fe(III)-bound or Fe(III)-binding siderophores specific to the bacteria, to effect a first binding of one or more of the bacteria, if present in the sample, to one or more of the first siderophores;

introducing a detection fluid comprising a plurality of gold nanoparticles, the nanoparticles comprising one or more covalently-attached second Fe(III)-bound or Fe(III)-binding siderophores specific to the bacteria, to effect a second binding of one or more of the bacteria, if bound to the first siderophores, to one or more of the second siderophores;

introducing an amplification fluid comprising a reductant and soluble Ag(I), to effect an electroless deposition of Ag metal onto one or more of the nanoparticles so bound;

detecting the presence or absence of Ag metal so deposited, to thereby detect the presence or absence of the bacteria;

wherein the siderophores are selected from the group consisting of one or more natural siderophores, siderophores having one or more of the following formulas, or combination thereof:

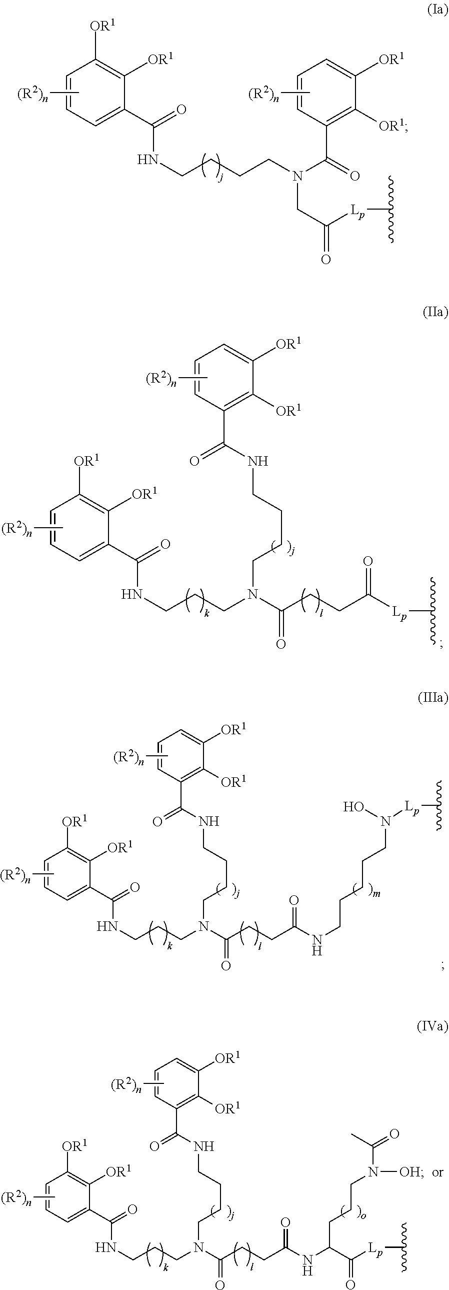

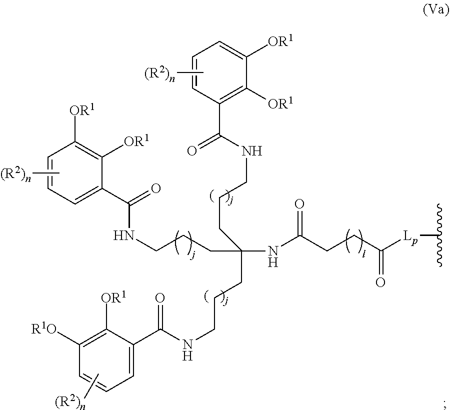

wherein

each L is independently a linker;

each R¹ is independently H, —C(═O)alkyl, —C(═O)aryl, or —C(═O)O-alkyl;

each R² is independently H, alkyl, alkoxy, hydroxy, carboxy, halo, nitro, amino, or cyano;

each n is independently 1, 2, or 3;

each p is independently 0-11;

each j is independently 0-11;

each k is independently 1-11;

each l is independently 1-11;

each o is independently 0-11; and

each m is independently 0-11;

pharmaceutically acceptable salt thereof, or combination thereof.

In one embodiment, the reductant comprises an aldehyde, glucose/dextrose, tartaric acid, formaldehyde, hydroquinone, or combination thereof.

In one embodiment, the detection comprises optical detection, optical transmission, optical reflectance, or combination thereof.

In one embodiment, one or more microfluidic channels may be disposed over the surface to direct a flow of the sample over the surface.

In one embodiment, the device also includes a power source and control for the IDE.

In one embodiment, the sample is liquid.

In one embodiment, the sample originates from an environment, a mammal, a culture, or combination thereof.

In one embodiment, the siderophore has one or more of the following formulas:

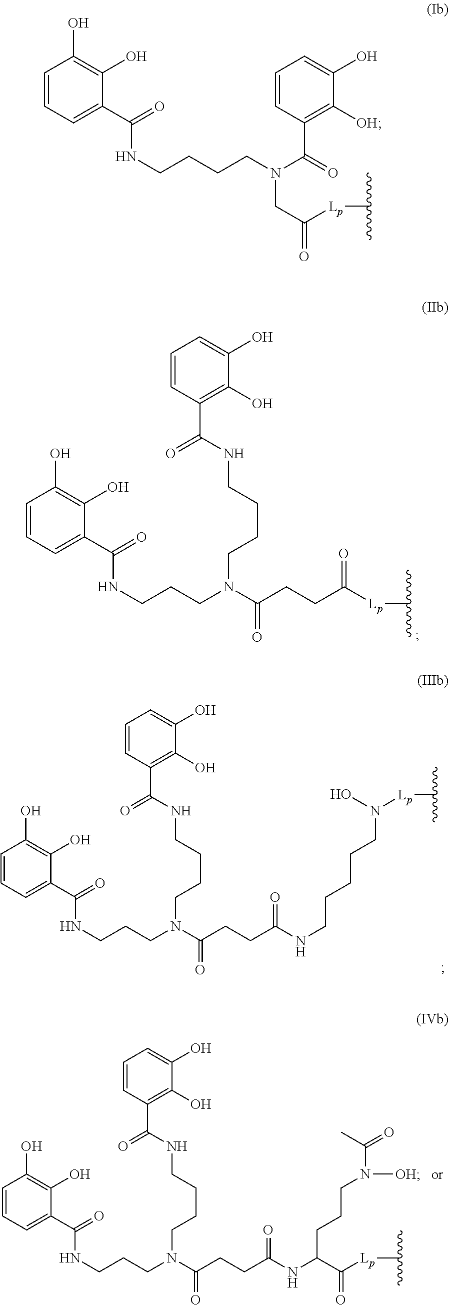

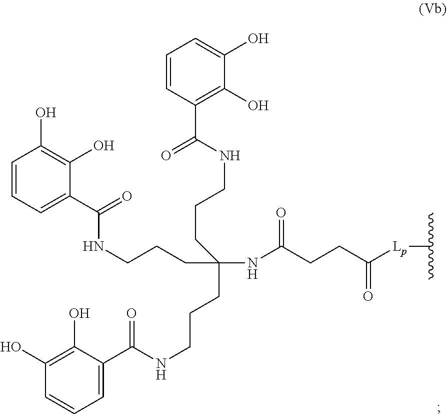

wherein

each L is independently a linker; and

each p is independently 0-11;

Fe(III)-binding form thereof, Fe(III)-bound form thereof, pharmaceutically acceptable salt thereof, or combination thereof.

Surface plasmon resonance (SPR), and especially second generation SPR techniques amenable to miniaturization, are expected to play a central role in chemical analysis of the future. Techniques which do not require microscopic imaging, such as phase-shift SPR, wavevector-resolved SPR, and others are the preferred technique for adapting to siderophore-mediated bacterial sensing. Finally, the technique of electroless deposition is anticipated to form the basis of a label-free test strip kit, which would not require a reader of any kind, and is thus deployable in resource-poor environments

In one embodiment, the selective recognition of siderophores (microbial iron chelators) by different types of bacteria and will be able to differentiate bacteria and allow for rapid diagnostics. The technology can be adapted for single strains of bacteria or multiple bacterial analyses from the same microfluid sample. In brief, the device will be a microfluidic multichannel affinity chromatography and detection system based on covalent attachment of bacteria specific siderophores and analogs to the surface of separate channels in the microfluidic device. Passage of microliter volumes of sample through the device will allow exposure to the adsorbed siderophores that specifically recognize and tightly bind the respective bacteria. The bacteria thus pulled down will be detected using one of various sensing techniques. In a primary development of the invention, label-free surface-plasmon (SPR) detection using an external reader will be developed (format 1). Alternatively, no reader will be required where the sensor is adapted to use electroless deposition of a metal onto a label-free test strip (format 2). The optimal device will be low-cost, easy to use and extraordinarily sensitive—down to the selective detection of a single bacteria cell. The following describes a representative application focusing on rapid diagnosis of tuberculosis to demonstrate the potential of the plan and then illustrates planned applications to detect multidrug-resistant organisms (MDROs) and/or nosocomial pathogens, particularly Acinetobacter baumannii, Pseudomonas aeruginosa and methicillin-resistant Staphylococcus aureus (MRSA).

Iron is essential for the growth of virtually all forms of life including Mtb, Acinetobacter baumannii, Pseudomonas aeruginosa and methicilin-resistant Staphylococcus aureus (MRSA). Since Fe(III) is very insoluble at physiological pH, microbes have evolved very specific processes for iron sequestration that often involve active transport through an otherwise impermeable outer membrane. Bacterial iron acquisition is essential for pathogenicity, and provides an attractive and heretofore little-used target for the development of microbe-selective antibiotics and biomarkers for selective detection. Acquisition of iron by bacteria at the infection site depends on the presence of soluble Fe(III) complexes generated from iron sources. These solubilized Fe(III)-complexes (generically called siderophores) must then be sequestered by the bacteria to initiate iron transport across the cell envelope. For example, the unequivocal importance of the specific siderophore mycobactin T to the growth of Mtb has been established by showing that a mutant of Mtb lacking a gene from mycobactin biosynthesis had a considerably decreased ability to grow in human macrophages. The Miller group has synthesized mycobactin T (1), the Mtb specific siderophore, analogs and, most recently, a conjugate (3) with artemisinin. Although the antimalarial agent, artemisinin (2) itself is not active against tuberculosis, conjugation to a Mtb specific siderophore (microbial iron chelator) analog induces significant and selective anti-tuberculosis activity, including activity against MDR and XDR strains of Mtb. Physicochemical and whole cell studies indicate that ferric to ferrous reduction of the iron complex of the conjugate initiates the expected bactericidal Fenton-type radical chemistry on the artemisinin component. Thus, this “Trojan Horse” approach demonstrates that new pathogen selective therapeutic agents can be generated in which the iron component of the delivery vehicle also participates in triggering the antibiotic activity. The result is that the critical iron uptake machinery of Mtb is demonstrably selective and thus is uniquely suited for design of a sensitive, selective and non-invasive diagnostic tool. As a further indication of its microbe selectivity, we found that conjugate 3 was not active against a broad set of Gram-positive and Gram-negative bacteria at the highest levels tested (2 mM). As still another indication of the unique anti-Mtb selectivity of 3, it was tested and found to have negligible activity (>100 fold less) against a number of fast growing strains of mycobacteria (M. vaccae, M smegmatis, M. aurum and M. fortuitum). Thus, the antibiotic activity of conjugate 3 is microbe-selective, because of exploitation of the unique and essential iron assimilation process, as anticipated. The Miller group also previously reported the design, syntheses and antimicrobial activity of unnatural carbacephalosporin siderophore conjugates 4-5 with separate hydroxamic acid-based and catechol-based siderophore components. As expected, detailed biological assays revealed that the hydroxamate- and catechol-containing conjugates utilized different outer membrane receptor proteins to initiate cellular entry (Fhu and cir, respectively) and exquisite bacteria selectivity, including remarkable activity against pathogens that cause serious health risks to military personnel.

Pseudomonas aeruginosa produces very specific siderophores, including pyoverdine (6, R═OH) and related studies indicate the potential for use as pseudomonally selective affinity agents. We have optimized fermentation processes to obtain natural pyoverdine free acid (R═OH) that is directly suitable for coupling to pegylated thiols needed for the selective detection methodology described below.

As described above, the diagnostic method of the invention targets a fundamental metabolic activity of specific bacteria, the siderophore-mediated metabolic uptake of iron, to mediate the capture and confinement of targeted pathogens. In both formats (1) and (2) the bacteria-specific siderophore (e.g., the siderophore component of 3-6) is anchored to a surface (gold or polymer) in such a way that the targeted bacteria, while attempting to ingest the siderophore, also become anchored to the surface—a process that will be sensitively detected using label-free SPR detection. For example, FIG. 3 illustrates particular realization of format (1) in which SPR imaging is used to distinguish between microfluidic channels that contain only the capture agent and those in which an analyte has been captured (sample). The siderophore-bioconjugates are functionalized to the capture surface (pegylated Au, chosen for resistance to non-specific adsorption) via a heterobifunctional linker, allowing us to simultaneously mitigate against non-specific adsorption, present competent capture motifs well-separated from the underlying protective layer and capture bacteria with both exquisite sensitivity and selectivity.

Furthermore, the potential high-cost driver derived from the use of Au can be circumvented either by constructing a demountable SPR platform in which the sampling is implemented with a “throw-away” plastic element that has the microfluidic channels embossed into it or by exploiting the localized surface plasmon effect with inexpensive Au colloid active layers. After collecting the sample directly on the disposable element, it is mated directly onto the field-deployable reader. The reader—essentially a miniaturized cabinet with light source, coupling optics, detector and readout electronics—is ruggedized so that it can be maintained by a semi-skilled person on a location-by-location basis.

As shown in FIG. 3, the format (1) detection platform combines (a) self-referencing microfluidic multi-lane arrays; (b) SPR imaging/angle shifts for readout; and (c) reusable fluidic chips. Furthermore, carrying out the recognition event in a microfluidic format accrues inherent mass transport advantages meaning that measurements can be cycled faster than with benchscale flow cells. In addition to the specificity provided by the siderophore, the plasmonic readout easily has the sensitivity to detect a single pathogen organism in the active area (typically 50 μm (micrometers) wide by 1 mm long). The ultimate solution-referenced limit of detection (LOD) is determined by the capture efficiency, and LODs of a few units mL⁻¹ are readily attainable. We are currently optimizing the surface derivatization chemistries used to anchor the siderophores for optimal bacterial recognition and capture.

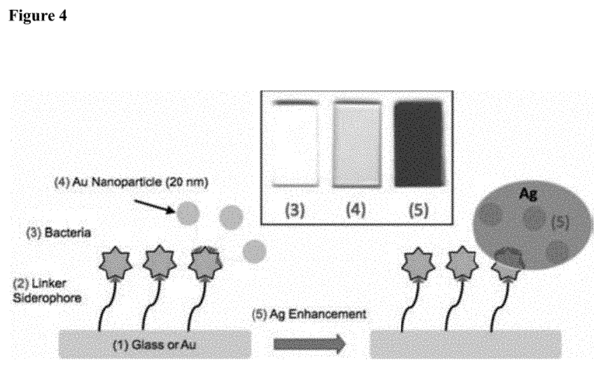

Strong motivation exists for a pathogen diagnostic test which requires no reader at all, and is usable in the field by personnel with no training, a paradigm known as point-of-care diagnostics. Format (2) embodies an alternative practice of the invention. As shown in FIG. 4, a test substrate is functionalized with an artificial siderophore, which is selective for the targeted pathogen. A bacterial cell is captured on the surface, similar to that described above. In a second step, the remaining species in solution are rinsed away in a buffer solution. In 4(C), a solution of functionalized metallic nanoparticles is introduced, which binds to the surface of the bacteria. The molecular recognition moiety in (C) may be a siderophore, an antibody, or some other species which binds to the bacteria present on the surface. Since the selection (identification) of the bacteria has already taken place by the immobilized siderophore in 4(A), the subsequent advantage of the nucleating metallic nanoparticles need not be species- or strain-selective, a distinct advantage in ease of use compared to format (1). The final step of the diagnostic test, the development step, involves a solution of metal ions (Ag for example) and an organic reductant. Such a solution is well-known to result in a thick film of metal wherever a nucleation site exists. Thus, the test strip described here is label-free, does not require a reader, and maintains the benefits of siderophore-mediated sensing described above.

In one embodiment, the siderophore is a natural siderophore, semi-synthetic siderophore, synthetic siderophore, or combination thereof. In one embodiment, the siderophore is a natural siderophore In one embodiment, the siderophore is a semi-synthetic siderophore. In one embodiment, the siderophore is a synthetic siderophore. One or more than one siderophore may be present. In one embodiment, only one type of siderophore is present on the surface. In another embodiment, a mixture of more than one type of siderophore is present on the surface. For example, in one embodiment a mixture of one or more different synthetic siderophores and one or more different natural siderophores are present on the surface.

In one embodiment, wherein mixtures of different siderophores are present, each type of siderophore may be specific to the same bacterium, or each type of siderophore may be specific to different bacterium.

In one embodiment, the siderophore is a synthetic siderophore having one of the formulas Ia, IIa, IIIa, IVa, or Va. One or more than one synthetic siderophore may be present. In one embodiment, only one type of synthetic siderophore is present on the surface. In another embodiment, a mixture of more than one type of synthetic siderophore is present on the surface.

In one embodiment, the siderophore is a synthetic siderophore having one of the formulas Ia, IIa, IIIa, IVa, or Va.

In one embodiment, the siderophore is a synthetic siderophore having the formula Ia.

In one embodiment, the siderophore is a synthetic siderophore having the formula IIa.

In one embodiment, the siderophore is a synthetic siderophore having the formula IIIa.

In one embodiment, the siderophore is a synthetic siderophore having the formula IVa.

In one embodiment, the siderophore is a synthetic siderophore having the formula Va.

In one embodiment, the siderophore is a synthetic siderophore having one of the formulas Ib, IIb, IIIb, IVb, or Vb.

In one embodiment, the siderophore is a synthetic siderophore having the formula Ib.

In one embodiment, the siderophore is a synthetic siderophore having the formula IIb.

In one embodiment, the siderophore is a synthetic siderophore having the formula IIIb.

In one embodiment, the siderophore is a synthetic siderophore having the formula IVb.

In one embodiment, the siderophore is a synthetic siderophore having the formula Vb.

Natural siderophores are known, and are not particularly limiting. In one embodiment, any natural siderophore with pendant functionality (for example amine, alcohol, carboxylic acid) for attachment to the linker, surface, or modified surface may be suitably used. Non-limiting examples of natural siderophores include Desferrioxamine A1, Desferrioxamine A2, Desferrioxamine B, Desferrioxamine D1, Desferrioxamine D2, Desferrioxamine E, Desferrioxamine G1, Desferrioxamine G2A, Desferrioxamine G2B, Desferrioxamine G2C, Desferrioxamine H, Desferrioxamine T1, Desferrioxamine T2, Desferrioxamine T3, Desferrioxamine T7, Desferrioxamine T8, Desferrioxamine X1, Desferrioxamine X2, Desferrioxamine X3, Desferrioxamine X4, Desferrioxamine Et 1, Desferrioxamine Et2, Desferrioxamine Et3, Desferrioxamine Te1, Desferrioxamine Te2, Desferrioxamine Te3, Desferrioxamine P1, Fimsbactin, Ferrichrome, Ferrichrome C, Ferricrocin, Sake Colorant A, Ferrichrysin, Ferrichrome A, Ferrirubin, Ferrirhodin, Malonichrome, Asperchrome A, Asperchrome B1, Asperchrome B2, Asperchrome B3, Asperchrome C, Asperchrome D1, Asperchrome D2, Asperchrome D3, Asperchrome E, Asperchrome F1, Asperchrome F2, Asperchrome F3, Tetraglycine ferrichrome, Des(diserylglycyl)-ferrirhodin, Basidiochrome, Triacetylfusarinine, Fusarinine C, Fusarinine B, Neurosporin, Coprogen, Coprogen B (Desacetylcoprogen), Triornicin (Isoneocoprogen I), Isotriornicin (Neocoprogen I), Neocoprogen II, Dimethylcoprogen, Dimethylneocoprogen I, Dimethyltriornicin, Hydroxycopropen, Hydroxy-neocoprogen I, Hydroxyisoneocoprogen I, Palmitoylcoprogen, Amphibactin B, Amphibactin C, Amphibactin D, Amphibactin E, Amphibactin F, Amphibactin G, Amphibactin H, Amphibactin I, Ferrocin A, Coelichelin, Exochelin MS, Vicibactin, Enterobactin (Enterochelin), Agrobactin, Parabactin, Fluvibactin, Agrobactin A, Parabactin A, Vibriobactin, Vulnibactin, Protochelin, Corynebactin, Bacillibactin, Salmochelin S4, Salmochelin S2, Rhizoferrin, Rhizoferrin analogues, Enantio Rhizoferrin, Staphyloferrin A, Vibrioferrin, Achromobactin, Mycobactin P, Mycobactin A, Mycobactin F, Mycobactin H, Mycobactin M, Mycobactin N, Mycobactin R, Mycobactin S, Mycobactin T, Mycobactin Av, Mycobactin NA (Nocobactin), Mycobactin J, Formobactin, Nocobactin NA, Carboxymycobactin, Carboxymycobactin 1, Carboxymycobactin 2, Carboxymycobactin 3, Carboxymycobactin 4, Pyoverdin 6.1 (Pseudobactin), Pyoverdin 6.2, Pyoverdin 6.3 (Pyoverdin Thai), Pyoverdin 6.4 (Pyoverdin 9AW), Pyoverdin 6.5,Pyoverdin 6.6, Isopyoverdin 6.7, (Isopyoverdin BTP1), Pyoverdin 6.8, Pyoverdin 7.1, Pyoverdin 7.2, (Pyoverdin BTP2), Pyoverdin 7.3, (Pyoverdin G +R), Pyoverdin 7.4, (Pyoverdin PVD), Pyoverdin 7.5, (Pyoverdin TII), Pyoverdin 7.6, Pyoverdin 7.7, Pyoverdin 7.8, (Pyoverdin PL8), Pyoverdin 7.9, (Pyoverdin 11370), Pyoverdin, Pyoverdin 7.11, (Pyoverdin 19310),Pyoverdin 7.12, (Pyoverdin 13525), Isopyoverdin 7.13, (Isopyoverdin 90-33), Pyoverdin 7.14, (Pyoverdin R′), Pyoverdin 7.15, Pyoverdin 7.16, (Pyoverdin 96-312), Pyoverdin 7.17, Pyoverdin 7.18, Pyoverdin 7.19, Pyoverdin 8.1, (Pyoverdin A214), Pyoverdin 8.2, (Pyoverdin P19), Pyoverdin 8.3, (Pyoverdin D-TR133), Pyoverdin 8.4, (Pyoverdin 90-51), Pyoverdin 8.5, Pyoverdin 8.6, (Pyoverdin 96-318), Pyoverdin 8.7, (Pyoverdin I-III), Pyoverdin 8.8, (Pyoverdin CHAO), Pyoverdin 8.9, (Pyoverdin E), Pyoverdin 9.1, Pyoverdin 9.2, (Pyoverdin Pau), Pyoverdin 9.3, Pyoverdin 9.4, Pyoverdin 9.5, (Pyoverdin 2392), Pyoverdin 9.6, Pyoverdin 9.7, (Pseudobactin 589A), Pyoverdin 9.8, (Pyoverdin 2461), Pyoverdin 9.9, Pyoverdin 9.10, (Pyoverdin 95-275), Pyoverdin 9.11, (Pyoverdin C), Pyoverdin 9.12, Pyoverdin 10.1, (Pyoverdin 2798), Pyoverdin 10.2, Pyoverdin 10.3, (Pyoverdin 17400), Pyoverdin 10.4, Pyoverdin 10.5, (Pyoverdin 18-1), Pyoverdin 10.6, (Pyoverdin 1, 2), Isopyoverdin 10.7, (Isopyoverdin 90-44), Pyoverdin 10.8, Pyoverdin 10.9, (Pyoverdin 2192), Pyoverdin 10.10, Pyoverdin 11.1, (Pyoverdin 51W), Pyoverdin 11.2, (pyoverdin 12), Pyoverdin 12.1, (Pyoverdin GM), Pyoverdin 12.2, (Pyoverdin 1547), Azoverdin, Azotobactin 87, Azotobactin D, Heterobactin A, Ornibactin-C4, Ornibactin-C6, Ornibactin-C8, Aquachelin A, Aquachelin B, Aquachelin C, Aquachelin D, Marinobactin A, Marinobactin B, Marinobactin C, Marinobactin D1, MarinobactinD2, Marinobactin E, Loihichelin A, Loihichelin B, Loihichelin C, Loihichelin D, Loihichelin E, Loihichelin F, Schizokinen, Aerobactin, Arthrobactin, Rhizobactin 1021, Nannochelin A, Nannochelin B, Nannochelin C, Acinetoferrin, Ochrobactin A, Ochrobactin B, Ochrobactin C, Snychobactin A, Snychobactin B, nychobactin C, Mugineic acid, 3-Hydroxymugineic acid, 2′-Deoxymugineic acid, Avenic acid, Distichonic acid, Deoxydistichonic acid, Rhizobactin, Staphyloferrin B, Alterobactin A, Alterobactin B, Pseudoalterobactin A, Pseudoalterobactin B, Petrobactin, Petrobactin sulphonate, Petrobactin disulphonate, Fusarinine A, Exochelin MN, Ornicorrugatin, Maduraferrin, Alcaligin, Putrebactin, Bisucaberin, Rhodotrulic acid, Dimerum acid, Amycolachrome, Azotochelin, (Azotobactin), Myxochelin, Amonabactin T789, Amonabactin P750, Amonabactin T732, Amonabactin P693, Salmochelin S1, Serratiochelin, Anachelin 1, Anachelin 2, Pistillarin, Anguibactin, Acinetobactin, Yersiniabactin, Micacocidin, Deoxyschizokinen, Heterobactin B, Desferrithiocin, Pyochelin, Thiazostatin, Enantio-Pyochelin, 2,3-Dihydroxybenzoylserine, Salmochelin SX, Citrate, Chrysobactin, Aminochelin, Siderochelin A, Aspergillic acid, Itoic acid, Cepabactin, Pyridoxatin, Quinolobactin, Ferrimycin A, Salmycin A, Albomycin, or combination thereof.

Other natural siderophores may be found in Robert C. Hider and Xiaole Kong Nat. Prod. Rep., 2010, 27, 637-657, and the appendices thereof, the entire contents of which are hereby incorporated by reference.

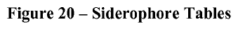

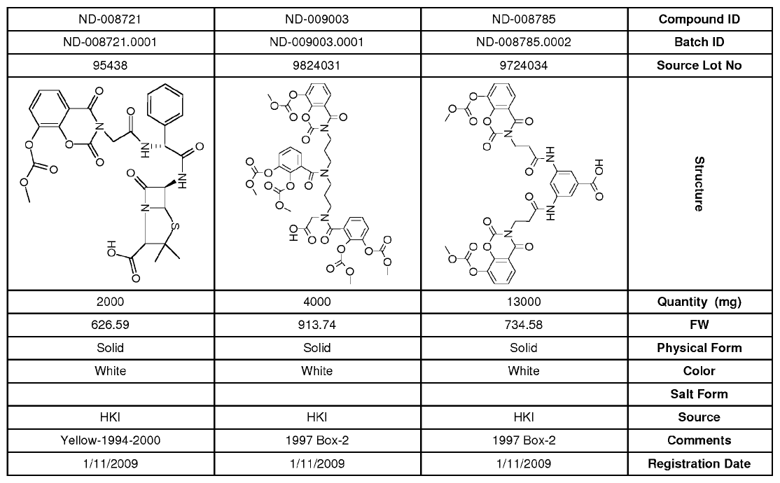

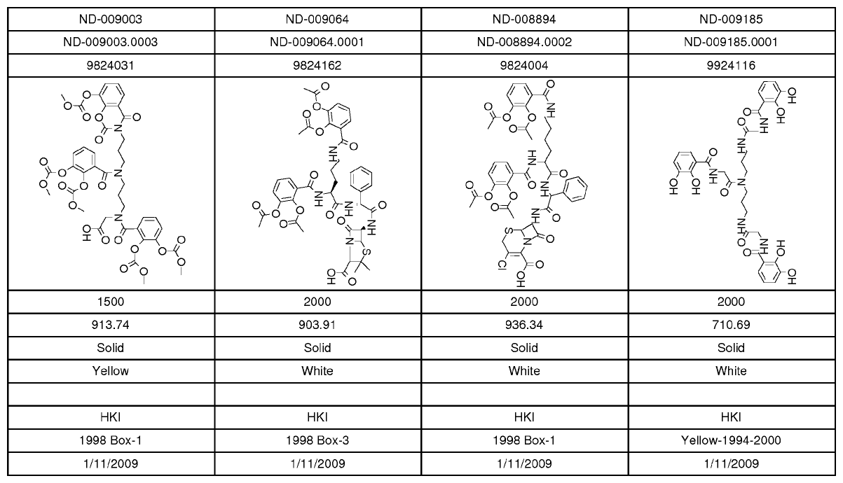

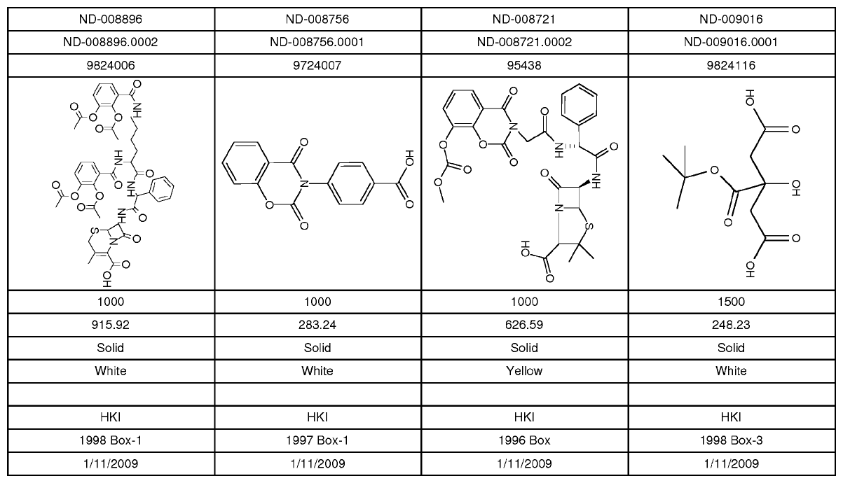

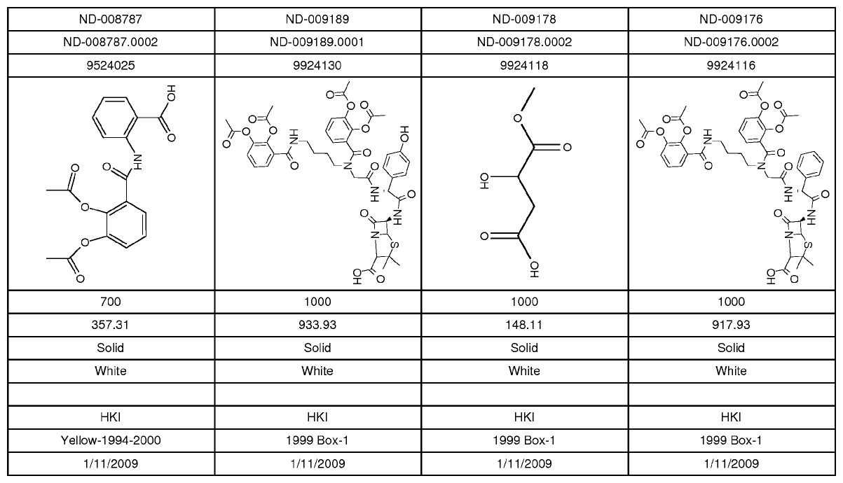

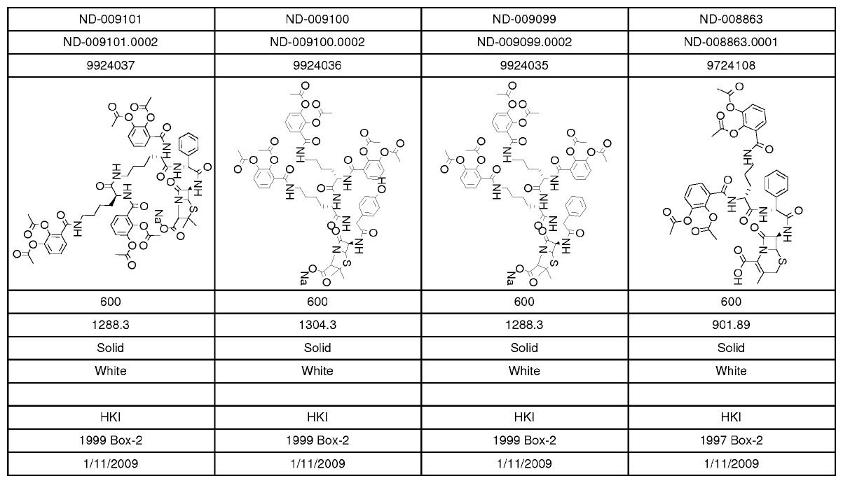

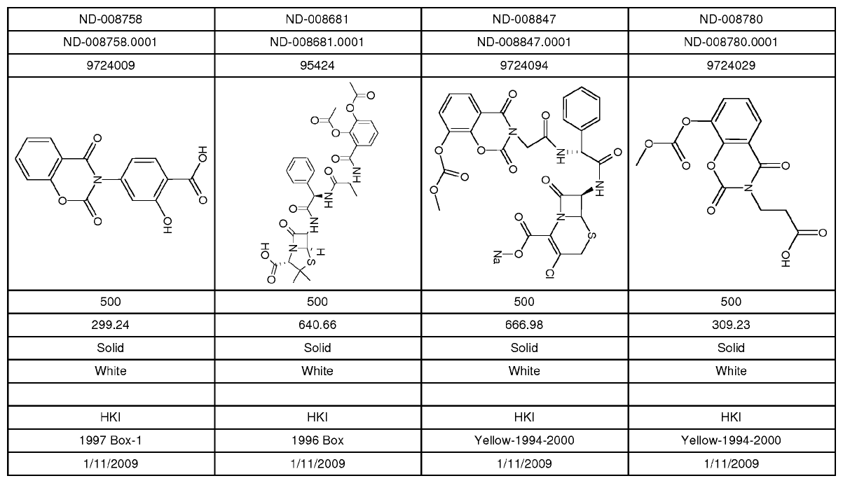

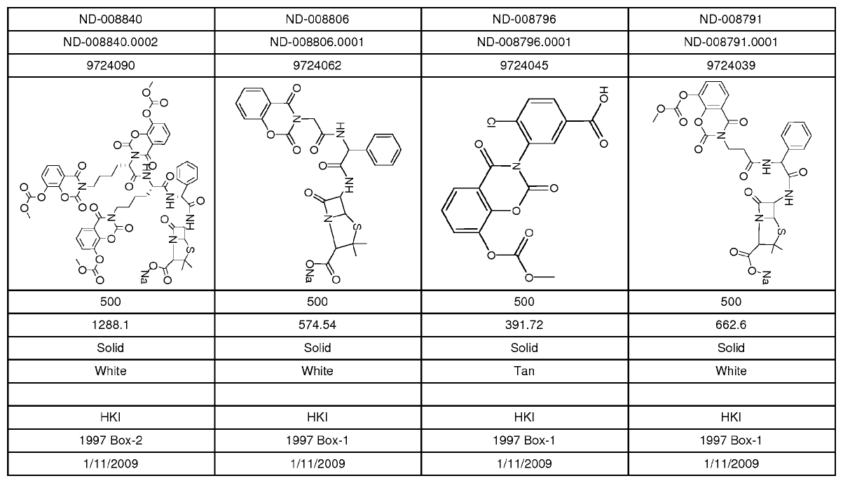

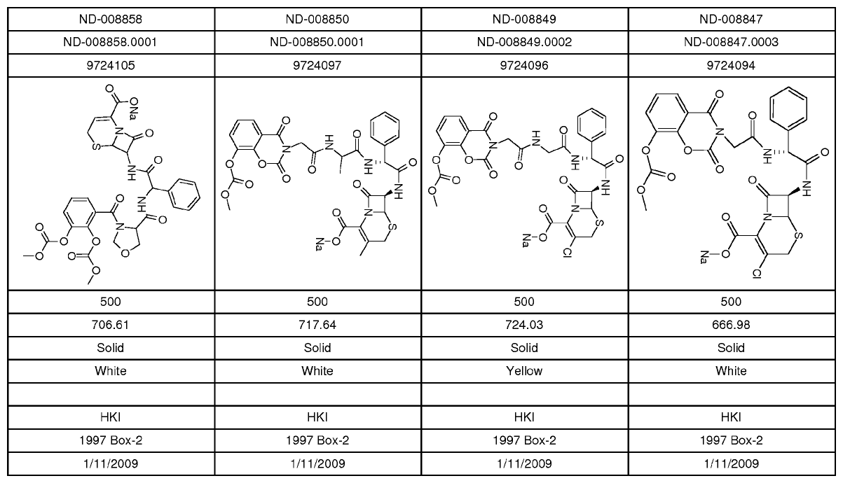

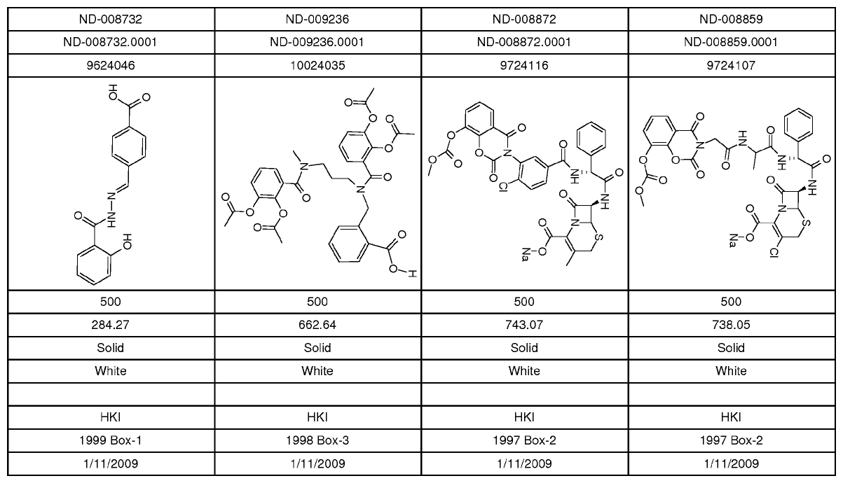

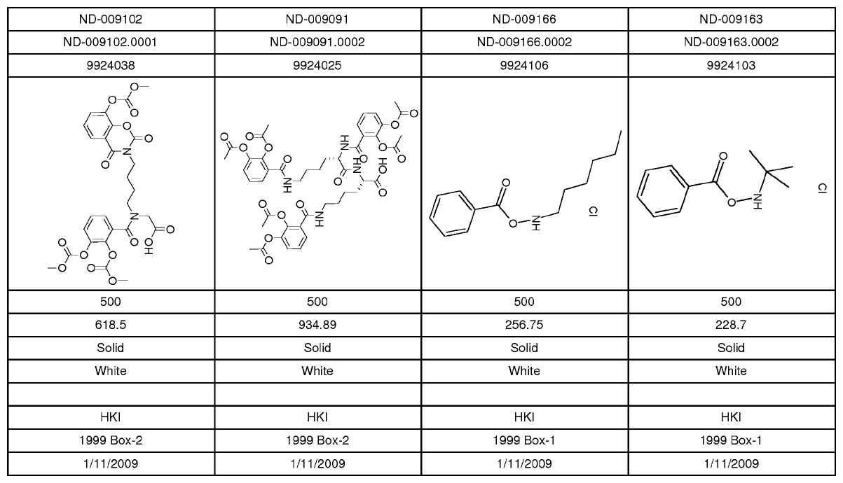

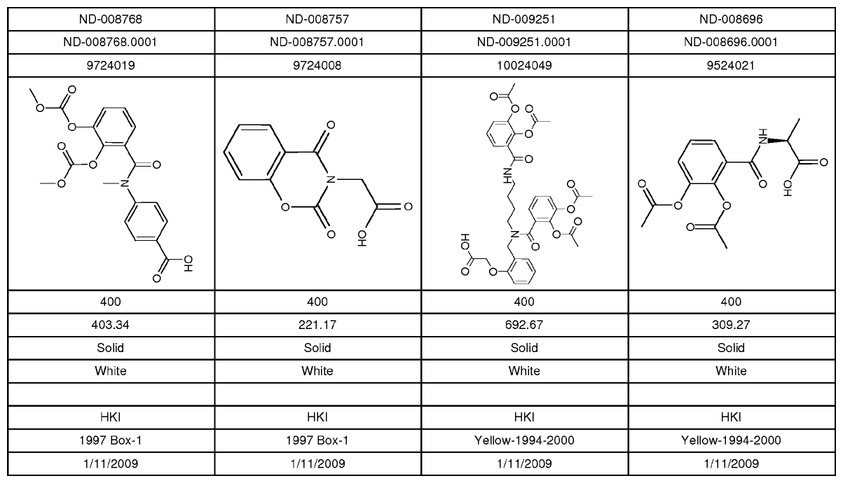

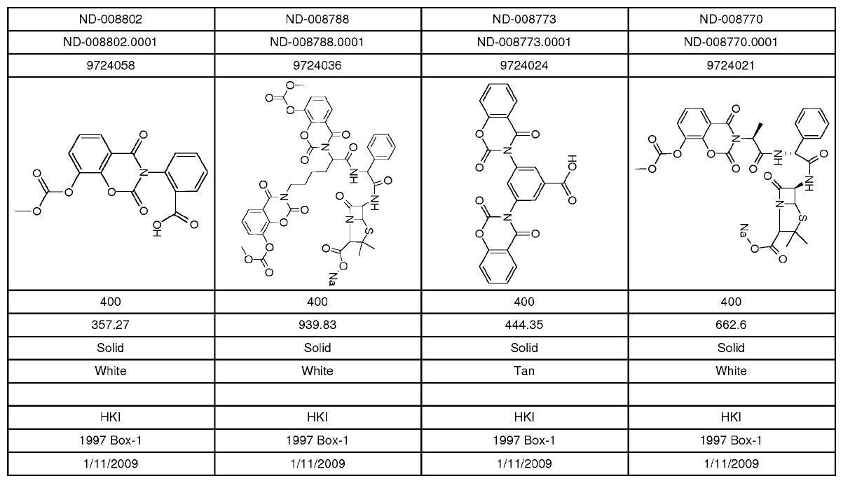

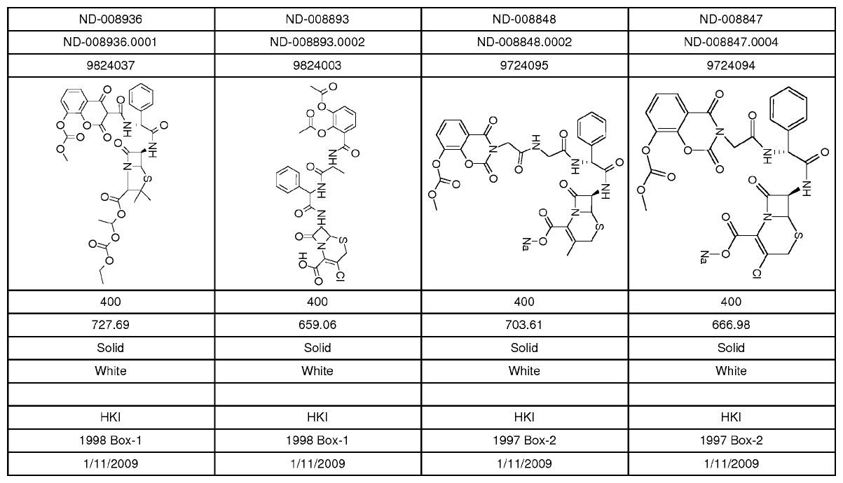

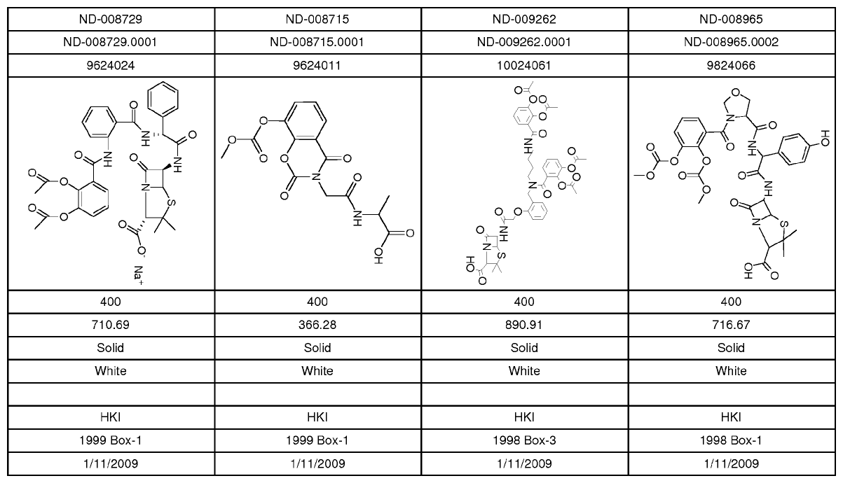

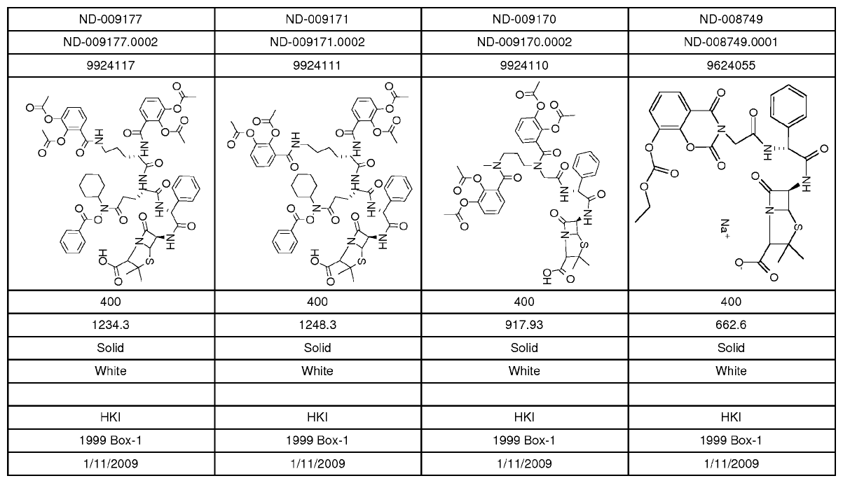

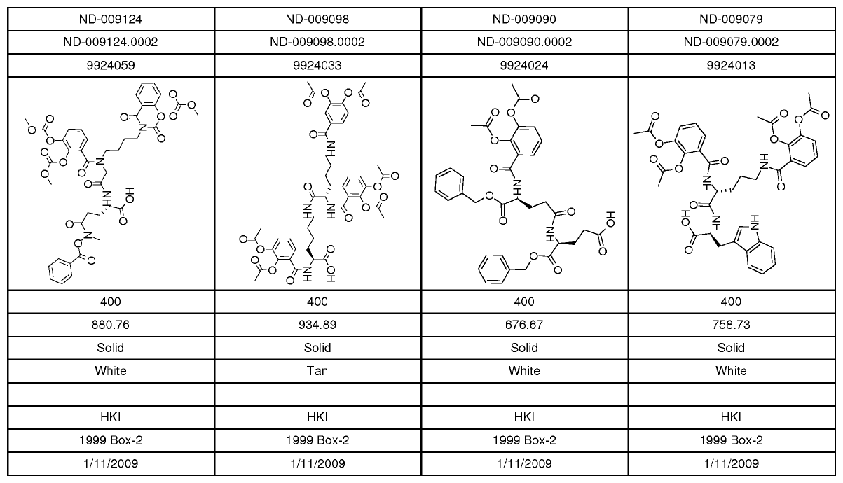

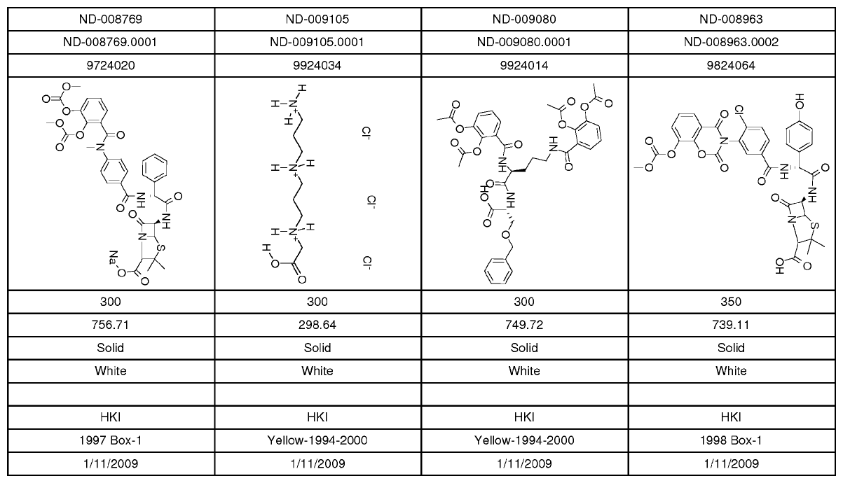

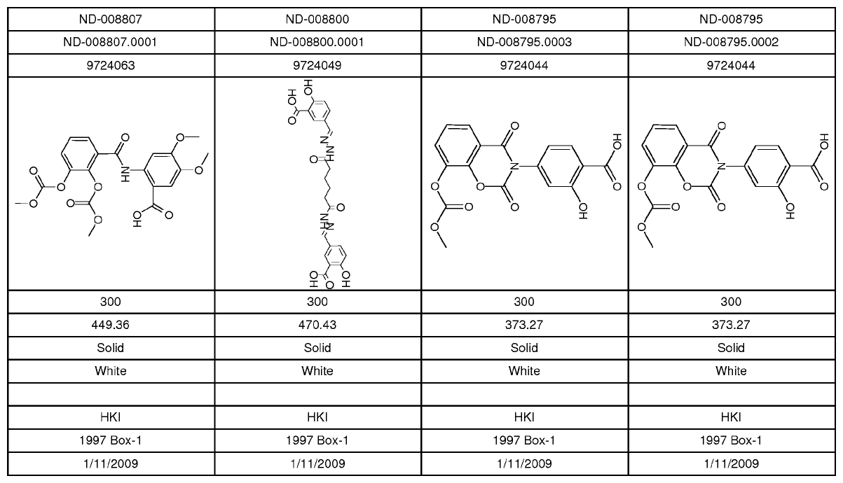

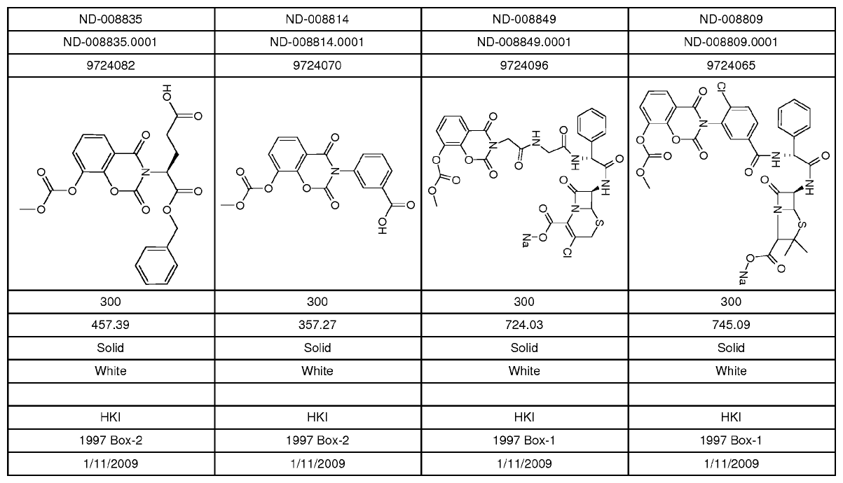

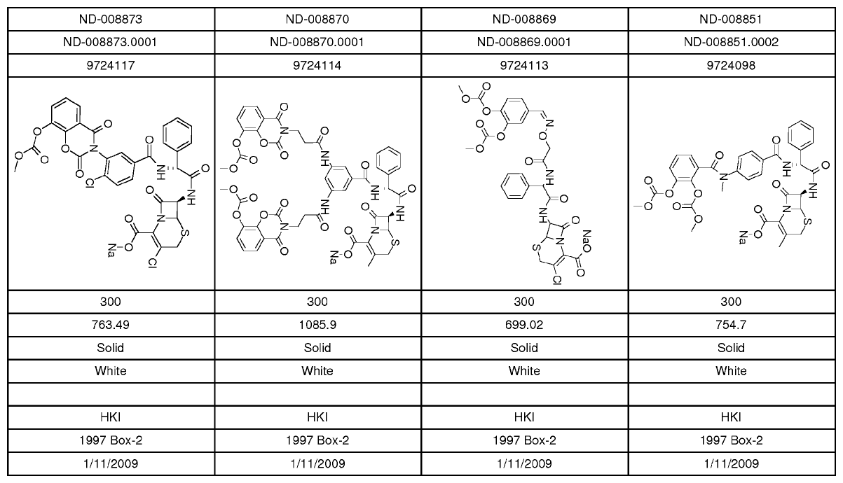

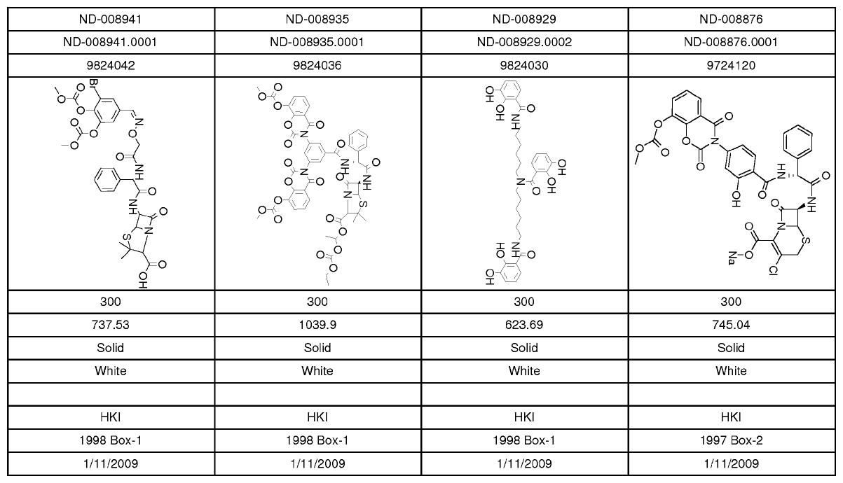

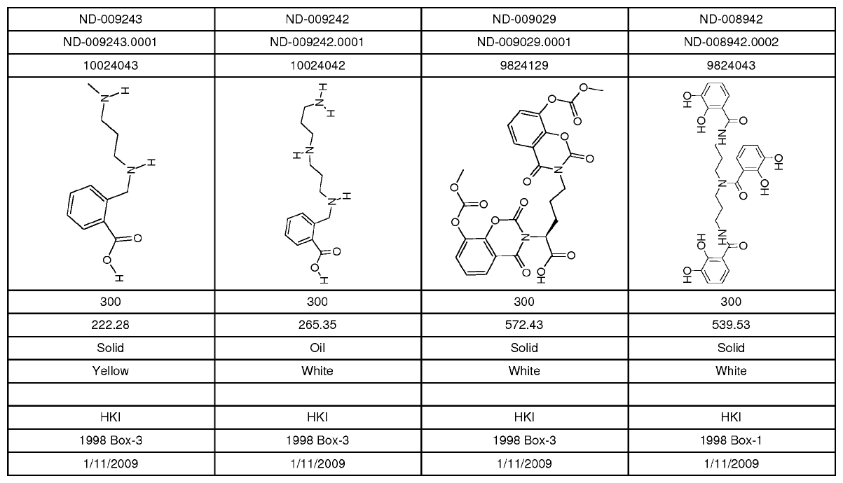

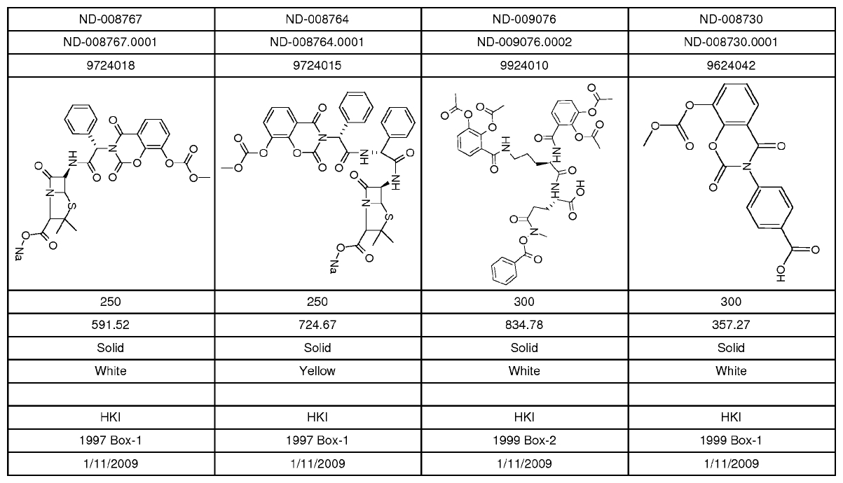

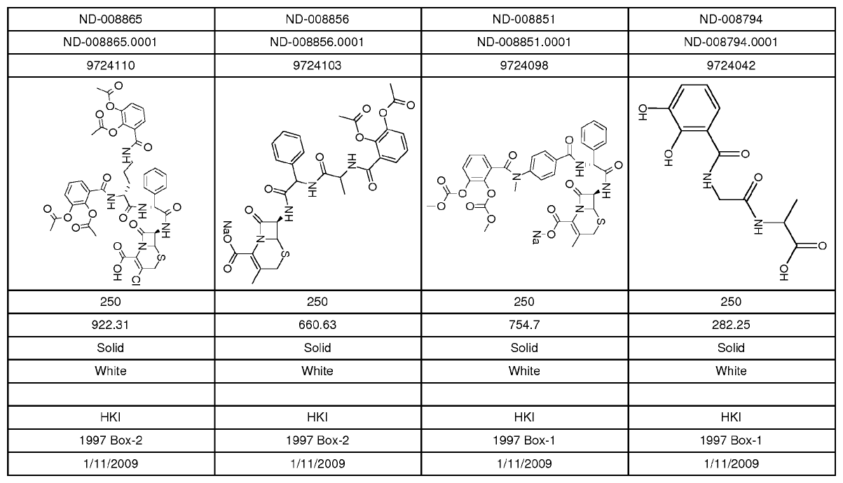

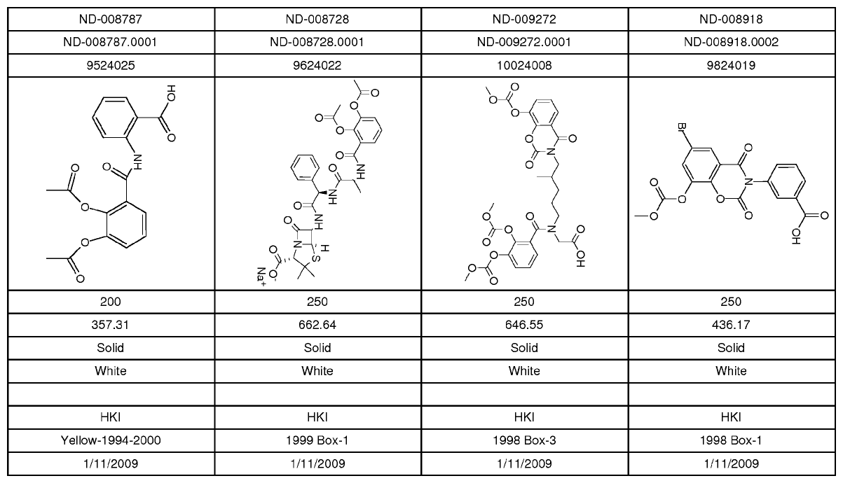

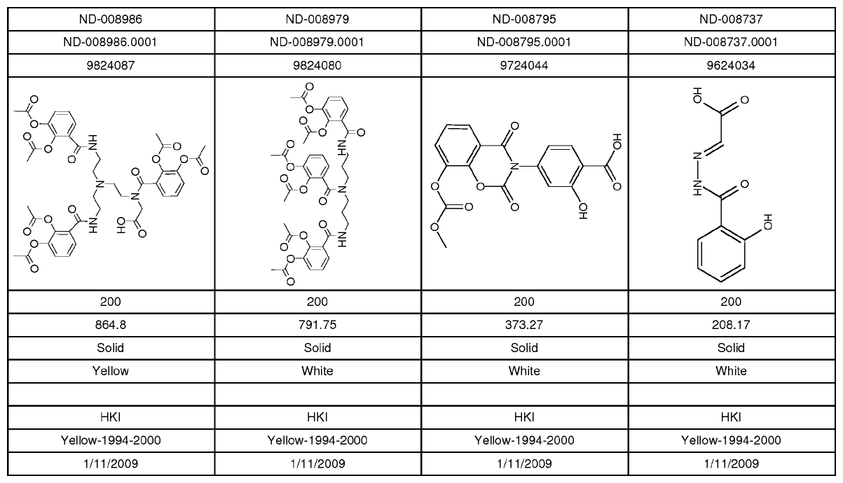

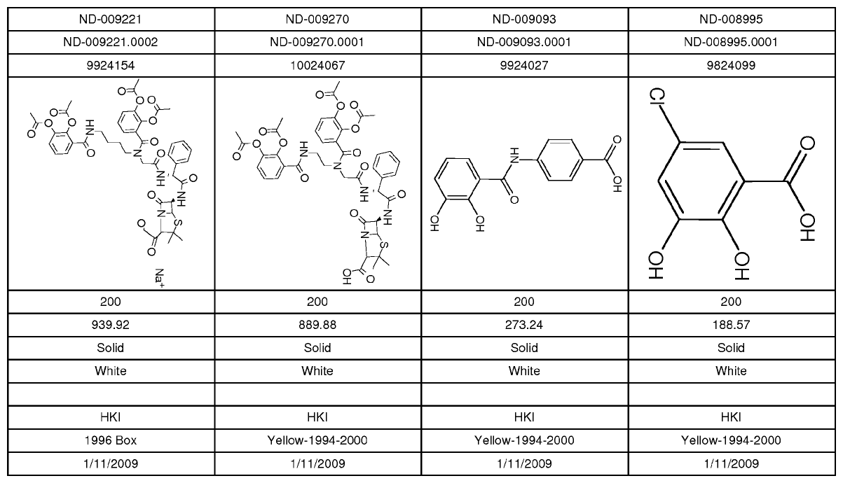

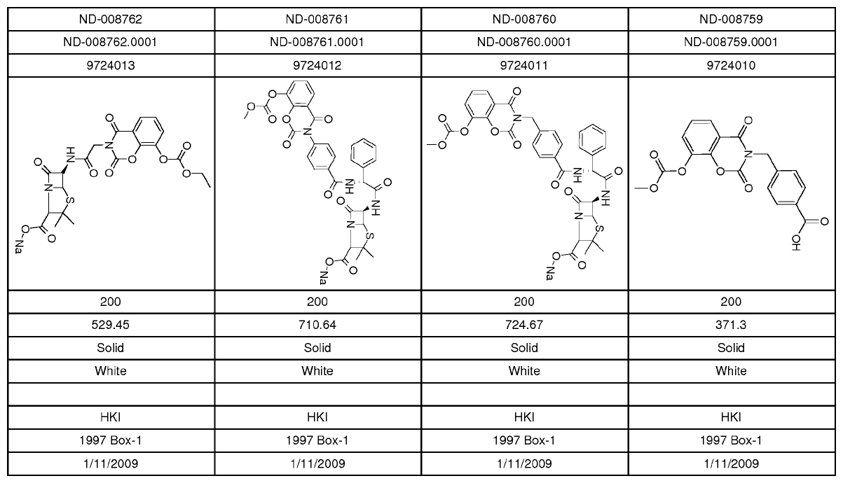

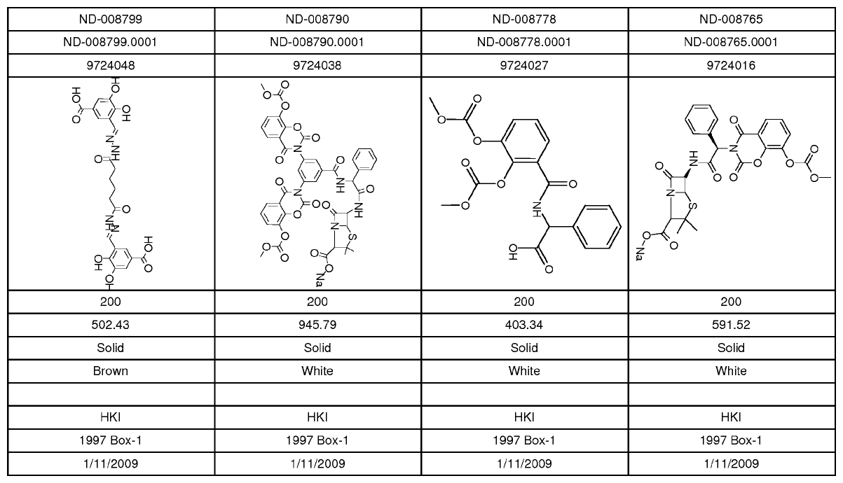

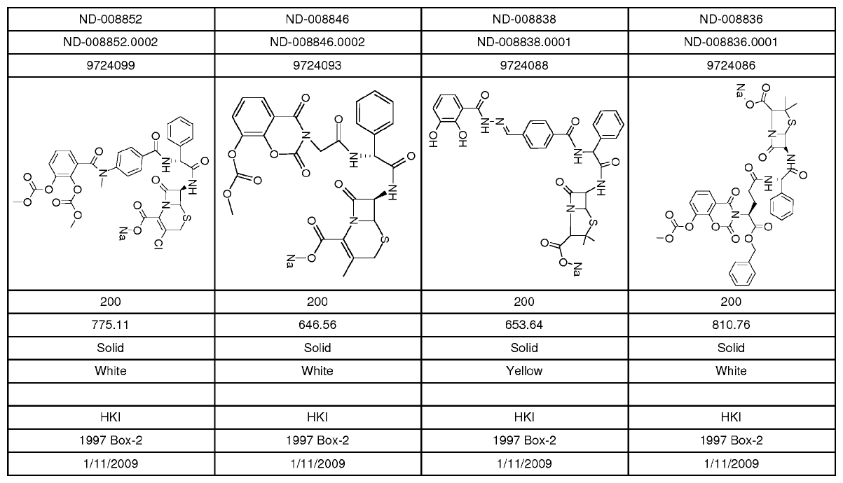

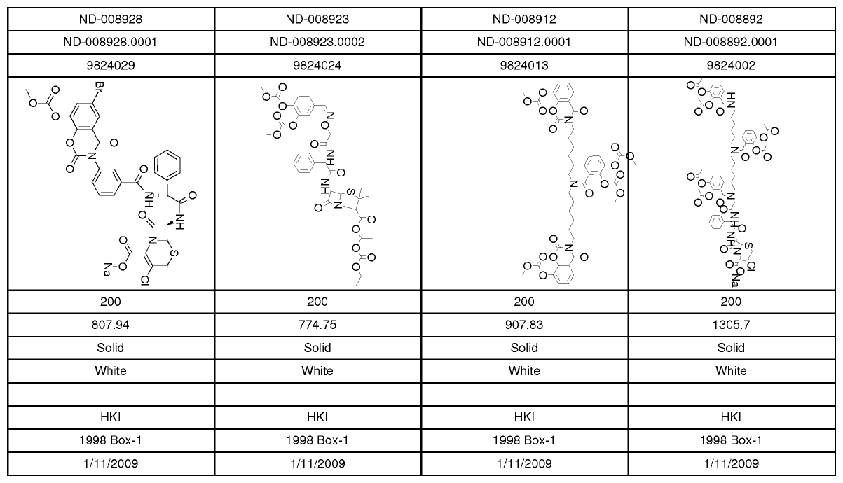

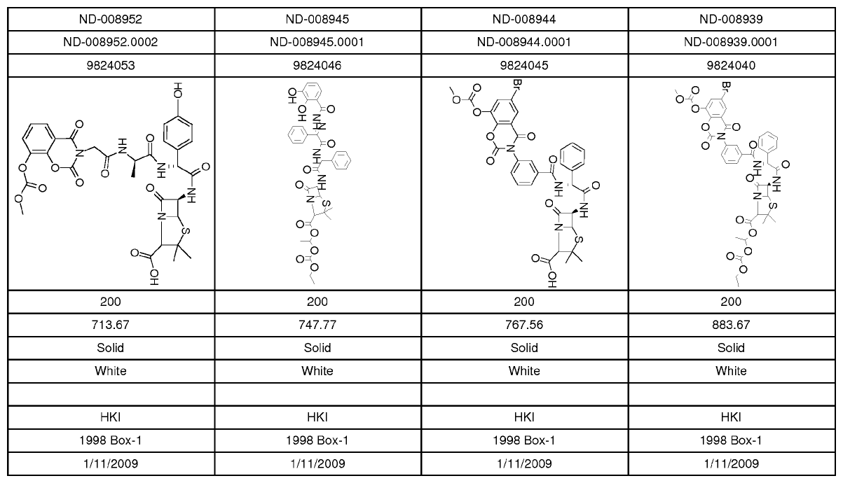

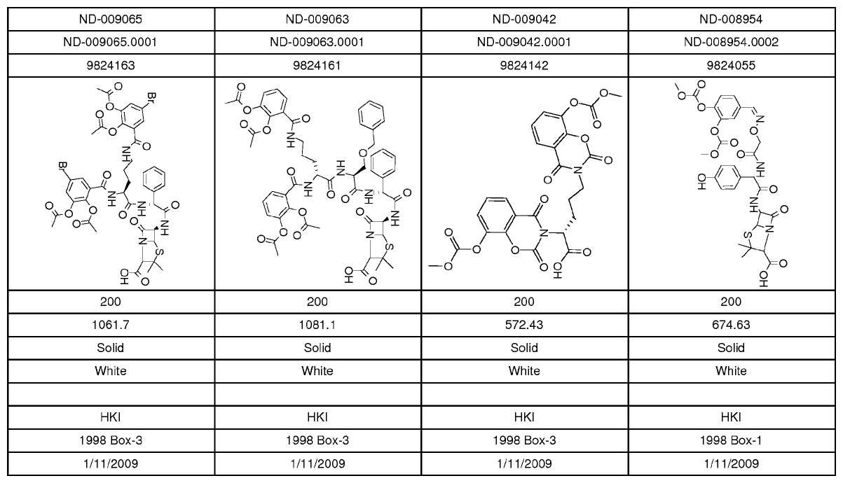

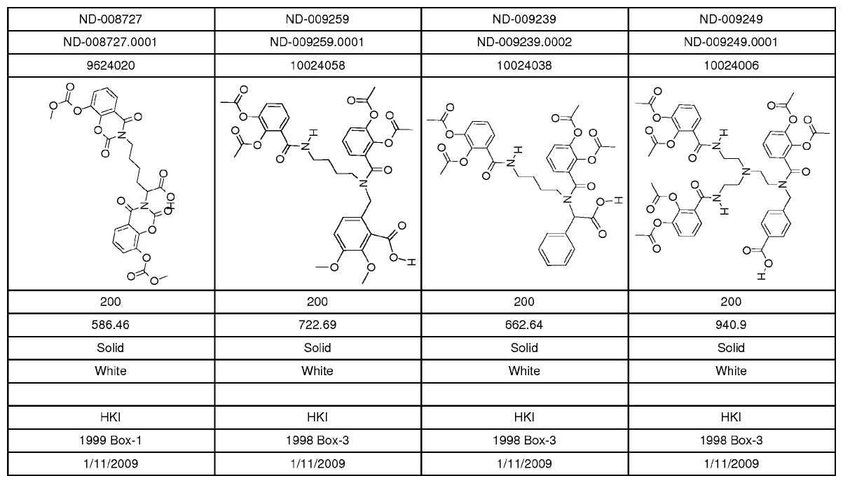

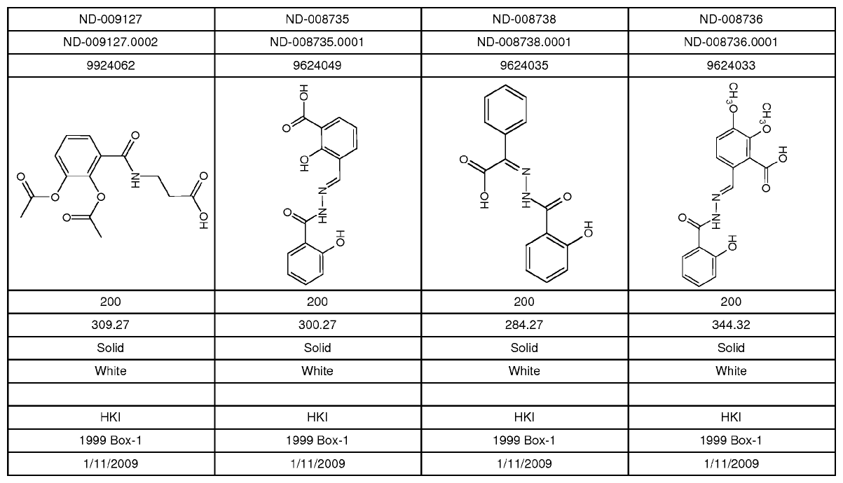

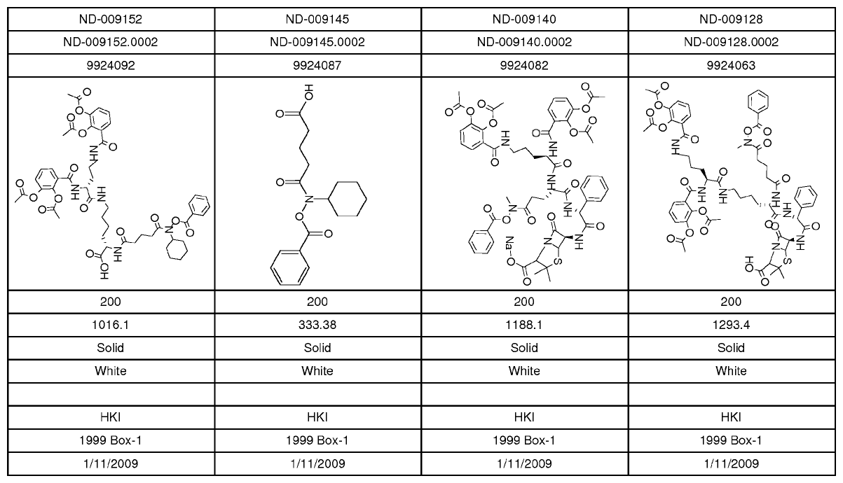

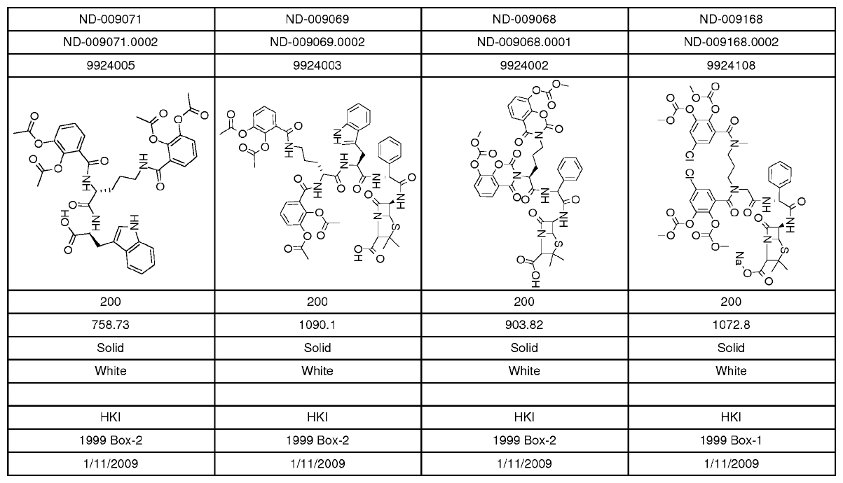

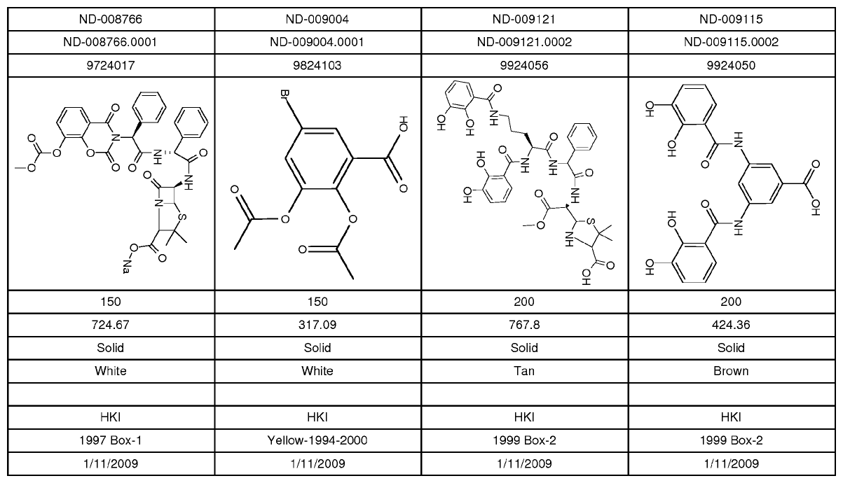

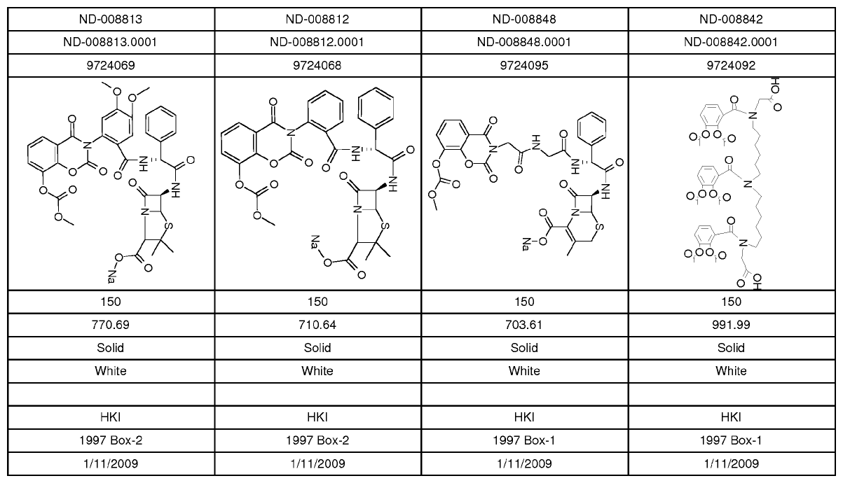

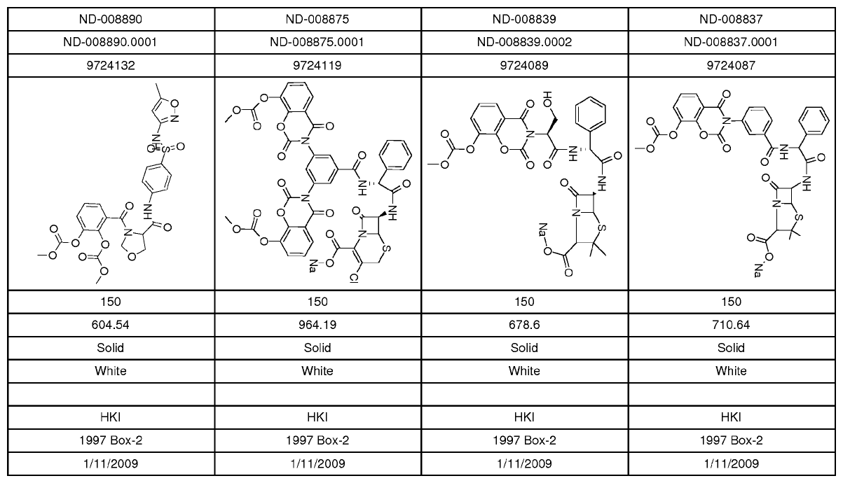

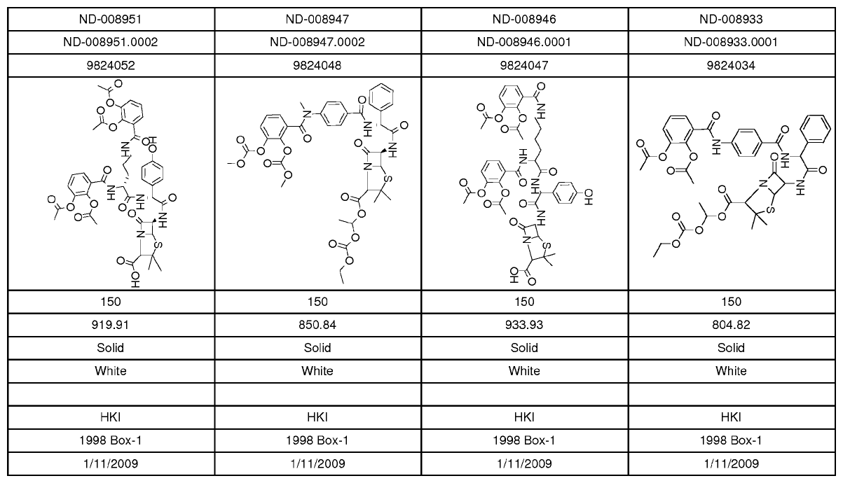

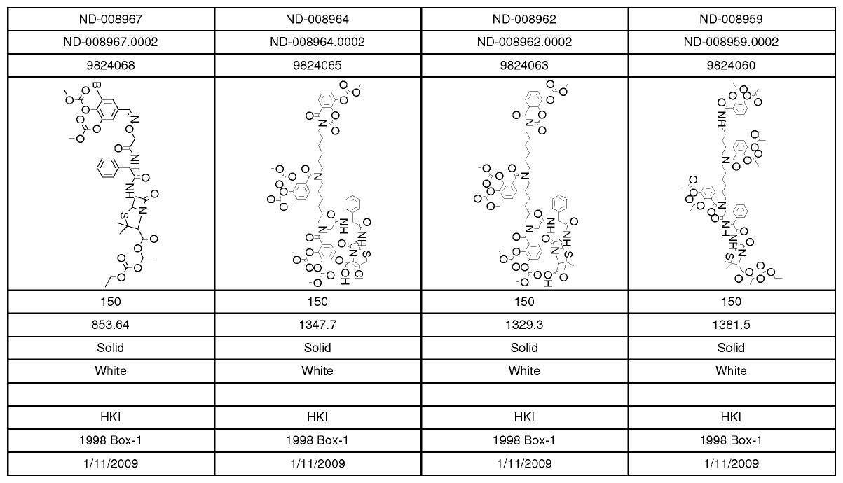

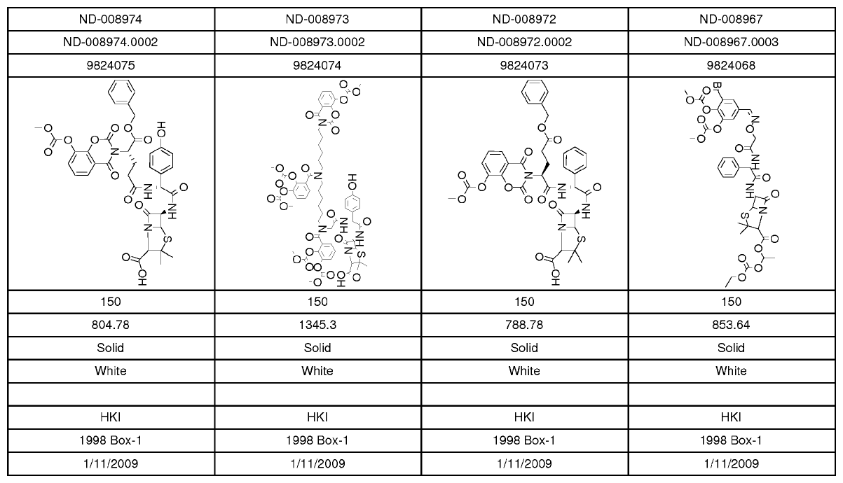

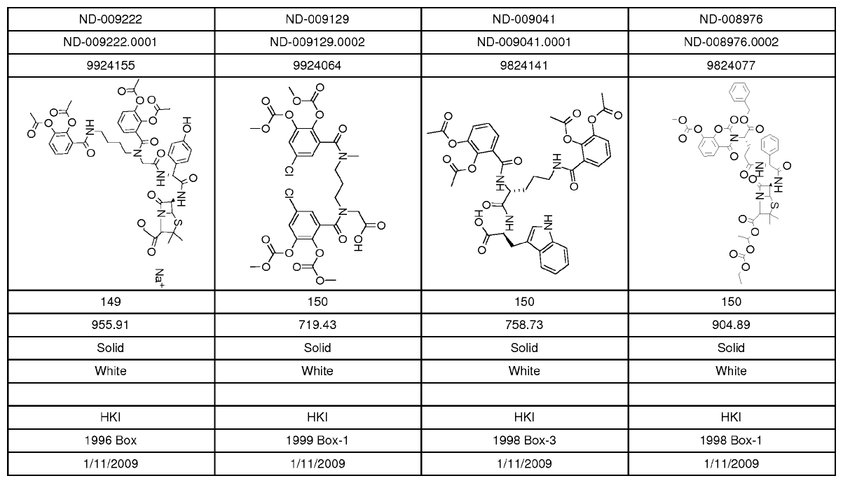

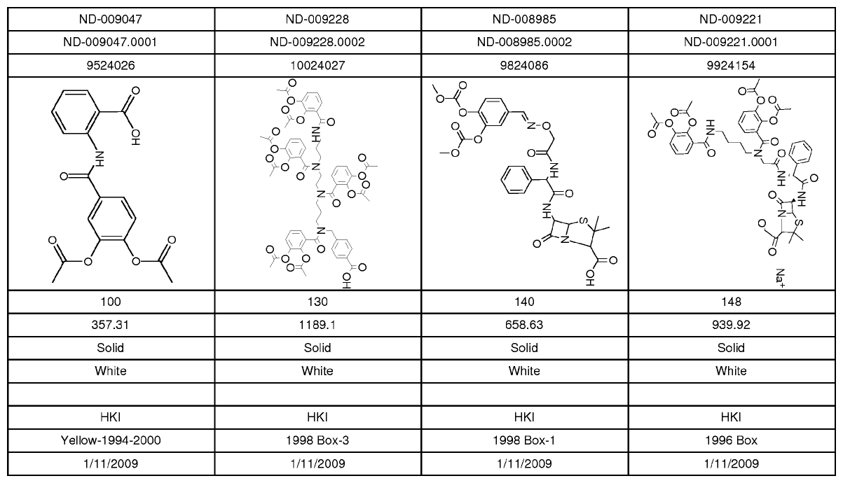

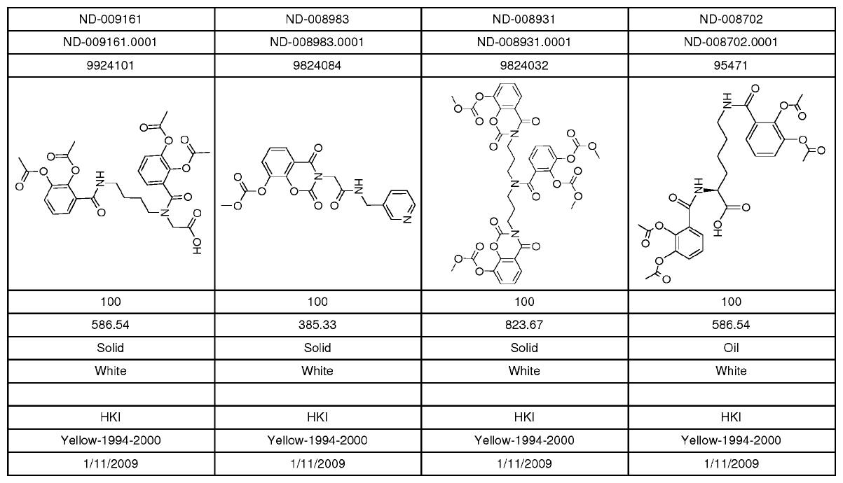

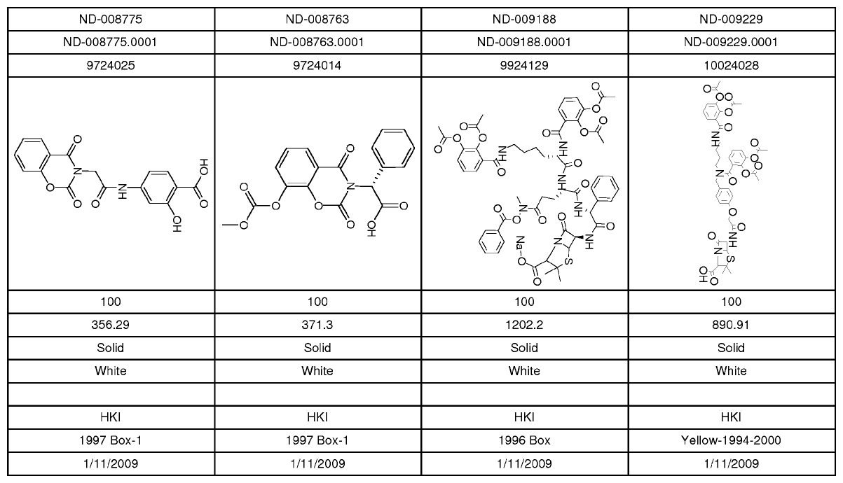

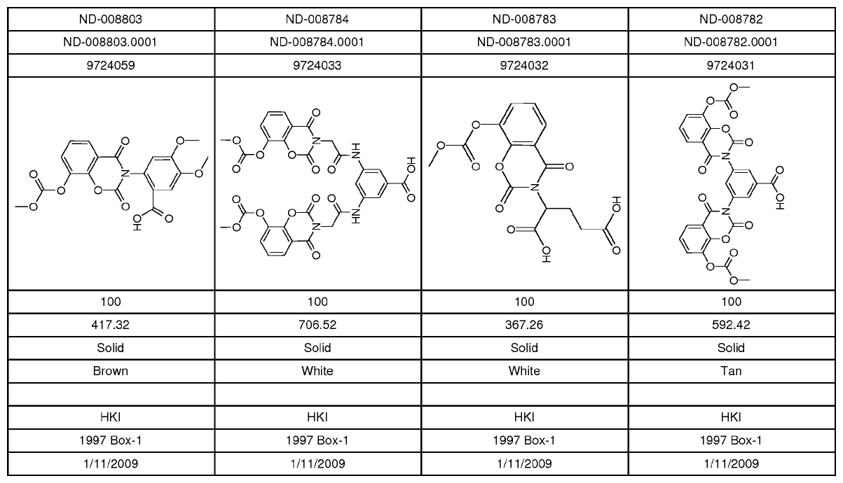

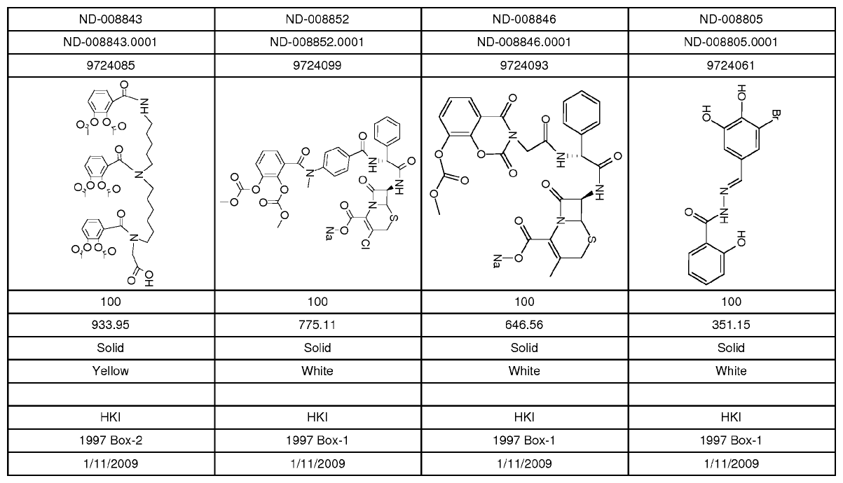

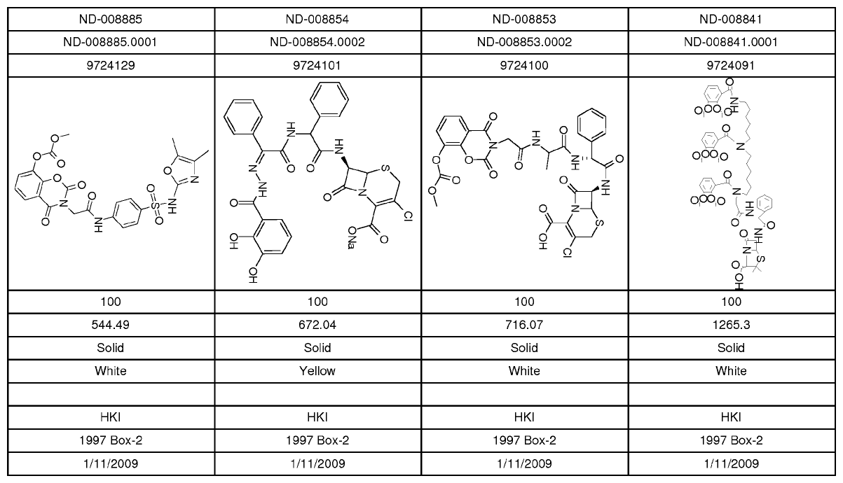

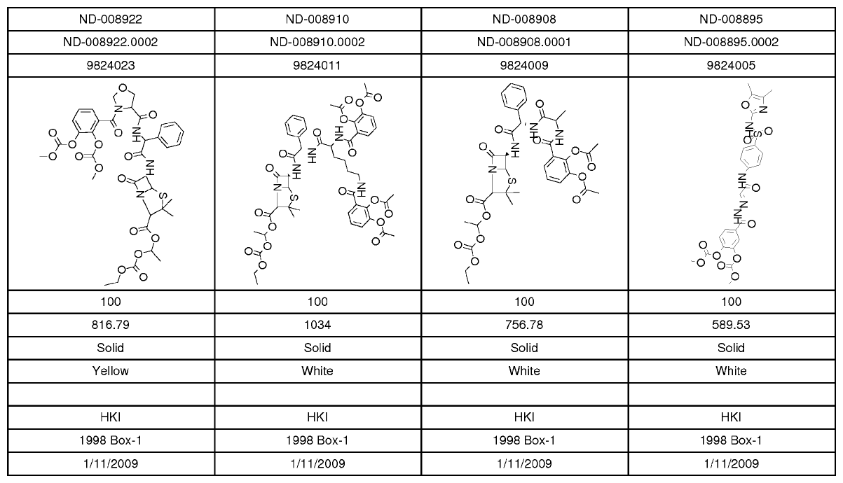

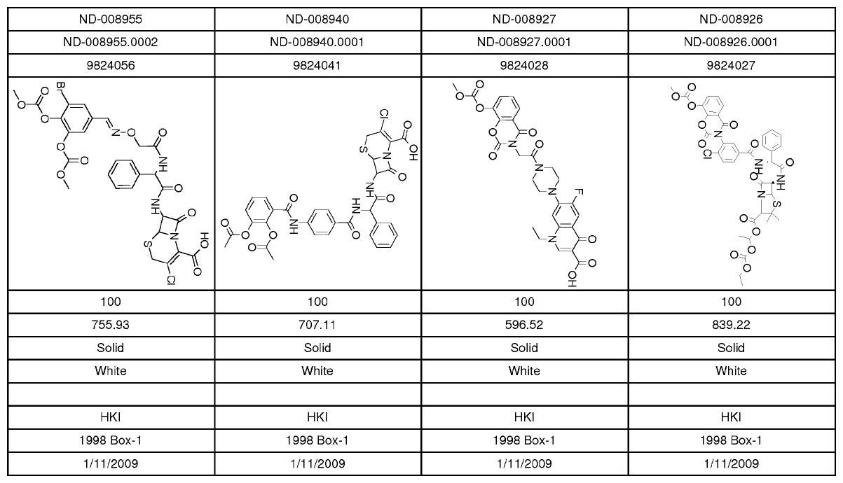

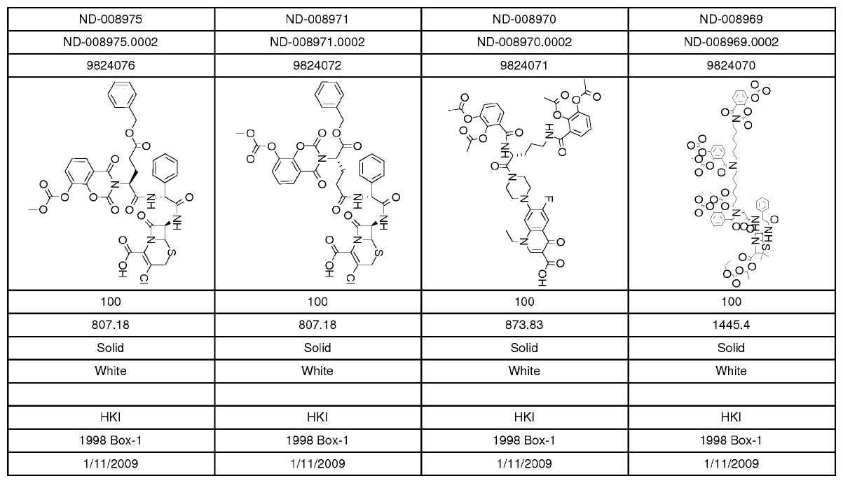

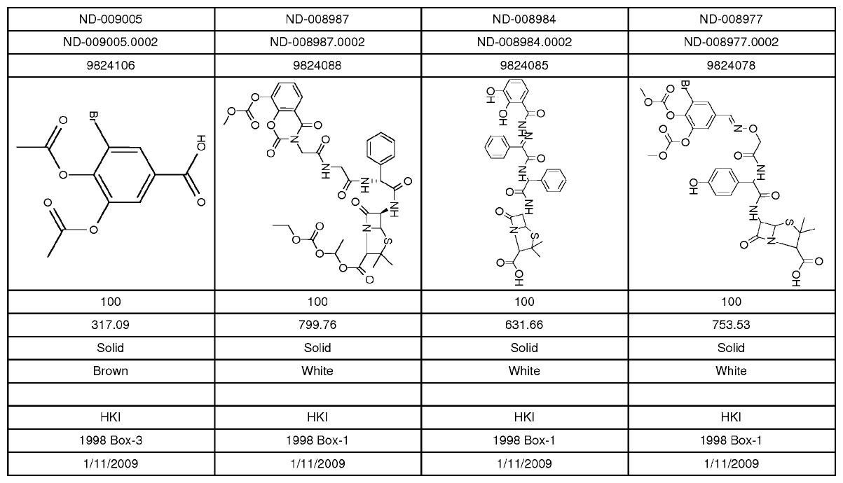

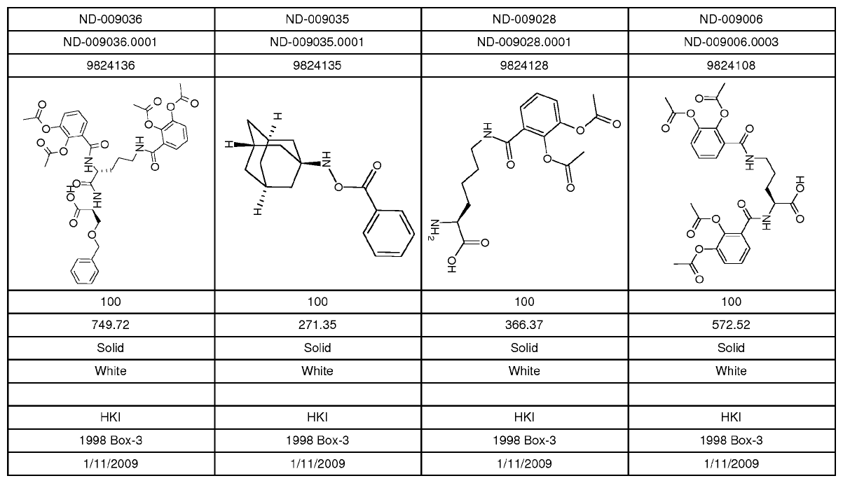

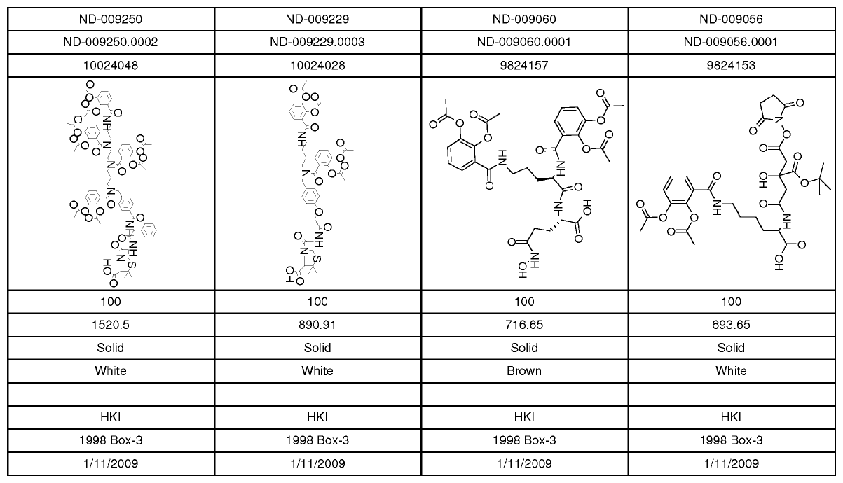

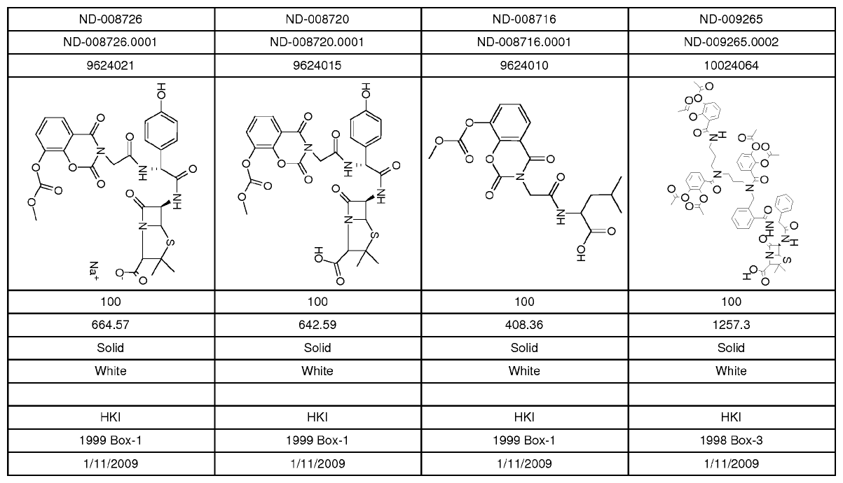

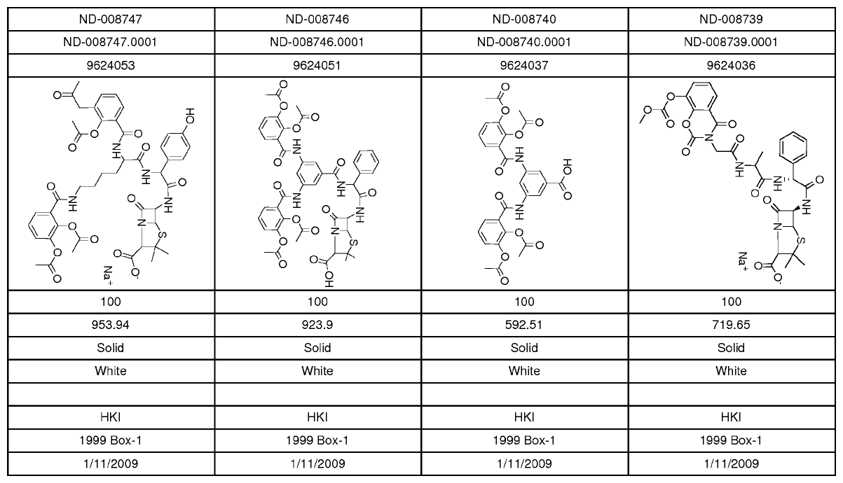

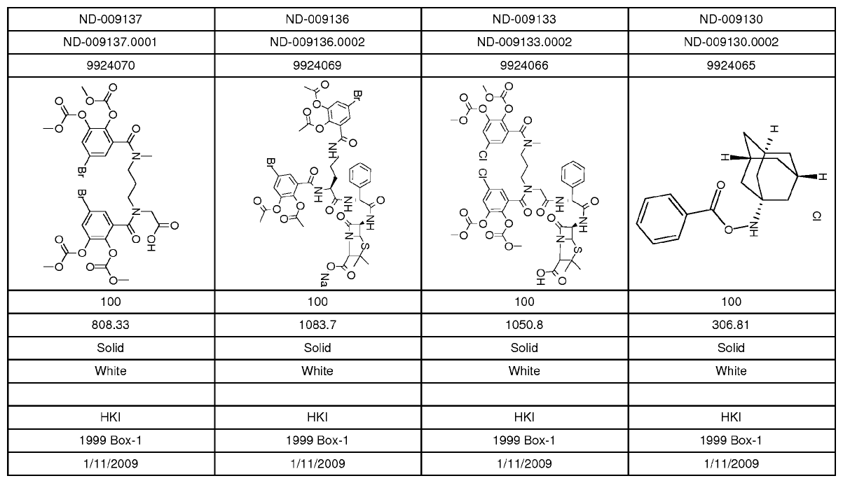

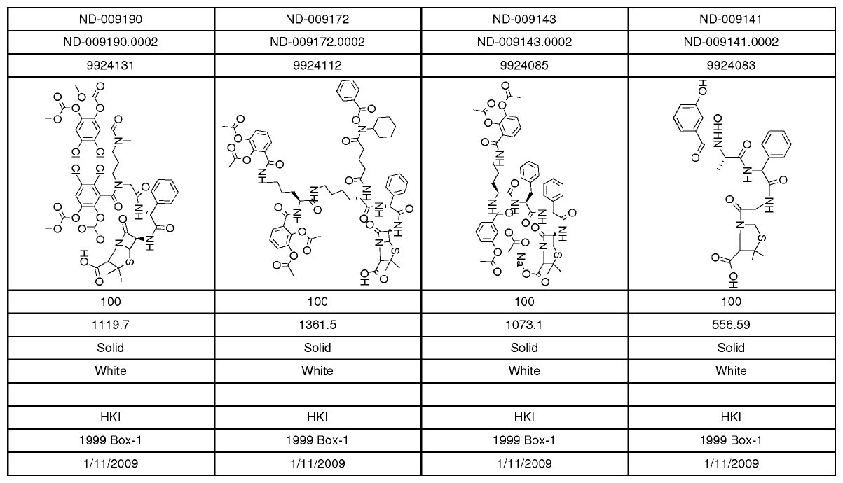

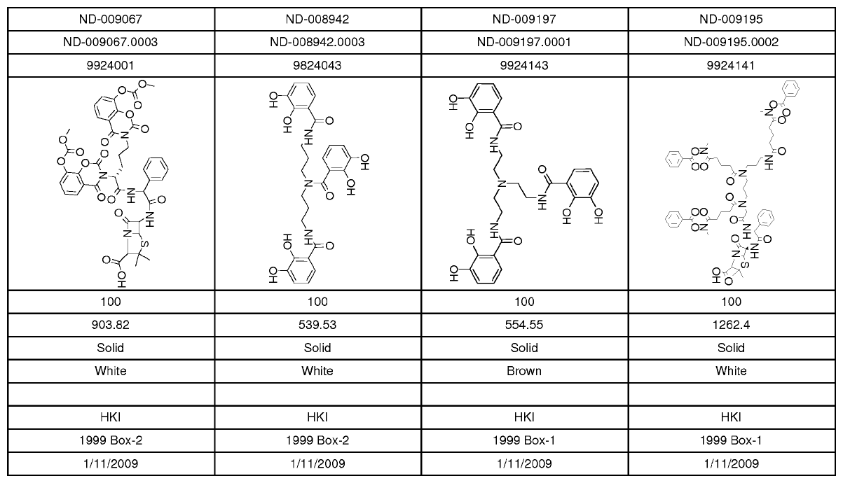

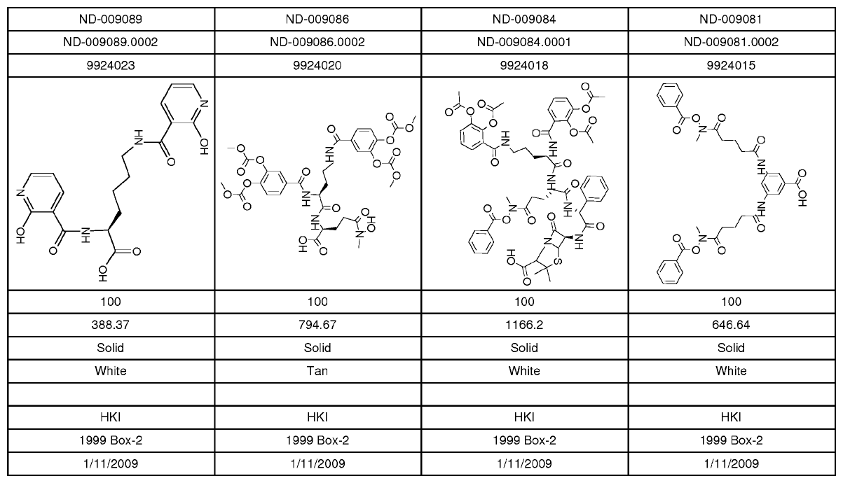

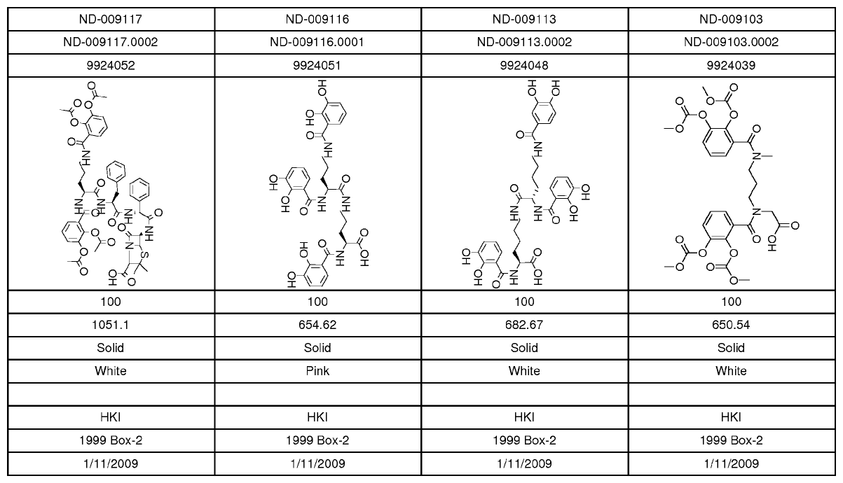

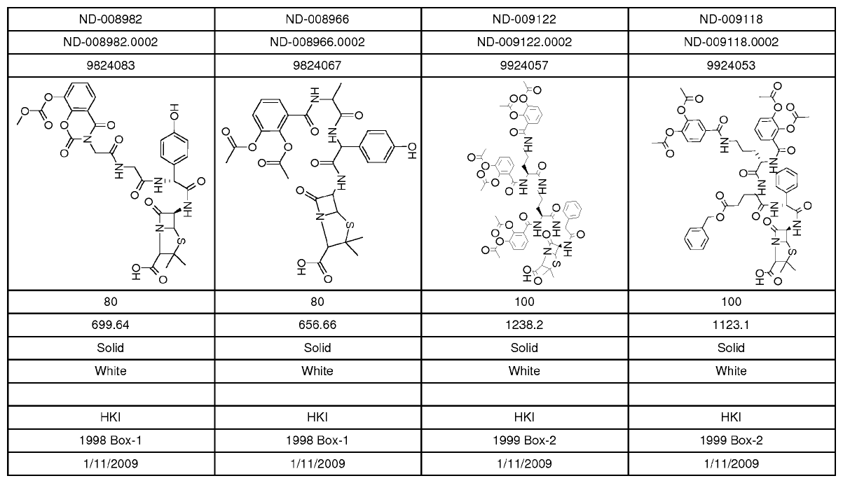

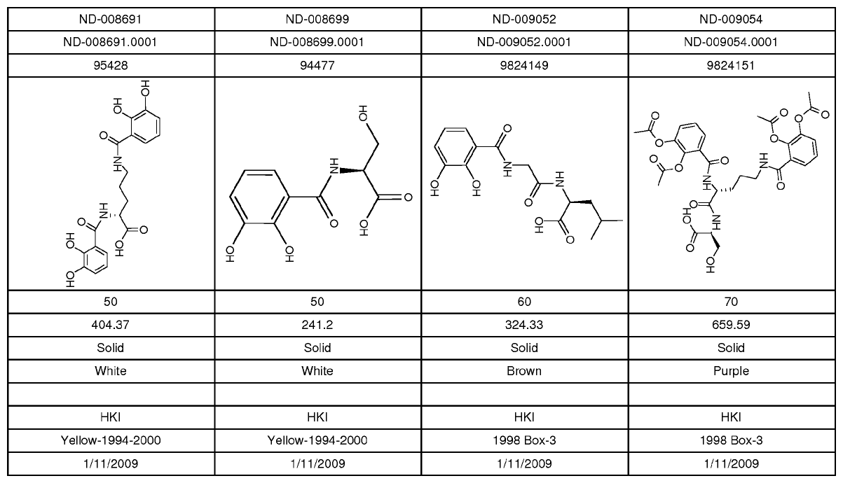

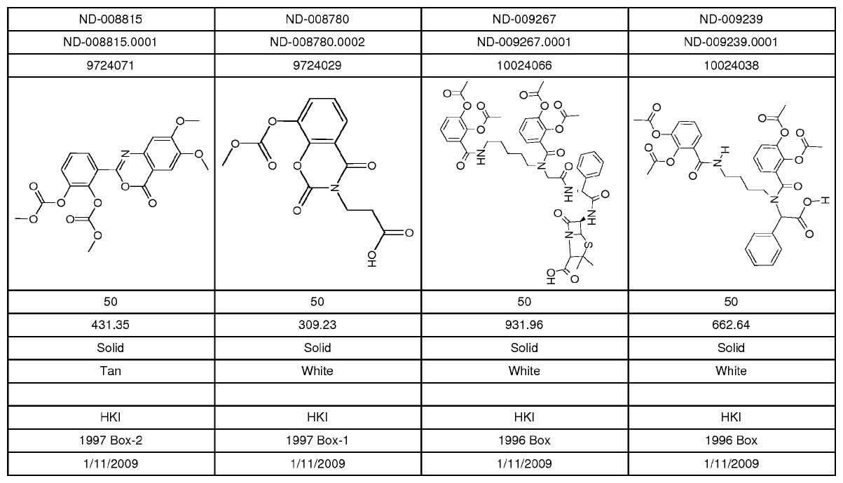

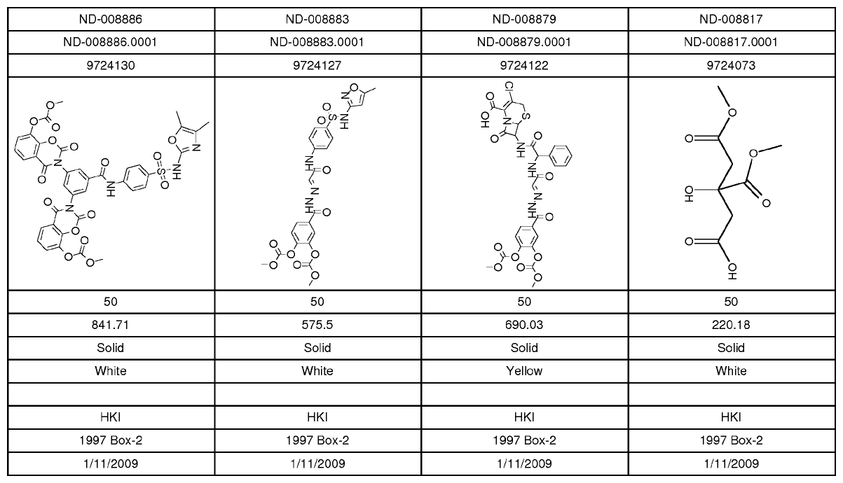

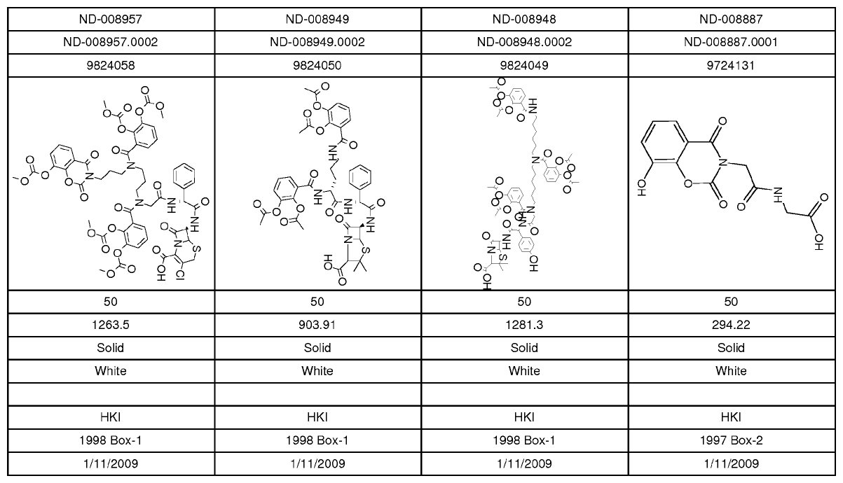

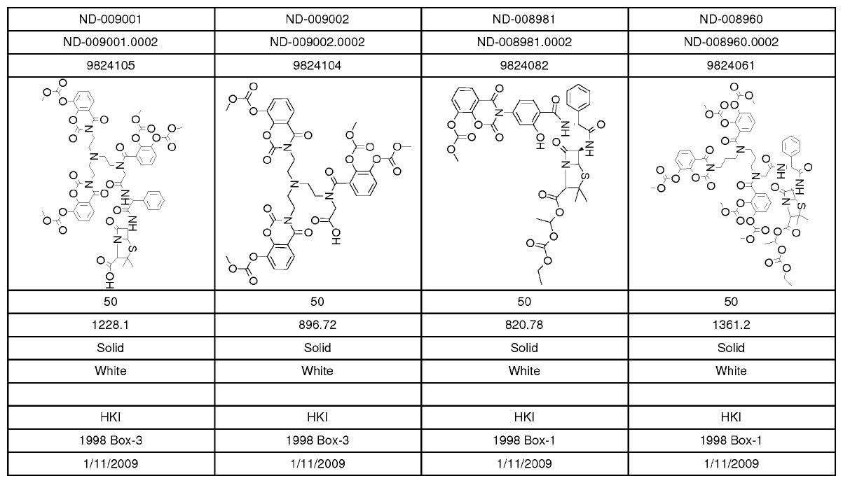

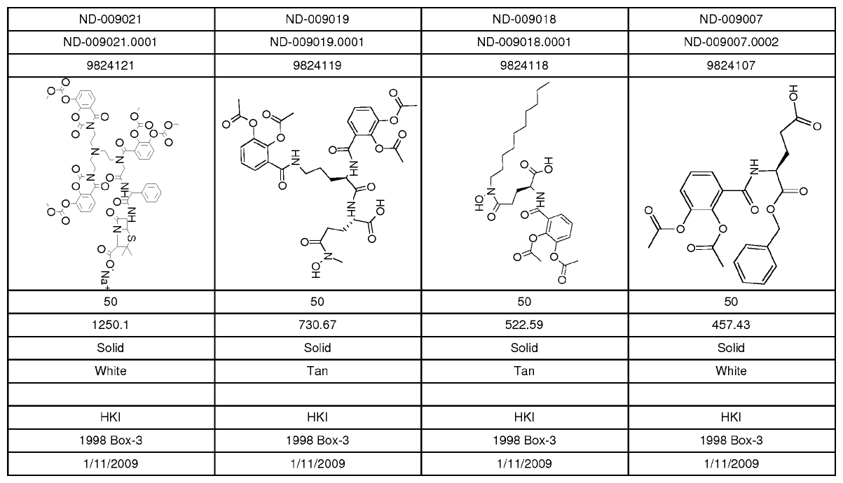

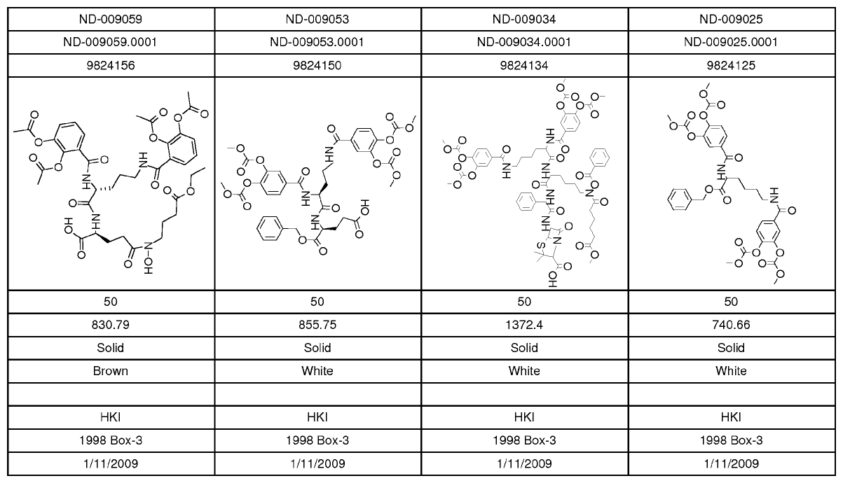

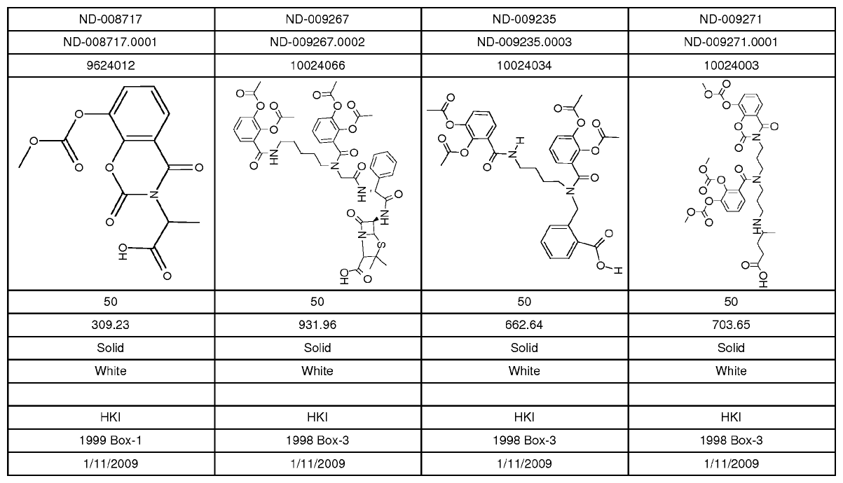

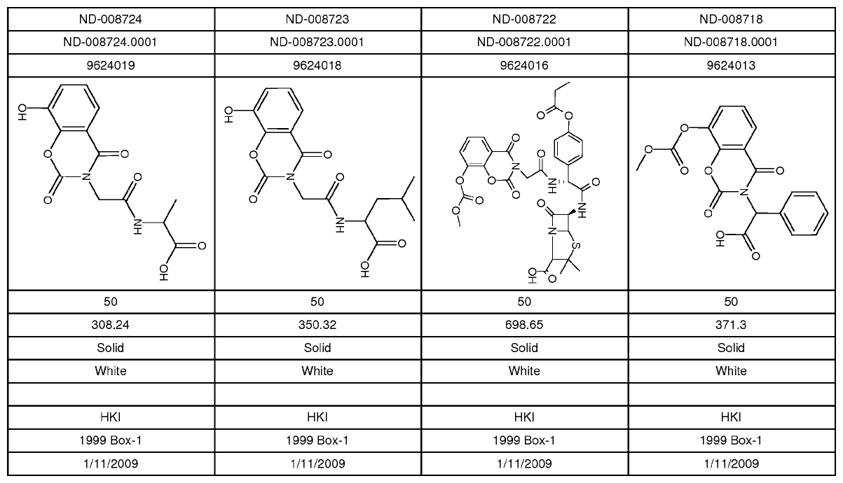

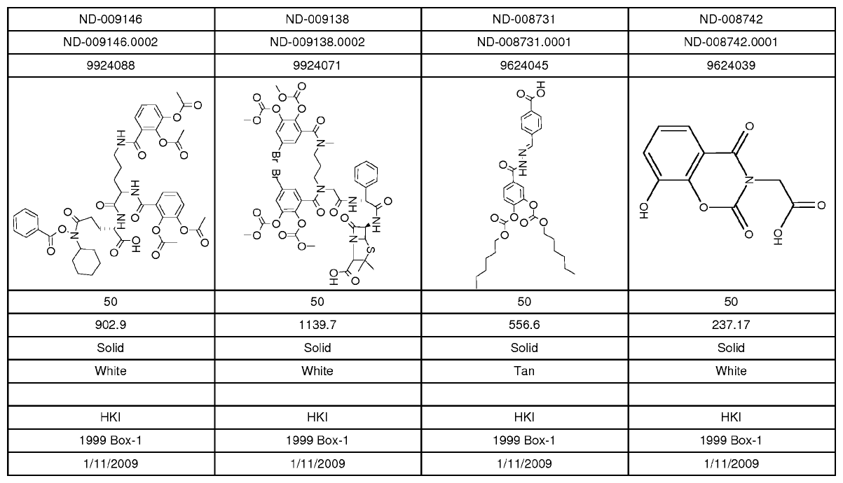

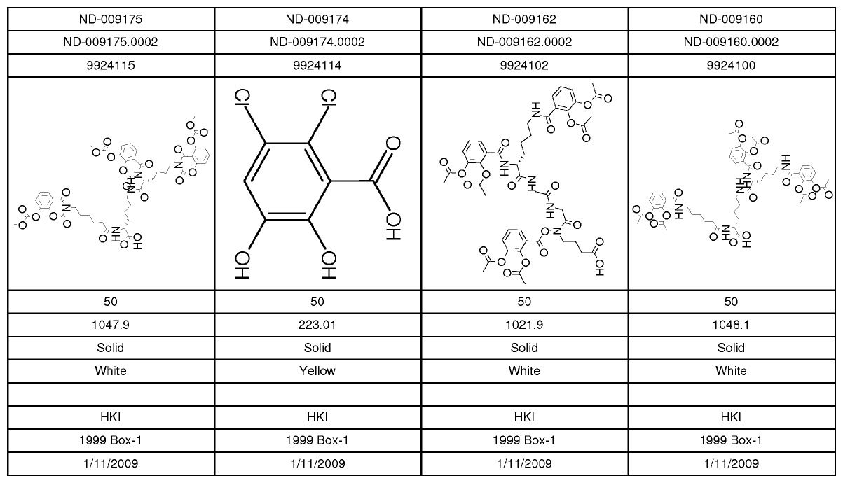

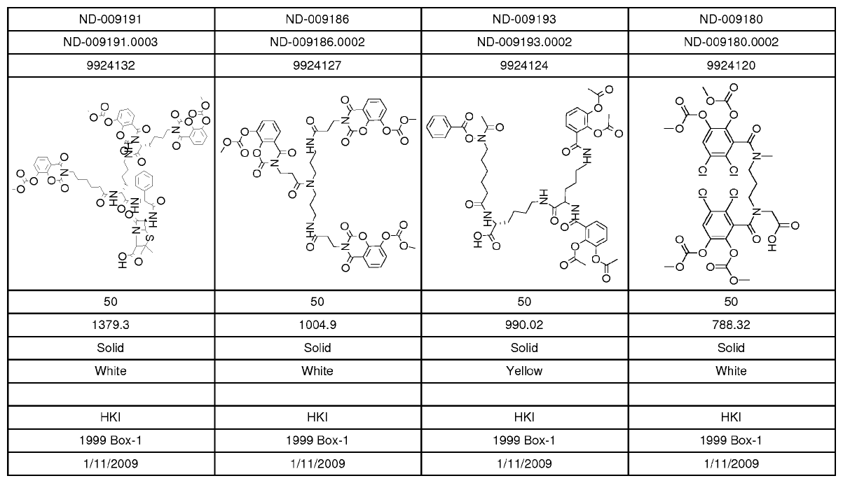

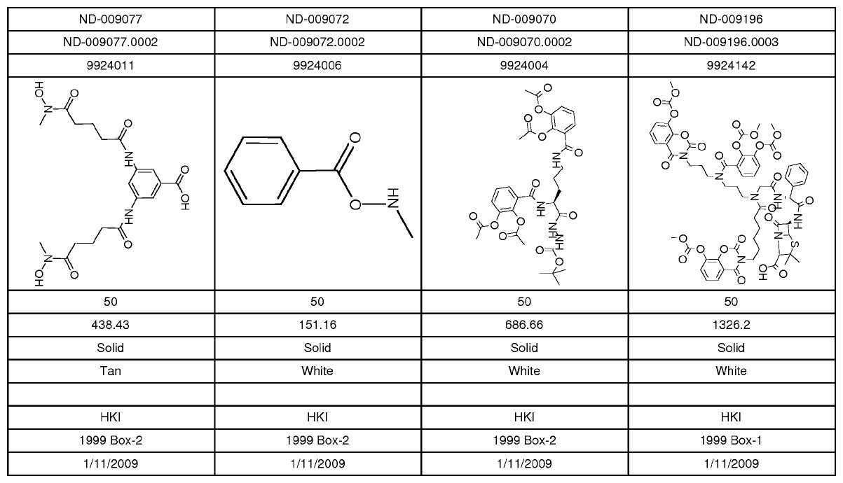

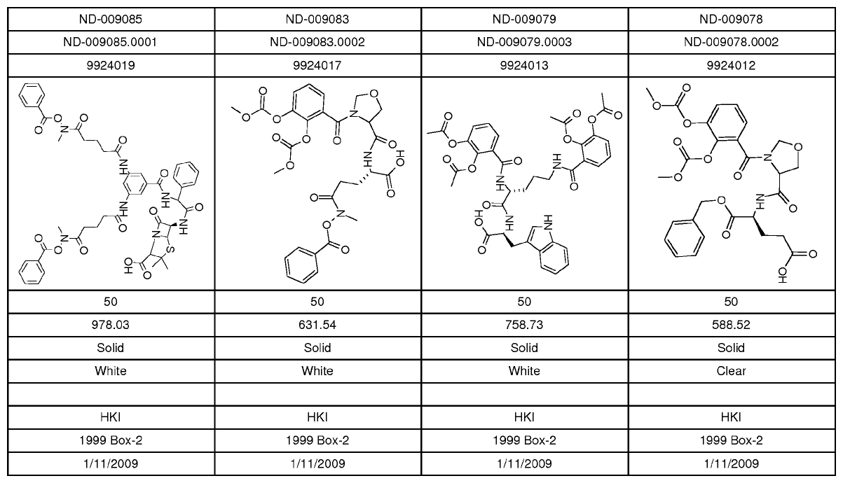

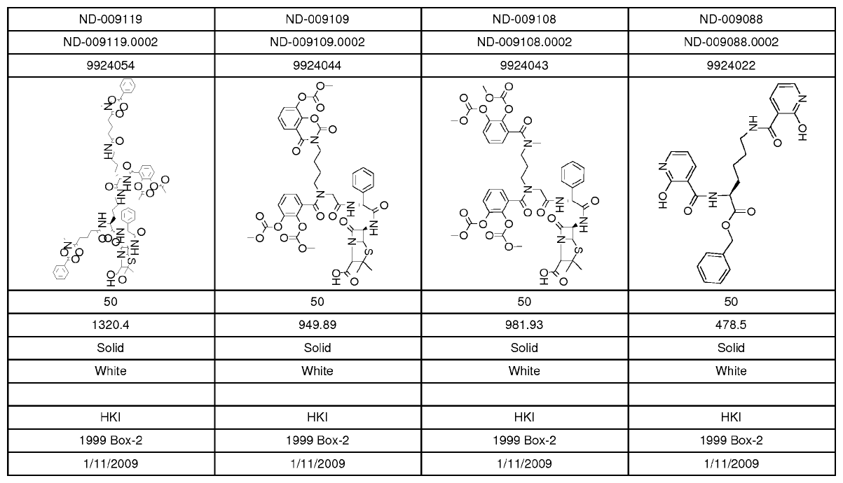

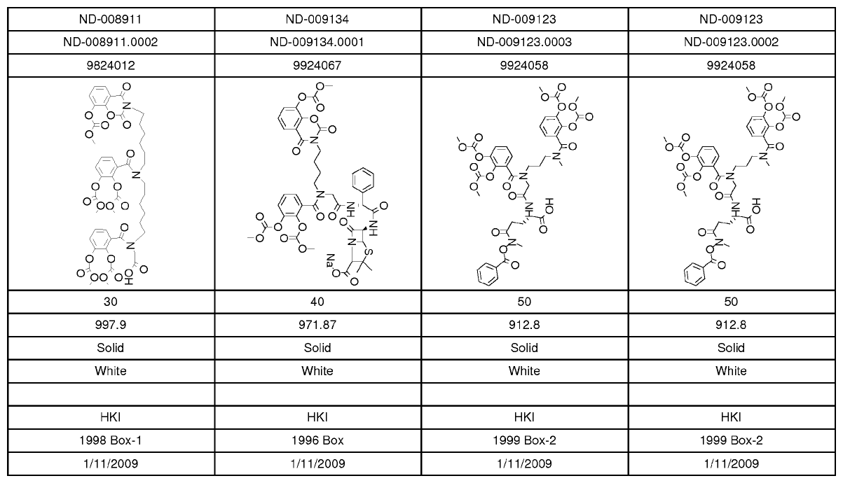

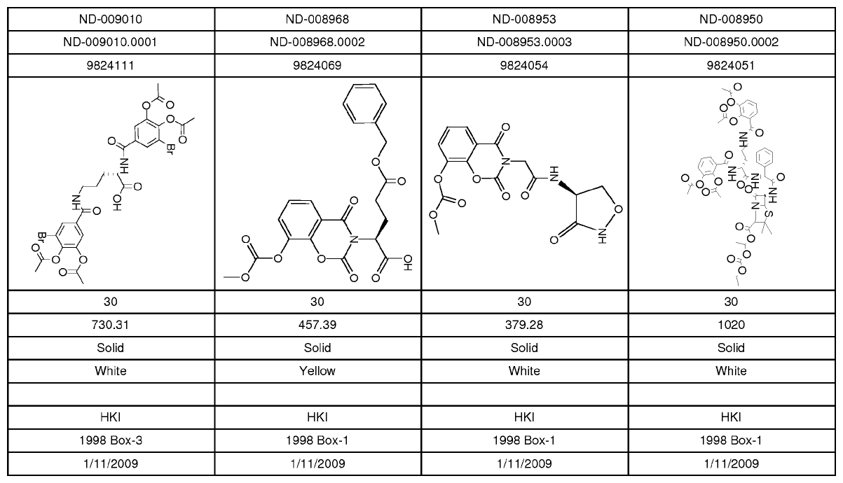

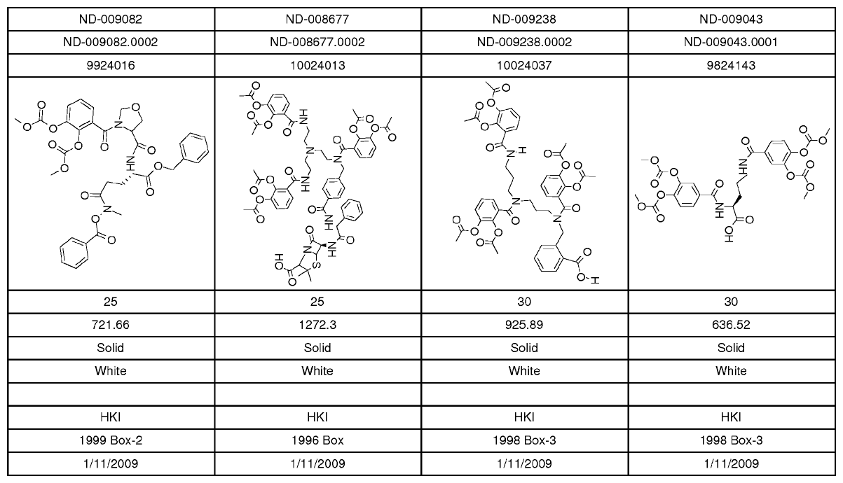

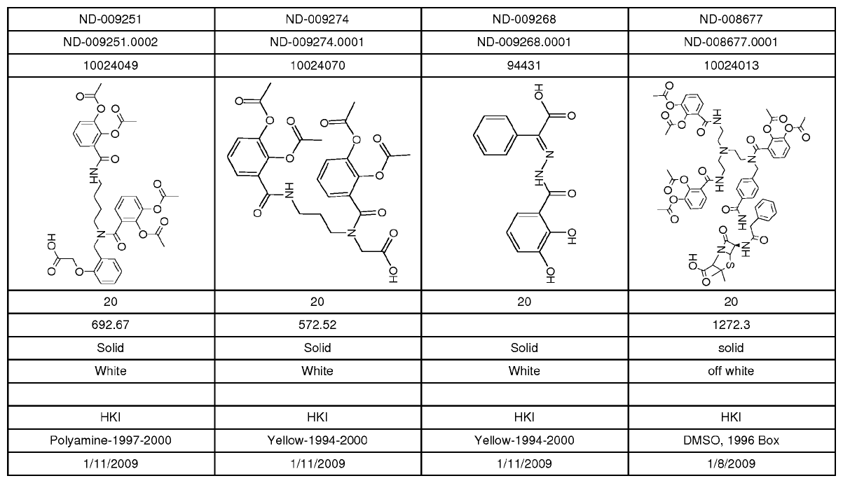

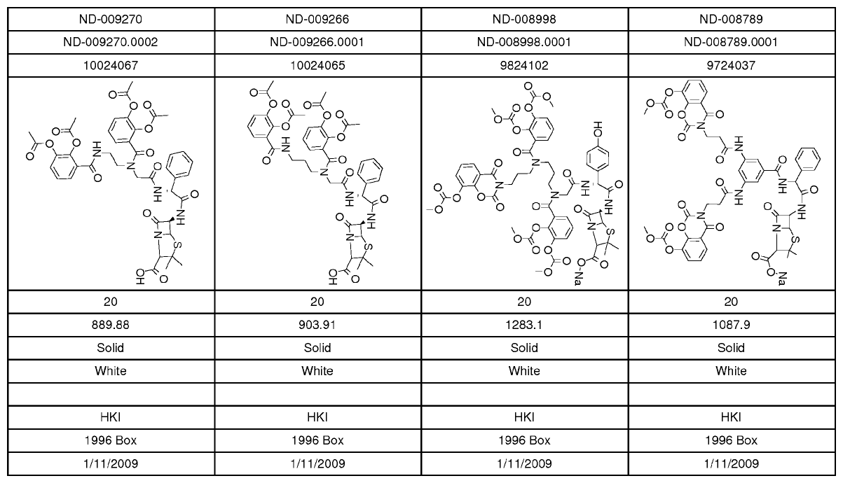

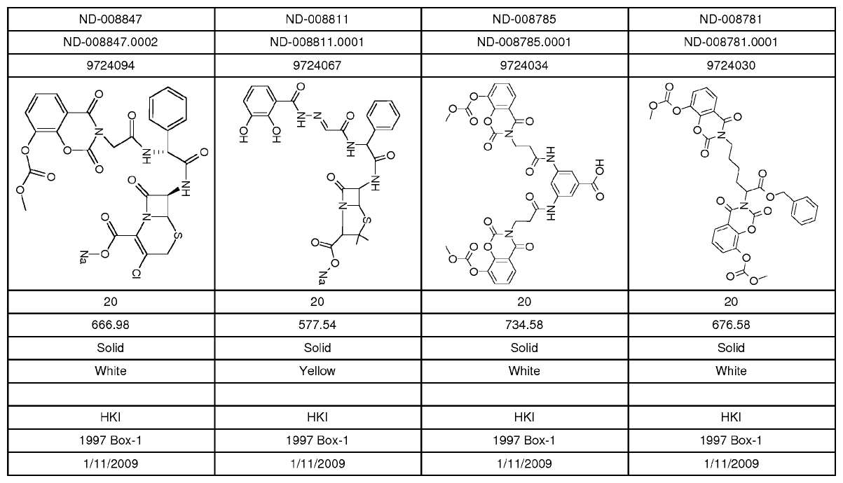

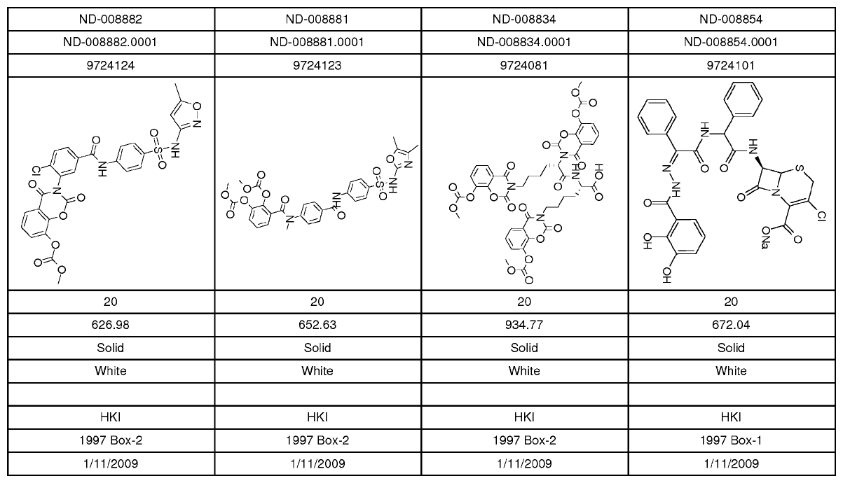

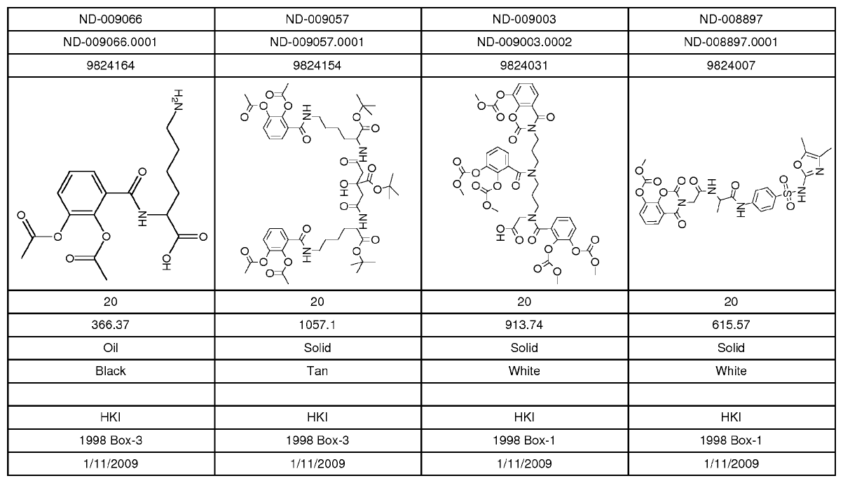

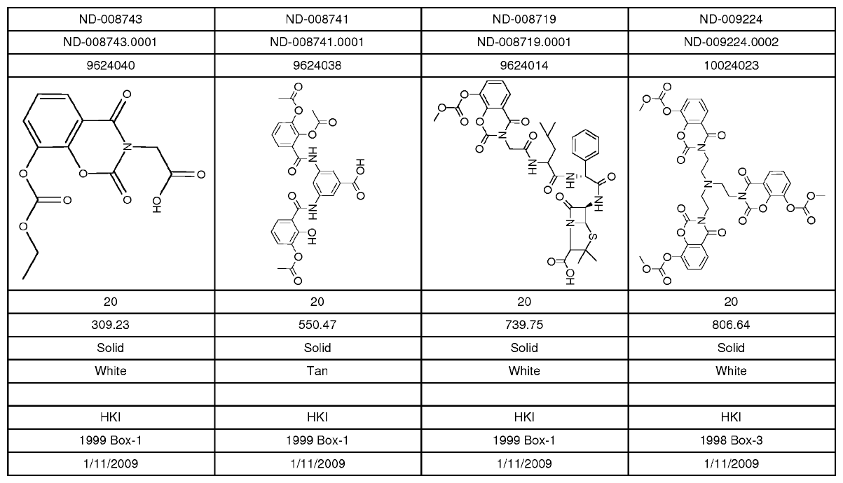

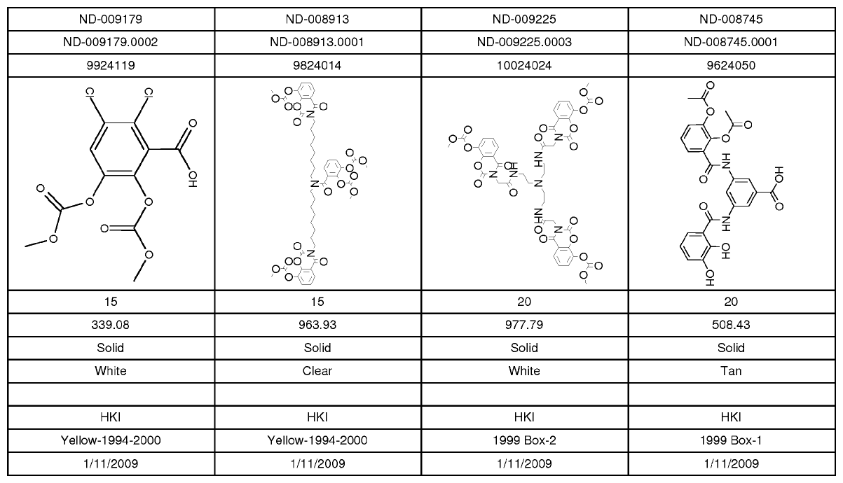

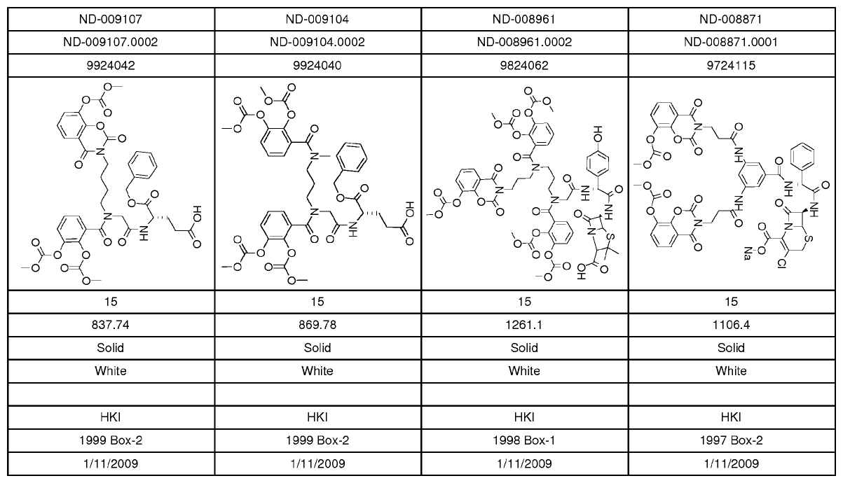

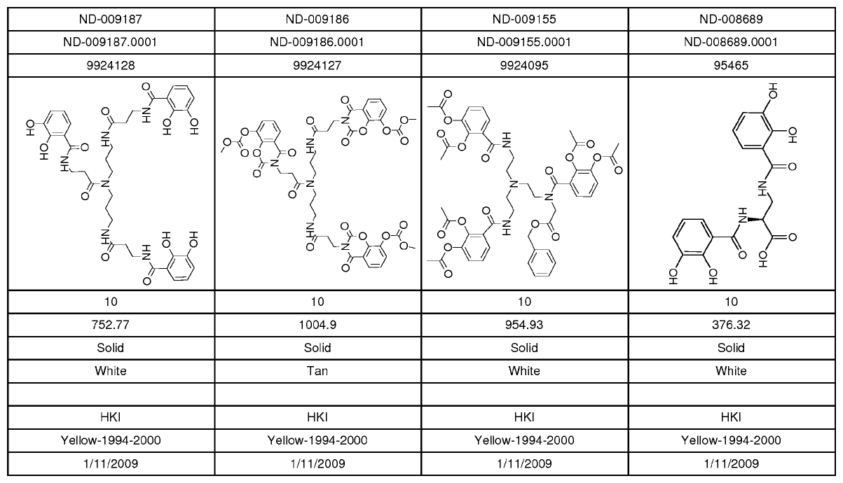

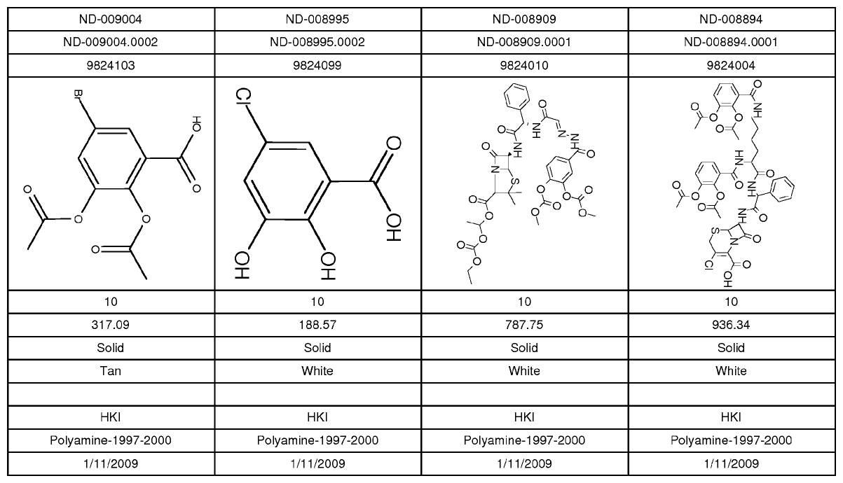

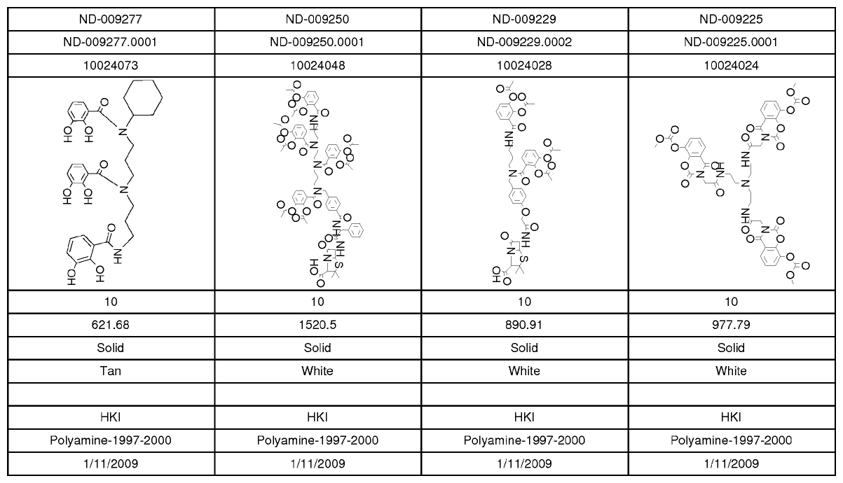

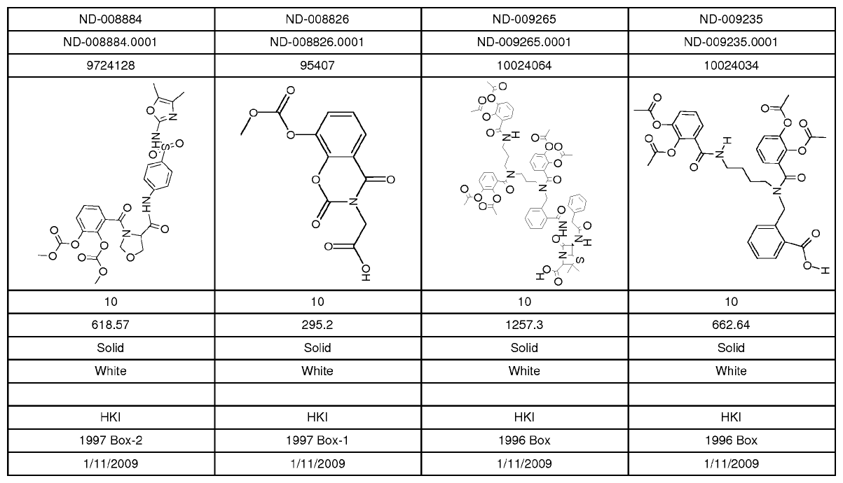

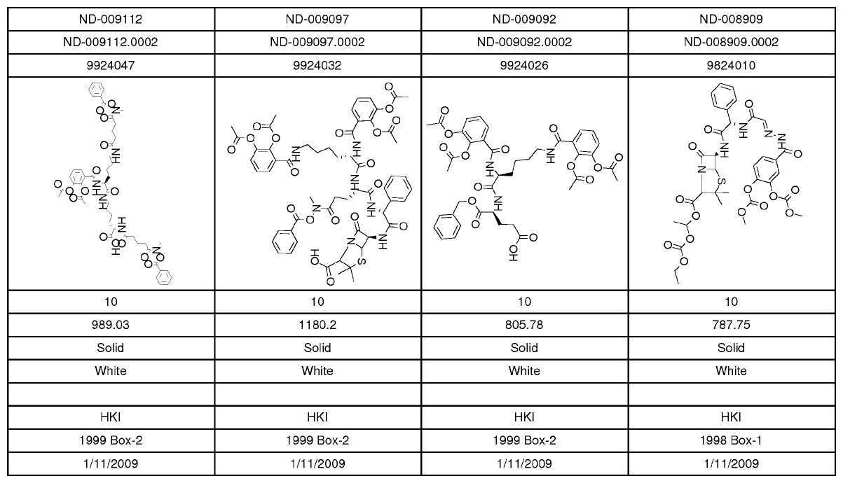

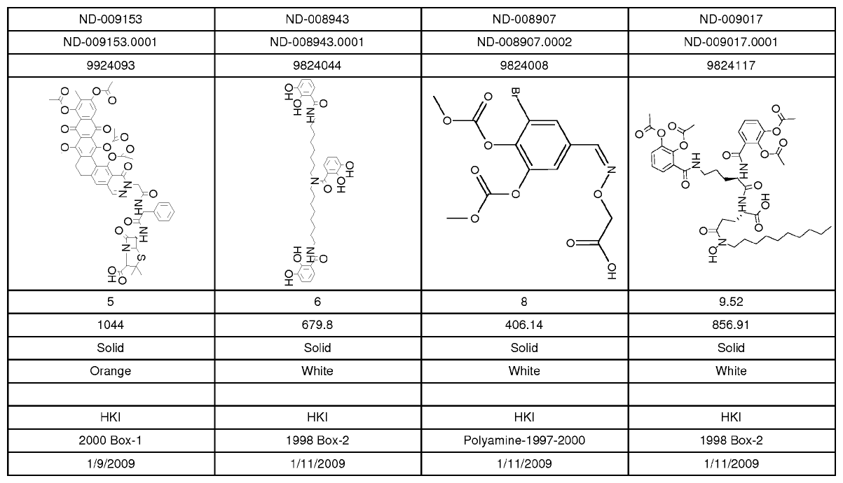

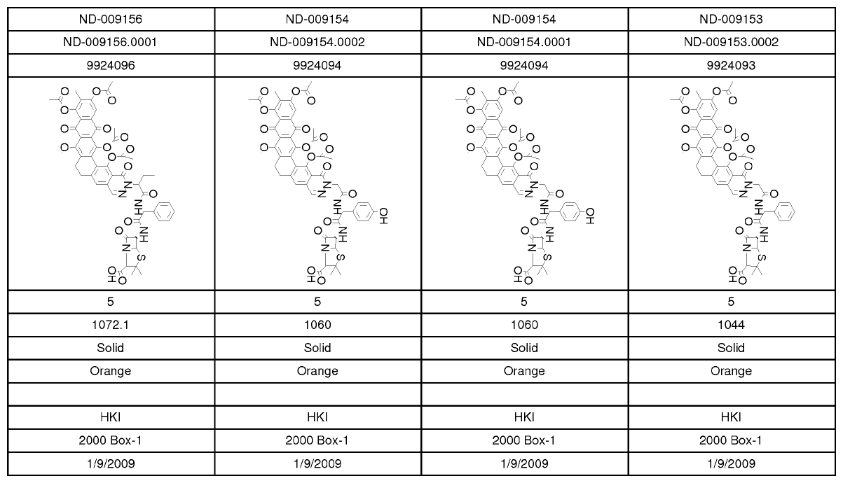

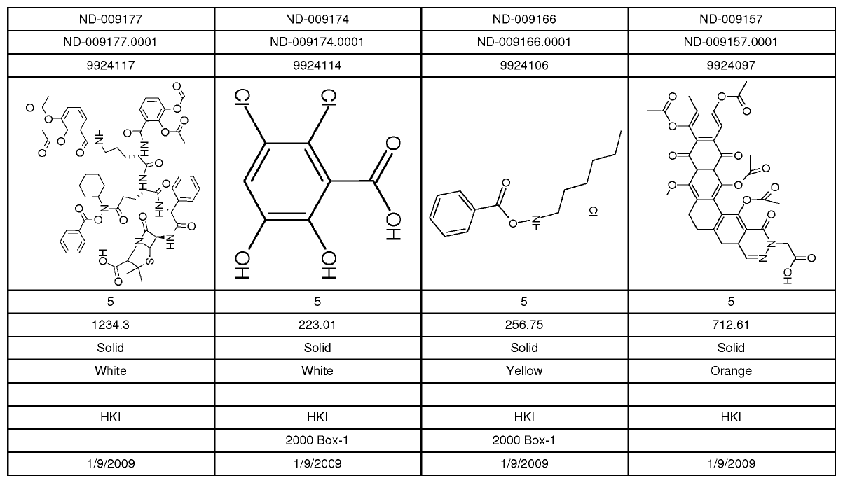

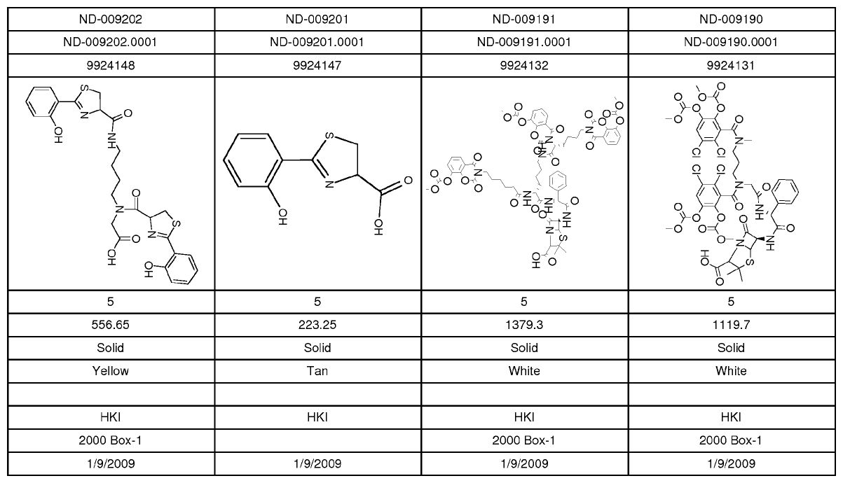

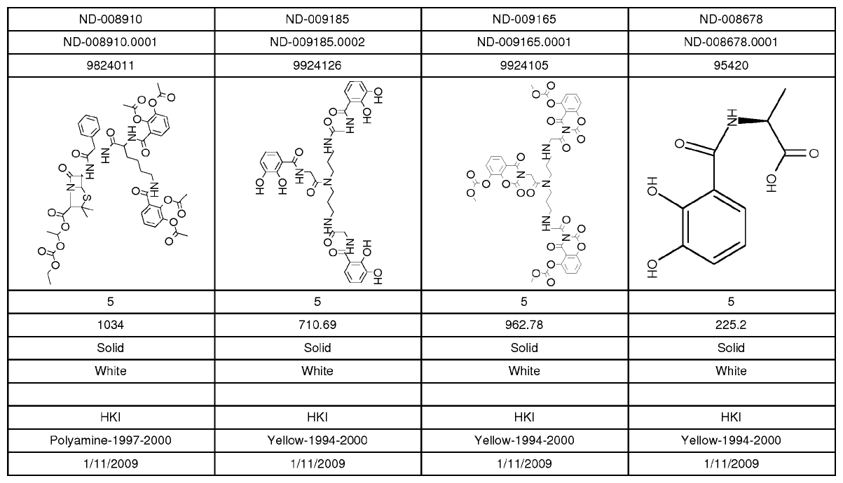

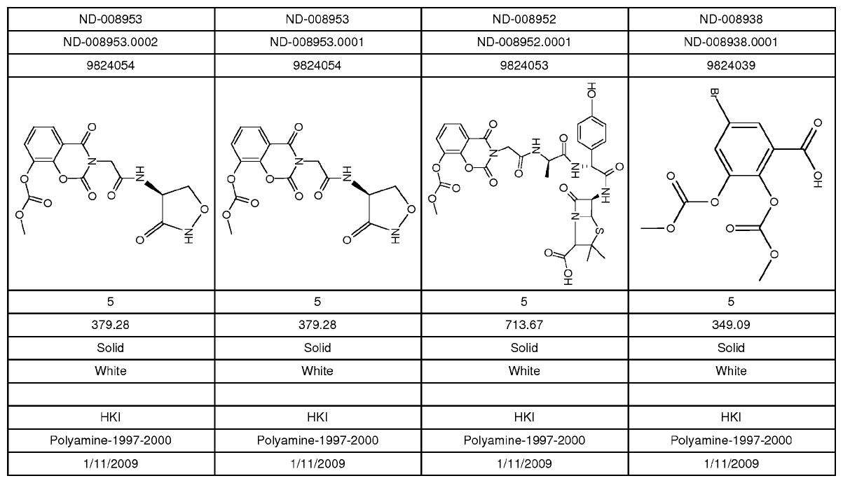

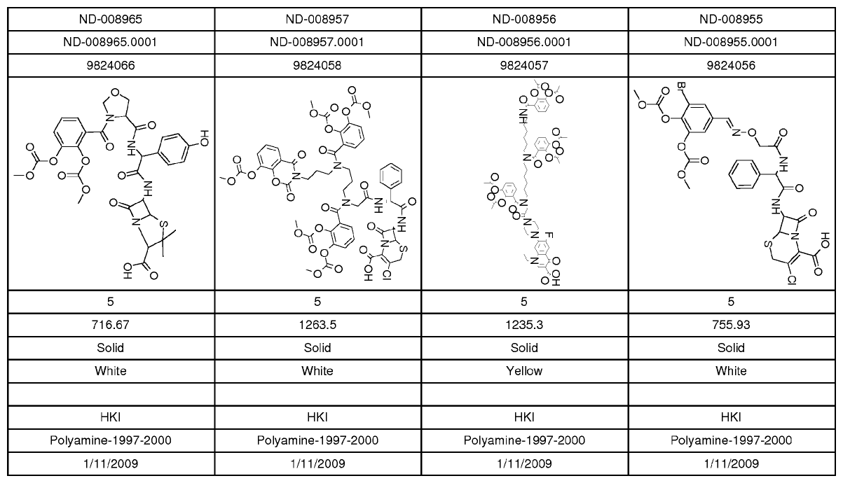

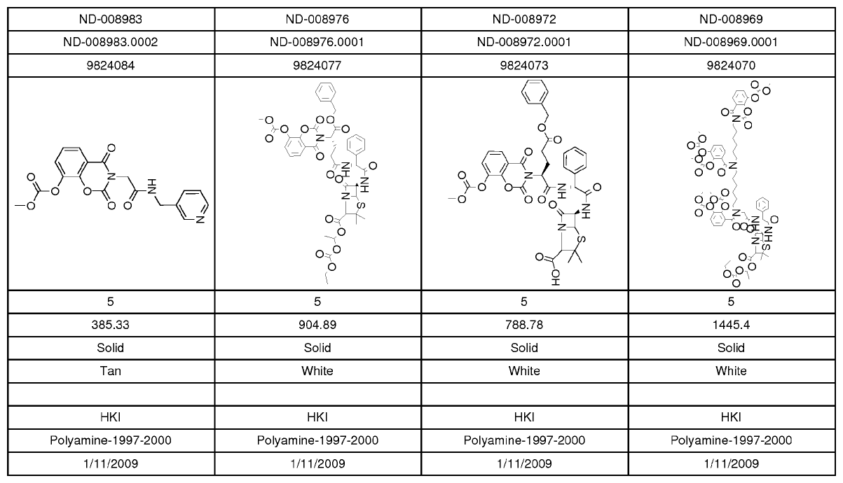

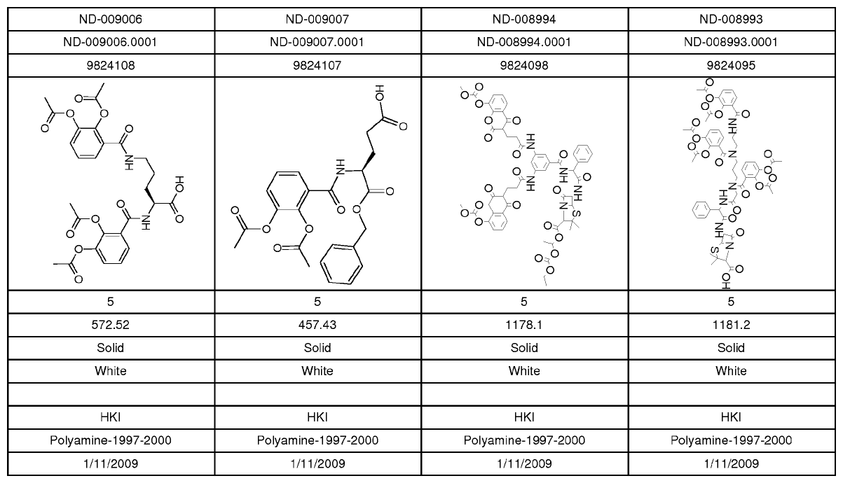

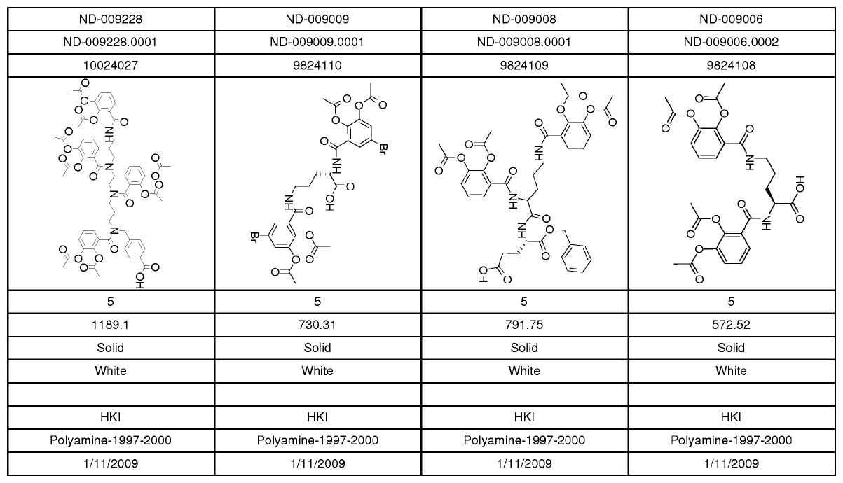

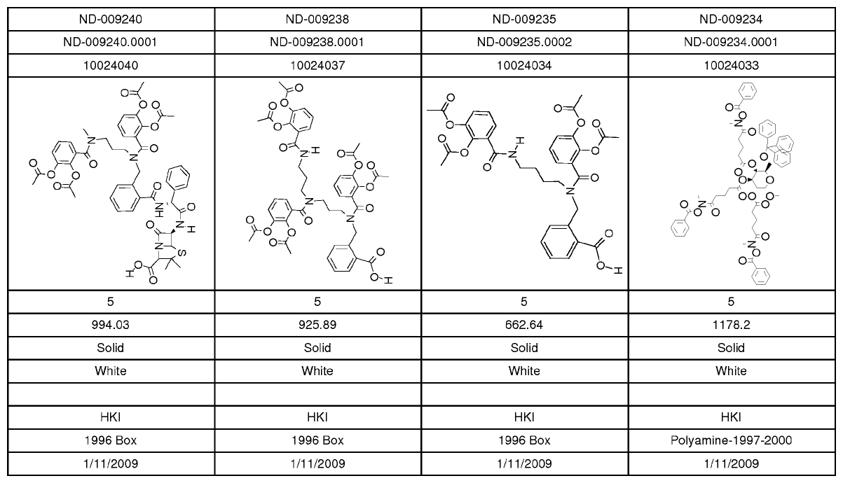

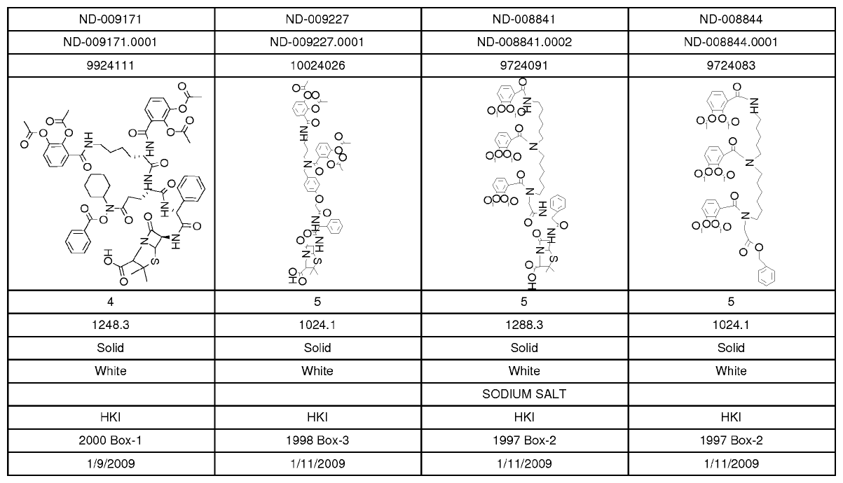

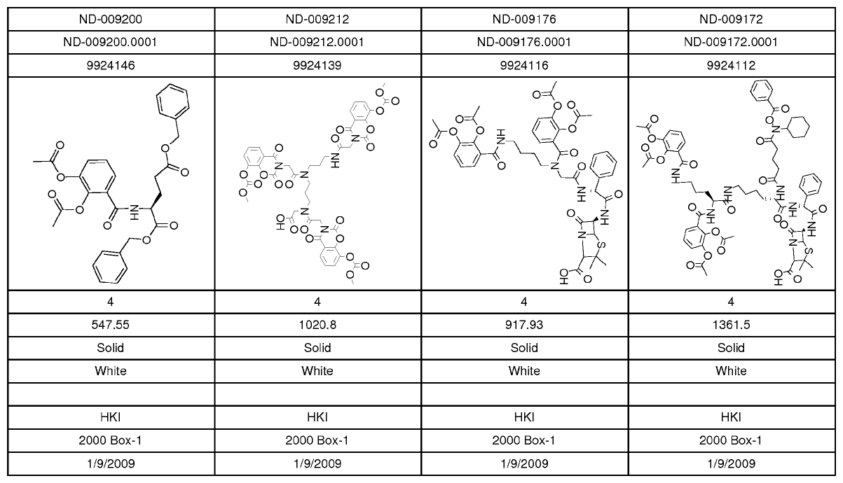

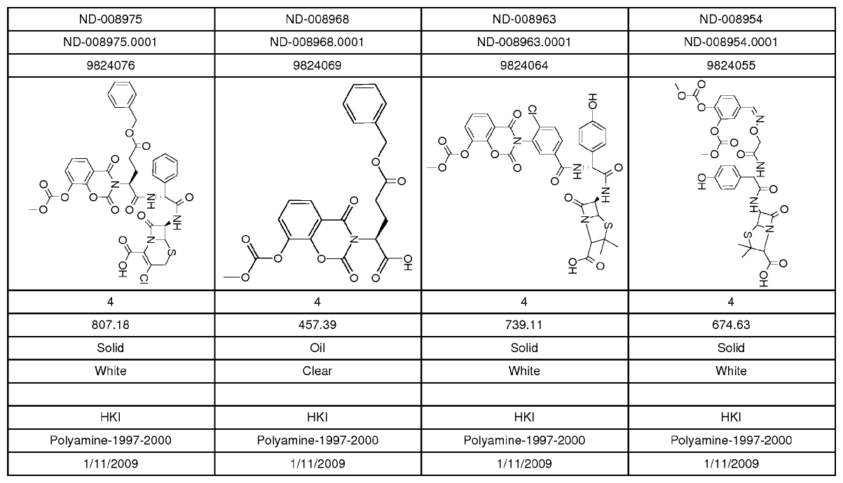

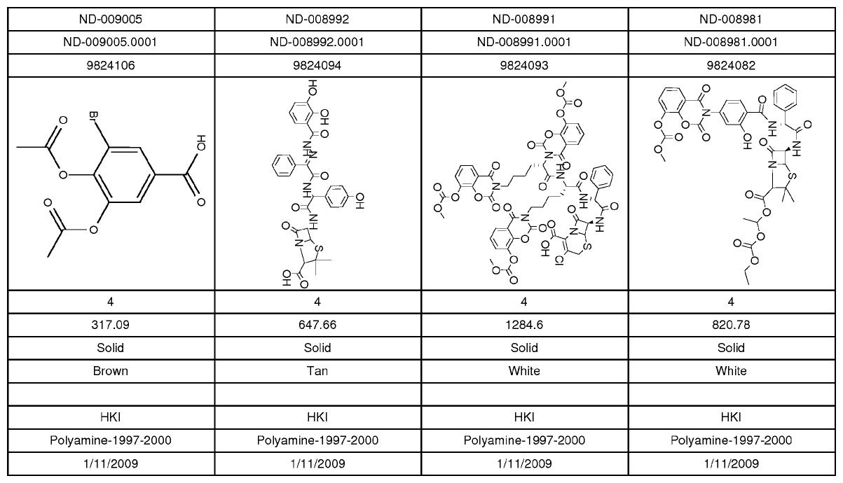

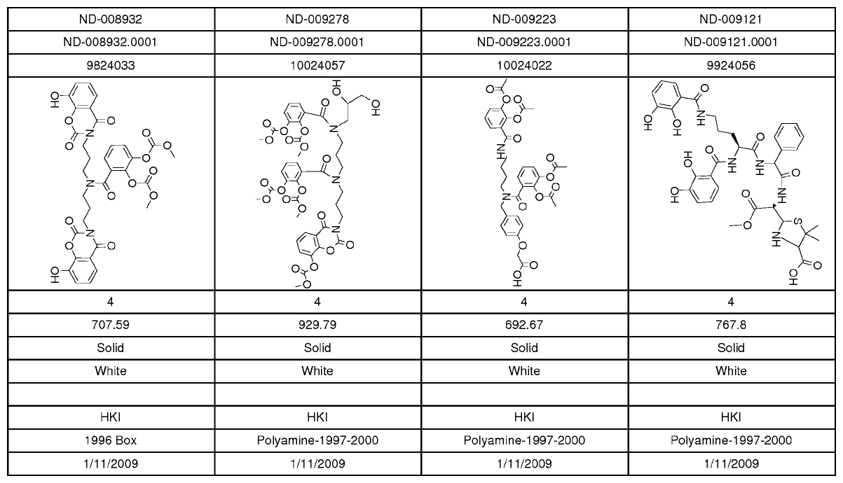

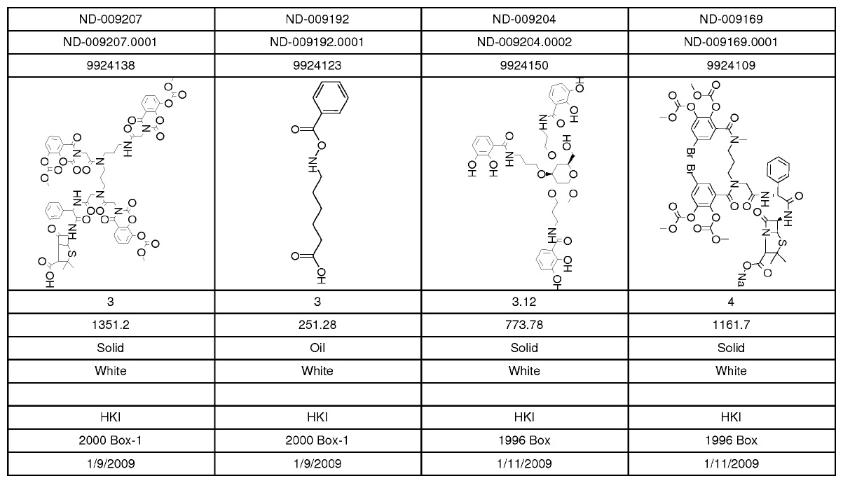

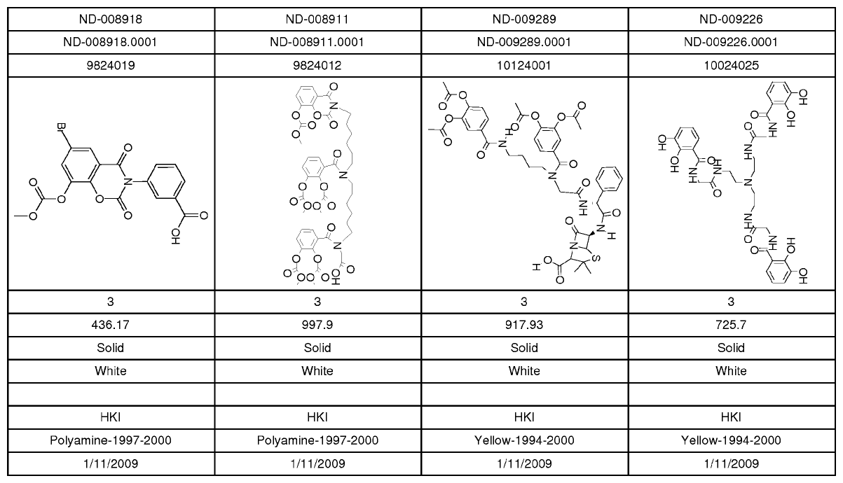

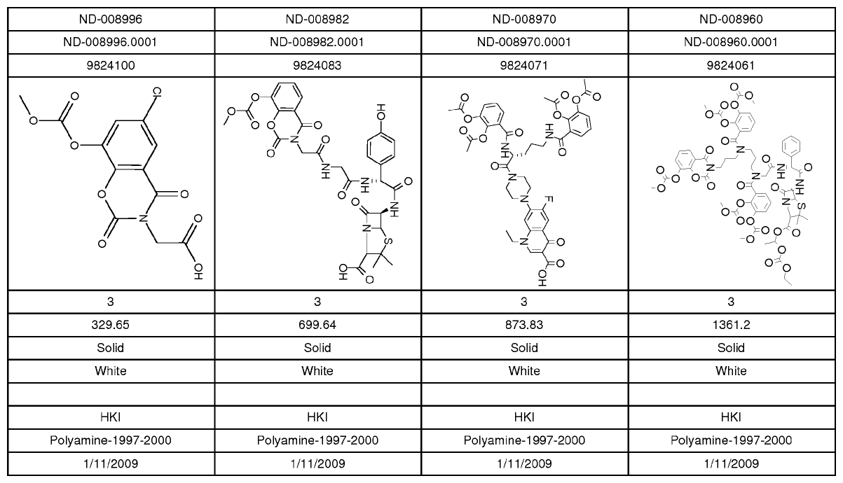

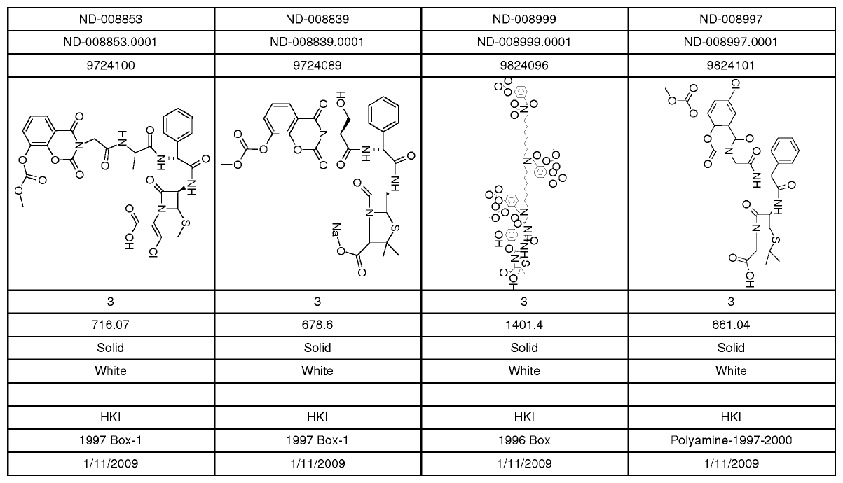

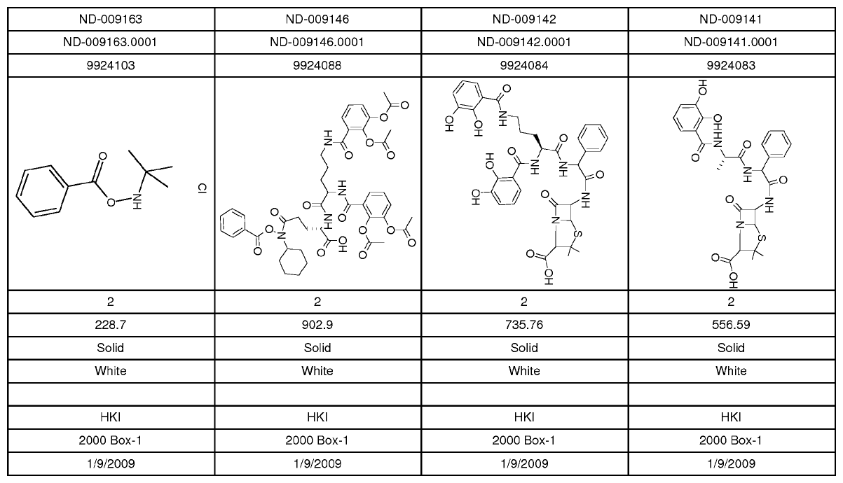

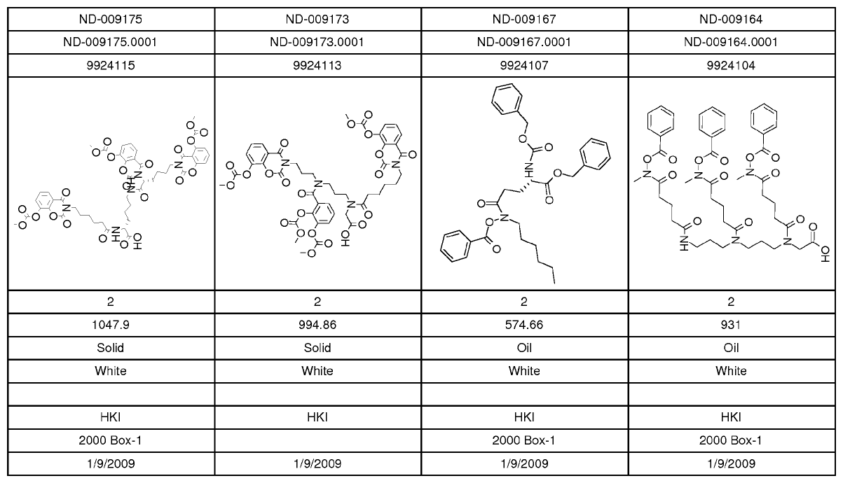

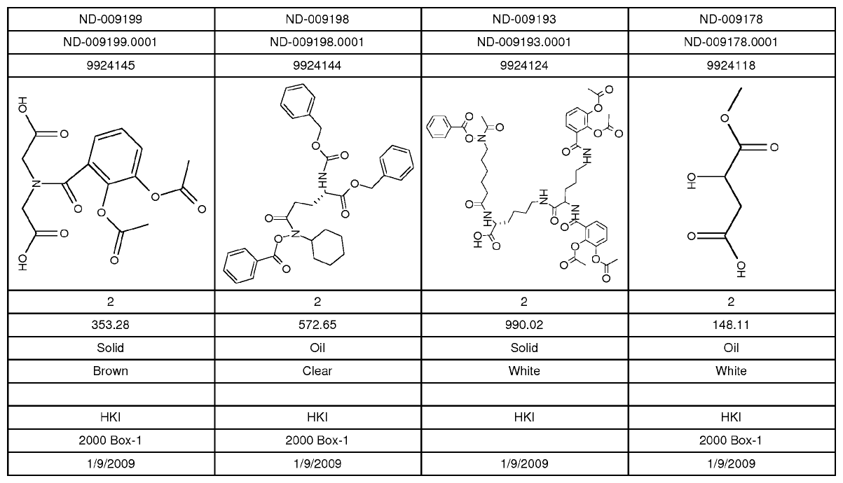

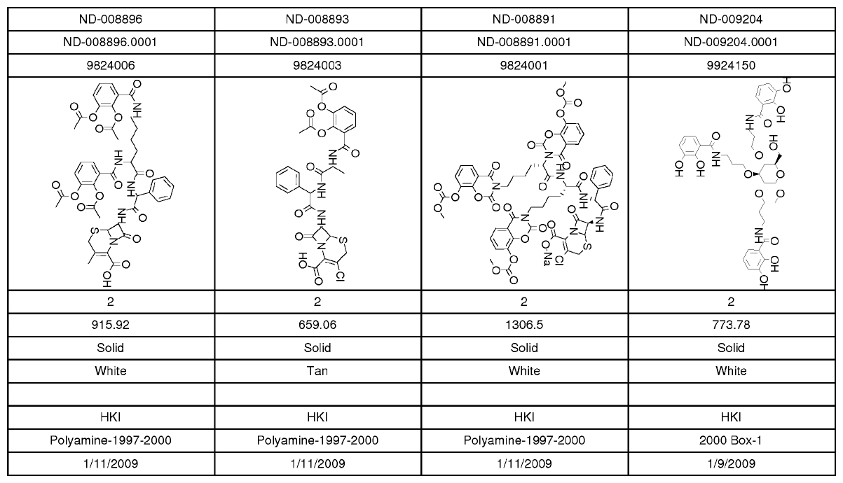

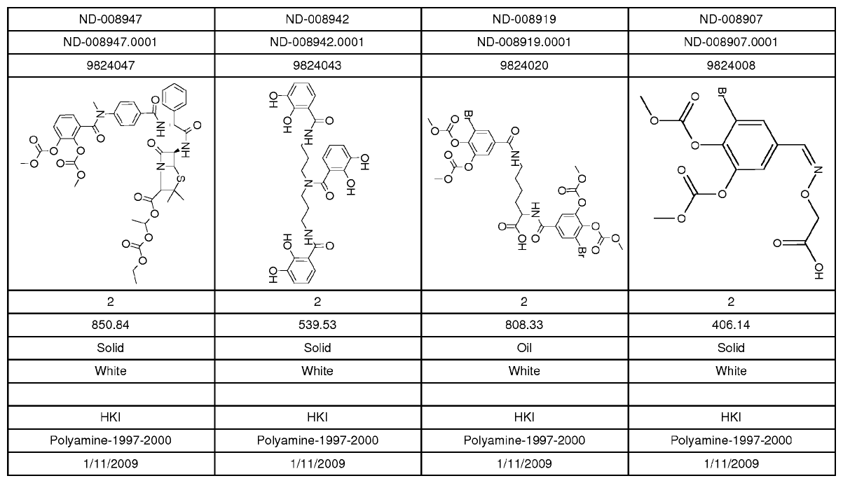

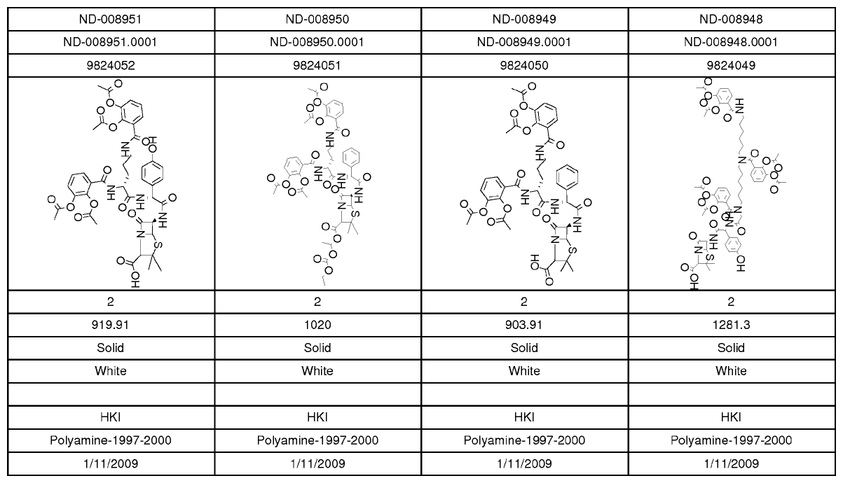

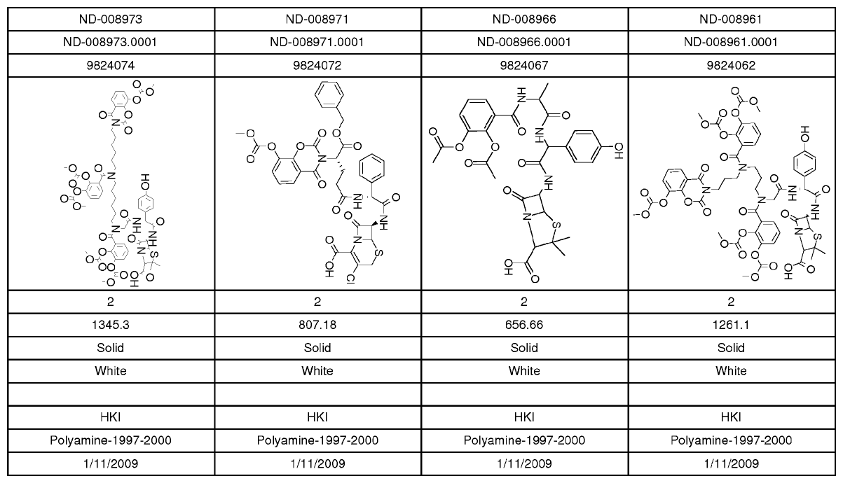

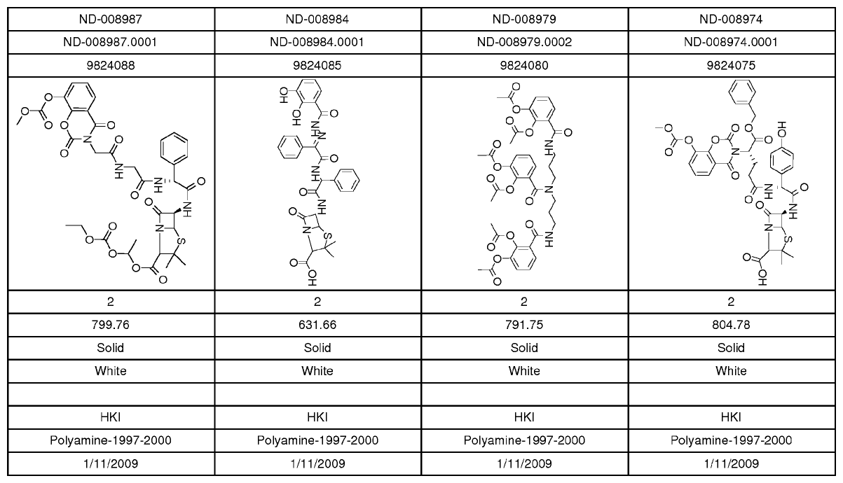

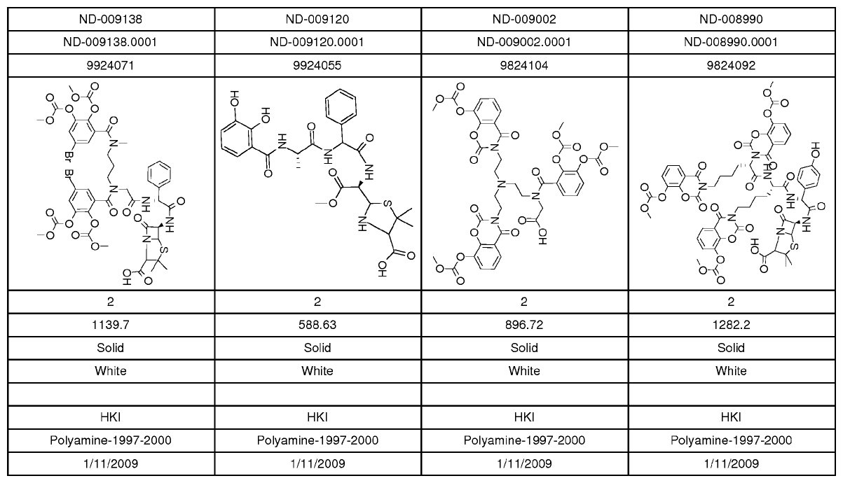

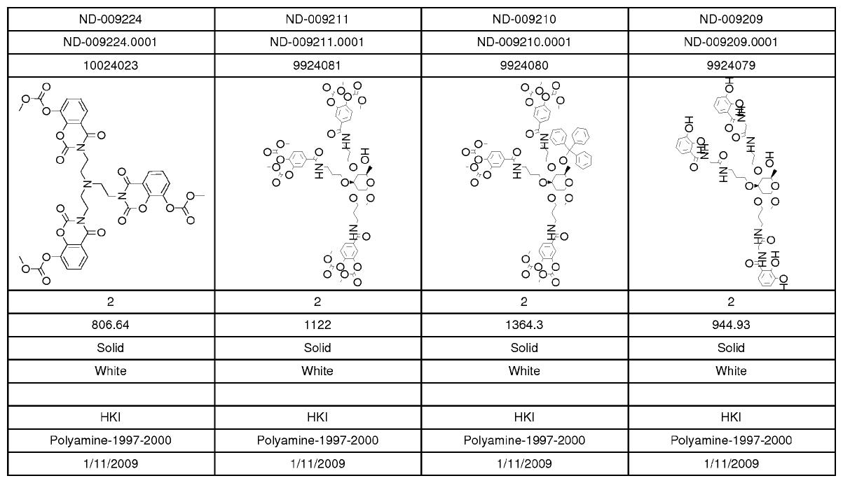

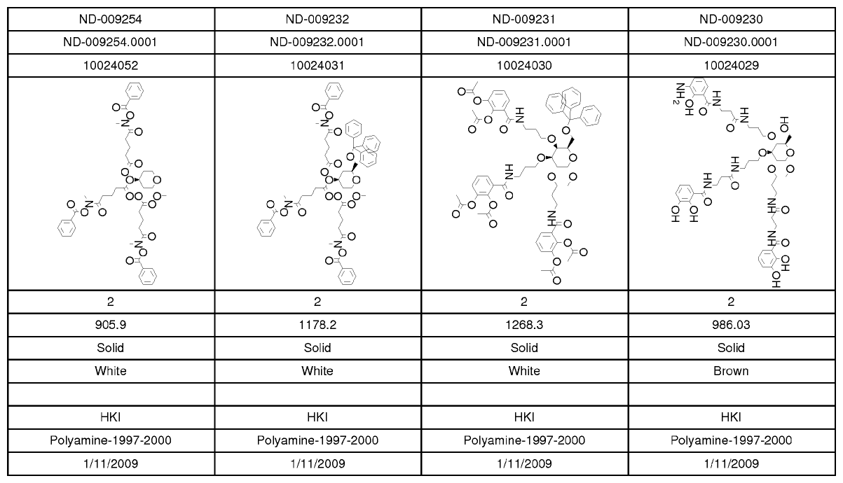

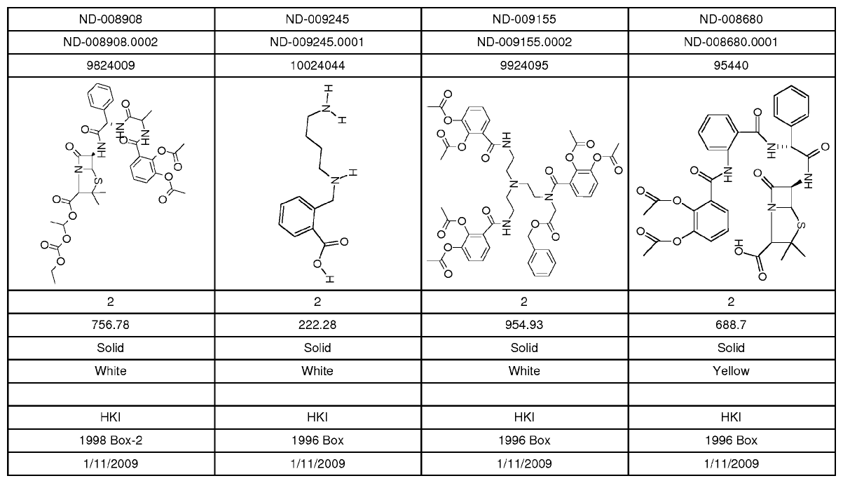

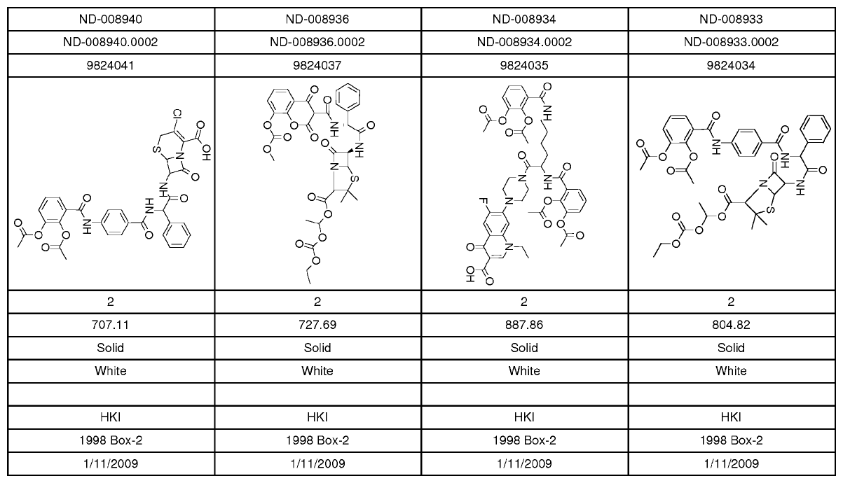

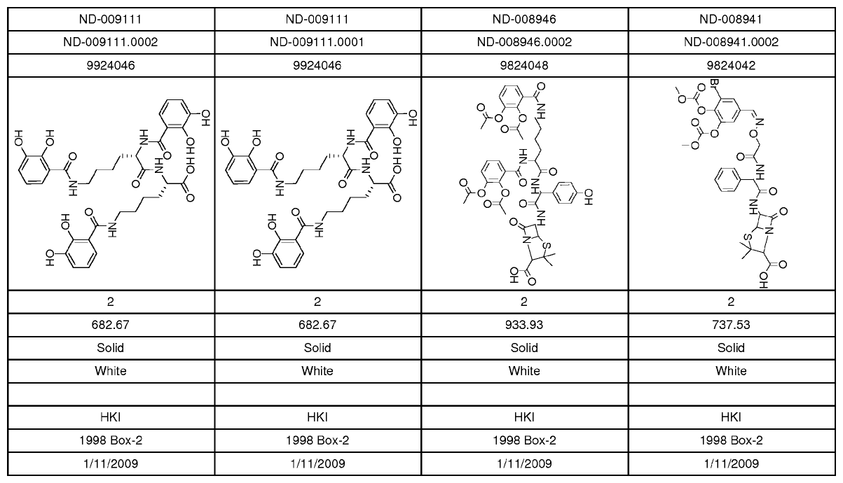

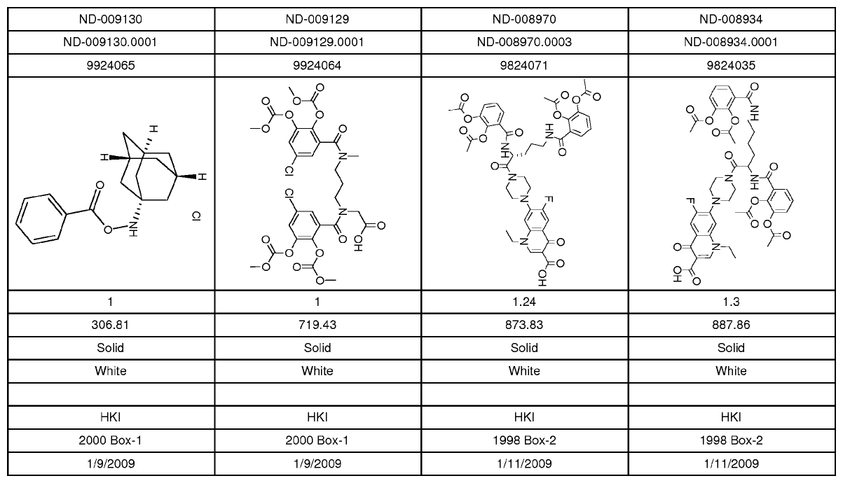

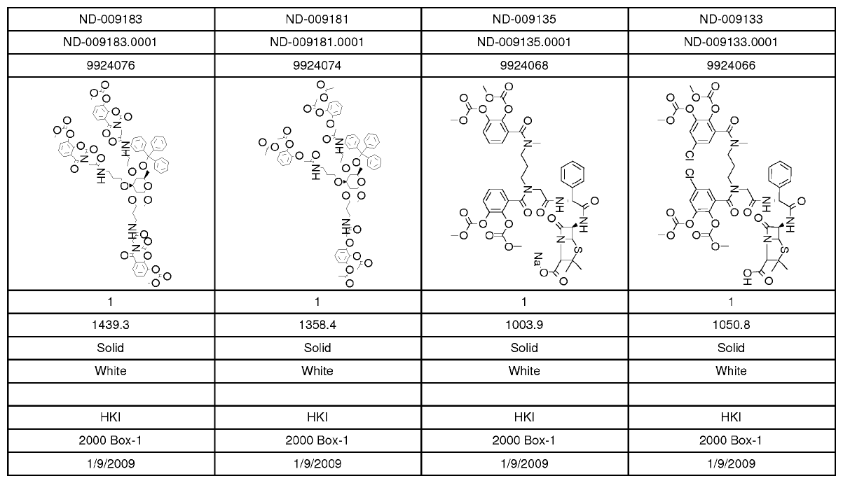

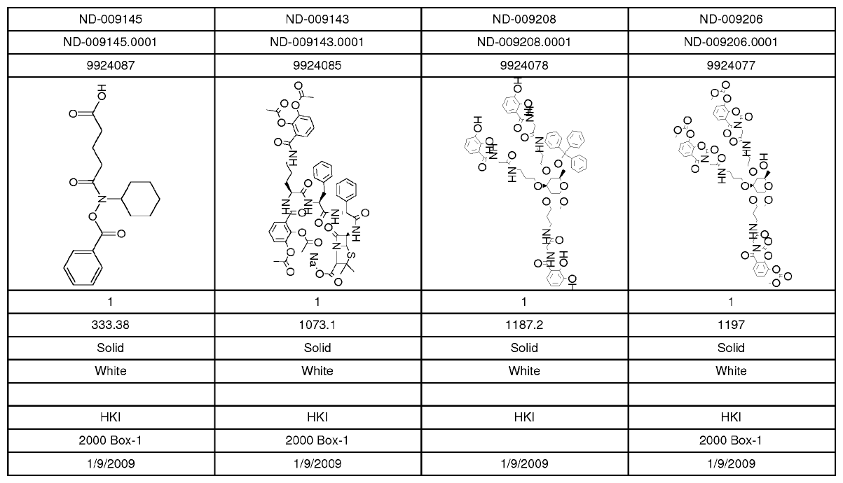

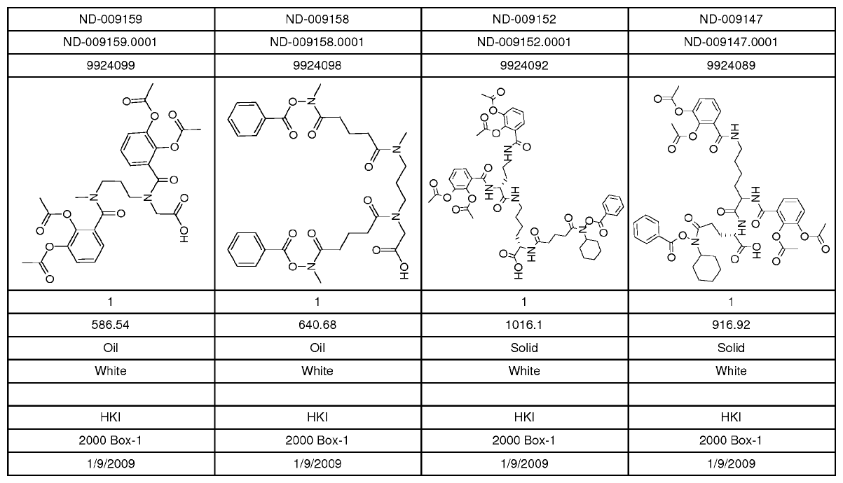

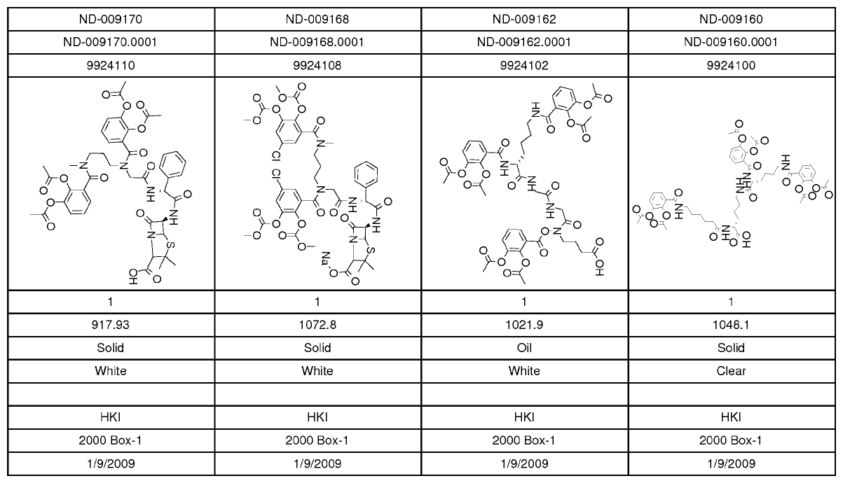

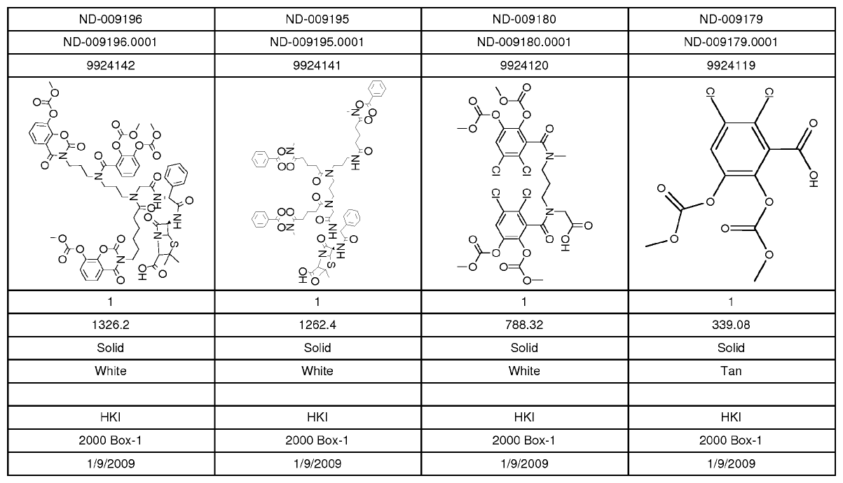

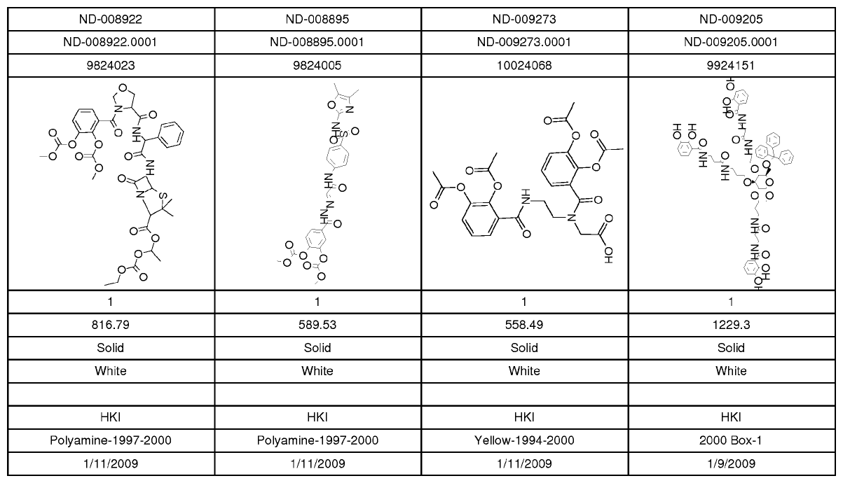

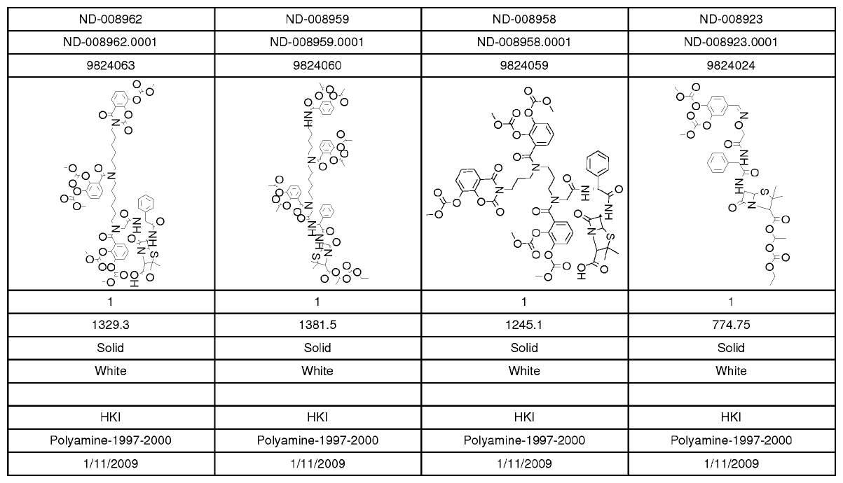

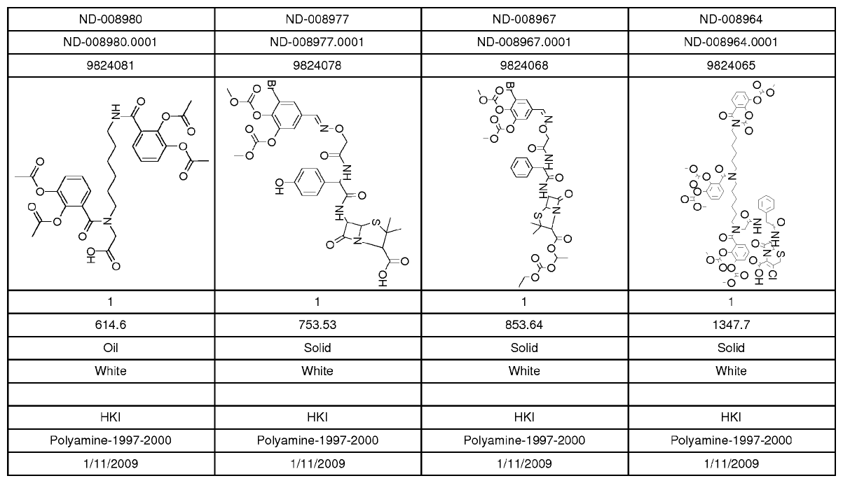

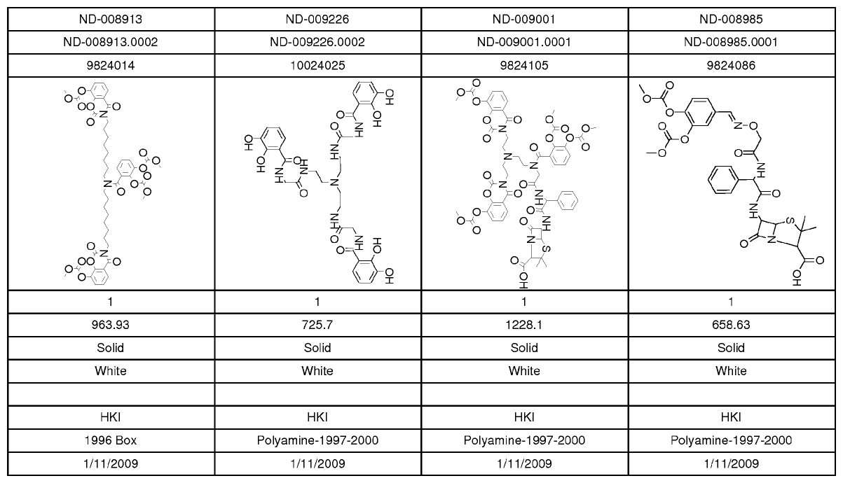

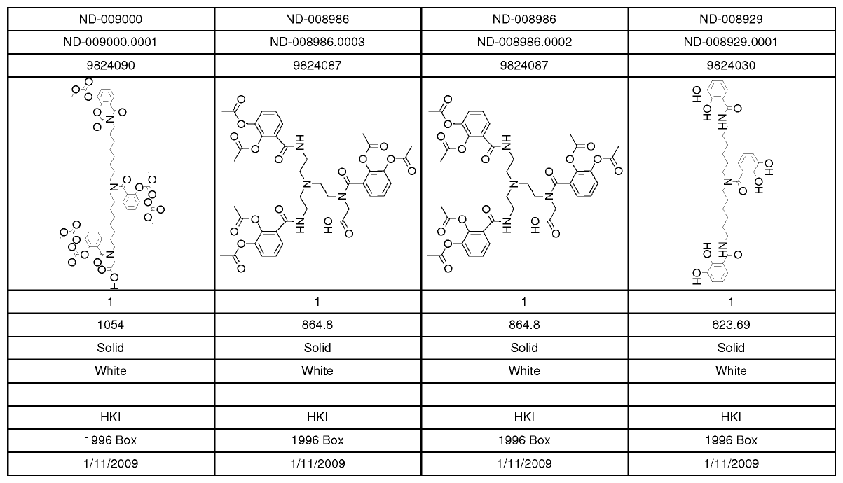

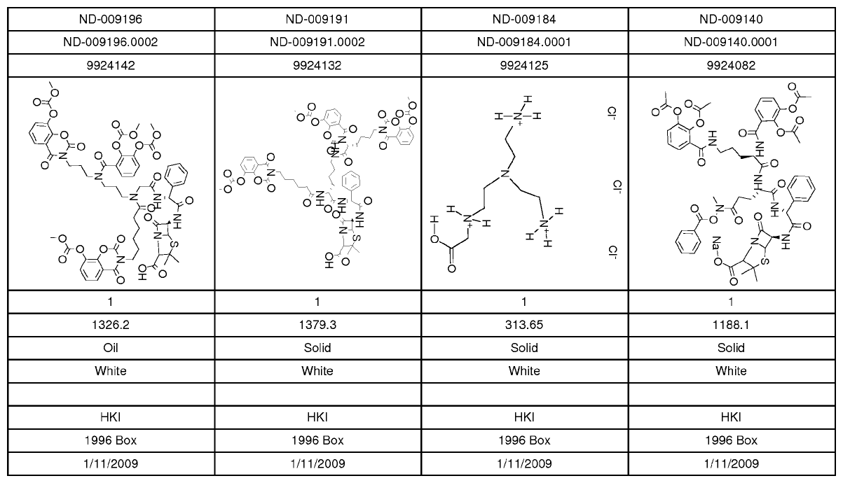

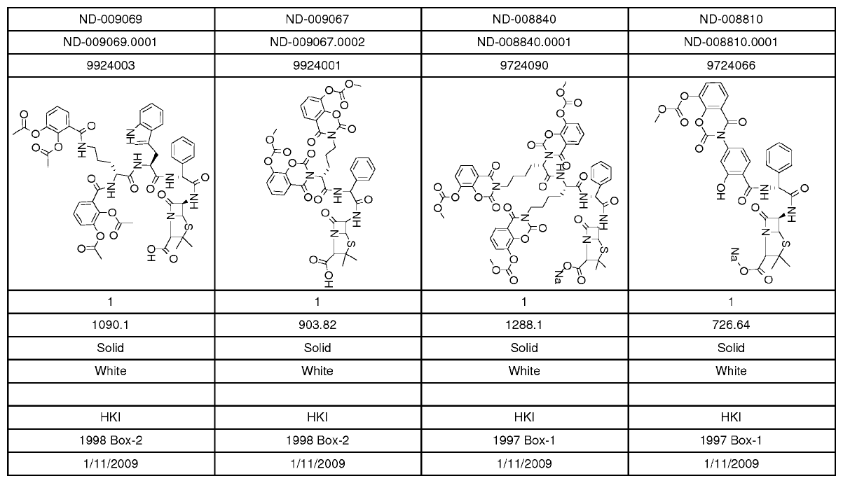

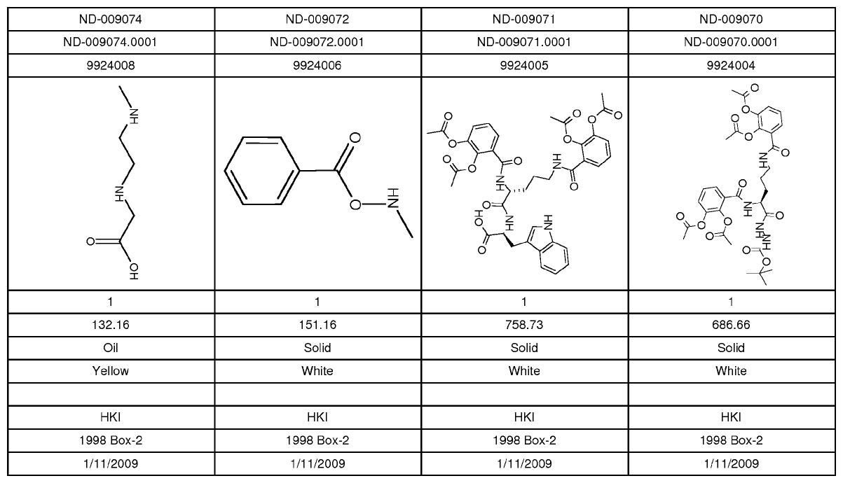

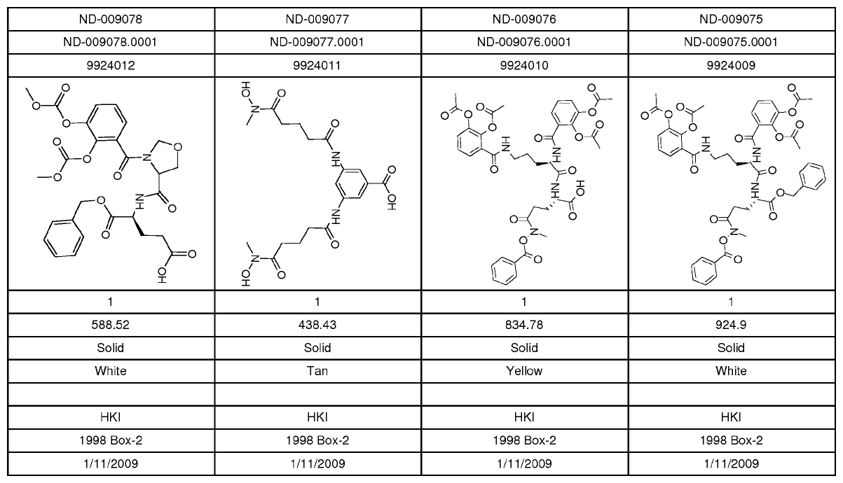

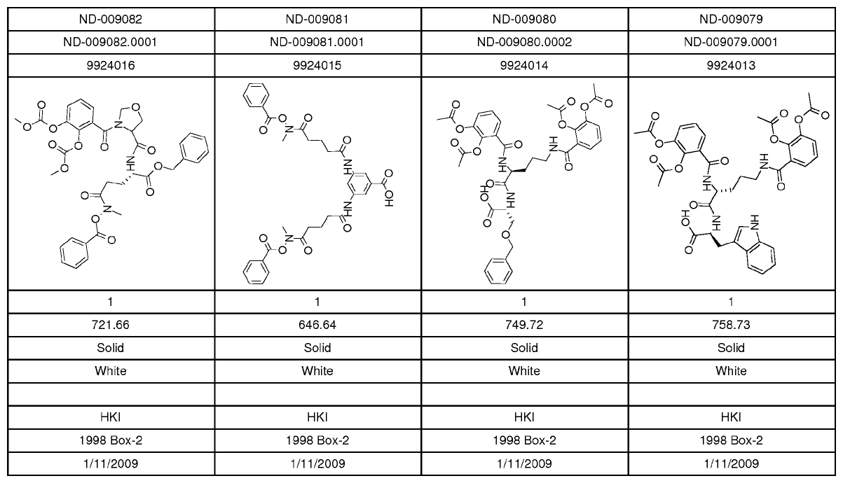

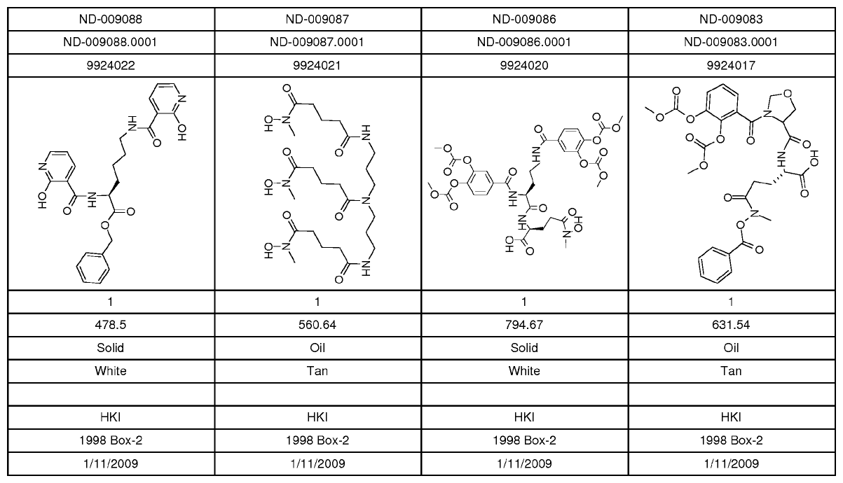

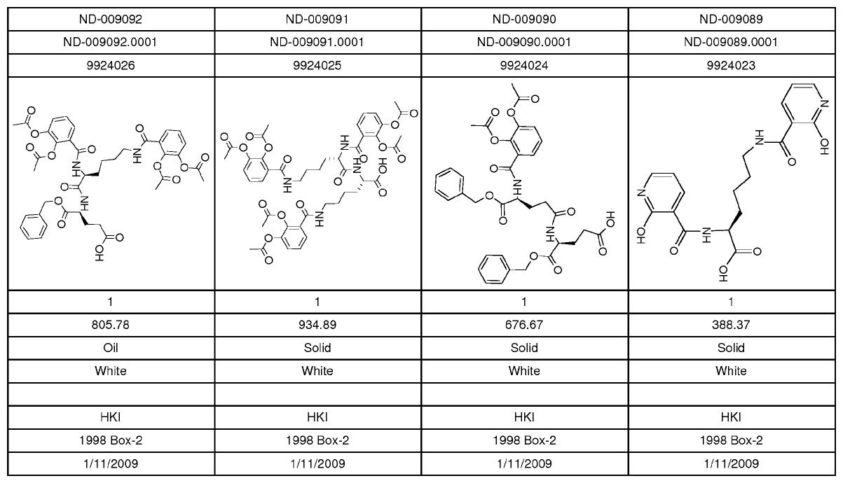

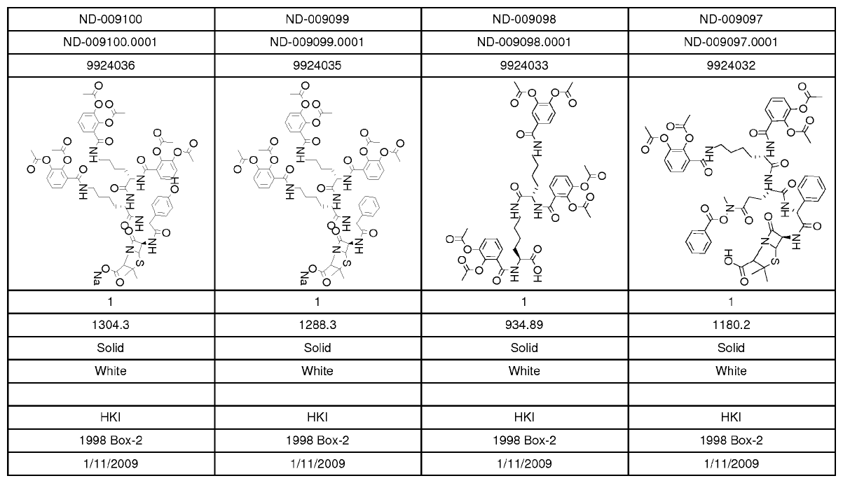

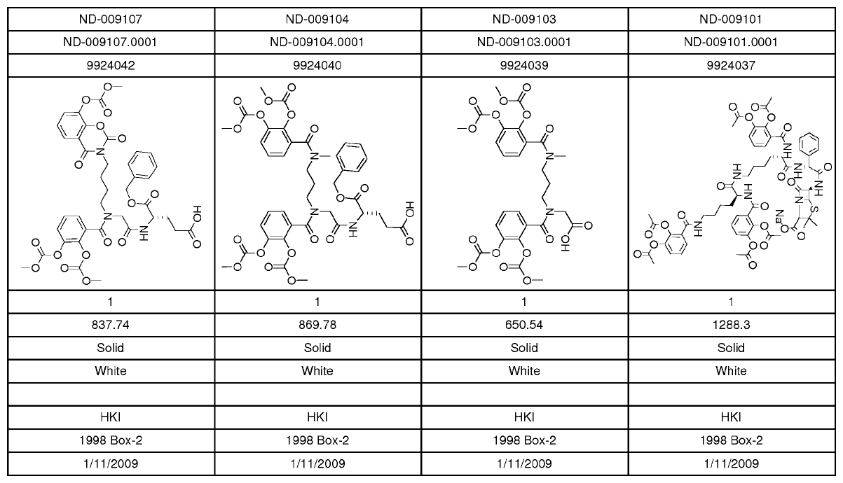

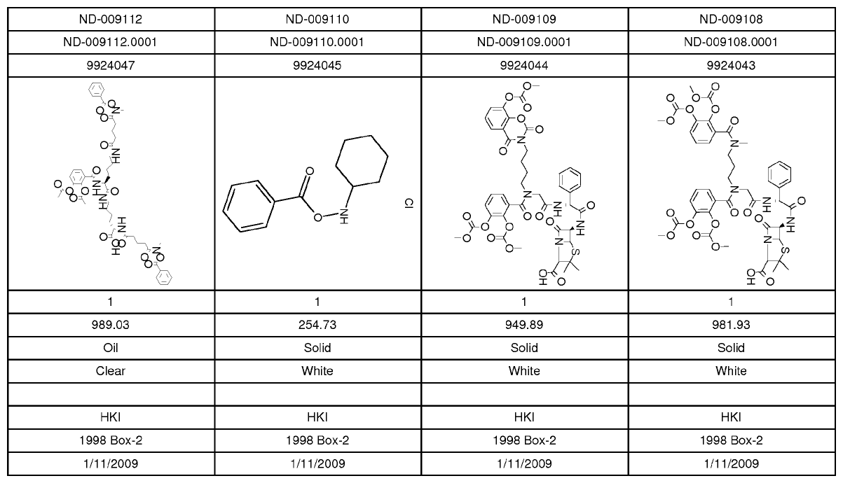

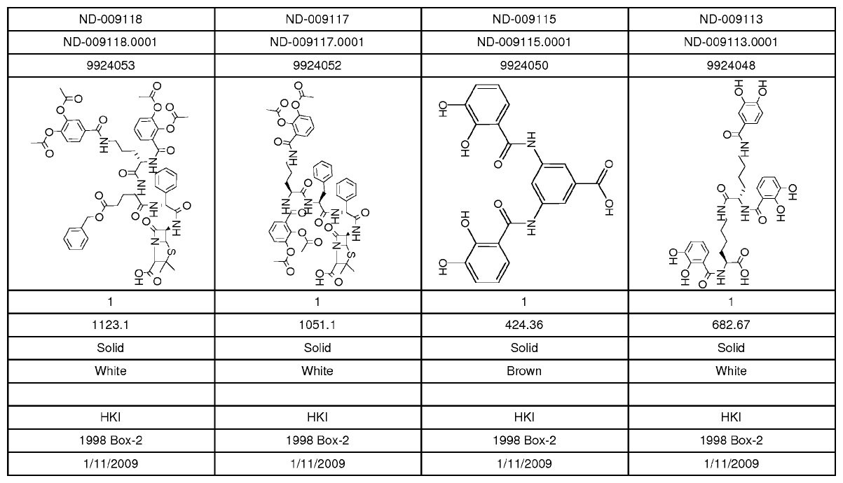

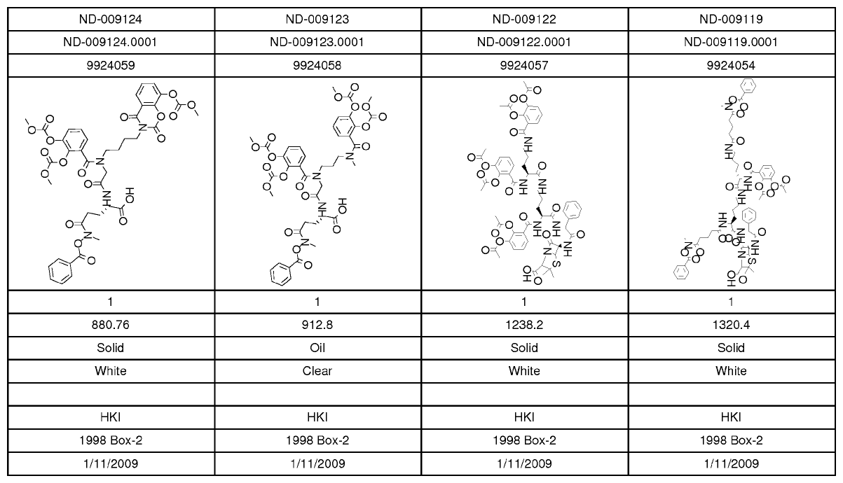

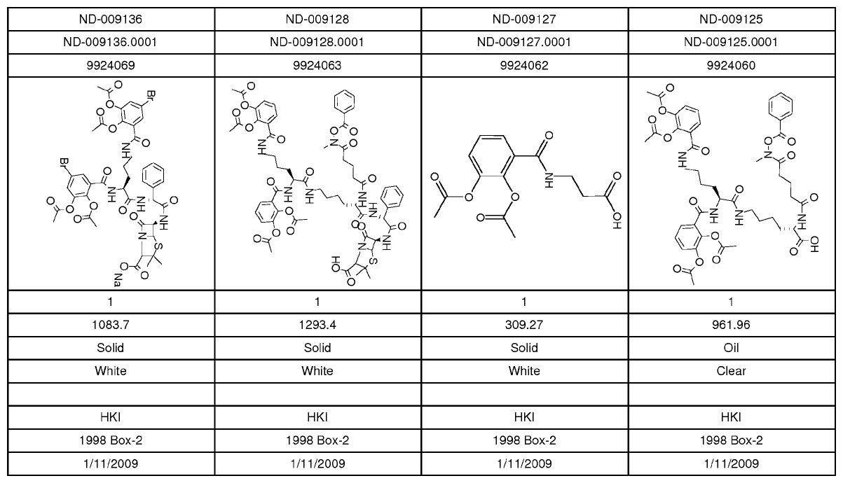

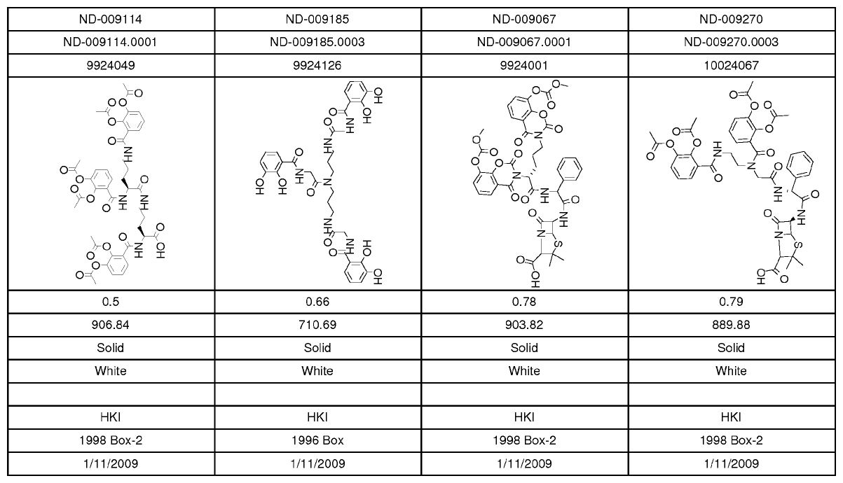

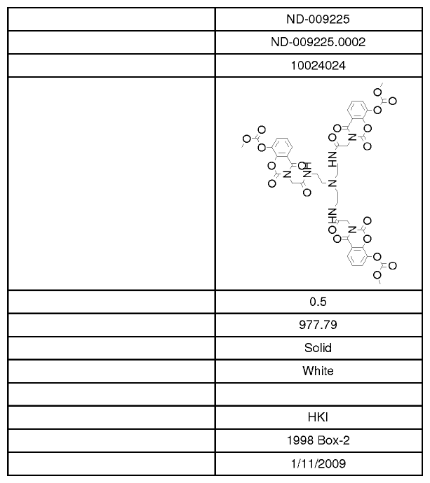

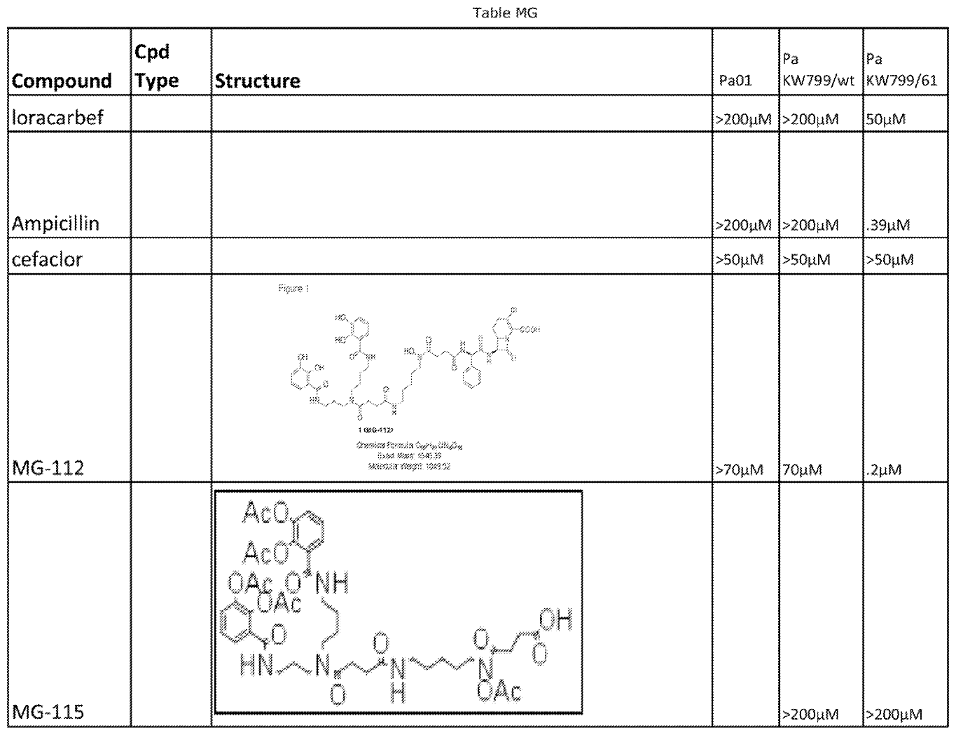

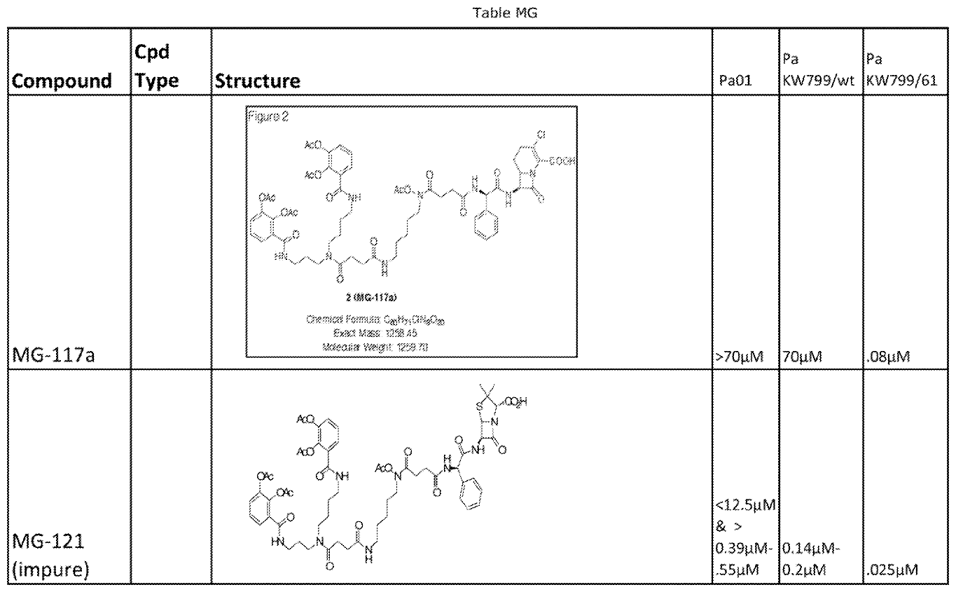

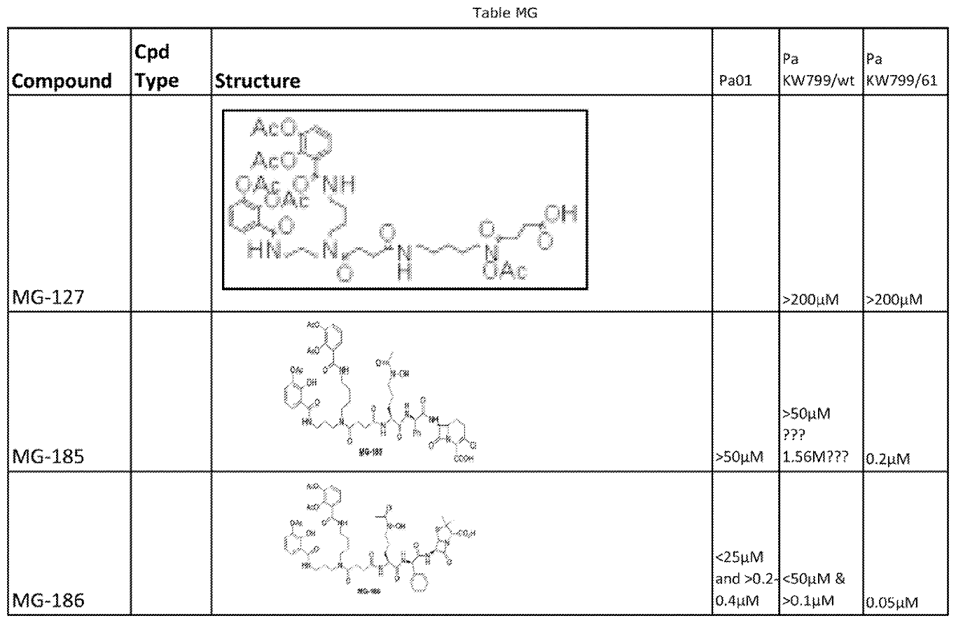

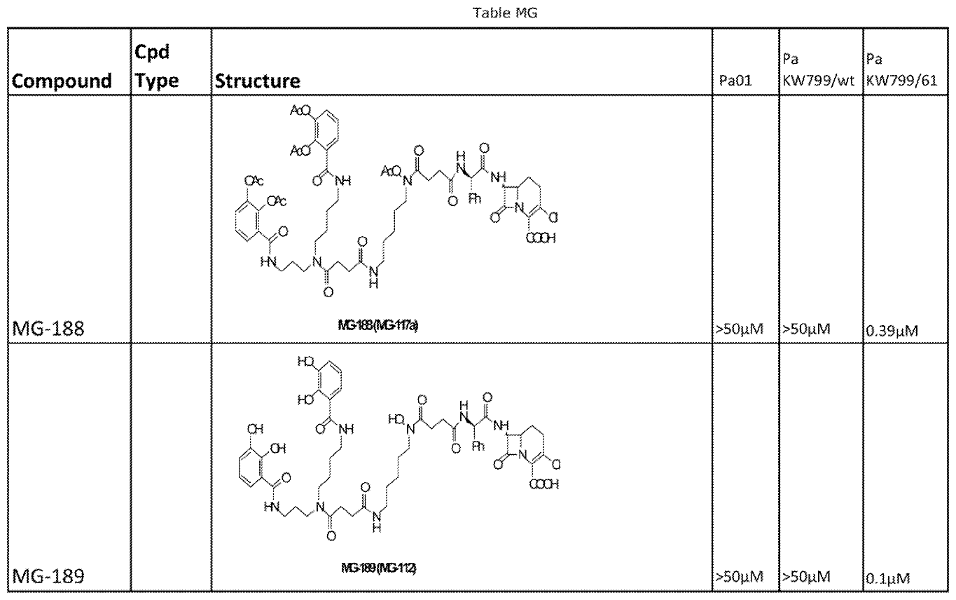

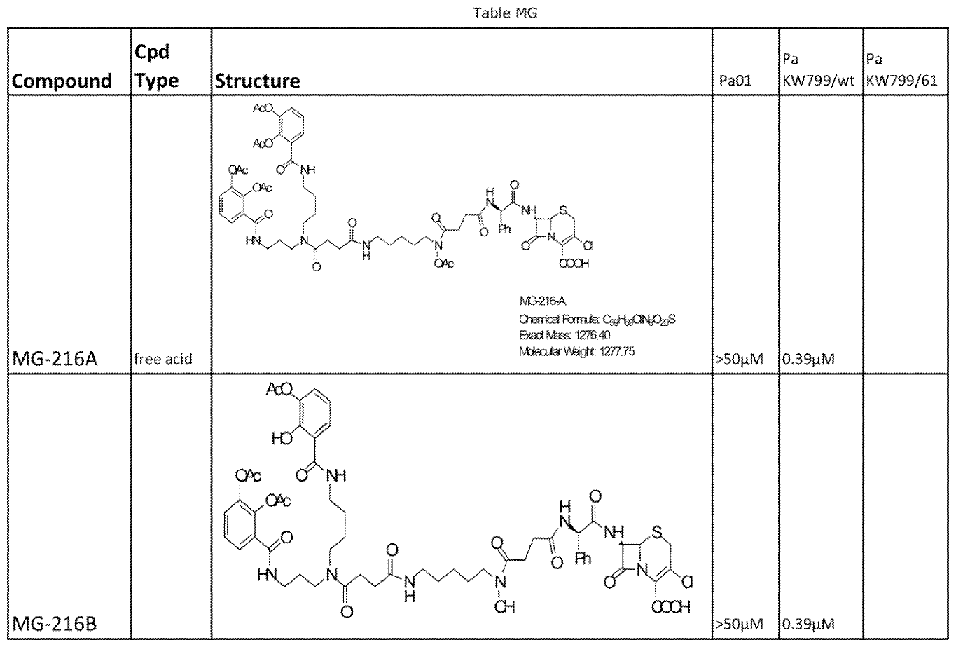

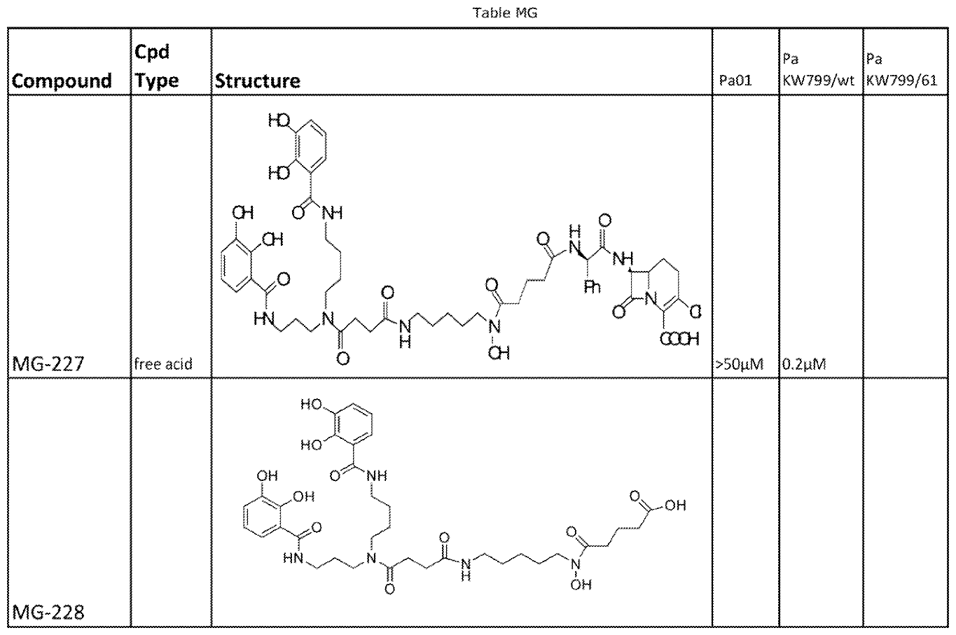

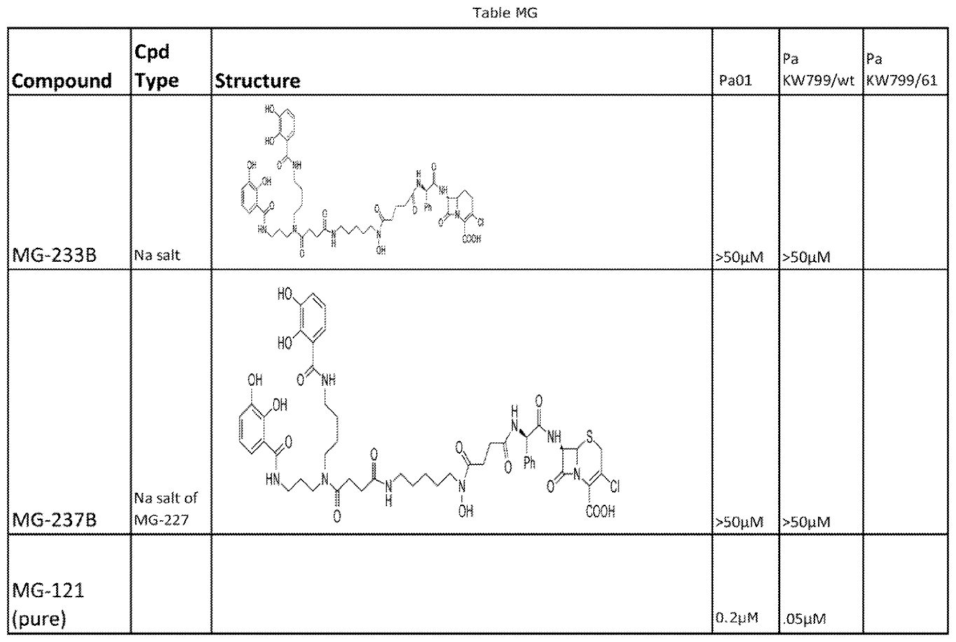

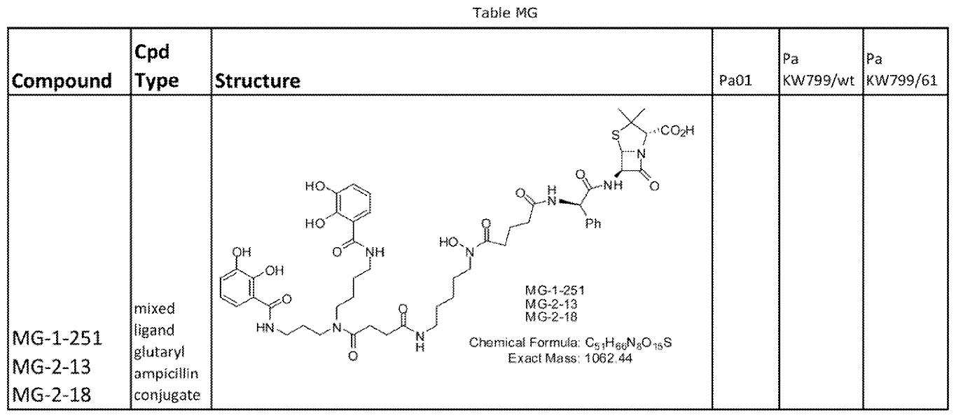

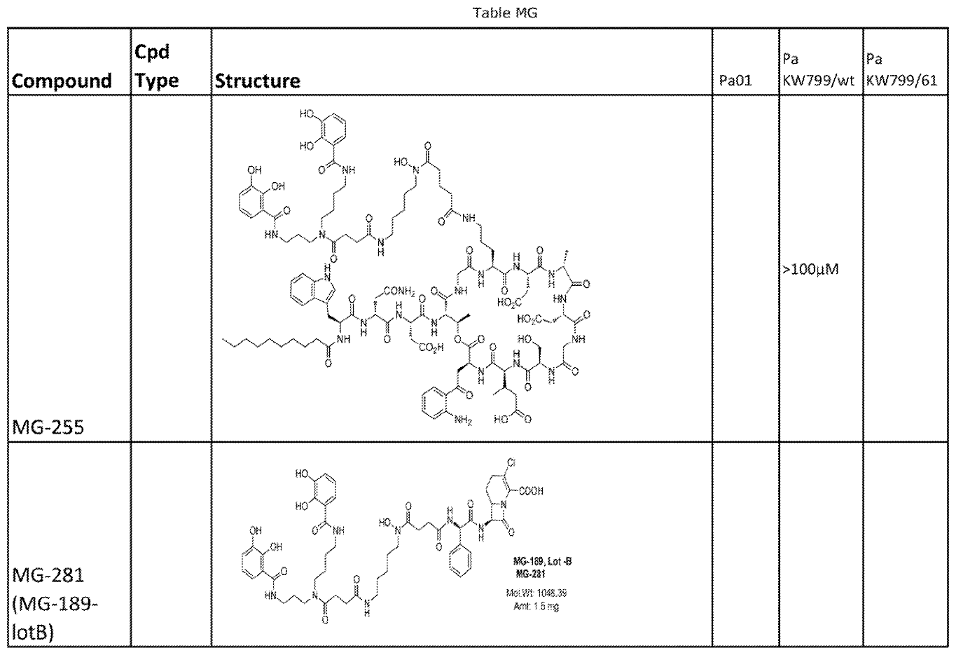

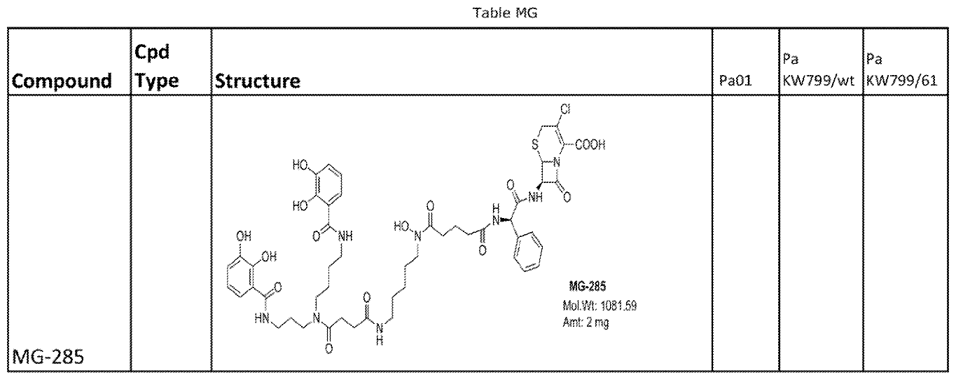

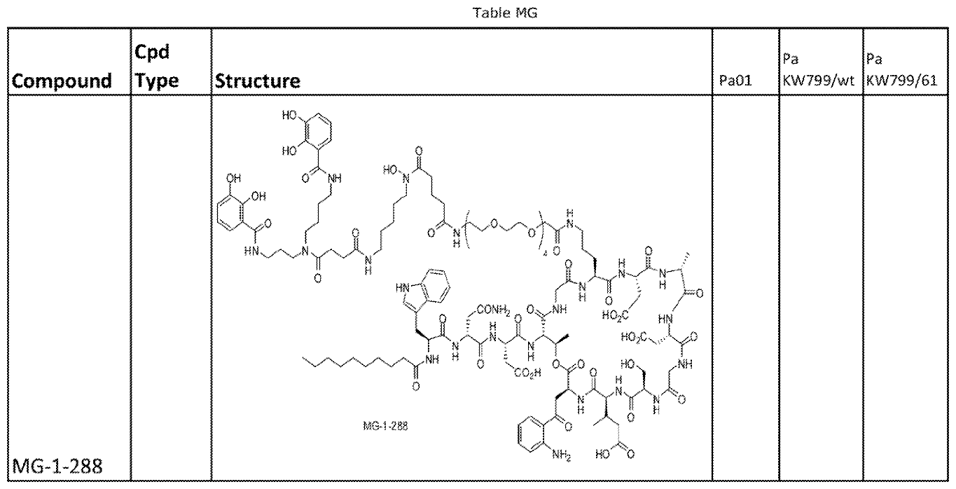

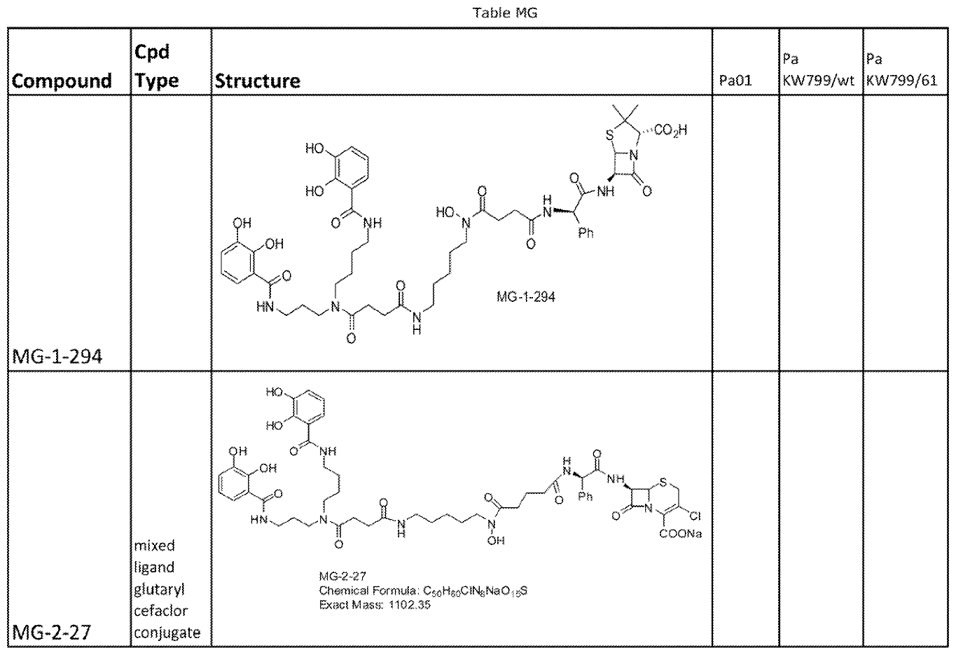

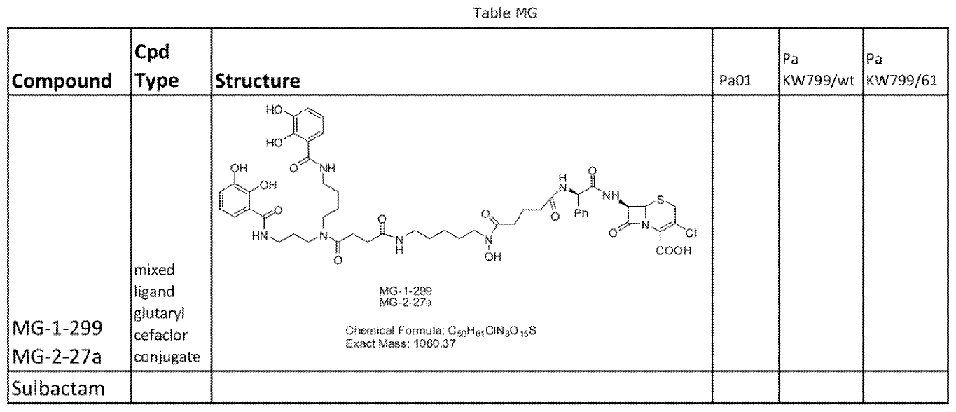

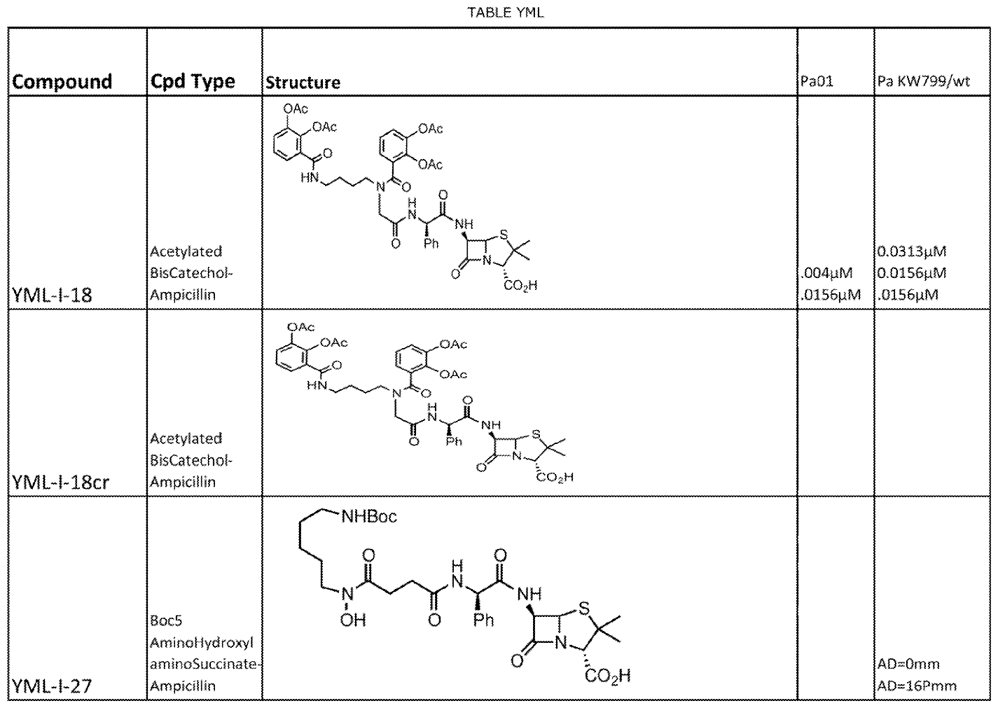

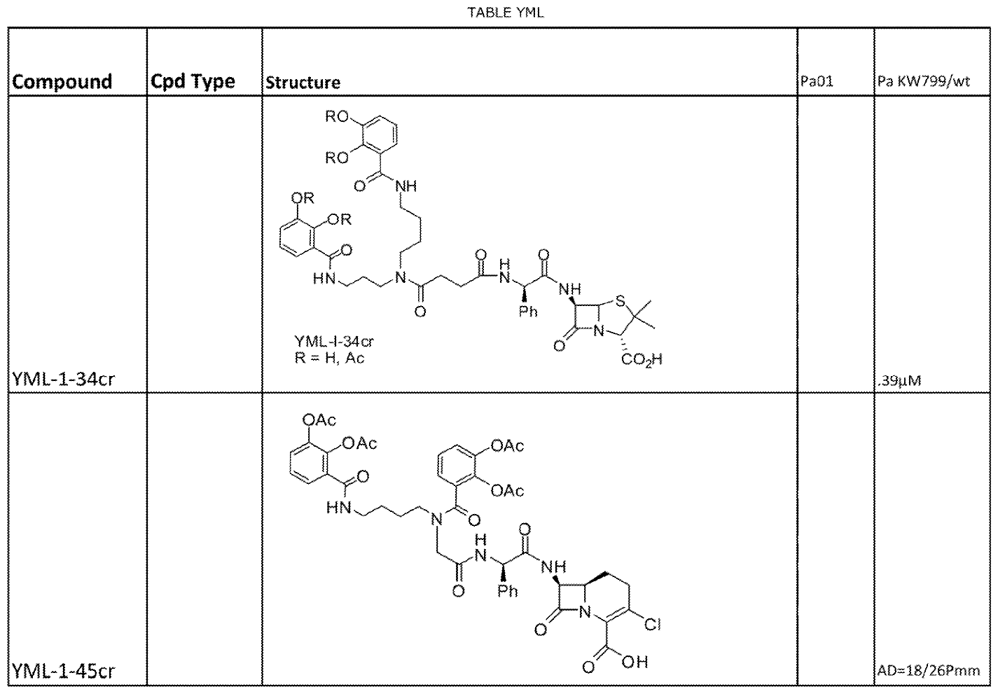

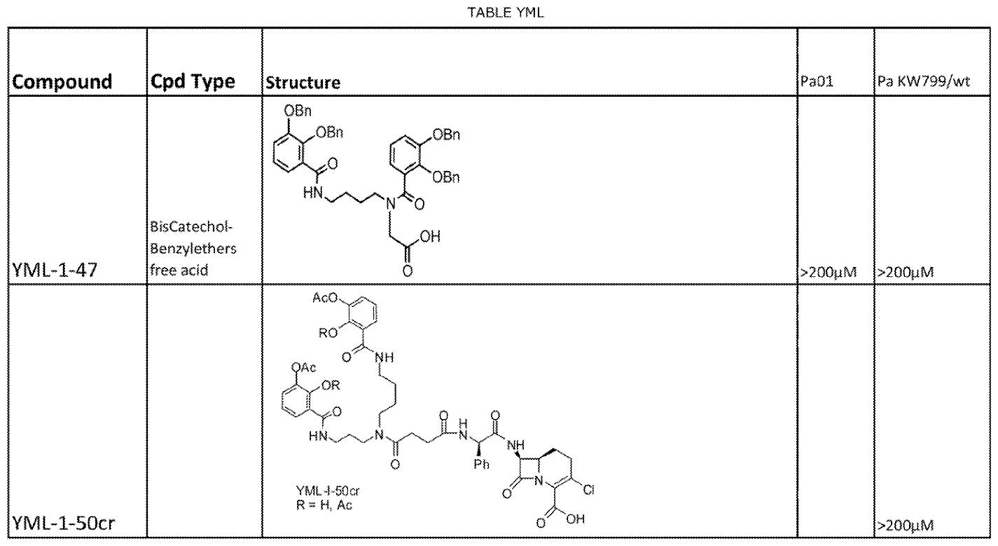

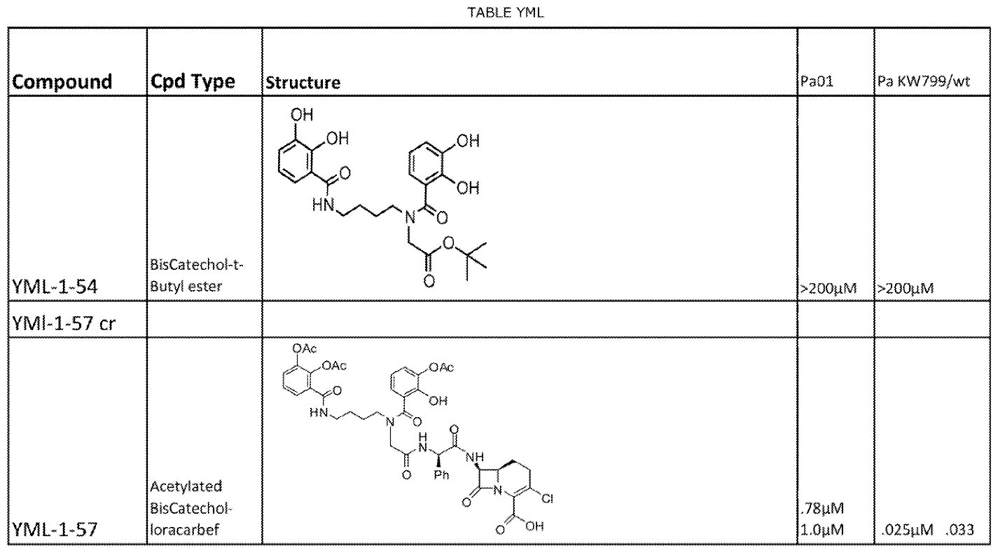

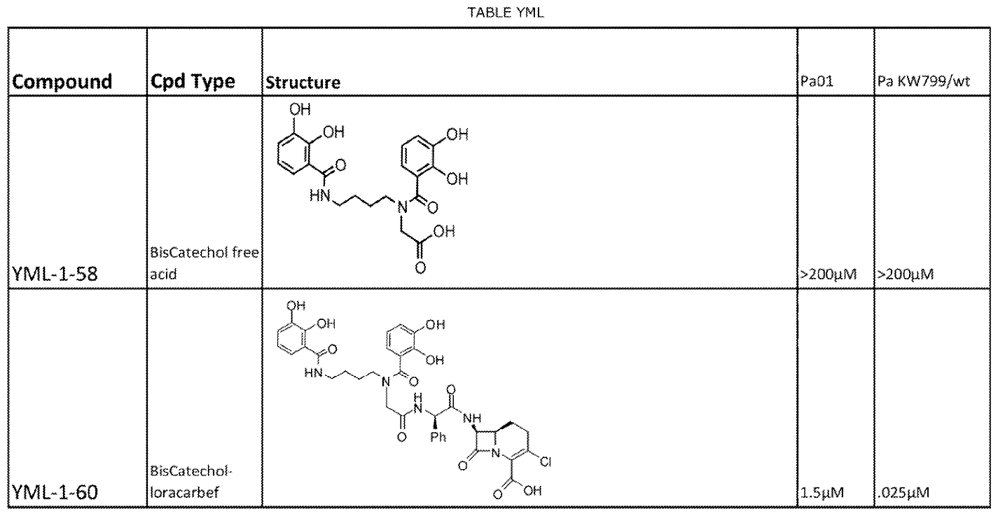

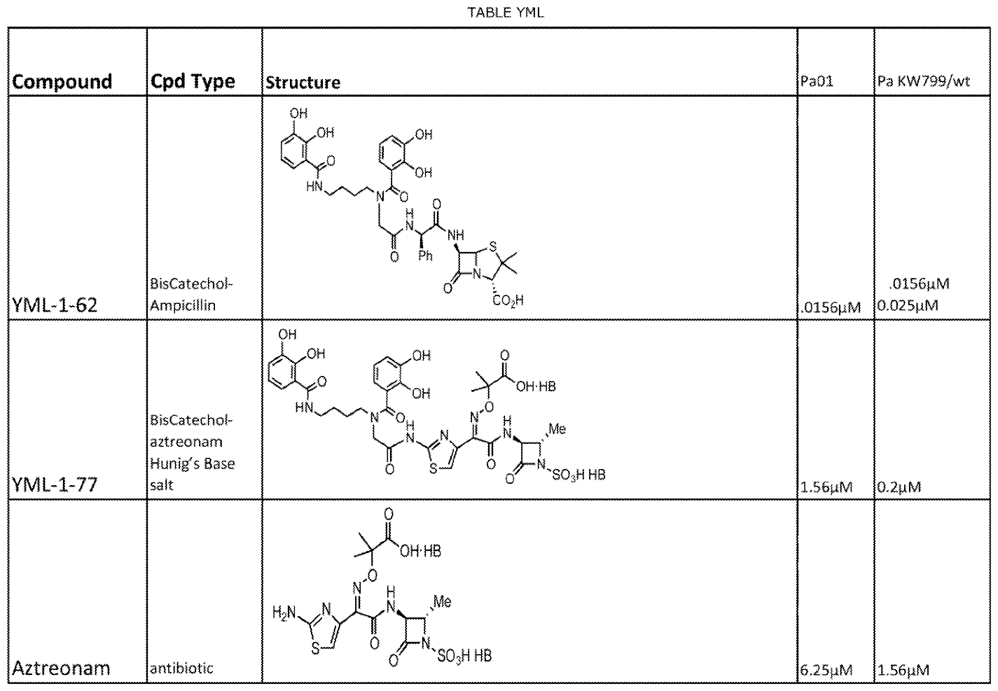

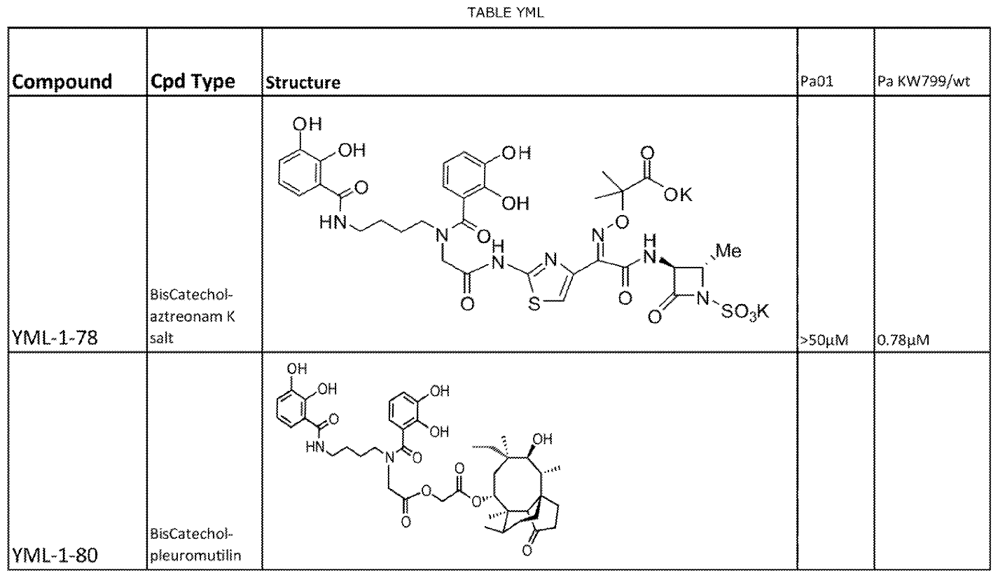

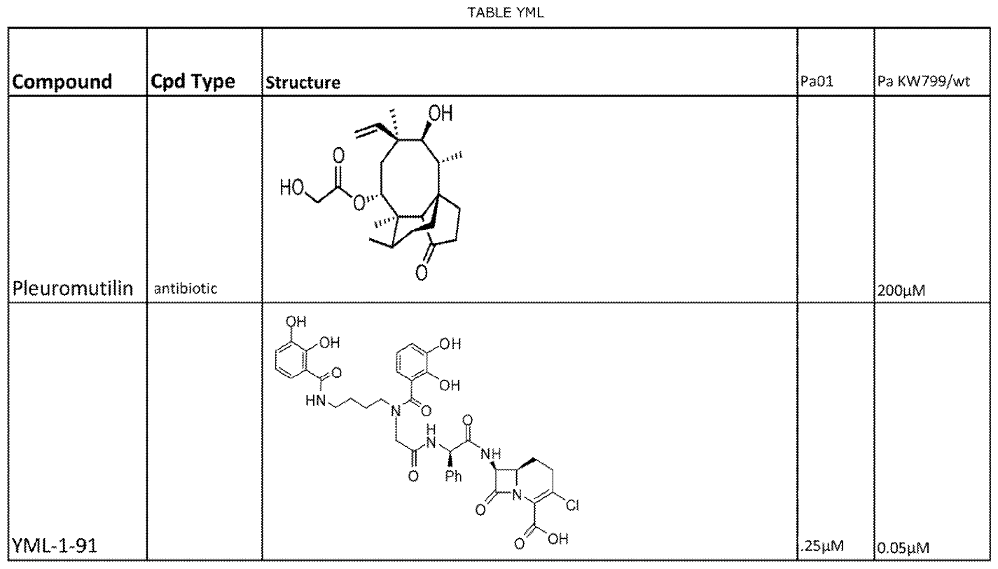

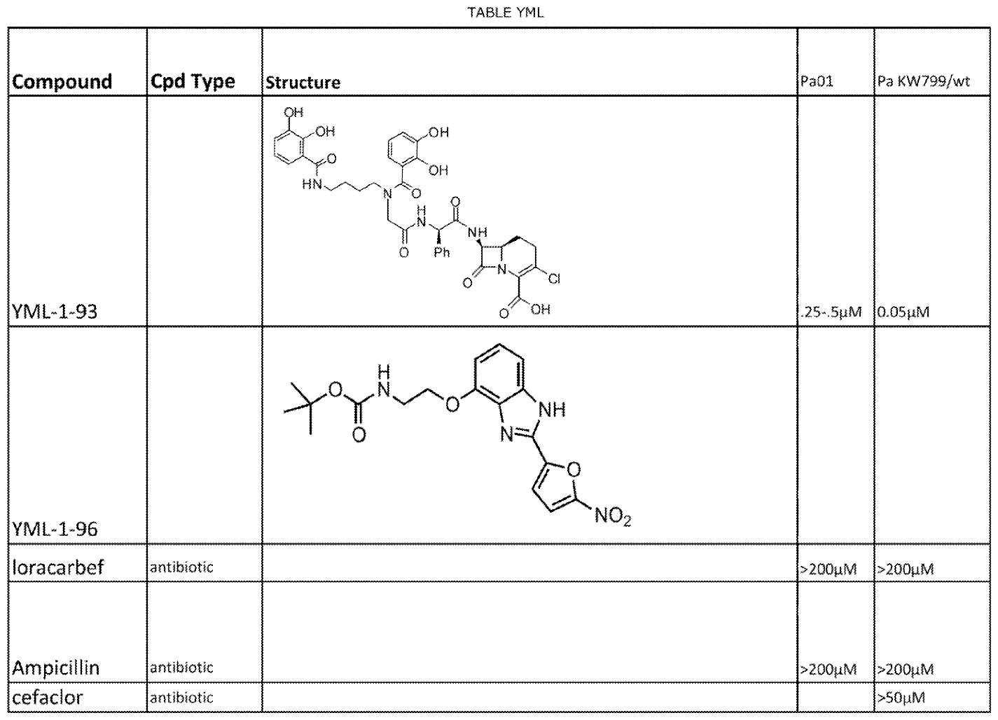

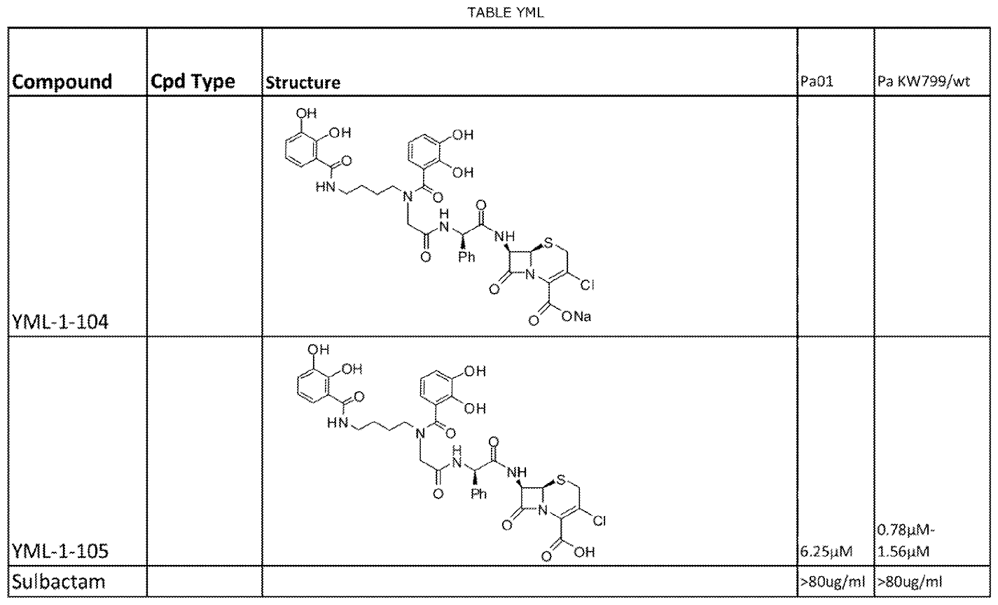

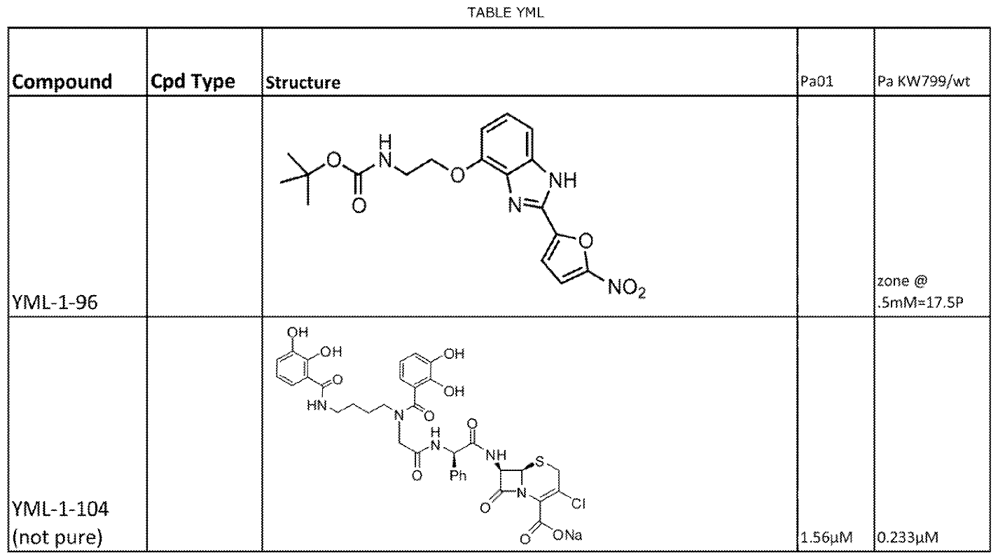

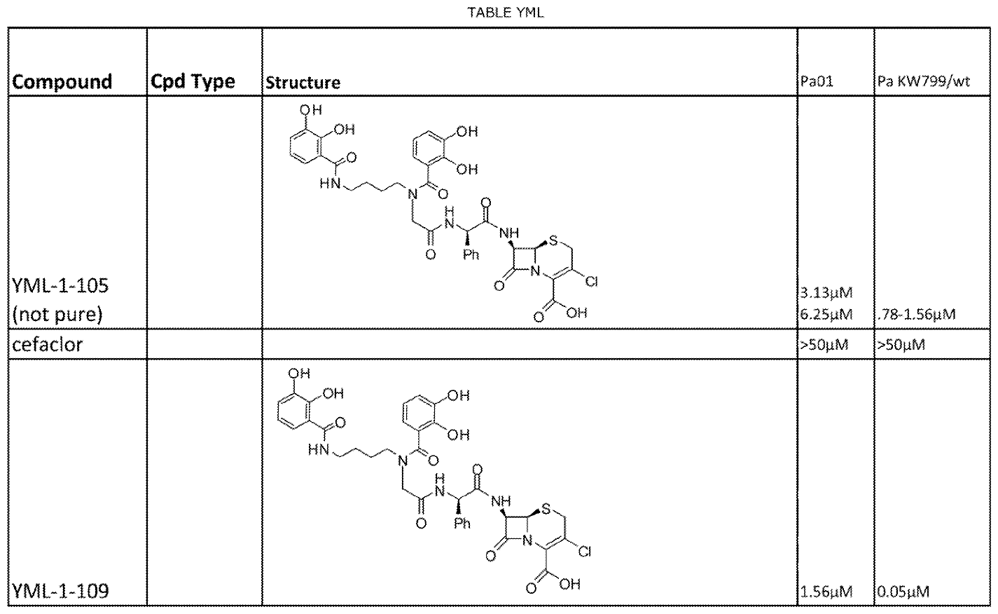

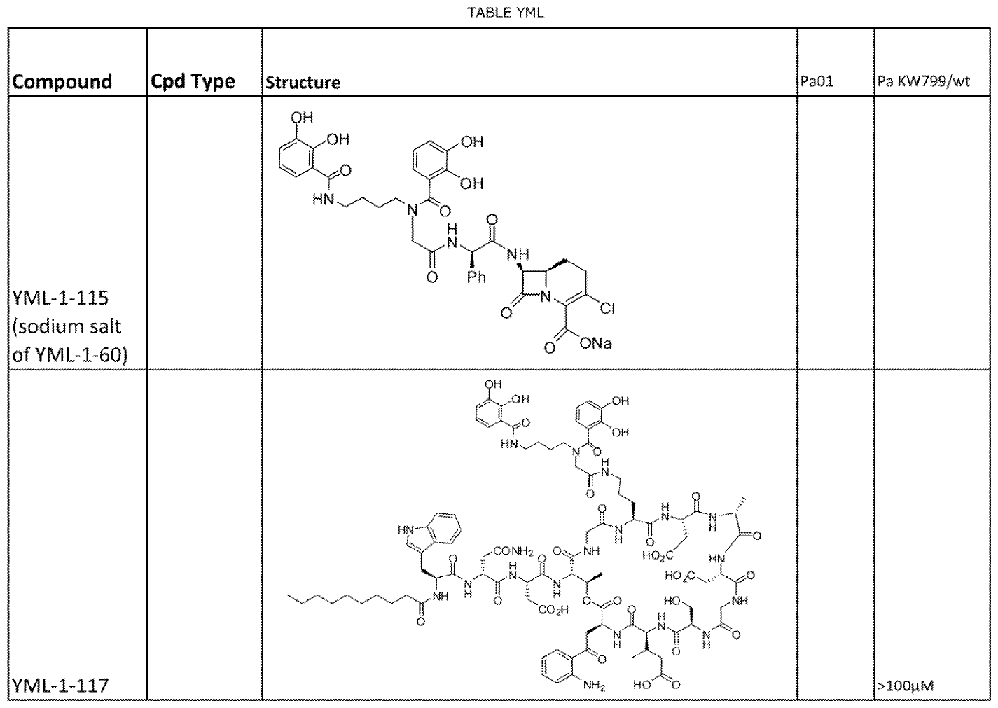

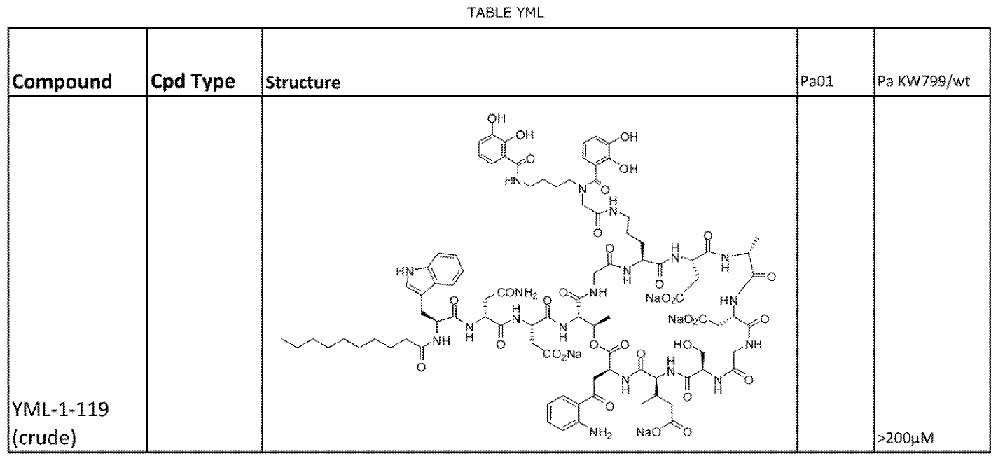

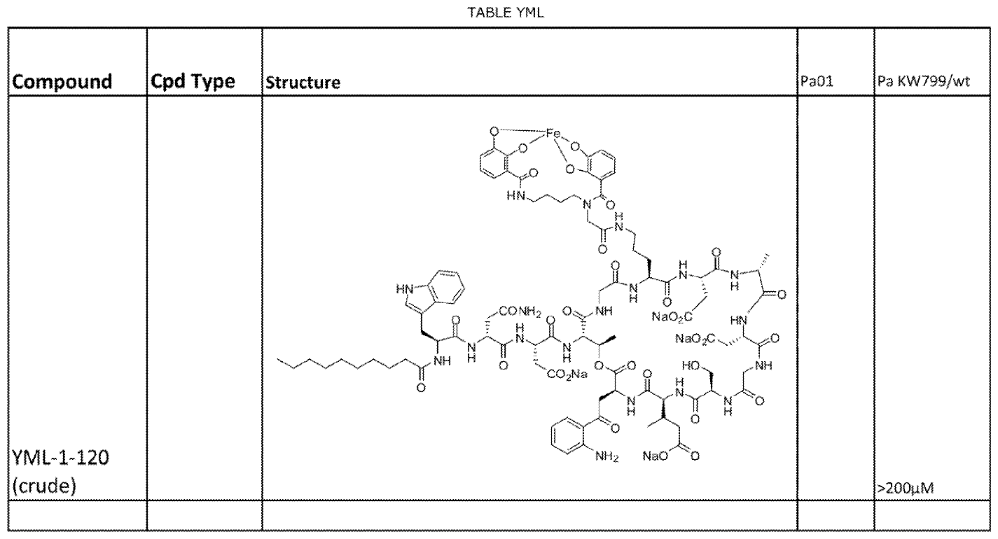

In one embodiment, the siderophore is a semi-synthetic or synthetic siderophore. Non-limiting examples of these siderophores may be found in the table in FIG. 20. In the figure, some siderophores have linkers and/or antibiotics attached, which linkers and/or antibiotics in some embodiments are not to be considered part of the siderophore. In such embodiments, the siderophore—without the linker and/or antibiotic shown in the table—may be suitably used in the compounds described herein.

In one embodiment, the siderophore comprises one or more iron(III)-binding or iron(III)-bound ligand.

In one embodiment, the siderophore comprises one or more iron(III)-binding or iron(III)-bound catechol, hydroxamic acid, beta-hydroxy acid, heteroaromatic ligand, or combination thereof.

In the formulas herein, each n is independently 1, 2, or 3.

In the formulas herein, each p is independently 0-11, which independently includes all values and subranges therebetween, including 0, 1, 2, 3, 4, 5, 6, 7, 8, 9, 10, and 11.

In the formulas herein, each j is independently 0-11, which independently includes all values and subranges therebetween, including 0, 1, 2, 3, 4, 5, 6, 7, 8, 9, 10, and 11.

In the formulas herein, each k is independently 1-11, which independently includes all values and subranges therebetween, including 0, 1, 2, 3, 4, 5, 6, 7, 8, 9, 10, and 11.

In the formulas herein, each l is independently 1-11, which independently includes all values and subranges therebetween, including 0, 1, 2, 3, 4, 5, 6, 7, 8, 9, 10, and 11.

In the formulas herein, each o is independently 0-11, which independently includes all values and subranges therebetween, including 0, 1, 2, 3, 4, 5, 6, 7, 8, 9, 10, and 11.

In the formulas herein, each m is independently 0-11, which independently includes all values and subranges therebetween, including 0, 1, 2, 3, 4, 5, 6, 7, 8, 9, 10, and 11.

In one embodiment, one or more than one (optional) linker is present. In one embodiment, more than one type of linker is present. In one embodiment, one linker is present. In one embodiment, no linker is present.

In one embodiment, the surface or modified surface contains a mixture of different siderophore—optional linker conjugates. In another embodiment, the surface or modified surface contains one type of siderophore—optional linker conjugate. In one embodiment, the surface or modified surface contains both Fe(III)-bound and Fe(III)-binding (i.e., the siderophore is not bound to Fe(III))—optional linker conjugates. In another embodiment, the surface or modified surface contains only one or more Fe(III)-bound siderophore—optional linker conjugates. In another embodiment, the surface contains only one or more Fe(III)-binding—optional linker conjugates.

One embodiment provides a siderophore—optional linker conjugate in which the siderophore includes one or more bi-dentate, tetra-dentate or hexadentate iron binding groups (catechols, ortho-hydroxy phenyl oxazolines, oxazoles, thiazolines, thiazoles, hydroxamic acids, alpha-hydroxy carboxylic acids or amides, pyridines, hydroxyl pyridones and combinations thereof). In one embodiment, the linker may include direct attachment of the siderophore component to linker either through a carboxylic acid of the siderophore attached to one or more amine components of the linker. Alternatively, the optional linker may include spacer groups commonly used in bioconjugation chemistry, including PEGylated groups of various lengths. Other attachment methods may suitably include “click chemistry”, carbohydrate linkages or other ligation.

Other non-limiting examples of siderophores include bis-catechols, tris-catechols, or derivatives of natural siderophores including entrobactin and derivatives, and mixed ligand siderophores, and natural siderophores including mycobactins.

In one embodiment, in the respective formulas Ia, IIa, IIIa, IVa, or Va, each IV is independently acetyl, propanoyl, or benzoyl. In one embodiment, each R¹ is acetyl. In another embodiment, each R¹ is H.

In one embodiment, in the respective formulas Ia, IIa, IIIc, IVa, or Va, each R² is independently H, alkyl, alkoxy, or hydroxy. In one embodiment, each R² is H. R² can also be a substituent as described herein.

In some embodiments, in the respective formulas Ia, IIa, IIIa, IVa, or Va, each R¹ is the same, while in other embodiments, R¹ groups can be different. Likewise, in various embodiments, each R² can be the same, while in other embodiments, R² groups can be different from each other, for example, depending on the starting material selected to prepare the compounds.

Terms and phrases used in this specification have their ordinary meanings as one of skill in the art would understand. Where appropriate, such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

The term “about” can refer to a variation of ±±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

Specific values listed below for radicals, substituents, and ranges, are for illustration only; they do not exclude other defined values or other values within defined ranges for radicals and substituents. Generic terms include each of their species. For example, the term halo includes and can explicitly be fluoro, chloro, bromo, or iodo.

The term “alkyl” refers to a branched, unbranched, saturated or unsaturated, linear or cyclic hydrocarbon having, for example, from 1-20 carbon atoms, and often 1-12, 1-10, 1-8, 1-6, or 1-4 carbon atoms. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, cyclopropyl, cyclobutyl, cyclopentyl, cyclohexyl, cycloheptyl, and the like. The alkyl can be unsubstituted or optionally substituted, for example, with a substituent described herein. The alkyl can also be optionally partially or fully unsaturated. As such, the recitation of an alkyl group can optionally include both alkenyl or alkynyl groups, linear or cyclic, in certain embodiments. The alkyl can be a monovalent hydrocarbon radical, as described herein, or it can be a divalent hydrocarbon radical (i.e., an alkylene), depending on the context of its use. In one embodiment, one or more carbons in the alkyl group may be replaced with one or more heteroatoms, e.g., O, N, S, P, combination thereof, and the like.

The term “alkoxy” refers to the group alkyl-O—, where alkyl is as defined herein. Examples of alkoxy groups include, but are not limited to, methoxy, ethoxy, n-propoxy, iso-propoxy, n-butoxy, tert-butoxy, sec-butoxy, n-pentoxy, n-hexoxy, 1,2-dimethylbutoxy, and the like. The alkoxy can be unsubstituted or substituted.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 20 carbon atoms, for example, about 6-10 carbon atoms, in the cyclic skeleton. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted, as described for alkyl groups. In one embodiment, one or more carbons in the aryl group may be replaced with one or more heteroatoms, e.g., O, N, S, P, combination thereof, and the like.

The term “amino acid” refers to alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, divalent radicals thereof, salts thereof, or combination thereof.

The term “carboxy” group refers to a univalent —CR″(═O) radical or a —CR″(═O)-containing substituent group. In one embodiment, the carboxy group suitably includes carboxylic acids, aldehydes, ketones, and combinations thereof. The R″ group is suitably chosen from any of the substituent groups. In one embodiment, the carboxy group may be attached to the parent structure through one or more independent divalent intervening substituent groups.

The term “amino” group refers to a univalent —NR″R″ radical or an —NR″R″ -containing subsituent group. The R″ groups may be the same or different and are suitably and independently chosen from any of the substituent groups. In one embodiment, the amino group may be attached to the parent structure through one or more independent divalent intervening substituent groups.

The term “nitro” group refers to a univalent —NO₂ radical or an —NO₂-containing substituent group. In one embodiment, the amino group may be attached to the parent structure through one or more independent divalent intervening substituent groups.

The term “cyano” group refers to a univalent —CN radical or a —CN-containing substituent group. In one embodiment, the cyano group may be attached to the parent structure through one or more independent divalent intervening substituent groups.

The term “peptide” refers to polypeptide, protein, oligopeptide, monopeptide, dipeptide, tripeptide, tetrapeptide, pentapeptide, hexapeptide, heptapentide, octapeptide, nonapeptide, decapeptide, undecapeptide, divalent radicals thereof, salts thereof, or combination thereof. In some embodiments, the term peptide may refer to a peptide bond, amide bond, or the like. For example, a peptide or amide bond is a covalent chemical bond formed between two molecules when the carboxyl group of one molecule reacts with the amino group of the other molecule forming a —C(O)NH— bond or peptide link.

A “linker” or “linking group” refers to an organic or inorganic chain or moiety that optionally connects the siderophore to surface or modified surface. The optional linker may be a molecule having end groups respectively tailored to covalently bond with the siderophore and the surface or modified surface. The linker is not particularly limited, so long as it can attach the siderophore to the surface or modified surface and not interfere or substantially interfere with the binding ability of the siderophore to the bacteria. In one embodiment, the optional linker may be covalently attached to the siderophore by an ester or amide bond. Nonlimiting examples of the optional linker include a group L where L is or is derived from one or more optionally substituted amino acid, peptide, alkylene, alkenylene, arylene, polyethylene glycol, polypropylene glycol, or combination thereof. Other nonlimiting examples of linkers include a group L where L is or is derived from a divalent radical of the formula -(W)ₐ-(Z)_{b}-(W)_{c}-; wherein a, b, and c are each independently 0-11; wherein each W is independently —N(R′)C(═O)—, —C(═O)N(R′)—, —OC(═O)—, —C(═O)O—, —O—, —S—, —S(O)—, —S(O)₂—, —N(R′)—, —C(═O)—, —(CR′₂)ₓ—, —(CX₂)_{y}—, —(CR′₂)ₓ—(CX₂)_{y}—, —(CR′₂CR′₂O)ₓ—, —(OCR′₂CR′₂)ₓ—, —N⁺(R′)₂(CR′₂)_{y}—, (C₁-C₁₂)alkylene, (C₂-C₁₂)alkenylene, (C₂-C₁₂)alkynylene, combination thereof, or a direct bond; and Z is a divalent moiety selected from (C₁-C₁₂)alkylene, (C₂-C₁₂)alkenylene, (C₂-C₁₂)alkynylene, (C₃-C₈)cycloalkylene, (C₆-C₁₀)arylene, —N(R′)C(═O)—, —C(═O)N(R′)—, —OC(═O)—, —C(═O)O—, —N(R′)—, —C(═O )—, —(CY₂)—, —(CR′₂)ₓ—(CY₂)_{y}—, —(OCR′₂—CR′₂)ₓ—, —(CR′₂CR′₂O)ₓ—, —C(O)NR′(CR′₂)_{y}—, —OP(O)(OR′)O—, —OP(O)(OR′)O(CR′₂)_{y}—, —OP(O)(OR′)OCR′₂CR′(OR′)CR′₂—, —N⁺(R′)₂(CR′₂)ₓ—, or (C₁-C₁₂)alkylene, (C₂-C₁₂)alkenylene, or (C₂-C₁₂)alkynylene, optionally interrupted between two carbons, or between a carbon and an oxygen, with a (C₃-C₈)cycloalkyl, heteroaryl, heterocycle, or (C₆-C₁₀)aryl group, divalent amino acid, divalent peptide, combination thereof, or Z is a direct bond; wherein x and y are each independently 0-11; wherein each X is independently a halogen (“halo”): F, Cl, Br, or I; and each R′ is independently H, alkyl, aryl, (aryl)alkyl (e.g., benzyl), heteroaryl, (heteroaryl)alkyl, heterocycle, heterocycle(alkyl), or a protecting group; wherein each of W, Z and R′ may be optionally substituted with one or more substituent groups; and each of W, Z, and R may have one or more carbons replaced with one or more heteroatoms, e.g., N, O, S, P, and the like.

Referring to the paragraph above, wherein a, b, and c are each independently 0-11, these ranges independently include all values and subranges therebetween, including 0, 1, 2, 3, 4, 5, 6, 7, 8, 9, 10, and 11. Referring also to the paragraph above, wherein x and y are each independently 0-11, these ranges independently include all values and subranges therebetween, including 0, 1, 2, 3, 4, 5, 6, 7, 8, 9, 10, and 11.

In one embodiment, one or more of the W and/or Z groups can independently form or originate from a part of the siderophore and/or the linker In another embodiment, one or more of the W and/or Z groups can independently form or originate from a part of the linker and/or surface or modified surface.

The term “substituted” indicates that one or more (e.g., 1, 2, 3, 4, or 5; in some embodiments 1, 2, or 3; and in other embodiments 1 or 2) hydrogen atoms on the group indicated in the expression using “substituted” is replaced with a “substituent”. The substituent can be one of a selection of the indicated group(s), or it can be a suitable group known to those of skill in the art, provided that the substituted atom's normal valency is not exceeded, and that the substitution results in a stable compound. Nonlimiting examples of substituent groups include, e.g., alkyl, alkenyl, alkynyl, alkoxy, halo, haloalkyl, hydroxy, hydroxyalkyl, aryl, aroyl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxyl amine, hydroxyl (alkyl)amine, and cyano, as well as the moieties illustrated in the schemes and Figures of this disclosure, and combinations thereof. Other nonlimiting examples of the substituent group include, e.g., —X, —R″, —O⁻, —OR″, —SR, —S⁻, —NR″₂, —NR″₃⁺, ═NR″, —CX₃, —CN, —OCN, —SCN, —N═C═O, —NCS, —NO, —NO₂, ═N₂, —N₃, —NC(═O)R″, —C(═O)R″, —C(═O)NR″R″, —S(═O)2O⁻, —S(═O)₂OH, —S(═O)₂R″, —OS(═O)₂OR″, —S(═O)₂NHR″, —S(═O)R″, —OP(═O)(OR″)₂, —P(═O)(OR″)₂, —OP(═O)(OH)(OR″), —P(═O)(OH)(OR″), —P(═O)(O⁻)₂, —P(═O)(OH)₂, —C(═O)R″, —C(═O)X, —C(S)R″, —C(O)OR″, —C(O)O⁻, —C(S)OR″, —C(O)SR″, —C(S)SR″, —C(O)NR″R″, —C(═S)NR″R″, —C(═NR″)NR″R″, wherein each X is independently a halogen (“halo”): F, Cl, Br, or I; and each R″ is independently H, alkyl, aryl, (aryl)alkyl (e.g., benzyl), heteroaryl, (heteroaryl)alkyl, heterocycle, heterocycle(alkyl), or a protecting group. As would be readily understood by one skilled in the art, when a substituent is keto (═O) or thioxo (═S), or the like, then two hydrogen atoms on the substituted atom are replaced. In some embodiments, one or more of the substituents above are excluded from the group of potential values for substituents on the substituted group.

One or more than one type of linker may be present. In one embodiment, the surface may include only one type of siderophore, wherein the same linker is used for each siderophore. In another embodiment, one type of siderophore is used, but wherein different types of linkers are used. Alternatively, different siderophores may be used, but wherein the same type of linker is used for each siderophore.

When more than one siderophore is used, for example, when it is desirable to target more than one type of bacterium, or even when it is desirable to target only a single type of bacterium, the amount of any given siderophore relative to the other siderophores is not particularly limited, and may suitably range from more than one to less than all of the siderophores present on a molar basis. This range includes all values and subranges therebetween, including >1, 2, 3, 4, 5, 6, 7, 8, 9, 10, 15, 20, 25, 30, 35, 40, 45, 50, 55, 60, 65, 70, 75, 80, 85, 90, 95, and 99 mol % or any combination thereof.

In one embodiment, the siderophore may contain a free OH (alcohol), amine, or carboxylic acid to which the linker may be attached via ester (on the OH), amide (on the amine) or reverse the ester or amide using the siderophore carboxyl. In one embodiment, the linker chain can be short or long with or without heteroatom substitution as desired. In one embodiment, the linker can terminate on the surface-binding side with a thiol, silane, alkylsilane, alkoxysilane, for example, or other reactive group which will react with a surface such as gold, glass, quartz, silicon, and the like. Alternatively, the linker can terminate with another alcohol, amine or acid which can then be attached to a corresponding functionality on the surface of choice. Non-limiting examples of suitable linkers for bioconjugation may be found in Bioconjugate Techniques by Greg T. Heranson, Academic Press, 1996, incorporated herein by reference.

If desired, the sample may be used neat, or it may be combined with a carrier. So long as it does not interfere with the desired binding, measurement, detection, readout, amplification, etc., the carrier is not particularly limited. For example, carriers such as water, saline, DMSO, methanol, ethanol, glycerol, liquid polyethylene glycols, triacetin, polyol (for example, glycerol, propylene glycol, liquid polyethylene glycols, and the like), vegetable oils, nontoxic glyceryl esters, pharmaceutically acceptable oil, or the like, or any combination thereof. In one embodiment, it may be suitable to include isotonic agents, for example, sugars, buffers, or sodium chloride.

Other examples of linkers with siderophores are given below:

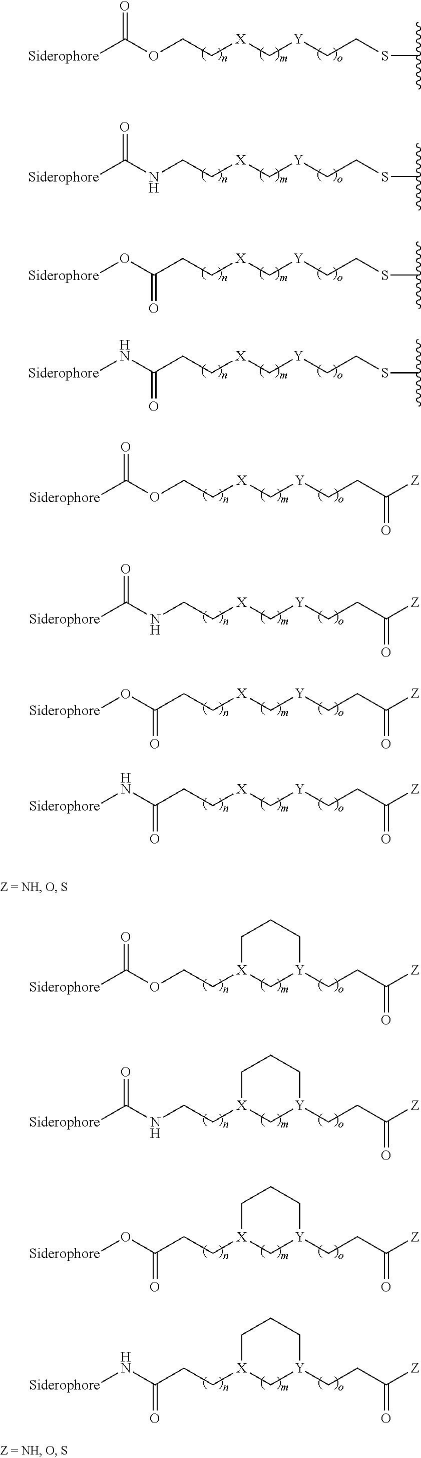

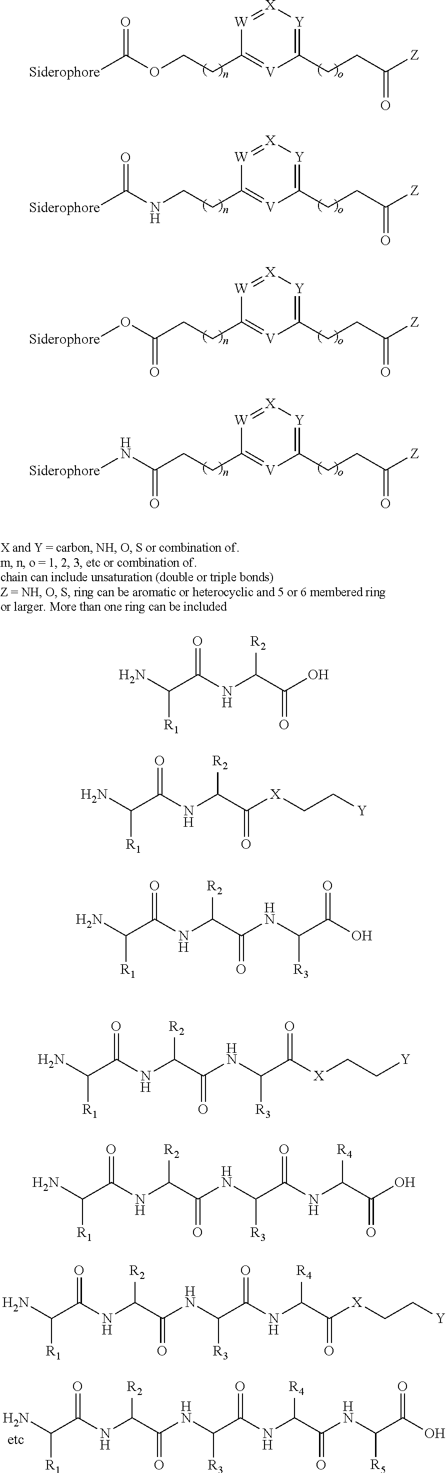

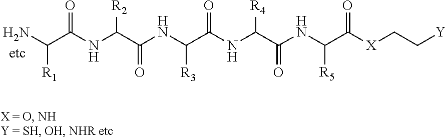

In the second group of formulas given above, the various R₁, R₂, R₃, R₄, and R₅ groups can each independently be hydrogen or any of the substituent groups described herein. In one embodiment, the R₁, R₂, R₃, R₄, and R₅ groups are hydrogen or C₁-₃ alkyl. In one embodiment, the R₁, R₂, R₃, R₄, and R₅ groups are hydrogen.

All of the compounds described herein can be easily prepared according to the methods in the Examples herein, or may be prepared according to known techniques in the art of organic synthesis. Many linking groups for conjugating the siderophore and/or linker and/or surface are commercially available, and/or can be prepared as described in the art. Information regarding general synthetic methods that may be used to prepare the compounds described herein, particularly with respect employing linking groups, may be found in Greg T. Hermanson, Bioconjugate Techniques, Academic Press, San Diego, Calif. (1996). Other non-limiting examples of useful linkers and conjugation techniques are further described by Roosenberg et al., Curr. Med. Chem. 2000, 7, 159; Wittmann et al., Bioorg. Med. Chem. 2002, 10, 1659; and Heinisch et al., J. Med. Chem. 2002, 45, 3032. Additional useful reactions well known to those of skill in the art are referenced in March's Advanced Organic Chemistry Reactions, Mechanisms, and Structure, 5^{th} Ed. by Michael B. Smith and Jerry March, John Wiley & Sons, Publishers; and Wuts et al. (1999), Protective Groups in Organic Synthesis, 3^{rd} Ed., John Wiley & Sons, Publishers. The entire contents of each of these references are hereby incorporated by reference.

The methods of preparing compounds of the invention can produce isomers in certain instances. Although the methods of the invention do not always require separation of these isomers, such separation may be accomplished, if desired, by methods known in the art. For example, preparative high performance liquid chromatography methods may be used for isomer purification, for example, by using a column with a chiral packing.

If desired, the compounds described herein can be used in the form of a salt. For example, in cases where compounds are sufficiently basic or acidic to form stable nontoxic acid or base salts, their use as salts may be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, a-ketoglutarate, and eta-glycerophosphate. Suitable inorganic salts may also be formed, including hydrochloride, halide, sulfate, nitrate, bicarbonate, and carbonate salts.

Pharmaceutically acceptable salts may also be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

In one embodiment, devices and methods of detecting and/or diagnosing a Gram-negative bacteria and/or bacterial infection are provided. In one embodiment, devices and methods of detecting and/or diagnosing a Gram-positive bacteria and/or bacterial infection are provided.

The bacteria or bacterial infection may be or may arise from Gram-negative bacteria, Gram-positive bacteria, antibiotic-resistant bacteria, multidrug-resistant organism (MDRO), methicillin-resistant pathogen, nosocomial pathogen, Pseudomonal bacterium, Bacillus bacterium, Acinetobacter bacterium, Staphylococcus bacterium, Escherichia bacterium, Micrococcus bacterium, Mycobacterium, Pseudomonas aeruginosa, Escherichia coli, Acinetobacter baumannii, Salmonella typhimurium, B. subtilis, S. aureus, M. luteus, Staphylococcus aureus, Mycobacterium tuberculosis (Mtb), E. faecium, Micrococcus luteus, E. aerogenes, K pneumonia, M. vaccae, M smegmatis, M aurum, M fortuitum, Yersinia pestis, Y. enterocolitica, M avium, M abscessus, M. kansasii, M paratuberculosis, MRSA, MDRAB, or any combination thereof.

Physicians are in need of an improved method for identifying pathogenic bacteria, especially those drug-resistant strains which currently cause the majority of deaths within health care facilities. Examples include methicillin-resistant Staphylococcus aureus (MRSA), multidrug resistant Myobacterium tuberculosis (Mtb), Pseudomonas aeruginosa, and multidrug-resistant Acinetohacter baumannii (MDRAB).

### EXAMPLES

Specific Aims:

Regardless of the environment, P. aeruginosa and A. baumannii species, like most aerobic and facultative anaerobic bacteria, require host iron for survival (1-3). Moreover, alterations in iron trapping are associated with diminished virulence (4). P. aeruginosa and A. baumannii have evolved specific small molecules called siderophores for this critical function of iron acquisition. P. aeruginosa acquires iron primarily via its specific siderophores, pyoverdin and pyochelin (5), and pyoverdin is required for Pseudomonas virulence (6). However, previous data have reported that, while P. aeruginosa does not make the siderophore, enterobactin, it can also use this siderophore for iron uptake (7). Acinetobacter uses fimsbactin and acinetobactin as its primary siderophores (8). As demonstrated by our published preliminary data, we have developed a tripodal catecholate siderophore that, when coupled with an aminopenicillin, has outstanding in vitro activity against most Pseudomonas aeruginosa strains tested (9). This binding to Pseudomonas strongly suggests that the tripodal catecholate can also be used as a diagnostic agent in which the tripodal catecholate molecular recognition motif is surface immobilized to facilitate recognition by bacterial siderophore receptors, but surface immobilization defeats the bacterial transporters, thus effecting surface capture of the bacteria. In addition to this siderophore, we are designing the synthesis of fimsbactin as an anchoring siderophore to use with the same technology for the detection of Aceinetobacter. Using the organisms' specific siderophores, we will make a highly selective and highly sensitive diagnostic device to detect both of these serious pathogens in biologic and environmental samples. We believe that this device will speed diagnosis in infected patients and help prevent disease when used for environmental surveillance. The specific aims for both devices are, broadly: creating a prototype (Aims a to e); scaling to clinical validation (Aims f to h); and commercializing the product (Aims i and j). The aims will be pursued in a staggered timeline, with the second aim building off the experience gained in the first.

AIM 1: Profile and Develop the Novel Tripodal Catecholate Siderophore (HD-01) as an Anchor for the Siderophore-Based Diagnostic for Pseudomonas

Aim 1a—Synthesize functionally active HD-01

Aim 1b—Couple HD-01 to a fabricated polymethylmethacrylate scaffold

Aim 1c—Optimize the reaction conditions for Pseudomonas capture

Aim 1d—Conjugate HD-01 to Au nanoparticies for development of capture signal

Aim 1e—Fabricate a working prototype of the device

Aim 1f—Scale up the GMP manufacture of the diagnostic device

Aim 1g—Assess fully the reproducibility, load detection, sensitivity, specificity and predictive accuracy of the device in laboratory settings

Aim 1h—Assess the usability and accuracy of the device in clinical settings

Aim 1i—Submit 510k for regulatory approval

Aim 1j—Launch product

AIM 2: Profile and Develop the Novel Fimsbactin analog (HD-02) as an Anchor for the Siderophore-Based Diagnostic for Acinetobacter

Aim 2a—Synthesize functionally active HD-02

Aim 2b—Couple HD-02 to a fabricated polymethylmethacrylate scaffold

Aim 2c—Optimize the reaction conditions for Acinetobacter capture

Aim 2d—Conjugate HD-02 to Au nanoparticles for development of capture signal

Aim 2e—Fabricate a working prototype of the device

Aim 2f—Scale up the GMP manufacture of the diagnostic device

Aim 2g—Assess fully the reproducibility, load detection, sensitivity, specificity and predictive accuracy of the device in laboratory settings

Aim 2h—Assess the usability and accuracy of the device in clinical settings

Aim 2i—Submit 510k for regulatory approval

Aim 2j—Launch product

The diagnostic technology described below will provide for the rapid and sensitive diagnosis of Pseudomonas and Acinetobacter that, upon sputum liquification, plasma separation or environmental swab preparation, can be used by either practitioners or patients on an outpatient basis. Body fluid preparation (sputum, urine, plasma, other) can be prepared using standard solutions and applied to the device. Further, the devices can be used to determine environmental contamination of Pseudomonas and Acinetobacter in hospital settings and in specialized treatment settings, such as respiratory therapy departments or intensive care units. These devices will be true point-of-care diagnostic devices with an obvious visual signal for detection. The target product attributes for both devices will be as follows:

portability (the dimensions of a standard playing card or smaller)

low cost per unit

easy to use by unskilled or semi-skilled workers

very highly predictive accuracy (>99%)

no need for specialized apparatus for reading the results

no need for refrigeration or electricity

speed (<2 hr)

In this last regard, the device will report within two hours using the following steps:

a) sputum liquification, plasma separation, or environmental swab preparation (10 to 20 min); b) sample loading (5 to 10 min); c) sample binding (10 to 20 min); d) sample rinsing (5 to 10 min); e) sample development (20 to 40 min); and f) sample reading (1 to min). After collecting the sample and then developing the capture signal, the result will be viewed either on a field-deployable reader, or, ideally, with a simple hand-held viewer.

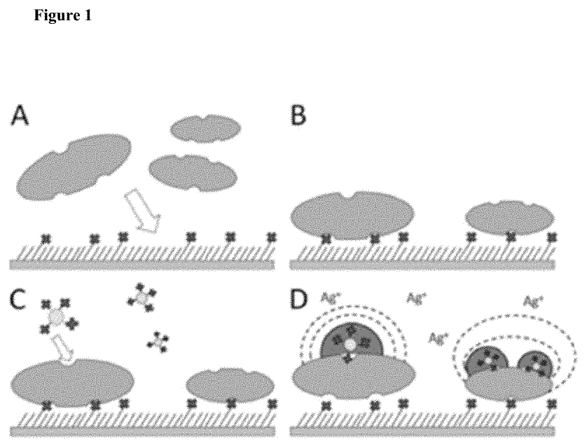

The technology translates P. aeruginosa's obligate iron needs and mechanisms for iron foraging into a diagnostic agent. Since Fe(III) is insoluble at physiological pH, microbes have evolved specific processes for iron sequestration that involve active transport through an otherwise impermeable outer membrane. Bacterial iron acquisition is essential for pathogenicity and provides an attractive and little-used target for developing microbe-selective biomarkers for selective detection. Acquisition of iron by bacteria at the infection site depends on the presence of soluble Fe(III) complexes generated from iron sources. These solubilized Fe(III)-binding complexes (generically called siderophores) must then be sequestered by the bacteria to initiate iron transport across the cell envelope. To do this, Gram negative bacteria express specific outer membrane receptor proteins that specifically recognize siderophore iron complexes and initiate active transport. This exquisite molecular recognition is essential and will be exploited in the development of our diagnostic technology. Because of selective recognition and transport needed for bacterial growth advantage, the technology will be developed to detect the presence of P. aeruginosa from a wide variety of biological samples. The general schematic of the Pseudomonas “pull down,” Pseudomonas binding detection and signal amplification is shown in FIG. 1. The final device will be a microfluidic, multichannel affinity recognition and detection system based on covalent attachment of P. aeruginosa-specific or modified siderophores to the surface of separate channels in the microfluidic device. Passage of microliter volumes of sample through the device will allow exposure to the bound siderophores that will specifically recognize and tightly bind P. aeruginosa. The optimal device will be low cost, easy to use and highly sensitive, compared to either standard gram staining and culture of Pseudomonas or fluorescently aided microscopy. This technology has the sensitivity to be able to detect a single bacterial cell and will also be semi-quantitative with varying signal intensity.

The proposed technology is a rapid, sensitive, whole-cell, diagnostic tool for P. aeruginosa that can be employed in physician's offices, patient care settings, in the field, or in a patient's home. The specific strategy of this proposal is to develop the prototype of this technology and take it through to registration and launch of a commercial product.

Clinical Importance Of Pseudomonas

Pseudomonas is a common aerobic, gram-negative, coccobacillis. Current concerns with P. aeruginosa are both the frequency of the organism as a very common cause of nosocomial pneumonia and the emerging difficulty in treating it. Since the advent of antibiotics, P. aeruginosa has developed progressive resistance to the usual treatments. Multidrug resistant (≥3 drugs) (MDR) Pseudomonas has been reported as high as 32% in some series and rose from 13% to 21% during clinical treatment in another. However, in more recent series, the emergence of multidrug resistance occurs at rates of 27% to 72%, depending on the geography and the health care setting (10). MDR Pseudomonas pneumonias are now so frequently resistant to standard antibiotics that colistin and rifampin are often used as drugs of final resort (11). Pseudomonas has multiple mechanisms of intrinsic, acquired and genetic resistance and these mechanisms include most of the known mechanisms of bacterial resistance, including decreased transporin diffusion and lowered outer membrane permeability, increased efflux pump activity, inactivating enzymes, including multiple beta-lactamases, and inactivation enzymes for aminoglycosides and alteration of drug targets with changes in penicillin-binding activity and target site mutations of DNA gyrases (12). Drug-resistant Pseudomonas is a major concern and therapies need to be administered early in the course of the infection. Hence, a rapid and cheap diagnostic is critical to realizing effective treatment.

Pseudomonas frequently causes serious infections in humans. P. aeruginosa is often responsible for nosocomial pneumonias and particularly, ventilator acquired pneumonias (13). The organism is also often present in surgical, cardiac, respiratory and neonatal intensive care units. Most diagnostic assays for Pseudomonas utilize culture-based standard microbiology and generally require at least 24 hours. Confirmatory techniques for cultures include fluorescent microscopy, PCR, Taqman and other methods, all of which have variable sensitivity and specificity (14-17). These techniques may or may not lend themselves to bacterial surveillance approaches, depending on the clinical setting and urgency for the surveillance. The current state of point-of-care diagnosis of P. aeruginosa infections in high-risk settings is a combination of patient symptoms, clinical judgment and a gram stain. After an overnight culture, newer technologies can dramatically reduce the time to confirm the P. aeruginosa diagnosis but these technologies cannot be used in a physician's office or in a patient's home. Detection of P. aeruginosa by gram staining requires a relatively concentrated sample for detection and this approach is non-specific.

We propose a novel approach to detect whole cell P. aeruginosa in sputum, blood or from any biological source and also in environmental sources, whether in-hospital or out of hospital (eg., in ventilators, water supplies, dialysis units, etc.). The proposed technology does not rely on an antibody- or aptamer-based approach for binding and detection of the bacteria. We are confident that removing the use of cultures and the requirement for sophisticated instrumentation will significantly increase the potential for this technology to be more widely applied. Speeding and simplifying the diagnostic process will allow us to better understand the onset and progress of clinical Pseudomonas infections and to understand the health care environment and the potential for Pseudomonas infection in that environment.

Clinical Importance Of Acinetobacter

The emergence of Acinetobacter baumannii strains resistant to antibiotics has become an increasing problem over the last twenty years. This bacterium is a frequent resident of intensive care units and is often associated with disease in patients in these units. Acinetobacter now causes approximately 1.5% of hospital-acquired blood infections and may also be found in wounds, urine and the lung. Approximately 30% of Acinetobacter isolates are resistant to >4 classes of antimicrobials (18). Annually, approximately 12,000 cases of serious infections are due to multi-drug resistant Acinetobacter in the U.S. (19). Acinetobacter resistance has increased dramatically since the early 1970s. At that time, the generally reliable therapies included gentamycin, minocycline, ampicillin and carbenicillin. In the late 1970s, the therapeutic armamentarium was narrowed to 3^{rd} and 4^{th} generation cephalosporins. In the 90s, carbapenams were generally effective but other agents needed to be tailored to the specific isolates. Currently, polymyxins and tigecycline are generally reliable but now pan-resistant Acinetobacter species have emerged (20). The mechanisms for A. baumannii resistance are primarily plasmid-mediated beta-lactamases and genes transferring the capability to de-repress the efflux pump (21). The combination of these two mechanisms has contributed to the fact that upwards of 63% of healthcare-associated Acinetobacter infections are caused by multidrug-resistant strains (19). Additionally, the ability of this organism to survive outside of the host makes its environmental detection critical in preventing additional spread. The large number of Acinetobacter wound infections in the Iraq war may well have been due to acquisition of the organism through contact with the field hospital environment (beds, countertops, etc.), rather than through soil acquisition or direct patient-to-patient spread (22). Although multiple drug treatment regimens are now used as standard therapy for Acinetobacter infections, there are significant concerns that even multiple drug regimens will fail as the organism develops additional resistance. Again, earlier detection will lead to earlier treatment and environmental detection may lead to prevention.

The echnology uses siderophores immobilized to a solid-state scaffold to capture bacteria of interest and then couples siderophores to Au nanoparticles that, with Ag(I) crystal formation, secondarily develops the capture signal (23). Although we intend to use the Au NP-Ag(I) technology development (24), we will also evaluate avidin-biotin systems and radio-isotopic detection systems. The general outline of the approach is demonstrated in the figures below.

FIG. 1. Key Steps for siderophore-based bacterial immobilization and signal detection amplification (A) Functionalized siderophore-modified surface is exposed to a population of Pseudomonas or Acinetobacter containing receptors for the siderophore. (B) Targeted bacteria are “pulled down” onto the surface from solution and non-specifically adsorbed bacteria are removed by stringent washing. (C) Captured bacteria are exposed to siderophore-modified Au nanoparticles (NPs). (D) Au NP-siderophore-bacteria complexes are exposed to Ag(I) solution in the presence of a reductant, e.g. formaldehyde, hydroquinone, effecting growth of Ag crystals at the Au NP nucleation sites and, thus, visual amplification of the bacteria pull-down event.

Our preliminary data demonstrate that:

a) The HD-01-aminopenicillin combination shows great activity against Pseudomonas (More than 4000 times the activity of the antibiotic alone! (9)

b) The siderophore, HD-01, can be bound to solid state surfaces and bind Pseudomonas (FIG. 3)

c) The Ag(I) crystal technology that we use for the bacteria capture signal is well established and has been demonstrated in other systems (FIG. 4)

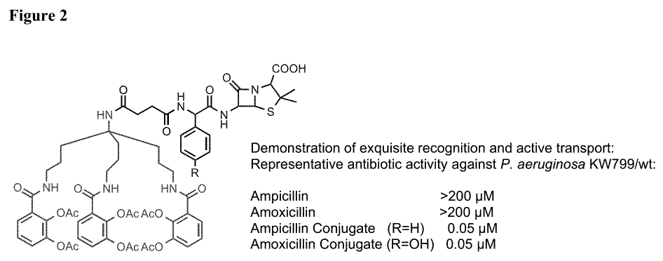

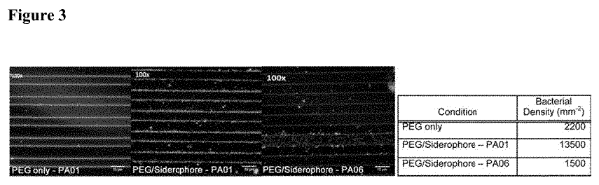

) Previous work from the Miller laboratory (9) has determined that the tripodal catecholate siderophore shown in FIG. 2, when coupled to an aminopenicillin, has outstanding antimicrobial activity against several P. aeruginosa strains (even when the base penicillins were inactive.) This unequivocally establishes that the bacteria recognize and bind the siderophore, and in the case of the antibiotic conjugate, actively transport it as well.

e) In addition to being able to bind to Pseudomonas in both semi-solid and broth dilution cultures as shown above and published previously, the surface bound siderophore is able to specifically immobilize the target Pseudomonas strain (PA01), while resisting both non-specific adsorption (PEG only) and capture of the non-target strain (PA06) on solid scaffolds as shown in FIG. 3. In this demonstration of Pseudomonas binding, the semi-quantitative nature of the binding is obvious with binding different quantities of Pseudomonas PA01 and PA6 (1.5 to 13.1×10³ bacteria/mm² enhanced with di-eletrophoretic pull-down and additional reactants).

FIG. 3

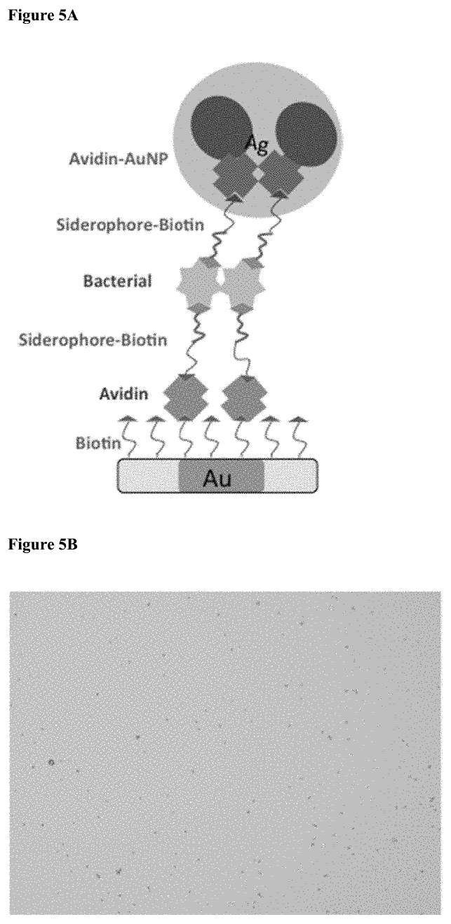

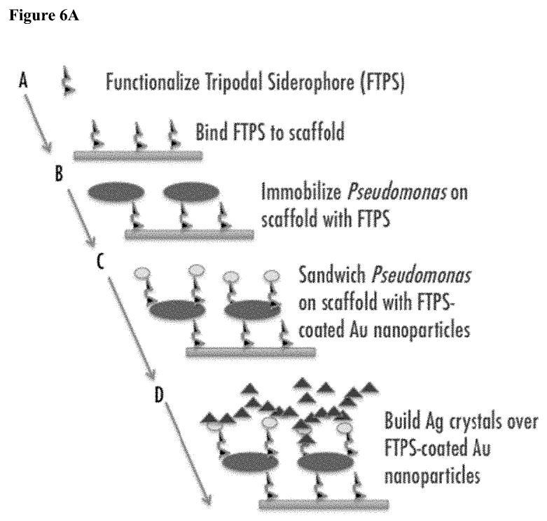

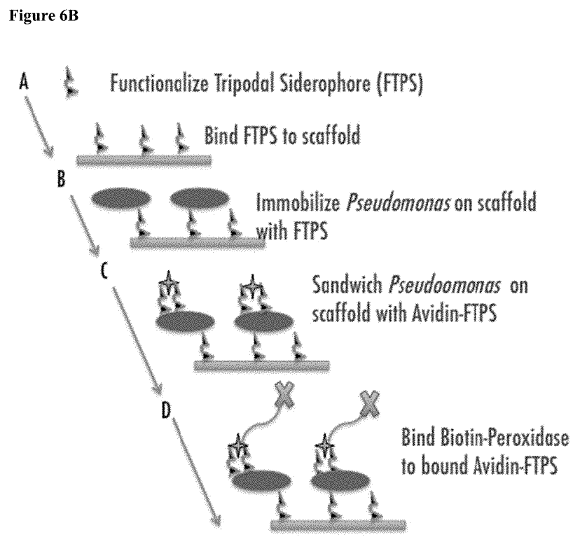

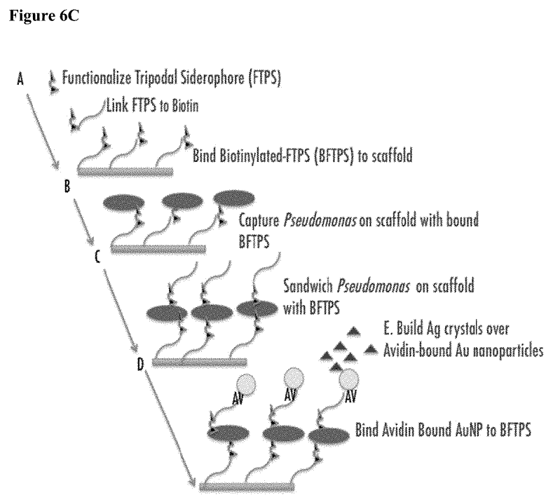

f) We have used Au nanoparticles binding with secondary development of Ag(I) microcrystals extensively in the past. FIG. 4 shows our surface chemistry/enhancement scheme along with photographs of the image of the substrate before (3) and after (4) Au nanoparticle decoration, and after enhancement by Ag(I) reduction (5). Variations of this sandwich technique and preliminary results with these techniques are outlined in FIG. 5 and FIG. 6 below.

Approach

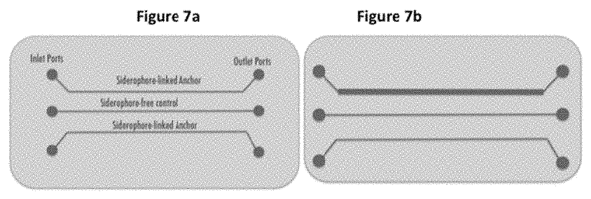

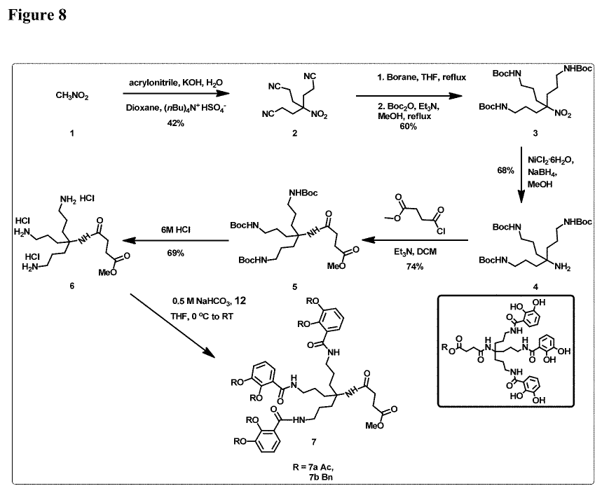

As shown in FIG. 7a-7c below, the detection platform combines self-referencing microfluidic multi-lane arrays and inexpensive, disposable fluidic chips. Use of a microfluidic format enhances mass transport, meaning that measurements can be cycled faster. The ultimate solution-referenced limit of detection (LOD) is determined by the capture efficiency and we believe that with well-designed microfluidic delivery formats LODs of a few bacteria per mL are readily attainable. We will develop the surface derivatization chemistries, starting from the Au-thiol self-assembly approach shown in FIGS. 4, 5, and 6 followed by characterization of the bacteria-engineered surfaces—including surface densities, structural properties, and quantification of binding activities—to optimally deploy the selective chemistries developed for both P. aeruginosa and A. baumannii recognition as outlined below. We expect to use this selective, naturally designed and evolved system to develop a uniquely sensitive diagnostic tool for rapid detection and identification of these bacteria in samples obtained from patients and from environmental sources.

FIG. 5. Current Proof of Concept with Pseudomonas: Visualization of trapped Pseudomonas PA01.

FIG. 5A. Schematic of Capture Motif FIG. 5B. Positive Capture Signal for Pseudomonas. Control slides show no capture “spots”

FIG. 5. In the prototype chip, the use of an ultra thin layer of Au is convenient because biotinyltated thiols that will self-assemble on Au are commercially available. In the future, activation and derivatization of polymethyl-methacrylate plastic (PMMA) is well described in the literature and will likely be used to link directly the siderophores. However, The more complex anchoring two-sandwich construction of Au-biotin-avidin-biotin-siderophore that is shown in FIG. 5A allows a great deal of flexibility in accessing the recognition site on the bacterium which may be quite important for deeper binding siderophore binding sites. Multiple approaches with these technologies will be assessed for their sensitivity, specificity, and product stability.

Depending on whether we achieve the best results with direct conjugation of the siderophore to the PMMA, use a Au coating on the scaffold, or link the siderophore to a biotin core, our diagnostic device may assume many configurations. We have outlined three additional approaches in FIGS. 6A, B, and C below FIG. 6A Gold NP/Silver Aggregation Reporter:

FIG. 6A. AuNP-Ag(1) aggregation technique: Key Steps for tripodal siderophore Pseudomonas immobilization and signal detection amplification. (A) Functionalized tripodal siderophore is linked to the PMMA scaffold. (B) Siderophore-modified surface is exposed to a population of bacteria containing receptors for the siderophore. Targeted bacteria are “pulled down” onto the surface from solution and non-specifically adsorbed bacteria are removed by stringent washing. (C) Captured target bacteria are exposed to tripodal siderophore-modified Au nanoparticles (NPs). (D) AuNP-tripodal siderophore-bacteria complexes are exposed to Ag(I) solution in the presence of a reductant, e.g. formaldehyde, hydroquinone, effecting growth.

FIG. 6B. Avidin-Biotin-Enzyme (Peroxidase) reporter:

FIG. 6B. Avidin-Biotin-Enzyme (Peroxidase) reporter: Steps (A) and (B) for this this reporter method are similar to the Ag(1) aggregation technique depicted in FIG. 6A. (C) Captured target bacteria are exposed to a tripodal sideropore-avidin complex and the avidin is bound on the Pseudomonas surface (D) Biotin conjugated to peroxidase or to a variety of other potential final visualization compound is applied and after conjugation, will be developed with diaminobenzidine-peroxide or another appropriate reagent.

FIG. 6C. Siderophore Sandwich with AuNP and Ag Crystal Reporter:

FIG. 6C. Dual Biotinylated Siderophore Sandwich with AuNP and Ag Crystal Reporter: Key Steps for tripodal siderophore-based Pseudomonas immobilization and signal detection amplification. (A) Tripodal siderophore is functionalized and conjugated with biotin. Biotin-tripodal siderophore is anchored to the PMMA scaffold. (B) Targeted bacteria are “pulled down” onto the surface from solution and non-specifically adsorbed bacteria are removed by stringent washing. (C) Captured target bacteria are exposed to biotin-conjugated tripodal siderophore (D) AuNP-avidin complexes are reacted with the anchored biotin (E) The surface is exposed to Ag(I) solution in the presence of a reductant, e.g. formaldehyde, hydroquinone, effecting growth of Ag crystals at the Au NP nucleation sites and, thus, visual amplification of the bacteria pull-down.

The final microfluidic device (as depicted in FIG. 7) will use a PMMA scaffold for ease of fabrication and for its properties to accept conjugates.

FIG. 7a shows a general schematic overview of a prototype polymethymethacrylate solid scaffold. Representative diagnostic siderophores will be immobilized on the surface of the PMMA in the outside lanes (below). A depiction of a postive response is shown in FIG. 7b. These chips are easily made and modified by channel length, channel volume, port volume and flow rates down the microfluidic channels.

AIM 1: Profile and Develop the Novel Tripodal Catecholate Siderophore (HD-01) as an Anchor for the Siderophore-Based Diagnostic for Pseudomonas

Aim 1a—Synthesize Functionally Active HD-01

The synthesis of the tri-catechol siderophore A, HD-01, follows the route previously described by the Miller group (9) and is summarized in FIG. 8/Scheme 1.

FIG. 8/Scheme 1 Synthesis of tri-catechol HD-01.

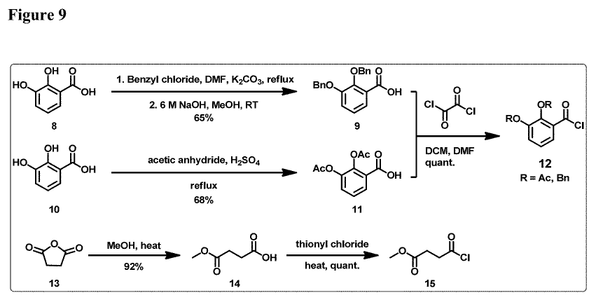

The synthesis began with commercially available nitro methane (1) that in a Michael fashion was reacted with acrylonitrile under basic conditions to yield compound (2) in 42% yield. Compound 2 was then reduced utilizing borane to yield a nitro tri-amine intermediate, which was subsequently Boc protected to give compound (3). Using nickel chloride and sodium borohydride, compound (3) was reduced to give free amine (4) in 68% yield Amine (4) was coupled to commercially available and easily synthesized methyl 4-chloro-4-oxobutanoate to afford compound (5) in 74% yield. Deprotection of the Boc group with 6 M HCl yielded HCl salt (6) in 69% yield. This intermediate was then coupled with compound (12) (FIG. 9/Scheme 2) under mild basic conditions to afford compound (7a). Syntheses of intermediates used towards the formation of compound (12) are illustrated in Scheme 2. Briefly, bis-protected benzoic acid (9) was synthesized from commercially available 2,3-dihydroxy benzoic acid (8) in 2 steps in 65% overall yield. Acid (11) was synthesized in a similar manner in 68% yield, however utilizing a different protecting group. Both compounds (9) and (11) were subjected to in situ formation of acid chloride (12) in quantitative yield, which was subsequently used in the synthesis of compound (7) as illustrated previously in Scheme 1. Additionally, compound (15) was synthesized in two steps beginning with commercially available succinic anhydride in excellent overall yield.

FIG. 9/Scheme 2. Synthesis of tri-catechol HD-01 precursors.

Aim 1b—Couple HD-01 to a Fabricated Polymethylmethactylate Scaffold

Immobilize and Functionalize the Tripodal Catecholate Siderophore on Poly(Methylmethacrylate) (PMMA) Plastic

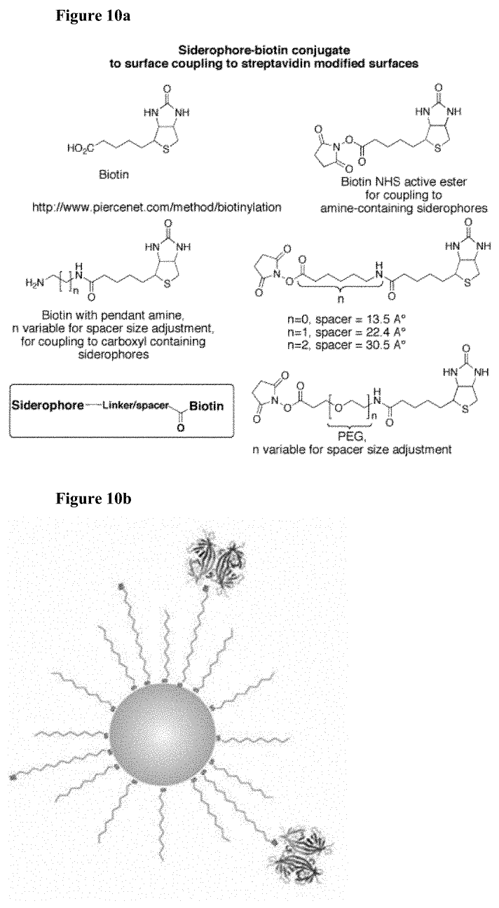

While the synthesis is compatible with large scale production of siderophore 7, the anticipated sensitivity of our diagnostic agents suggests that relatively small quantities (mg) will be needed for our use. Still, we anticipate preparation of gram quantities for use in fully optimizing standard immobilization chemistries. Principal among these, we will prepare versions of 7 terminated with biotin for recognition in standard biotin-avidin surface immobilization schemes. The standard approach we will use to construct the siderophore-biotin conjugagtes is outlined in FIG. 10a and with a depiction of the biotin attached to the sandwiched siderophore shown in FIG. 10b.

As shown in FIG. 10b, a thiolated linker presenting a terminal biotin (Bt) can be used to immobilize avidin (ribbon structure), which in turn can then recruit additional biotinylated reagents, such as biotinylated siderophore. This facile route can be adapted to a wide variety of surfaces—(PMMA, SiO₂, etc.) simply by changing the headgroup chemistry—and different recognition schemes, so it represents a platform on which a large number of bacterial pull-down schemes can be supported. In particular, the same surface-biotin-avidin construct can be used in the recognition regions of the multilane microfluidic chamber. In addition to providing a well-characterized synthetic handle, the biotin-avidin construct should optimize reactivity by moving the siderophore sufficiently far from the surface to minimize any steric constraints to recognition by bacterial receptors. To determine the sensitivity selectivity of the binding of this catecholate siderophore, we will perform standard siderophore-mediated growth promotion studies with targeted strains of Pseudomonas and other Gram positive and Gram negative bacteria in our collection.

We will fabricate multiwell microplates out of PMMA, with dimensions comparable to standard commercial 96 well microplates with round bottoms, well volumes of 330 μL, and lower surface areas of 0.36 cm². These microplates will be used to assess the binding conditions (e.g. concentration, pH, medium, temperature, time, etc.) of HD-01. Although microplates made with other plastics (e.g., polystyrene) are commercially available, they are not optimal materials for microfluidics as they are difficult to form and machine, have poor solvent compatibility, have generally undesirable mechanical properties and are a poor match to other materials used in microfluidics. We will also assess non-specific HD-01 binding directly to the plastic and to plastic treated with an adhesion-resistant coating, such as BSA or covalently bound poly(ethylene glycol). We will use radiolabelled catecholate siderophore to assess both the kinetics and the durability of the binding. We will determine the amount of siderophore bound to the reaction vessel and assess the impact of that binding density on subsequent P. aeruginosa binding in the chambers. This will inform the choice of the process to be used when we eventually fabricate the microfluidics chamber for prototype development.

HD-01 was found to be a selective inhibitor of P. aeruginosa with potency 30-90 times better than select strains of E. coli and no effect was observed when tested in an agar-diffusion assay against a panel of gram-positive and other gram-negative bacteria. Control studies indicated that non metal-binding precursors (O-benzyl protected catecholate conjugates) do not display any antibiotic activity, thus confirming that the siderophore-antibiotics are recognized and actively transported as designed. In summary, we have demonstrated that we can synthesize tripodal catecholate derivatives that are selectively recognized and are suitably functionalized for immobilization on the proposed microfluidic device using standard linker technologies (amide coupling and maleimide-mediated conjugation).

Aim 1c—Optimize the Reaction Conditions for Pseudomonas Capture

After establishing and characterizing the system to anchor the trapping siderophores, we will quantify the system's ability to capture bacteria. We will use several strains of P. aeruginosa as positive controls and non-Pseudomonas Gram-negative bacteria as negative controls. Each of the isolates will be cultured in a quantitative fashion to determine their immobilization capacity. In addition, isolates will undergo serial dilutions and the application volume and dwell time in the device will be optimized for sensitivity and specificity to detect P. aeruginosa. We will determine the binding capacity of the system, the reproducibility of the binding, the optimal detection conditions and the specificity of the technology for P. aeruginosa. We will also determine the lower levels of detection of the system with bacteria grown in C¹⁴-labeled valine (25).

Preparation of the system with HD-01 tripodal catecholate bioconjugates:

Organisms:

P. aeruginosa strains (KW799/wt, KW799/61, PA01, Pa4, Pa6) and other gram negative bacteria (e.g. E. coli ATCC 25922, E. coli H1443, E. coli H1876, K pneumonia ATCC 8303 X68) will be quantified in cultures by standard microdilution methods. The specific binding of the organisms will be determined and varying inocula will added to the microwell chambers in aliquots of 100 μL.

Sample Loading and Presentation:

The initial number of bacteria per 100 μL aliquot will be adjusted to be 10⁴ organisms. The system will be assessed for detection limits of half-log decrements from the initial load concentration. Room temperature incubations of 10, 30, and 60 mn with each sample load will be evaluated to determine if binding is time-dependent over this period. After incubation, the numbers of bacteria “pulled down” by the siderophore bound to the plates and the numbers of bacteria that were not bound to the system will be determined by measuring the radioactivity of the bound siderophores and supernatant and calculating the numbers of bacteria. This aim will help to determine the capacity and detection sensitivity of the system, variables around the methodology and binding conditions and the specificity of the system for P. aeruginosa. A mixing experiment will also be performed to evaluate specificity. This will entail adding different concentrations of radiolabeled P. aeruginosa to a mixture of unlabeled gram-positive and gram-negative bacteria or radiolabeled negative controls and then measuring the binding as described above.

After the initial assessments of bacterial binding in the system have been determined, we will alter several variables (temperature, time, bacterial concentrations, pH, buffer strength, etc.) to determine their effects on the overall performance of the system. We will anchor the tripodal catecholate P. aeruginosa-specific siderophore, i.e., the siderophore component, to a surface (gold or polymer) nanoparticle so that the captured bacteria will bind the siderophore and also anchor the Au-catecholate siderophore nanoparticle to the surface. The siderophore-bioconjugates will be functionalized to a nanoparticle capture surface via a heterobifunctional linker, allowing us simultaneously to: (a) mitigate against non-specific adsorption; (b) present competent capture motifs well-separated from the underlying protective layer; and (c) effect binding to P. aeruginosa with exceptionally high sensitivity and selectivity.

Aim 1d—Conjugate HD-01 to Au Nanoparticles for Development of Capture Signal

After establishing optimal pull-down conditions, we will identify the optimum conditions for binding 20 nm Au nanoparticles to the immobilized bacteria (step (3) to (4) in FIG. 4). We anticipate that these will be similar to those for the binding step to the PMMA scaffold. Bacterial recognition and labeling by the siderophore-Au NP construct will be quantified and specifications for lot-to-lot variation will be established with additional synthesis prior to manufacturing larger lots of devices. Although Au is a potentially expensive reagent, the volumes needed for the microfluidic labeling are tiny (picoliters), so that minimal Au NP reagent will be needed—we estimate 0.2 pg of Au would be needed to react a full microfluidic channel.

Once formed, Au NP-tripodal catecholate-bacteria complexes will be exposed to Ag(I) solution in the presence of a reductant, e.g. formaldehyde, hydroquinone, etc., effecting growth of Ag crystals at the Au NP nucleation sites (step (5) in FIG. 4) and, thus, visual amplification of the bacteria pull-down event. The specifics of these reactants, concentration, and reaction conditions will be optimized. After collecting the sample, the capture signal will be viewed either on a field-deployable reader or, ideally, with a simple hand-held viewer.

Aim 1e—Fabricate a Working Prototype of the Device

Ongoing design modifications with appropriate design control documentation will proceed through the first year of funding. By that time, we anticipate that we will have settled on a final design and will be evaluating material availability, costs and industrial design. Design control will be performed in accordance with the FDA CDRH Guidance Documentation. We anticipate having a fully validated design and will be identifying a manufacturer by Q4. We will refine these designs in consultation with the manufacturer and anticipate being in the production phase by Q8.

Aim 1f—Scale up the GMP Manufacture of the Diagnostic Device

We will determine the performance of multiple chip designs and will refine the manufacturing specifications for their production. Manufacture will be performed by a third party with appropriate experience, in accordance with FDA CDRH guidance and with full implementation of necessary quality regulation systems,

We anticipate that process refinement for the development kits of the point-of-care system will take approximately 6 months. Initially, the process will be determining the appropriate chemical reagents to accomplish the multiple tasks of sample preparation, chip binding, chip washing and chip developing. Ideally this can be done with two solutions but it may require more. When a reproducible process has been validated, we will ensure stability of the reagents under various conditions over time. We will conduct final stability tests in conjunction with the manufacturer, who will manufacture the clinical trial supplies and perform stability testing on the manufactured commercial lots.

Aim 1g—Assess Fully the Reproducibility, Load Detection, Sensitivity, Specificity and Predictive Accuracy of the Device in Laboratory Settings

In this objective, we will retest the optimized binding conditions for Pseudomonas, including the specifics of specimen preparation, bacterial load, buffers and solutions to be used, incubation periods and washout conditions. We will then assess the sensitivity and specificity of the system in trials in clinical laboratories with appropriate and accepted standards. We will also assess the specificity of the system with a variety of other bacterial species, including Proteus, Serratia, Klebsiella and E. coli. In addition to multiple strains of Pseudomonas from biological sources, we will assess the sensitivity of the system to detect Pseudomonas from environmental sources. We will work with hospital infection control staff for this environmental assessment. The Miller group has an established collaboration with Dr. James Harris at the South Bend Clinic and St. Joseph's Hospital for the provision of clinical isolates from cystic fibrosis patients. We will also confirm the sensitivity of the system to detect bacteria from a variety of biological sources.

Aim 1h—Assess the Usability and Accuracy of the Device in Clinical Settings

As part of the clinical development program, we will ensure that staff can use the device and that the field testing of the device in their hands is adequate. The size of the clinical trial, supply manufacturing program and the microbiological studies will be designed in consultation with CDRH at the FDA. Statistical support will be hired on a consulting basis. The extent of the clinical program will depend on whether CDRH allows registration under a 510k or requires a Premarket Approval Application (PMA). Our early conversations with regulatory experts suggest that a 510k application will be allowed.

Aim 1i—Submit 510k for Regulatory Approval

Complete the regulatory registration dossier and refine manufacturing processes (if necessary) for commercial supplies. Based on the consistency of the system's performance with multiple lots, we will either progress to a wider manufacturing effort (initial commercial supplies) or will review our fabrication processes to establish that consistency. When the constancy of the system and processes have been established, we will begin a wider manufacturing effort. The regulatory submission will be assembled with the help of regulatory consultants experienced in approvals from CDRH as well as approvals in markets outside of the U.S.

Aim 1j—Launch Product

We will most likely launch the product in conjunction with companies established in this area. Marketing, pricing, distribution, inventory, and customer support strategies will be determined at a later date.

AIM 2: In general, with the exception of the siderophore (fimsbactin and a synthetic fimsbactin mimic) chemistry, the steps required for the Acinetobacter diagnostic product will be the same as with the Pseudomonas diagnostic product, The Acinetobacter program will he conducted over a staggered timeline as success emerges with the Pseudomonas program.

Aim 2a—Synthesize the functionally active fimsbactin analog, HD-02 and simplified mixed ligand mimic HD-02A.

The Miller group recently reported the design, syntheses and studies of a mixed ligand siderophore conjugate of the carbacephalosporin, Lorabid. While Lorabid itself is not active against Acinetobacter baumannii, the conjugate is extremely potent and selective with an MIC value of 0.0078 against A. baumannii ATCC 17691. The antibacterial activity of the β-lactam sideromycin was inversely related to the iron(III) concentration in the testing media and was antagonized by the presence of the competing parent siderophore. These data suggested that active transport of the mixed ligand β-lactam sideromycin across the outer cell membrane of A. baumannii via siderophore-uptake pathways was responsible for selective and potent antibacterial activity (26.).

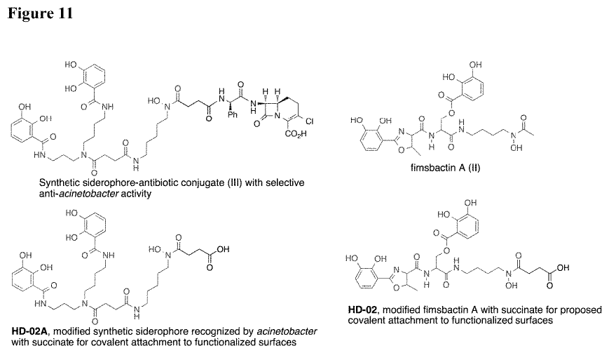

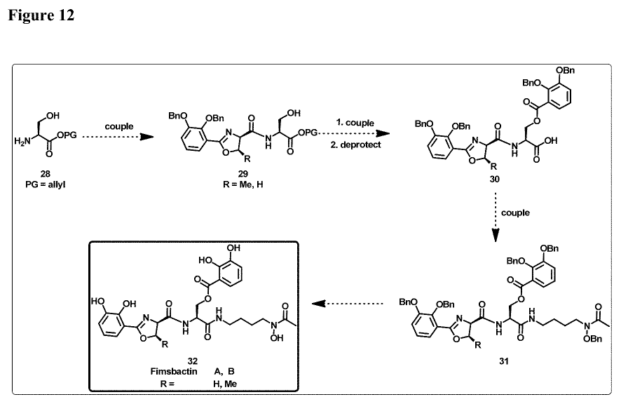

Another group reported the isolation of fimsbactins as important siderophores for growth and virulence of A. baumannii (27). The structure similarity to the synthetic mixed ligands and the natural products (fimsbactin) is remarkable and most likely accounts from the impressive select anti-Acinetobacter activity of the mixed ligand-lorabid conjugate. Both the natural product and our synthetic mixed ligand siderophore contain two catechols and a hydroxamic acid with similar molecular frameworks. As shown in FIG. 11 above, the mixed ligand siderophore has already been prepared with a succinate linker set for immobilization chemistry. The synthesis of the natural product, fimsbactin, is summarized below and is in progress. The final stages of the synthesis can be modified to replace the terminal acetyl group of the natural product with a succinate for subsequent linkage chemistry.

FIG. 12/Scheme 3. Synthesis of Fimsbacins A and B, HD-02.

The synthesis of fimsbactin A and B, whose forward synthesis is illustrated in FIG. 12/Scheme 3. Synthesis of allyl protected serine 28 and its immediate coupling with compound 38 will afford compound 29. Compound 29 will then be subjected to coupling with acid 9 and after deprotection will afford compound 30. Intermediate 30 will then be subjected to coupling with the free amine of 36, which will yield intermediate 31. Upon global deprotection, synthesis of siderophore 32 will be accomplished.

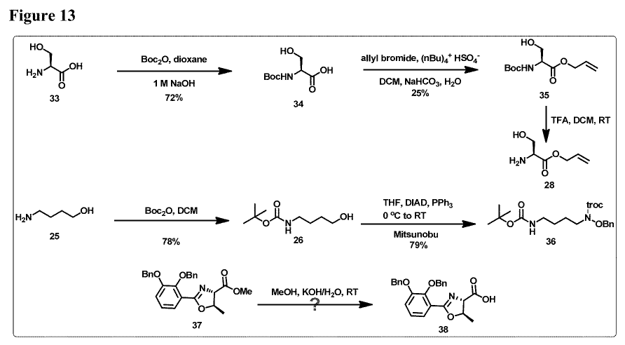

This project is in early stages of development; however, the progress thus far is shown in FIG. 13/Scheme 4. Using L-serine as the starting material, compound 34 was synthesized in 72% yield. Compound 34 was then subjected to O-allyl protection under basic conditions to afford compound 35. Deprotection under acidic conditions yielded O-allyl protected L-serine intermediate 28. Intermediate 36 that is one of the coupling partners in the synthesis of the fimsbactins was synthesized starting from commercially available 4-amino-butanol 25. This amine was Boc protected in good yield to give alcohol (26), which was then subjected to Mitsunobu reaction conditions to yield compound (36) in 79% yield. Oxazolidine 37 can be easily obtained from coupling of acid 9 and either protected L-serine or L-threonine, which upon coupling can be cyclized using DAST to yield the oxazolidine moiety of the fimsbactin core. At this point, we have multigram quantities of both oxazolines. Saponification is anticipated to generate the free carboxylic acid of the oxazoline components which can then be coupled to the remaining fragments.

FIG. 13/Scheme 4. Synthesis of Fimsbactins A and B, HD-02

At the conclusion of this project we anticipate having launched two novel and necessary products for the detection of Pseudomonas and Acinetobacter in both body fluids and health care settings. This will speed both the treatment of disease and, we hope, the prevention of disease with better environmental surveillance.

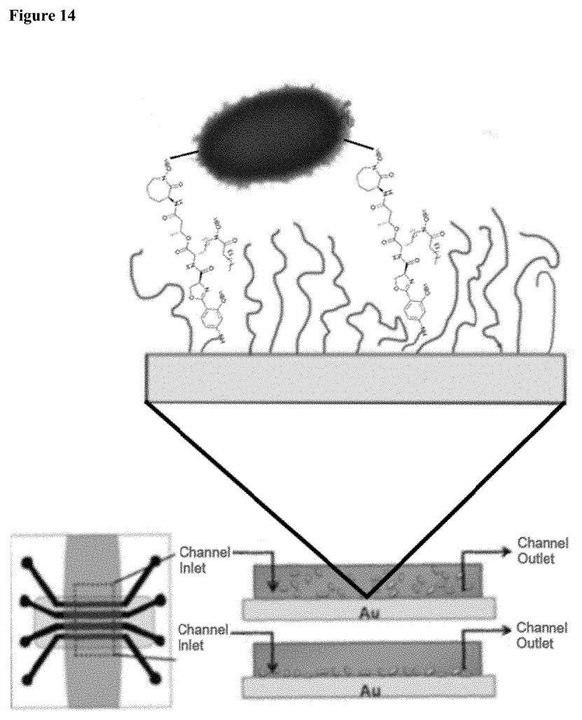

As shown in FIG. 14, the detection platform combines (a) self-referencing microfluidic multi-lane arrays; (b) surface plasmon imaging/angle shifts for readout and (c) reusable or disposable fluidic chips. Carrying out recognition in a microfluidic format enhances mass transport, meaning that measurements can be cycled faster. The ultimate solution-referenced limit of detection (LOD) is determined by the capture efficiency, and we believe that the LODs of a few mycobacteria per ml are readily attainable. We will develop the surface derivatization chemistries followed by characterization of the bacteria-engineered surfaces—including surface densities, structural properties, and quantification of binding activities—so as to optimally deploy the selective chemistries developed for M.tb recognition. We expect to use this selective naturally designed and evolved system to develop a uniquely sensitive diagnostic tool for rapid detection of and identification of M.tb in samples obtained from patients.

FIG. 14. Schematic diagram of a simple four-lane surface plasmon reader construct the Au NPs fabricated into the PMMA scaffold. The long red lines represent non-specific inert moieties, such as oligo (ethylene glycol) to diminish non-specific absorption. Mycobactin molecules are bound to the PMMA scaffold via a linker (short red lines) and they “pull down” M.tb via specific receptors on the mycobacterium.

AIM 1: Synthesis of Mycobactin T and Mycobactin Analogs

To explore how the mycobactin immobilization pathway may be used to co-opt the bacterial machinery for organism detection, visualization, and ultimately treatment (7-9), the Miller group has synthesized mycobactin T (1), the M.tb specific siderophore, mycobactin analogs and, most recently, a conjugate (3) of a mycobactin analog with artemisinin (10).

Although the antimalarial agent, artemisinin (2) itself is not active against tuberculosis, conjugation to a M.tb specific siderophore (microbial iron chelator) analog induces significant and selective anti-tuberculosis activity, including activity against MDR and XDR strains of M.tb. Physicochemical and whole cell studies indicate that ferric to ferrous reduction of the iron complex of the conjugate initiates the expected bactericidal Fenton-type radical chemistry on the artemicinin component. Thus, this “Trojan Horse” approach demonstrates that new pathogen selective therapeutic agents can be generated in winch the iron component of the delivery vehicle also participates in triggering the antibiotic activity. The result is that the critical iron uptake machinery of M.tb is demonstrably selective and thus is uniquely suited for design of a sensitive, selective and non-invasive diagnostic tool.

AIM 1: Synthesize mycobactin T derivatives and analogs with appropriate peripheral functionality to allow the siderophore to be anchored to the surface of a microfluidic device.

As described in the section on preliminary data, we have extensive experience related to the syntheses of mycobactin T. Mycobactin analogs will be synthesized using methods we have described previously (10, 11). Only one mycobactin T moiety will be advanced beyond this point at a time for purposes of reproducibility and design control.

AIM 2: Immobilize and Functionalize Mycobactin T on Poly(Methylmethacrylate) (PMMA) Plastic

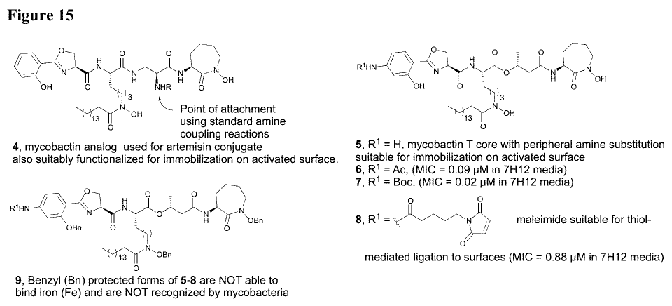

We propose to use mycobactin T analogs for immobilization on PMMA. As shown in FIG. 15, for surface modification in the proposed microfluidic devices, we can derivatize the same mycobactin analog (4) used for synthesis of conjugate 3. However, we have also already prepared additional derivatives of mycobactin T with peripheral functionalization that may be more amenable to appropriate derivatization (12). In order to perform functionalization through the aryl-oxazoline moiety, an amino group and a maleimide linker (8) were incorporated at the phenyl ring. The mycobactin core was not further modified. The amine (5) was then separately acetylated and protected as a Boc derivative to give derivatives 6 and 7, respectively. To determine if these new mycobactin T derivatives were recognized by targeted mycobacteria, they were screened against replicating M.tb. The synthetic analogs were found to be potent growth inhibitors in the Microplate Alamar Blue Assay (MABA), 6 (MIC=0.09 μM in 7H12 media, MIC=0.43 μM in GAS), 7 (MIC=0.02 μM in 7H12 media, MIC=2.88 μM in GAS), 8 (MIC=0.88 μM in 7H12 media, MIC=1.02 μM in GAS). These analogs were also found to be specific inhibitors of M.tb as no effect was observed when tested in an agar-diffusion assay against a panel of gram-positive and gram-negative bacteria. It is important to indicate that the mycobactin analogs must be interfering with the iron acquisition system considering that non metal-binding precursors (O-benzyl protected hydroxamates) do not display any antibiotic activity. In summary, we have demonstrated that we can synthesize mycobactin T derivatives that are selectively recognized and are suitably functionalized for immobilization on the proposed microfluidic device using standard linker technologies (amide coupling and maleimide-mediated conjugation).

FIG. 15. Amine (4 & 5) and maleimide (8)-containing mycobactin T analogs suitable for surface modification. Activity of derivatives 6-8 demonstrate mycobacterial recognition and. selectivity.

We intend to fabricate a multiweli microplates out of poly(methylmethacrylate) (PMMA). Microplates will be made with dimensions comparable to standard commercial 96 well microplates with round bottoms, well volumes of 330 μl, and lower surface areas of 0.36 cm². These micoplates will be used to assess the binding conditions (e.g. concentration, pH, medium, temperature, time, etc.) of the mycobactin T analogs. Although microplates made with other plastics (e.g., polystyrene) are commercially available, they are not optimal materials for microfluidics as they are difficult to form and machine, have poor solvent compatibility, have generally undesirable mechanical properties and are a poor match to other materials used in microfluidics. Therefore, we will use PMMA with which we have considerable experience (13). We will assess mycobactin binding directly to the plastic. We will utilize radiolabelled mycobactin to assess both the kinetics and the durability of the binding. We will determine the amount of mycobactin bound to the reaction vessel and assess the impact of that binding density on subsequent M.tb binding in the chambers. This will inform the choice of the process to be used when we eventually fabricate the microfluidics chamber for prototype development.

AIM 3: Optimize the binding conditions for mycobacteria, and define the specificity, and selectivity of the siderophore derivatized system for multiple strains of radiolabeled M.tb and NTM.

Preparation of the system with the mycobactin bioconjugates:

The mycobactin bioconjugates synthesized in AIM 1 will be immobilized on the PMMA microwells. Incubation and binding conditions for mycobactin will be determined in AIM 2 and will be followed in preparing the microwells in AIM 3.

Organisms:

M.tb strains (H37Rv and CDC 1551) and NTM species (M. avium 101, M. abscesses, M. kansasii, and M. paratuberculosis) will be radiolabeled by methods previously described (14). Briefly mycobacteria will be grown from single cell suspensions to an OD of 0.7 at b00 nm in salt medium containing 0.05% Tween and 2 μCi/ml of 1-³H-Glc (sp activity of 40-60 mCi/mmol. The specific radioactivity of the organisms will be determined and varying inocula will added to the microwell chambers in aliquots of 100 μl.

Sample Loading and Presentation:

The initial number of mycobacteria per 100 μl aliquot will be adjusted to be 10⁴ organisms. The system will be assessed for detection limits of half-log decrements from the initial load concentration. Room temperature incubations of 10, 30, and 60 min with each sample load will be evaluated to determine if binding is time dependent over this period. After incubation, the numbers of mycobacteria “pulled down” by the mycobactin bound to the plates and the numbers of mycobacteria that were not bound to the system will be determined by measuring the bound and supernatant radioactivity and calculating the numbers of mycobacteria. This aim will help determine the capacity and detection sensitivity of the system, variables around the methodology and binding conditions, and the specificity of the system for M.tb. A mixing experiment will also be performed to evaluate specificity. This will entail adding different concentrations of labeled M.tb to a mixture of unlabeled gram positive and gram negative bacteria and then measuring the binding as described above.

After the initial assessments of mycobacterial binding in the system have been determined, we will alter several variables (temperature, time, mycobacterial concentrations, pH, buffer strength, etc.) to determine their effects on the overall performance of the system.

Expected Results of Phase I:

At the completion of this Phase I SBIR, we anticipate that we will have demonstrated the proof of concept for the sensitivity and specificity of binding M.tb on a solid-state matrix suitable for detection signal amplification. We will then assemble our results and apply for Phase II SBIR support. Among the goals of the Phase H SBIR will be to a) create a prototype device suitable for rapid laboratory detection of M.tb, b) refine biological sample preparation methods, c) refine biological sample administration methods, d) perform additional testing on specificity of detection, particularly with additional non-tuberculous mycobacterial species, e) assess the sensitivity of the device on a large number of clinical samples of M.tb, and f) determine the conditions and modifications needed to enable the device to be deployed as a point of care diagnostic.

At the end of the Phase II SBIR, it is expected that we will have a device that will be ready for laboratory testing and that we will have defined the critical parameters needed to optimize the prototype for field-testing.

The key to effectively instituting prophylactic measures is early and reliable recognition of the problem. In a mass casualty setting, it will quickly become obvious of the likely problem and the probably etiologic agent. However, inn a smaller setting, with a potentially occult bioterror attack, such as an airport, with future casualties dispersing widely after the initial exposure, the recognition of the problem may be delayed. Since the clinical symptoms of the resulting pneumonic plague would only start to develop one to three days post-exposure (8), it may be very difficult to define the point outbreak of the problem. Pneumonic plague will initially appear as a severe pneumonia that could easily be mistaken initially as a usual bacterial or viral pneumonia (9).

Although microbiologic studies would be helpful in confirming the diagnosis, there will be a diagnostic delay while cultures are growing. There is no widely available rapid environmental test for plague and the biologic tests for Y. pestis such as Ft antigen detection, IgM immunoassay, immunostaining, PCR, and fluorescent microscopy may be available only at specific research laboratories, the CDC and some military laboratories. Further, these may not be satisfactory in determining the environmental source of the organism. With the anticipated sensitivity of the proposed technology, it will be possible to “rule out” a specific pathogen, particularly if there a combination of a baseline low level of suspicion and a significant risk of secondary infections as there might be with pneumonic plague. It will also be helpful to determine the source and location of the bioterror attack.

Potential for a Faster Confirmed Diagnosis—

Our technology is based on the absolute need of iron by Y. pestis and therefore its evolved mechanism for high affinity binding of its preferred siderophore, yersiniabactin The potential for the siderophore-based Yersinia pestis diagnostic is the rapid confirmation of the presence of organisms that will bind yersiniabactn. This would represent the majority if not all strains of Y pestis (10). Also, since Yersinia requires yersiniabactin for virulence in both the bubonic and the pneumonic forms (11), any weaponized form of the organism would not be a yeresiniabactin-negative mutant. For purposes of our diagnostic device, the source of the organism could be from any body fluid, from skin swabs, or from environmental samples. Further, depending on the degradation time of the organism, even though it is not an ensporulating bacterial species, there should be sufficient siderophore available to trigger its recognition at the attack site even after the organisms are no longer viable (12).

Having confirmation that the bioterror weapon was Yersinia and confirming the potential source will lead to: a) earlier and more rigorous respiratory isolation and protection of health care workers, b) faster specification and sensitization of the organism because of immediate escalation to higher level reference laboratories, c) rapid public education, d) rapid dissemination of prophylactic medications, and e) potentially, since the environmental source can be tested with our technology, a more rapid apprehension of the attackers and disruption of further attacks.

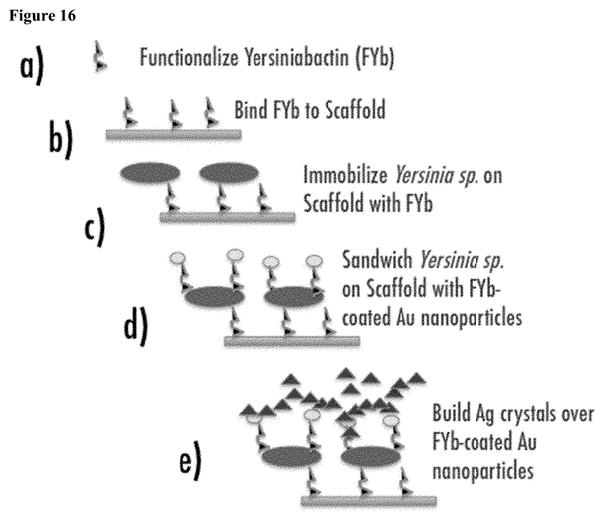

FIG. 16. a) chemically modify yersinabactin to bind to scaffold, b) bind functionalized yersinabactin to scaffold, c) apply bacterial sample to scaffold, d) sandwich trapped yersinabactin with Au nanoparticles coated with functionalized yersiniabactin, e) develop visual signal with Ag nanocrystais

The specificity of the technology depends on how unique the anchoring siderophor bacteria. Yersiniabactin is a large molecule made up of a salicylate, malonate, and three cy cysteine residues and was isolated and identified through x-ray crystallography in 2006(13). It is present for both Y. pestis and Y. enterocolitica. It appears for the vast majority of species, yersiniabactin is absolutely required by Y. pestis for its ability to infect both in the bubonic variety and in the pneumonic variety (11). It does appear that the vast majority of species of Yersinia will be trapped by this siderophore and even if the species do not utilize it as its primary siderophore, yersiniabactin may well still be an appropriate ligand to immobilize the bacteria in the diagnostic device. The data suggest that yersiniabactin in a siderophore that is shared among other members of the enterobacteriaceae family (14). Species of klebsiella and e. coli utilize this siderophore and they presumably have external binding sites for its attachment. However, these diseases would be readily separated from yersinia on the basis of a second phase 24 hour culture, epidemiology, clinical course, gram stain. Further, the sensitivity of the system will allow for very early diagnosis and detection prior to the emergence of clinical symptoms.

In the project summary, we will utilize Yersinia enterocolitica for the initial diagnostic organism. This will allow refinement of the system without the concerns of handling Yersinia pestis. We anticipate that. Y. entrocolitica will provide an acceptable surrogate for Y. pestis in the early stages of product development. The insights that we gain from Y. entrocolitica will be tested with Y. pestis.

Research Plan Objectives
1. Synthesize functionalized Yersiniabactin (FYb)
2. Bind FYb to poly(mahlymethacrylate) microfluidics chamber
3. Bind FYb to Au nanoparticles
4. Prove concept with Yersinia enterocolitica
5. Confirm concept with Yersinia pestis*
6. Characterize binding capacity and consistency across multiple lots and Yersinia strains, as well as determining the activity in a murine model of Y. pestis
7. Begin wider manufacture of diagnostic devices
*—sub-contract required **—manufacturing facility to be built or sub-contract manufacturing

General Outline of the Research

The technology centers around translating Yersinia's obligate iron needs and iron foraging biochemical machinery into a diagnostic agent. Since Fe(III) is insoluble at physiological pH, microbes have evolved specific processes for iron sequestration that involve active transport through an otherwise impermeable outer membrane (15). Bacterial iron acquisition is essential for pathogenicity and provides an attractive and little-used target for developing microbe-selective biomarkers for selective detection. Acquisition of iron by bacteria at the infection site depends on the presence of soluble Fe(III) complexes generated from iron sources, These solubilized Fe(M)-binding complexes (generically called siderophores) must then be sequestered by the bacteria to initiate iron transport across the cell envelope. For Yersinia, the unequivocal importance of the specific siderophore yersiniabactin has been established by showing that a mutant of Yersinia lacking a gene from yersiniabactin biosynthesis had could not infect hosts in either the bubonic or the pneumonic scenario. The sensitive binding of this siderophore to Yersinia will be exploited for the technology described.

The major unknowns relate to the absolute specificity of yersiniabactin for Yersina. The general chemistry and the amplification techniques have been tested previously and we have shown laboratory proof-of-concept of our technology for the detection of Pseudomonas aeruginosa using the Pseudomonas siderophore, pyoverdin.

The functionalized yersiniabactin bioconjugates synthesized in Objective 1 will be immobilized on the poly(methylmethacrylate) chambers in Objective 2. Binding of FYb to Au nanoparticles will be optimized and quantified in Objective 3. Incubation and binding conditions for Yersinia enterocolitica will be determined in Objective 4 and will be compared to the binding of other enterobacteriaceae such as klehsiella and e. coli. Assessment of the activity in Yersinia pestis and optimization of the conditions for the performance of the system will be done in Objective 5. In Objective 6, we will extend these observations and apply them to biological specimens from animal models. in Objective 7, we will establish the specifications for the manufacturing of the diagnostic device.

The proposed technology will take advantage of the exquisitely sensitive recognition of yersiniabactin by Yersinia pestis. This will allow for the development of a rapid (<1 h) and simple to use diagnostic technology. The technology will be developed to detect the presence of Yersinia from a wide variety of biological samples. Also, in contrast to the F1 antigen detection, the whole cell detection technology of this proposal will also be able to detect samples from environmental sources. The final device will be a microfluidic multichannel affinity recognition and detection system based on covalent attachment of yersinia-specific siderophores and analogs to the surface of separate channels in the microfluidic device. Passage of microliter volumes of sample through the device will allow exposure to the adsorbed siderophores that specifically recognize and tightly bind Y. pestis. The Yersinia thus immobilized on the device will be detected by using a label free surface-plasmon (SPR) detection methodology for which we have considerable experience (16-19). The optimal device will be low cost, easy to use, and highly sensitive, compared to either standard gram-staining staining of Yersinia or fluorescently aided microscopy. In theory, this technology has the sensitivity to be able to detect a single bacterial cell.

The general schematic of the yersinia “pull down,” Yersinia binding detection, and signal amplification is shown in FIG. 2.

FIG. 2. Key Steps for yersiniabactin-based Yersinia immobilization and signal detection amplification (A) Siderophore-modified surface is exposed to a population of bacteria containing receptors for the siderophore. (B) Targeted bacteria are “pulled down” onto the surface from solution and non-specifically adsorbed bacteria are removed by stringent washing. (C) Captured target bacteria are exposed to yersiniabactin-modified Au nanoparticles (NPs). (D) Au NP-yersiniabactin-bacteria complexes are exposed to Ag(I) solution in the presence of a reductant, e.g. formaldehyde, hydroquinone, effecting growth of Ag crystals at the Au NP nucleation sites and, thus, visual amplification of the bacteria pull-down event.

In the first phase of the project, we will synthesize yersiniabactin or an analog for later functionality. Depending on the iron binding after we have functionalized yersin1abactin. we may consider substituting the carboxylate with an alternate moiety such as a hydroxamate, amide, etc. that would allow another modification and linker attachment. With multiple syntheses, and attempted functionalization, we will determine the appropriate base molecule and then consider the functional modifications for PMMA attachment. In the next phase, yersiniabactin, e.g. the siderophore component of 3-6 and 8 (see below), will be anchored initially to the PMMA surface and then to a surface (gold or polymer nanoparticie) so that the yersiniabactin siderophore, will also anchor the Au nanoparticle to the surface—a process that will be detected using label free SPR detection (20, 21). The siderophore-bioconjugate will be functionalized to a capture surface (pegylated Au, chosen for resistance to non-specific adsorption) via a heterobifunctional linker, allowing us simultaneously to: (a) mitigate against non-specific adsorption, (b) present competent capture motifs well-separated from the underlying protective layer, and (c) capture Yersinia with exceptionally high sensitivity and selectivity. The potential high-cost driver derived from the use of Au in the prototype device can eventually be circumvented. The localized surface plasmon effect can be used in transmission with inexpensive Au colloid active layers. After collecting the sample and then developing the capture signal, it will be viewed either on a field-deployable reader, or ideally with a hand-held magnifying glass.

### Objective 1—Synthesize functionalized Yersiniabactin (FYb)

We will synthesize yersiniabactin derivatives and analogs with appropriate peripheral functionality to allow the siderophore to be anchored to the surface of a microfluidic device.

We have extensive experience related to the syntheses and modification of siderophores (22-25). Yersiniabactin analogs will be synthesized using modifications of methods described previously (13). Only one yersiniabactin analog will be advanced beyond this point at a time for purposes of reproducibility and design control.

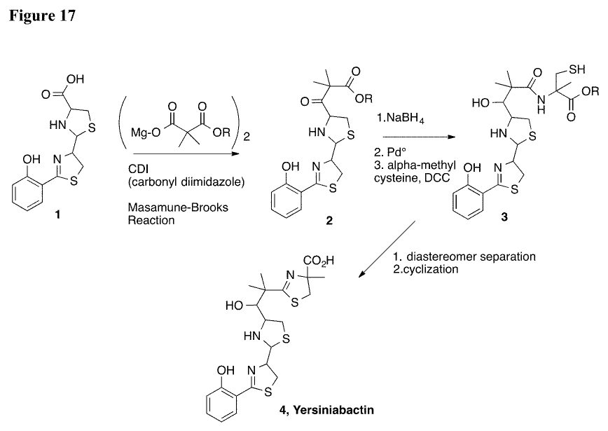

The general scheme for the synthesis of yersiniabacdn in outlined in FIG. 17. Starting with pyochetin,1, a Masamune-Brooks reaction gives rise to 2. Reduction of the carbonyl is followed by deprotection and peptide coupling to alpha methyl cysteine. The diastereotners of 3 are then separated and the side chain cyclized to give yersiniabactin, with the carboxyl moiety open to couple to a linker which will then be coupled to the device.

FIG. 17: Proposed Synthetic Sequence for the Synthesis of Yersiniabactin

Expected Results from Objective 1: At the conclusion of Objective 1, we anticipate having a full understanding of the synthetic chemistry pathways and reaction conditions necessary for synthesizing functionalized yersiniabactin suitable for linking to the plastic scaffold.

### Objective 2—Bind FYb to Poly(Methlymethacrylate) the Microfluidics Chamber

We propose to use yersiniabactin analogs for immobilization on poly(methylmethacrylate) plastic. As done with other siderophore, for surface modification in the proposed microtluidic devices, we can derivatize the same yersiniabactin analog (4) used for synthesis of conjugate 3. Functionalization through the carboxyl moiety, an amino group and a maleimide linker (8) will be incorporated. The yersiniabactin core will not be further modified for the initial work. The amine will then be separately acetylated and protected as a Boc. To determine if these new yersiniabactin derivatives will be recognized by targeted Yersinia, they will be screened against replicating Y. enterocolitica.

Poly(methylmethacrylate) scaffolds will be used to assess the binding conditions (e.g. concentration, pH, medium, temperature, time, etc.) of the yersiniabactin analogs. We will assess yersiniabactin binding directly to the plastic. We will determine the amount of yersiniabactin bound to the reaction vessel and assess the impact of that binding density on subsequent Yersinia enterocolitica binding in the scaffold. This will inform the choice of the process to be used when we eventually fabricate the microfluidics chamber for prototype development.

Expected Results from Objective 2: At the conclusion of Objective 2, we anticipate having a full understanding of the chemistry and binding conditions necessary for linking yersiniabactin to the plastic scaffold.

### Objective 3: Bind Functionalized Ybt to Au Nanoparticles.

The binding conditions of this reaction will be essentially that of the binding step to the poly(methylmethacrylate) scaffold. Unbound yersiniobactin will be separated from the bound Au nanoparticles by physical separation techniques. Yersiniabactin binding to the nanoparticles will be quantified and specifications for lot to lot variation will be established with additional synthesis prior to manufacturing larger lots of devices.

Expected Results from Objective 3: At the end of this objective, we anticipate having established the reproducible standard conditions for linking the Au nanoparticles to the yersiniabactin-bound yersinia.

### Objective 4: Prove Concept with Yersinia Enterocolitica

In this objective, we will optimize the binding conditions for Yersinia entreocolitica, including the specifics of specimen preparation, bacterial load, buffers and solutions to be used, incubation periods, and washout conditions. We will then assess the sensitivity and specificity of the system. In addition to multiple strains of yersinia, we will assess the specificity of the system with wide variety of enterobacteriacieae including klebsiella and e. coli species.

Expected Results from Objective 4: At the end of this objective, we will have established the optimal binding conditions for Yersinia enterocolitica to the yersiniabactin scaffold. We will established the sensitivity of the system and established the limits of detection. We will have established the relative specificity for the system for yersinia. If there are other organisms the system detects, we will determine the characteristics of the device.

### Objective 5: Confirm Concept with Yersinia Pesitis

Optimize the binding conditions for Yersinia pestis and define the sensitivity of the siderophore derivatized system for multiple strains of yersinia from both bacterial and environmental sources.

Expected Results from Objective 5: At the end of this objective, we will have confirmed that the system is sensitive for Yersinia pestis as well as Yersinia enterocolitica. We will confirm the sensitivity of the system to detect bacteria from a variety of biological and environmental sources.

Objective 6: Characterize Binding Capacity and Consistency Across Multiple Lots and Yersinia strains, as well as Determine the Activity in a Murine Model of Y. Pestis

Although the literature reports some variability in murine modes, we will use a mouse model of primary pulmonary yersinia (26). These animals will be evaluated during the course of their disease. The sensitivity of the system in detecting yersinia will be evaluated as their disease progresses. This will be compared both to culture results and to the positivity of the F1 antigen detection. The sensitivity of the system will be evaluated over several lots of preliminary device manufacture. We will also evaluate the sensitivity of the system with multiple fabrication lots to detect a wide number of enterobacteriacieae including klebsiella and e. coli species.

Expected Results from Objective 6: At the end of this objective, we will have an understanding as to how sensitive the system is for detecting yersinia during the course of yersina infection in a murine model, Lot to lot variation of the system will also be explored for both yersina and for other entrobacteriacieae.

### Objective 7: Begin Wider Manufacture of Diagnostic Devices

Based on the consistency of the system's performance with multiple lots, we will either progress to a wider manufacturing effort or will review our fabrication processes to establish that consistency. When the precision or reproducibility of the system and the processes have been established, we will be ready for a wider manufacturing effort.

Expected Results from Objective 7: At the end of this objective, we hope to have established the manufacturing conditions that will allow larger production of the device and device kits for commercial distribution if required.

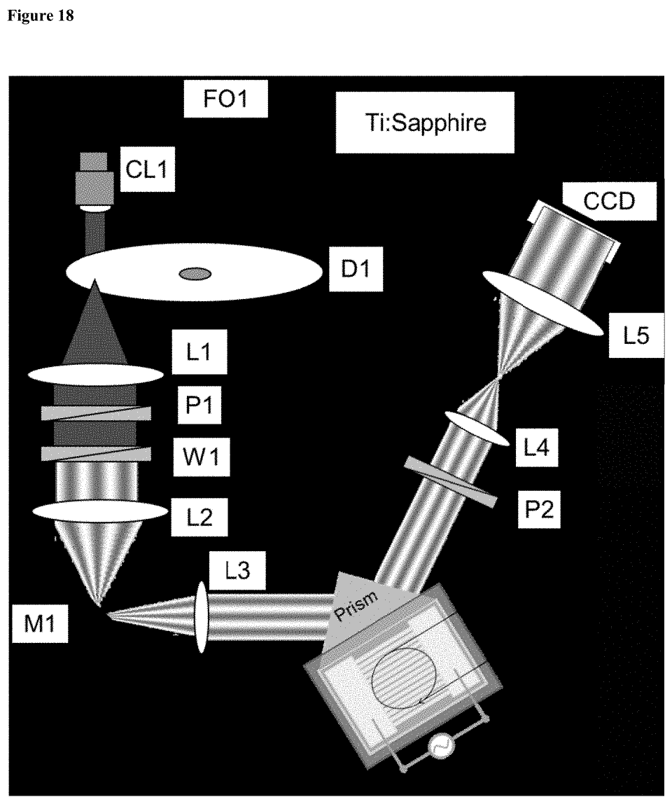

For SPR-PI quantification and transduction, the setup is shown schematically in FIG. 18. The light source is a Ti:sapphire laser operated at 770 nm to excite surface plasmons on the surface of the sensor. The laser was coupled to the rest of the optical system by a fiber optic patch cable, terminating in a collimation lens (CL1). A rotating diffuser (D1) was used to reduce coherence artifacts from the laser by approximating a randomly scattering surface. Since the light from the diffuser is incoherent, lens LI was added to create a wide collimated beam. Polarizer P1 and wedge depolarizer W1 were used to create a periodic collimated pattern of illumination across the width of the beam. This allowed simple modeling of phase shifts from the measured adsorption. Prior to light entering a SF10 prism (refractive index of 1.72), lenses L2 and L3 were used to reduce the size of the incident beam to the size of the sensor on the prism. After light was reflected from the sensor, it was treated with polarizer P2 to eliminate any residual s-polarized light. Finally, lenses L4 and L5 were used to magnify the beam to fill the CCD, which is 1.4 cm wide.

IDEs patterned on the prism served as a detector surface for adsorption of bacteria. Surface functionalization and. DEP experimental setups were implemented the same as on glass slide substrates. However, during bacteria exposure, a flow cell instead of a PDMS well was used for the prism. This allowed greater security of the prism during positioning adjustment for the SPR intensity dip. After SPR-PI and DEP, dark field microscopy was used for visual characterization.

FIG. 18 Optical setup for phase-contrast SPR system modeled after Zhou et al. The Ti:sapphire laser is coupled to the optical system by a fiber optic, terminating in a collimation lens (CL1). A rotating diffuser (D1) reduces coherence artifacts. Lens L1 collects the incoherent light from the diffuser to create a wide collimated beam. Polarizer P1 and wedge depolarizer W1 create a periodic collimated pattern of illumination across the width of the beam. Lenses L2 and L3 reduce the size of this beam to the size of sensor patterned on the prism. Polarizer P2 eliminates any s-polarized light that reflects from the prism. Lenses L4 and 15 magnify the beam to fill the CCD.

Examples of bacteria-siderophore combinations:

Pseudomonas—pyoverdin, pyochelin

Salmonella—salmochelin

Burkholderia—ornibactin

Acinetobacter—fimsbactin

Burkholderia pseudomallei—malleobctin

Legionella—legiobactin

E. coli (and others)—entereobactin

Yersinia—yersiniabactin

Bacillus anthracis—petrobactin, bacillibactin

Preparation of Derivatized Au Colloids

The procedure described below was originally developed to graft a low molecular weight peptide, CKWAKWAK onto the surface of Au colloids. These procedures most closely describe how we approach the grafting of thiolated siderophores onto the colloids. Colloids (citrate-stabilized) were purchased from British Biocell International and were sized to an average diameter of 23 nm by transmission electron microscopy (TEM).

To extend the characterization of surface adsorption to gold colloid from a small molecule to a larger biomolecule, an octapeptide was designed, The peptide sequence, H₂N-Cys-Lys-Trp-Ala-Lys-Trp-Ala-Lys-CO₂NH₂ (CKWAKWAK) was synthesized and purified by the Protein Sciences Facility at the University of Illinois. The structure of CKWAKWAK is depicted below.

The surface coverage of CKWAKWAK was determined by mixing 119 pM of 23 nm diameter gold colloid with differing concentrations of CKWAKWAK in 15 μM tris-(2-carboxyethyl)phosphine (TCEP). TCEP is a reductant and is used to prevent oxidation of the tryptophan groups that leads to the reduction in fluorescence of CKWAKWAK. The concentrations of CKWAKWAK that were used were 0.25, 0.50, 0.75, 1.00, 1.25, 1.50, 1.75, and 2.00 μM. The isotherm was compiled for the adsorption of CKWAKWAK onto colloidal Au at various equilibrium concentrations of CKWAKWAK, [S]_{free}. The surface coverage of CKWAKWAK reaches a saturation coverage beyond an equilibrium concentration, [CKWAKWAK]=0.5 μM. Fitting the adsorption data to a Langmuir adsorption isotherm, shown below, yields Γₘₐₓ=3.1±0.1×10¹⁴ molecules cm⁻², and K_{L}=8.0±1.2×10⁶ M⁻¹. The free energy of adsorption for CKWAKWAK gives ΔG_{ads}=−49.2±0.4 kJ/mol. The behavior of CKWAKWAK indicates that it is reasonably surface active.

Immobilization to a Polymer

The procedure given here is appropriate to the derivatization of siderophore to a carboxylic acid-containing polymer, e.g. poly(acrylic acid). Gold substrates containing a thiol SAM presenting a terminal carboxylate were immersed in a freshly-prepared aqueous solution of 75 mM EDC and 15 mM NHS for 15 min. This step is used to generate an active succinimidyl ester. After rinsing with water, samples were exposed to protein (or siderophore) at a concentration of 20μg/mL in 10 mM phosphate buffer (pH 6.0) for 1 hr. The samples were rinsed with water and placed in a petri dish in 0.1 M NaOH on an orbital shaker at a speed of 40 rpm for 1 hour.

Preparation of Microfluidic Devices

Material below describes the construction of a sophisticated multilayer PMMA-polycarbonate multilayer microfluidic. The IP for the following process belongs to the Univ of Illinois, and this material is taken verbatim from Flachsbart et al, Lab Chip 2006, 6, 667. The overall fabrication scheme of the multilayer device shown below consists of: (a) beginning with an essentially rigid substrate on which to build the device; (b) individually processing each distinct labile polymer layer on a separate carrier plate, including if necessary spinning and curing the polymer layer, patterning, etching, and applying the adhesive; (c) transferring, aligning, and bonding the labile polymer layer on the substrate; (d) releasing the carrier plate; and (e) repeating with subsequent layers to form a multilayer stack. After a brief overview of the assembly of the multilayer stack, sections 2.1 to 2.4 detail the major issues addressed in order to fabricate the device.

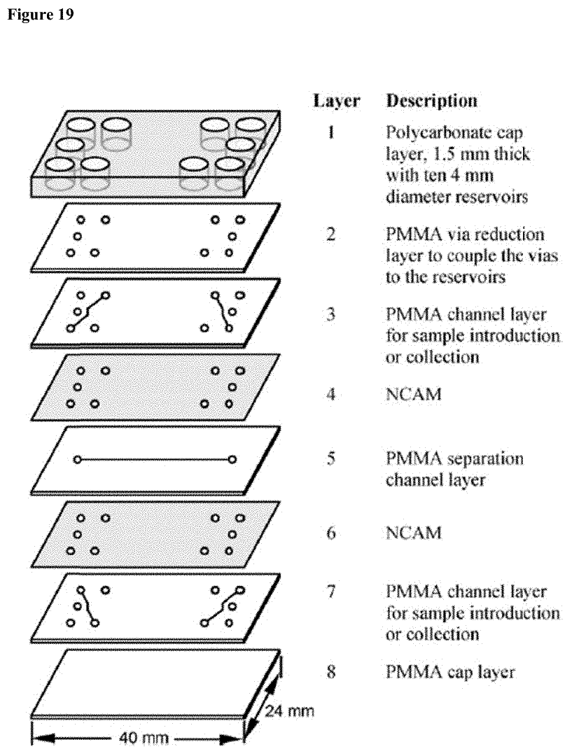

The assembly of the layers into the device below consists of the sequential operations of contact printing adhesive layers, bonding, and releasing the bonded. PMMA layers from their temporary coverglass carriers. An adhesive is contact printed onto the top surface of PMMA layer #2 in FIG. 19, which is then bonded to the polycarbonate (PC) top piece (layer #1 in FIG. 19) at 130 uC and 5.2 MPa of applied pressure under vacuum for 10 minutes. PMMA layer #2 is processed while affixed to a temporary coverglass carrier, which, after bonding, is released by submersion in a hot water bath at approximately 50 uC for 5 min The next PMMA layer #3 is bonded to the device stack in the same way that layer #2 is bonded, i.e. the top surface of PMMA layer #3 is coated with an adhesive, whereby it is bonded to the device stack, and its temporary carrier released using a hot water bath. Bonding NCAM layers requires a slightly different approach since adhesive cannot be applied directly to the NCAM layer without plugging the nanoscale pores. Thus the adhesive is to be applied to each of the layers facing the NCAM layer. Accordingly, the bottom surface of PMMA ayes #3 and the top surface of the PMMA layer #5 are coated with adhesive, A NCAM layer #4 is placed between thein, aligned and bonded together. After the bonding process, the coverglass carrier for PMMA layer #5 is released. The process is repeated for the second NCAM layer #6 and the PMMA layer #7. The final, unpatterned PMMA layer #8 is bonded to the device after coating the bottom of PMMA layer #7. The final step is a 12 h vacuum-oven cure at 130 uC at a temperature and time sufficient to fully crosslink all the epoxy adhesive layers without allowing the remaining solvents or curing byproducts to coalesce.

### REFERENCES

1) Hider, R .C. and Kong, X. Chemistry and biology of siderophores. Natural Product Reports 2010; 27:637-657.

2) Ji, C., Juarez-Hernandez, R. E., and Miller, M. J. Exploiting bacterial iron acquisition: siderophore conjugates. Future Med. Chem. 2012; 4:297-313.

3) Lamont, I., Beare, P. A., Ochsner, O., Vasil, A. L., Vasil, M. Siderophore-mediated signaling regulates virulence factor production in Pseudomonas aeruginosa. Proc. National Acad. Sci. 2002; 99:7072-7077.

4) Miethke, M. Maraheil, M. A. Siderophore-Based Iron Acquisition and Pathogen Control. Microbiol. Mol. Biol. Rev. 2007; 71:413.

5) Mislin, G. L. A. and Schalk, I. J. Siderophore-dependent iron uptake systems as gates for antibiotic Trojan horse strategies against Pseudomonas aeruginosa Metallomics 2014; 6:408-420.

6) Meyer, J-M., Neely, A., Stintzi, A., Georges, C., Holder, I. A, Pyoverdin is essential for virulence of Pseudomonas aeruginosa. Infection and Immunity 1996; 64:518-521.

7) Poole, K, Young, L., Neshat, S. Enterobactin-mediated iron transport in Pseudomonas aeruginosa. J. Bacteriology 1990; 172:6991-6996 .

8) Mortensen, B. L., and Skaar, E. P. The contribution of nutrient metal acquisition and metabolism of Acinetobacter baumannii survival within the host. Frontiers in Cellular and Infection Microbiology 2013; doi 10.3389/fcimb.2013 00095.

9) Ji C, Miller P A, Miller M J. Iron transport-mediated drug delivery: practical syntheses and in-vitro antibacterial studies of tris-catecholate siderophore-aminopenicillin conjugates reveals selectively potent antipseudomonal activity. J Am Chem Society 2012,134:9898-9901.

10) Obritsch, M. D., Fish, D. N., MacLaren, R., Jung, R. Nosocomial infections due to multidrug-resistant Pseudomonas aeruginosa: epidemiology and treatment options. Pharmacotherapy 2005; 25:1353-1364.

11) Solh, A. A., Alhajhusain, A. Update on the treatment of Pseudomonas pneumonia. J Antimicrobial Chemotherapy 2009; 64:229-238.

12) Hancock, R. E. W. Resistance mechanisms in Pseudomonas aeruginosa and other nonfermentative gram-negative bacteria. Clinical Infect. Dis. 1998; 27 (Suppl):S93-S99.

13) Niederman, M. S. and Craven, D. E. and the ATS/IDSA Committee. Guidelines for the management of adults with hospital-acquired, ventilator-associated, and healthcare-associate pneumonia. Am J. Respiratory and Critical Carew Medicine 2005; 171:388-416.

14) Tramper-Standers, G. A., van der Ent, C. K., Slieker, M. G., Terheggen-Lagro, S. W. J., Teding van Berkhout, F., Kimpen, J. L. L., and Wolfs, T. F. W. Diagnostic value of serological tests against Pseudomonas aeruginosa in a large cystic fibrosis population. Thorax 2006; 61:689-693.

15) Pedersen, S. S., Espersen, F., Hoiby, N. Diagnosis of chronic Pseudomonas aeruginosa infection in cystic fibrosis by enzyme-linked immunosorbent assay. J Clinical Microbiol 1987; 25:1830-1836.

16) Lee, C. S., Wetzel, W. K., Buckley, T., Wozniak, D., and Lee, J. Rapid and sensitive detection of Pseudomonas aeruginosa in chlorinated water and aerosols targeting gyrB gene using real time PCR. J. Appl Microbiol 2011; 111:893-903.

17) Applied Biosystems technical overview. TaqMan® pseudomonas aeruginosa detection kit. Applied Biosystems, Foster City, Calif.

18) Wisplinghoff, H., Edmond, M. B., Pfaller, M. A., Jones, R. J., Wenzel, R. P., Seifert, H. Nosocomial bloodstream infections caused by Acinetobacter species in United States hospitals; clinical features, molecular epidemiology, and antimicrobial susceptibility. Clinical Infectious Disease 2000; 31:690-697

19) Center for Disease Control and Prevention Report. Antibiotic resistance threats in the United States 2013:59-60.

20) Manchanda, V., Sanchaita, S., Singh, N. P. Multidrug resistant Acinetobacter. J. Global Infectious Disease 2010; 2:291-304.

21) Peleg, A. Y., Seifert, H., Paterson, D. L. Acinetobacter baumannii: emergence of a successful pathogen. Clinical Microbiology Reviews 2008; 21:538-582

22) Bergogne-Berezin, E., and Towner, K. J. Acinetobacter spp. as nosocomial pathogens: microbiological, clinical, and epidemiological features. Clinical Microbiology Reviews 1996; 9:148-165.

23) Doorneweerd D D, Henne W A, Reifenberger R G, Low P S: Selective capture and identification of pathogenic bacteria using an immobilized siderophore. Langmuir 2010, 26, 15424-15429.

24) Wang, Q.; Bohn, P. W., Surface composition gradients of immobilized cell signaling molecules. Epidermal Growth Factor on Gold. Thin Solid Films 2006, 513, 338-346.

25) Sokatch, J. R. Alanine and aspartate formation during growth on Valine-C14 by Pseudomonas aeruginosa. J. Bacteriology 1966; 92:72-75

26) Wencewicz, T. A.; Miller, M. J. Biscatecholate-Monohydroxamate Mixed Ligand Siderophore-Carbacephalosporin Conjugates are Selective Sideromycin Antibiotics that Target Acinetobacter baumannii,” J. Med. Chem. 2013, 56, 4044-4052.

27) Proschak, A.; Lubuta, P.; Grün, P.; Löhr, F.; Wilharm, G.; De Berardinis, V.; Bode, H. B., Structure and Biosynthesis of Fimsbactins A-F, Siderophores from Acinetobacter baumannii and Acinetobacter baylyi. ChemBioChem 2013, 14, 633-638.

The entire contents of each of the references above and also the following are hereby incorporated by reference, the same as if set forth at length: U.S. Appl'n. Nos. 61/796,044, filed Nov. 1, 2012, 61/894,770, filed Oct. 23, 2013, and 62/039,405, filed Aug. 19, 2014; and Int'l. Appl'n. No. PCT/US13/68175, filed Nov. 1, 2013.

1-4. (canceled)

5. A diagnostic test strip for detecting bacteria in a sample, comprising:
a substrate having a surface other than gold or glass; and
a plurality of Fe(III)-bound or Fe(III)-binding siderophores specific to the bacteria and covalently attached to the surface;
wherein the siderophores are selected from the group consisting of one or more natural siderophores, siderophores having one or more of the following formulas, or combination thereof: 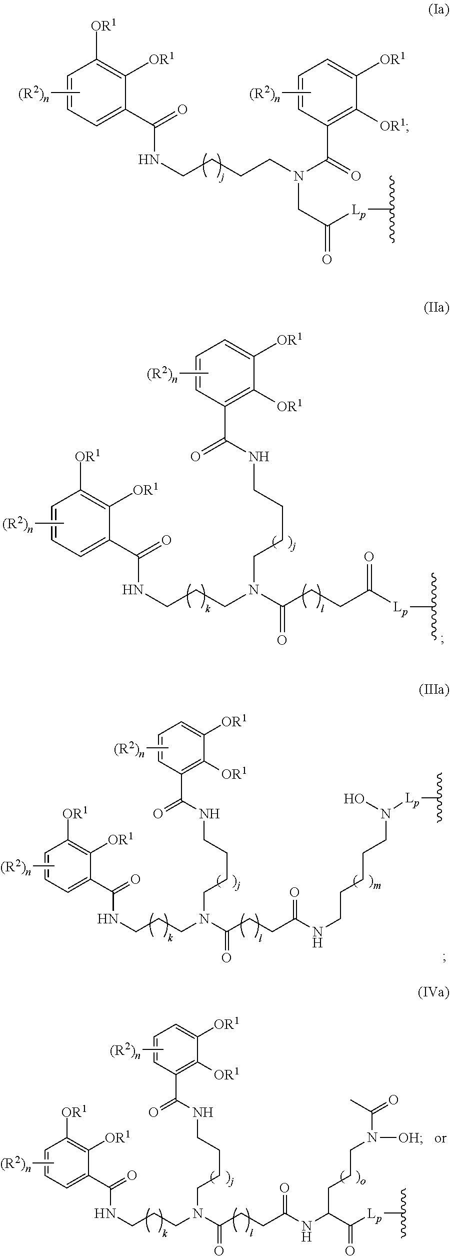 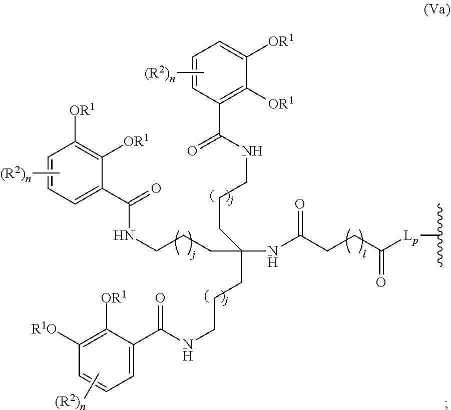
wherein
each L is independently a linker;
each R¹ is independently H, —C(═O)alkyl, —C(═O)aryl, or —C(═O)O-alkyl;
each R² is independently H, alkyl, alkoxy, hydroxy, carboxy, halo, nitro, amino, or cyano;
each n is independently 1, 2, or 3;
each p is independently 0-11;
each j is independently 0-11;
each k is independently 1-11;
each l is independently 1-11;
each o is independently 0-11; and
each m is independently 0-11;
pharmaceutically acceptable salt thereof, or combination thereof.

6. The test strip of claim 5, wherein the substrate surface is paper, polymer, silica, quartz, or combination thereof.

7. The test strip of claim 5, wherein the siderophores are attached directly or indirectly through a linking group.

8. The test strip of claim 5, wherein the siderophore is naturally occurring or synthetic.

9-26. (canceled)

27. A method for detecting bacteria in a sample, comprising:
contacting the sample with a substrate surface comprising a plurality of covalently-attached first Fe(III)-bound or Fe(III)-binding siderophores specific to the bacteria, to effect a first binding of one or more of the bacteria, if present in the sample, to one or more of the first siderophores;
introducing a detection fluid comprising a plurality of gold nanoparticles, the nanoparticles comprising one or more covalently-attached second Fe(III)-bound or Fe(III)-binding siderophores specific to the bacteria, to effect a second binding of one or more of the bacteria, if bound to the first siderophores, to one or more of the second siderophores;
detecting the presence or absence of the nanoparticles so bound, to thereby detect the present or absence of the bacteria;
wherein the siderophores are selected from the group consisting of one or more natural siderophores, siderophores having one or more of the following formulas, or combination thereof: 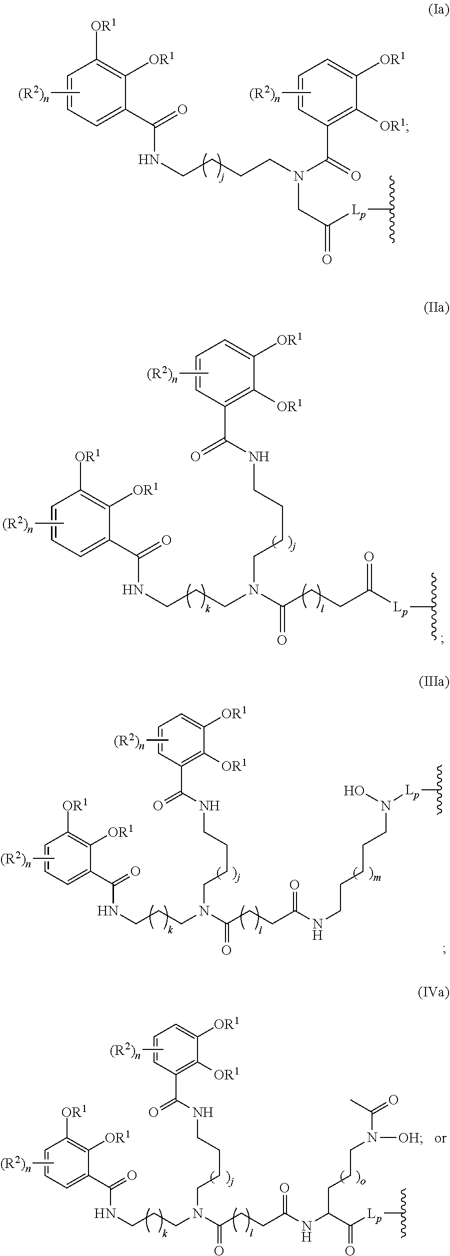 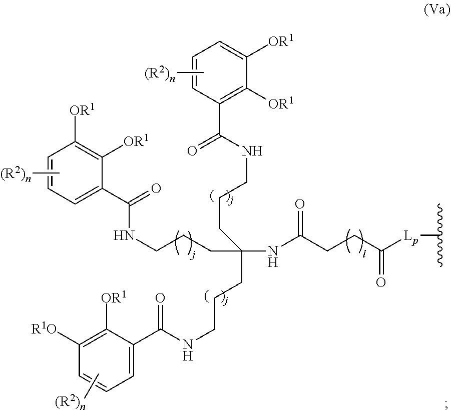
wherein
each L is independently a linker;
each R¹ is independently H, —C(═O)alkyl, —C(═O)aryl, or —C(═O)O-alkyl;
each R² is independently H, alkyl, alkoxy, hydroxy, carboxy, halo, nitro, amino, or cyano;
each n is independently 1, 2, or 3;
each p is independently 0-11;
each j is independently 0-11;
each k is independently 1-11;
each l is independently 1-11;
each o is independently 0-11; and
each m is independently 0-11;
pharmaceutically acceptable salt thereof, or combination thereof.

28. The method of claim 27, wherein the surface further comprises paper, polymer, silica, quartz, glass, gold, or a combination thereof.

29-33. (canceled)

34. The method of claim 27 further comprising quantifying the detected bacteria.

35. The method of claim 27, further comprising one or more washing steps between the contacting, introducing and detecting.

36. The method of claim 27, wherein the gold nanoparticles have a size ranging from 1 nm to 2 microns.

37. (canceled)

38. The method of claim 27, wherein the gold nanoparticles further comprise a radiolabel, a fluorescent label, a colorimetric label, a UV-Vis label, or combination thereof. 39-40. (Canceled).

41. A method for detecting bacteria in a sample, comprising:
contacting the sample with a substrate surface comprising a plurality of covalently-attached first Fe(III)-bound or Fe(III)-binding siderophores specific to the bacteria, to effect a first binding of one or more of the bacteria, if present in the sample, to one or more of the first siderophores;
introducing a detection fluid comprising a plurality of gold nanoparticles, the nanoparticles comprising one or more covalently-attached second Fe(III)-bound or Fe(III)-binding siderophores specific to the bacteria, to effect a second binding of one or more of the bacteria, if bound to the first siderophores, to one or more of the second siderophores;
introducing an amplification fluid comprising a reductant and soluble Ag(I), to effect an electroless deposition of Ag metal onto one or more of the nanoparticles so bound;
detecting the presence or absence of Ag metal so deposited, to thereby detect the presence or absence of the bacteria;
wherein the siderophores are selected from the group consisting of one or more natural siderophores, siderophores having one or more of the following formulas, or combination thereof: 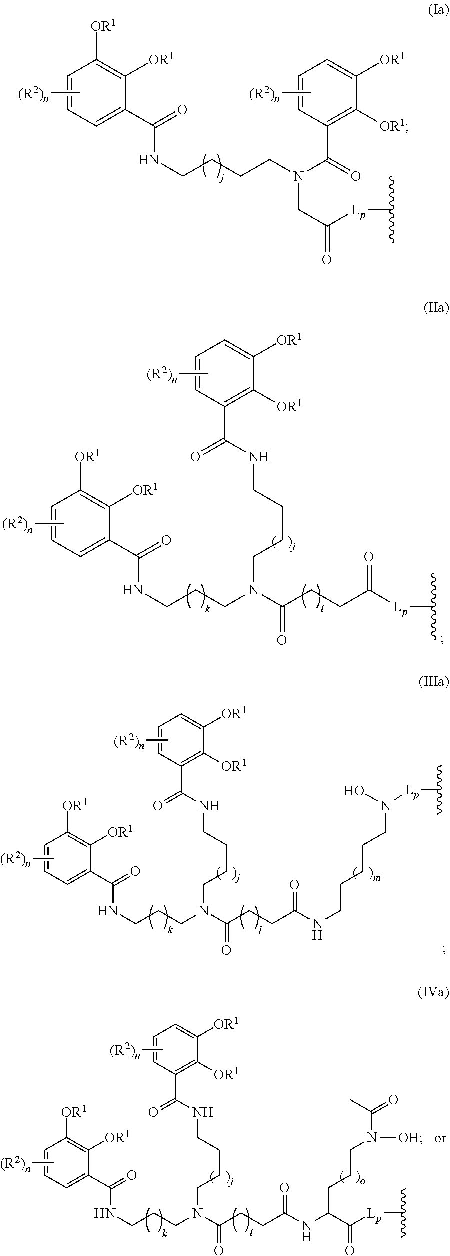 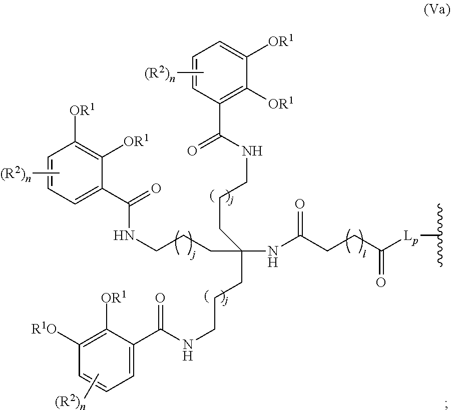
wherein
each L is independently a linker;
each R¹ is independently H, —C(═O)alkyl, —C(═O)aryl, or —C(═O)O-alkyl;
each R² is independently H, alkyl, alkoxy, hydroxy, carboxy, halo, nitro, amino, or cyano;
each n is independently 1, 2, or 3;
each p is independently 0-11;
each j is independently 0-11;
each k is independently 1-11;
each l is independently 1-11;
each o is independently 0-11; and
each m is independently 0-11;
pharmaceutically acceptable salt thereof, or combination thereof.

42. The method of claim 41, wherein the surface further comprises paper, polymer, silica, glass, quartz, or a combination thereof.

43-44. (canceled)

45. The method of claim 41, wherein the bacteria is present in the sample and is detected.

46. The method of claim 41, wherein the bacteria is not present in the sample and is not detected.

47. (canceled)

48. The method of claim 41 further comprising quantifying the detected bacteria.

49-50. (canceled)

51. The method of claim 41, wherein the reductant comprises an aldehyde, glucose/dextrose, tartaric acid, formaldehyde, hydroquinone, or combination thereof.

52. The method of claim 41, wherein the detection comprises optical detection, optical transmission, optical reflectance, or combination thereof.

53. The device of claim 1, further comprising one or more microfluidic channels disposed over the surface to direct a flow of the sample over the surface.

54-56. (canceled)

57. The strip of claim 5, wherein the natural siderophore is one or more selected from the group consisting of Desferrioxamine A1, Desferrioxamine A2, Desferrioxamine B, Desferrioxamine D1, Desferrioxamine D2, Desferrioxamine E, Desferrioxamine G1, Desferrioxamine G2A, Desferrioxamine G2B, Desferrioxamine G2C, Desferrioxamine H, Desferrioxamine T1, Desferrioxamine T2, Desferrioxamine T3, Desferrioxamine T7, Desferrioxamine T8, Desferrioxamine X1, Desferrioxamine X2, Desferrioxamine X3, Desferrioxamine X4, Desferrioxamine Et1, Desferrioxamine Et2, Desferrioxamine Et3, Desferrioxamine Te1, Desferrioxamine Te2, Desferrioxamine Te3, Desferrioxamine P1, Fimsbactin, Ferrichrome, Ferrichrome C, Ferricrocin, Sake Colorant A, Ferrichrysin, Ferrichrome A, Ferrirubin, Ferrirhodin, Malonichrome, Asperchrome A, Asperchrome B1, Asperchrome B2, Asperchrome B3, Asperchrome C, Asperchrome D1, Asperchrome D2, Asperchrome D3, Asperchrome E, Asperchrome F1, Asperchrome F2, Asperchrome F3, Tetraglycine ferrichrome, Des(diserylglycyl)-ferrirhodin, Basidiochrome, Triacetylfusarinine, Fusarinine C, Fusarinine B, Neurosporin, Coprogen, Coprogen B (Desacetylcoprogen), Triornicin (Isoneocoprogen I), Isotriornicin (Neocoprogen I), Neocoprogen II, Dimethylcoprogen, Dimethylneocoprogen I, Dimethyltriornicin, Hydroxycopropen, Hydroxy-neocoprogen I, Hydroxyisoneocoprogen I, Palmitoylcoprogen, Amphibactin B, Amphibactin C, Amphibactin D, Amphibactin E, Amphibactin F, Amphibactin G, Amphibactin H, Amphibactin I, Ferrocin A, Coelichelin, Exochelin MS, Vicibactin, Enterobactin (Enterochelin), Agrobactin, Parabactin, Fluvibactin, Agrobactin A, Parabactin A, Vibriobactin, Vulnibactin, Protochelin, Corynebactin, Bacillibactin, Salmochelin S4, Salmochelin S2, Rhizoferrin, Rhizoferrin analogues, Enantio Rhizoferrin, Staphyloferrin A, Vibrioferrin, Achromobactin, Mycobactin P, Mycobactin A, Mycobactin F, Mycobactin H, Mycobactin M, Mycobactin N, Mycobactin R, Mycobactin S, Mycobactin T, Mycobactin Av, Mycobactin NA (Nocobactin), Mycobactin J, Formobactin, Nocobactin NA, Carboxymycobactin, Carboxymycobactin 1, Carboxymycobactin 2, Carboxymycobactin 3, Carboxymycobactin 4, Pyoverdin 6.1 (Pseudobactin), Pyoverdin 6.2, Pyoverdin 6.3 (Pyoverdin Thai), Pyoverdin 6.4 (Pyoverdin 9AW), Pyoverdin 6.5,Pyoverdin 6.6, Isopyoverdin 6.7, (Isopyoverdin BTP1), Pyoverdin 6.8, Pyoverdin 7.1, Pyoverdin 7.2, (Pyoverdin BTP2), Pyoverdin 7.3, (Pyoverdin G+R), Pyoverdin 7.4, (Pyoverdin PVD), Pyoverdin 7.5, (Pyoverdin TII), Pyoverdin 7.6, Pyoverdin 7.7, Pyoverdin 7.8, (Pyoverdin PL8), Pyoverdin 7.9, (Pyoverdin 11370), Pyoverdin, Pyoverdin 7.11, (Pyoverdin 19310), Pyoverdin 7.12, (Pyoverdin 13525), Isopyoverdin 7.13, (Isopyoverdin 90-33), Pyoverdin 7.14, (Pyoverdin R′), Pyoverdin 7.15, Pyoverdin 7.16, (Pyoverdin 96-312), Pyoverdin 7.17, Pyoverdin 7.18, Pyoverdin 7.19, Pyoverdin 8.1, (Pyoverdin A214), Pyoverdin 8.2, (Pyoverdin P19), Pyoverdin 8.3, (Pyoverdin D-TR133), Pyoverdin 8.4, (Pyoverdin 90-51), Pyoverdin 8.5, Pyoverdin 8.6, (Pyoverdin 96-318), Pyoverdin 8.7, (Pyoverdin I-III), Pyoverdin 8.8, (Pyoverdin CHAO), Pyoverdin 8.9, (Pyoverdin E), Pyoverdin 9.1, Pyoverdin 9.2, (Pyoverdin Pau), Pyoverdin 9.3, Pyoverdin 9.4, Pyoverdin 9.5, (Pyoverdin 2392), Pyoverdin 9.6, Pyoverdin 9.7, (Pseudobactin 589A), Pyoverdin 9.8, (Pyoverdin 2461), Pyoverdin 9.9, Pyoverdin 9.10, (Pyoverdin 95-275), Pyoverdin 9.11, (Pyoverdin C), Pyoverdin 9.12, Pyoverdin 10.1, (Pyoverdin 2798), Pyoverdin 10.2, Pyoverdin 10.3, (Pyoverdin 17400), Pyoverdin 10.4, Pyoverdin 10.5, (Pyoverdin 18-1), Pyoverdin 10.6, (Pyoverdin 1, 2), Isopyoverdin 10.7, (Isopyoverdin 90-44), Pyoverdin 10.8, Pyoverdin 10.9, (Pyoverdin 2192), Pyoverdin 10.10, Pyoverdin 11.1, (Pyoverdin 51W), Pyoverdin 11.2, (pyoverdin 12), Pyoverdin 12.1, (Pyoverdin GM), Pyoverdin 12.2, (Pyoverdin 1547), Azoverdin, Azotobactin 87, Azotobactin D, Heterobactin A, Ornibactin-C4, Ornibactin-C6, Ornibactin-C8, Aquachelin A, Aquachelin B, Aquachelin C, Aquachelin D, Marinobactin A, Marinobactin B, Marinobactin C, Marinobactin D1, Marinobactin D2, Marinobactin E, Loihichelin A, Loihichelin B, Loihichelin C, Loihichelin D, Loihichelin E, Loihichelin F, Schizokinen, Aerobactin, Arthrobactin, Rhizobactin 1021, Nannochelin A, Nannochelin B, Nannochelin C, Acinetoferrin, Ochrobactin A, Ochrobactin B, Ochrobactin C, Snychobactin A, Snychobactin B, nychobactin C, Mugineic acid, 3-Hydroxymugineic acid, 2′-Deoxymugineic acid, Avenic acid, Distichonic acid, Deoxydistichonic acid, Rhizobactin, Staphyloferrin B, Alterobactin A, Alterobactin B, Pseudoalterobactin A, Pseudoalterobactin B, Petrobactin, Petrobactin sulphonate, Petrobactin disulphonate, Fusarinine A, Exochelin MN, Ornicorrugatin, Maduraferrin, Alcaligin, Putrebactin, Bisucaberin, Rhodotrulic acid, Dimerum acid, Amycolachrome, Azotochelin, (Azotobactin), Myxochelin, Amonabactin T789, Amonabactin P750, Amonabactin T732, Amonabactin P693, Salmochelin S1, Serratiochelin, Anachelin 1, Anachelin 2, Pistillarin, Anguibactin, Acinetobactin, Yersiniabactin, Micacocidin, Deoxyschizokinen, Heterobactin B, Desferrithiocin, Pyochelin, Thiazostatin, Enantio-Pyochelin, 2,3-Dihydroxybenzoylserine, Salmochelin SX, Citrate, Chrysobactin, Aminochelin, Siderochelin A, Aspergillic acid, Itoic acid, Cepabactin, Pyridoxatin, Quinolobactin, Ferrimycin A, Salmycin A, Albomycin, or combination thereof.

58. The strip of claim 5, wherein the siderophore has one or more of the following formulas: 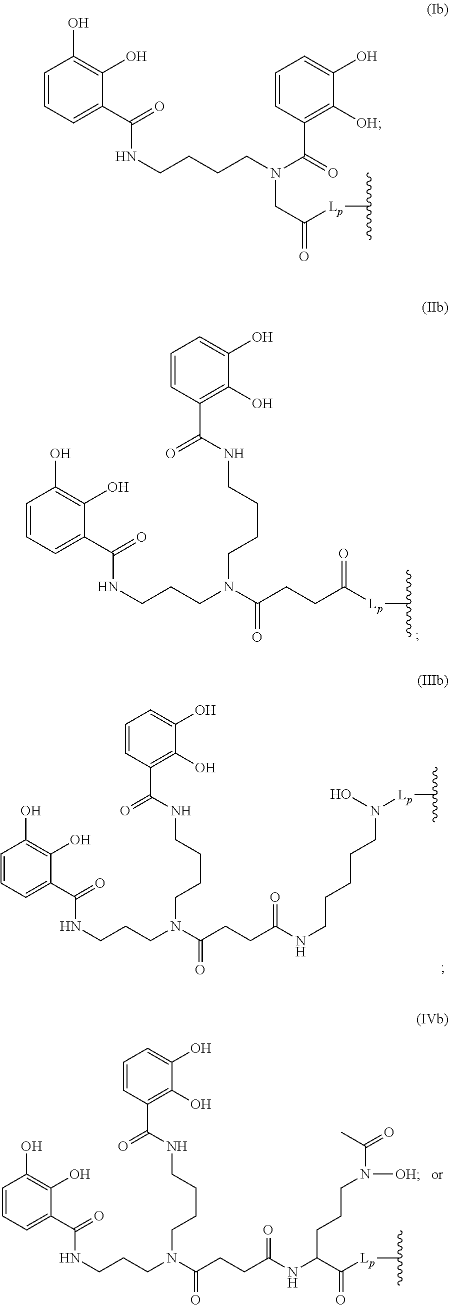 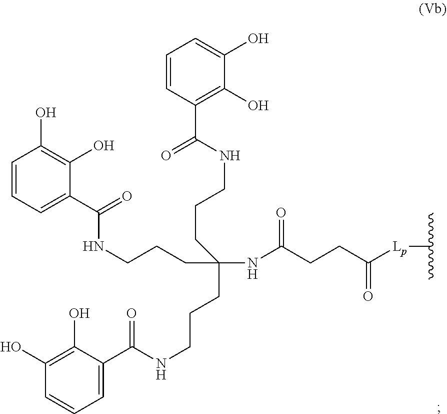
wherein
each L is independently a linker; and
each p is independently 0-11;
Fe(III)-binding form thereof, Fe(III)-bound form thereof, pharmaceutically acceptable salt thereof, or combination thereof.
